(19) United States
(12) Patent Application Publication
(10) Pub. No.: US 2024/0398784 A1
(43) Pub. Date: Dec. 5, 2024

(54) C-LINKED ISOQUINOLINE AMIDES AS LRRK2 INHIBITORS, PHARMACEUTICAL COMPOSITIONS, AND USES THEREOF

(71) Applicant: Merck Sharp & Dohme LLC, Rahway, NJ (US)

(72) Inventors: Peter H. Fuller, Ashland, MA (US); Anmol Gulati, Watertown, MA (US); Solomon D. Kattar, Wakefield, MA (US); Mitchell H. Keylor, Malden, MA (US); Kaila A. Margrey, Cambridge, MA (US); Luis Torres, Bridgewater, MA (US); Xin Yan, Newton Highlands, MA (US)

(73) Assignee: Merck Sharp & Dohme LLC, Rahway, NJ (US)

(21) Appl. No.: 18/685,357

(22) PCT Filed: Sep. 26, 2022
(86) PCT No.: PCT/US2022/044670

§ 371 Date: Feb. 21, 2024

Related U.S. Application Data
(60) Provisional application No. 63/251,193, filed on Oct. 1, 2021.

(51) Int. Cl. A61K 31/472; A61K 31/4725; A61K 31/5377; C07D 217/22; C07D 401/12; C07D 401/14; C07D 405/12; C07D 405/14; C07D 409/14; C07D 413/12; C07D 491/048

(52) U.S. Cl. CPC A61K 31/472; A61K 31/4725; A61K 31/5377; C07D 217/22; C07D 401/12; C07D 401/14; C07D 405/12; C07D 405/14; C07D 409/14; C07D 413/12; C07D 491/048

(57) ABSTRACT

The present invention is directed to certain 2-aminoquinzaoline derivatives of Formula (I): Formula (I) and pharmaceutically acceptable salts thereof, wherein R¹, R², and R³ are as defined herein, which are potent inhibitors of LRRK2 kinase and may be useful in the treatment or prevention of diseases in which the LRRK2 kinase is involved, such as Parkinson's Disease and other diseases and disorders described herein. The invention is also directed to pharmaceutical compositions comprising these compounds and the use of these compounds and compositions in the prevention or treatment of such diseases in which LRRK-2 kinase is involved.

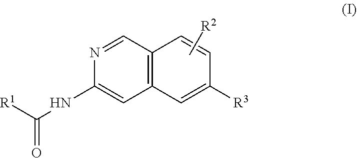

### BACKGROUND OF THE INVENTION

Parkinson's disease (PD) is a common neurodegenerative disease caused by progressive loss of mid-brain dopaminergic neurons leading to abnormal motor symptoms such as bradykinesia, rigidity and resting tremor. Many PD patients also experience a variety of non-motor symptoms including cognitive dysfunction, autonomic dysfunction, emotional changes and sleep disruption. The combined motor and non-motor symptoms of Parkinson's disease severely impact patient quality of life.

While the majority of PD cases are idiopathic, there are several genetic determinants such as mutations in SNCA, Parkin, PINKI, DJ-1 and LRRK2. Linkage analysis studies have demonstrated that multiple missense mutations in the Leucine-Rich Repeat Kinase 2 (LRRK2) gene lead to an autosomal late onset form of PD. LRRK2 is a 286 kDa cytoplasmic protein containing kinase and GTPase domains as well as multiple protein-protein interaction domains. See for example, Aasly et al., Annals of Neurology, Vol. 57(5), May 2005, pp. 762-765; Adams et al., Brain, Vol. 128, 2005, pp. 2777-85; Gilks et al., Lancet, Vol. 365, Jan. 29, 2005, pp. 415-416, Nichols et al., Lancet, Vol. 365, Jan. 29, 2005, pp. 410-412, and U. Kumari and E. Tan, FEBS journal 276 (2009) pp. 6455-6463.

In vitro biochemical studies have demonstrated that LRRK2 proteins harboring the PD associated proteins generally confer increased kinase activity and decreased GTP hydrolysis compared to the wild type protein (Guo et al., Experimental Cell Research, Vol, 313, 2007, pp. 3658-3670) thereby suggesting that small molecule LRRK2 kinase inhibitors may be able to block aberrant LRRK2-dependent signaling in PD. In support of this notion, it has been reported that inhibitors of LRRK2 are protective in models of PD (Lee et al., Nature Medicine, Vol 16, 2010, pp. 998-1000).

LRRK2 expression is highest in the same brain regions that are affected by PD. LRRK2 is found in Lewy bodies, a pathological hallmark of PD as well as other neurodegenerative diseases such as Lewy body dementia (Zhu et al., Molecular Neurodegeneration, Vol 30, 2006, pp. 1-17).

Further, LRRK2 mRNA levels are increased in the striatum of MPTP-treated marmosets, an experimental model of Parkinson's disease, and the level of increased mRNA correlates with the level of L-Dopa induced dyskinesia suggesting that inhibition of LRRK2 kinase activity may have utility in ameliorating L-Dopa induced dyskinesias. These and other recent studies indicate that a potent, selective and brain penetrant LRRK2 kinase inhibitor could be a therapeutic treatment for PD. (Lee et al., Nat. Med. 2010 September; 16(9):998-1000; Zhu, et al., Mol. Neurodegeneration 2006 Nov. 30; 1:17; Daher, et al., J Biol Chem. 2015 Aug. 7; 290(32):19433-44; Volpicelli-Daley et al., J Neurosci. 2016 Jul. 13; 36(28):7415-27).

LRRK2 mutations have been associated with Alzheimer's-like pathology (Zimprach et al., Neuron. 2004 Nov. 18; 44(4):601-7) and the LRRK2 R1628P variant has been associated with an increased risk of developing AD (Zhao et al., Neurobiol Aging. 2011 November; 32(11):1990-3). Mutations in LRRK2 have also been identified that are clinically associated with the transition from mild cognitive impairment to Alzheimer's disease (see WO2007149798). Together these data suggest that LRRK2 inhibitors may be useful in the treatment of Alzheimer's disease and other dementias and related neurodegenerative disorders.

LRRK2 has been reported to phosphorylate tubulin-associated tau and this phosphorylation is enhanced by the kinase activating LRRK2 mutation G2019S (Kawakami et al., PLoS One. 2012; 7(1):e30834; Bailey et al., Acta Neuropathol. 2013 December; 126(6):809-27.). Additionally, over expression of LRRK2 in a tau transgenic mouse model resulted in the aggregation of insoluble tau and its phosphorylation at multiple epitopes (Bailey et al., 2013). Hyperphosphorylation of tau has also been observed in LRRK2 R1441G overexpressing transgenic mice (Li et al., Nat Neurosci. 2009 July; 12(7):826-8.). Inhibition of LRRK2 kinase activity may therefore be useful in the treatment of tauopathy disorders characterized by hyperphosphorylated of tau such as argyrophilic grain disease, Picks disease, corticobasal degeneration, progressive supranuclear palsy, inherited frontotemporal dementia and parkinson's linked to chromosome 17 (Goedert and Jakes Biochim Biophys Acta. 2005 Jan. 3.).

A growing body of evidence suggests a role for LRRK2 in immune cell function in the brain with LRRK2 inhibitors demonstrated to attenuate microglial inflammatory responses (Moehle et al., J Neurosci. 2012 Feb. 1; 32(5):1602-11.). As neuroinflammation is a hallmark of a number of neurodegenerative diseases such PD, AD, MS, HIV-induced dementia, ALS, ischemic stroke, MS, traumatic brain injury and spinal cord injury, LRRK2 kinases inhibitors may have utility in the treatment of neuroinflammation in these disorders. Significantly elevated levels of LRRK2 mRNA have been observed in muscle biopsy samples taken from patients with ALS (Shtilbans et al., Amyotroph Lateral Scler. 2011 July; 12(4):250-6.). LRRK2 inhibitors have been disclosed in the art, e.g., WO2016036586.

LRRK2 is also expressed in cells of the immune system and recent reports suggest that LRRK2 may play a role in the regulation of the immune system and modulation of inflammatory responses. LRRK2 kinase inhibitors may therefore be of utility in a number of diseases of the immune system such as lymphomas, leukemias, multiple sclerosis rheumatoid arthritis, systemic lupus erythematosus autoimmune hemolytic anemia, pure red cell aplasia, idiopathic thrombocytopenic pupura (ITP), Evans Syndrome, vasculitis, bullous skin disorder, type I diabetes mellitus, Sjorgen's syndrome, Delvic's disease, inflammatory myopathies (Engel at al., Pharmacol Rev. 2011 March; 63(1):127-56; Homam et al., Homam et al., Clin Neuromuscluar disease, 2010) and ankylosing spondylitis (Danoy et al., PLoS Genet. 2010 Dec. 2; 6(12).). Increased incidence of certain types of non-skin cancers such as renal, breast, lung, prostate, and acute myelogenous leukemia (AML) have been reported in patients with the LRRK2 G2019S mutation (Agalliu et al., JAMA Neurol. 2015 January; 72(1); Saunders-Pullman et al., Mov Disord. 2010 Nov. 15; 25(15):2536-41.). LRRK2 has amplification and overexpression has been reported in papillary renal and thyroid carcinomas. Inhibiting LRRK2 kinase activity may therefore be useful in the treatment of cancer (Looyenga et al., Proc Natl Acad Sci USA. 2011 Jan. 25; 108(4):1439-44).

Genome-wide association studies also highlight LRRK2 in the modification of susceptibility to the chronic autoimmune Crohn's disease and leprosy (Zhang et al., The New England Jopuranl of Medicine, Vol 361, 2009, pp. 2609-2618; Umeno et al., Inflammatory Bowel Disease Vol 17, 2011, pp. 2407-2415).

### SUMMARY OF THE INVENTION

The present invention is directed to certain C-linked isoquinoline amide derivatives, which are collectively or individually referred to herein as “compound(s) of the invention” or “compounds of Formula (I)”, as described herein. LRRK2 inhibitors have been disclosed in the art, e.g., WO2016036586. Applicant has found, surprisingly and advantageously, that the compounds of Formula (I), each of which possess a C-substituted isoquinoline amide moiety, the amino substituent attached to a carbon atom of a C₃₋₈ carbocyclic, exhibit excellent LRRK2 inhibitory activity. In some embodiments, the compounds of the invention exhibit unexpectedly superior potency as inhibitors of LRRK2 kinase, as evidenced by the data reported herein. The compounds of the invention may be useful in the treatment or prevention of diseases (or one or more symptoms associated with such diseases) in which the LRRK2 kinase is involved, including Parkinson's disease and other indications, diseases and disorders as described herein. The invention is also directed to pharmaceutical compositions comprising a compound of the invention and to methods for the use of such compounds and compositions for the treatments described herein.

### DETAILED DESCRIPTION OF THE INVENTION

For each of the following embodiments, any variable not explicitly defined in the embodiment is as defined in Formula (I). In each of the embodiments described herein, each variable is selected independently of the other unless otherwise noted.

In one embodiment, the compounds of the invention have the structural Formula (I):

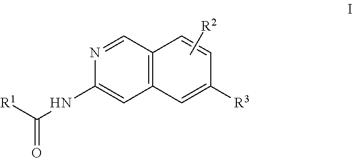

or a pharmaceutically acceptable salt thereof, wherein:
R¹ is selected from C₁₋₆ alkyl, —(R)-phenyl, monocyclic, spirocyclic or bicyclic —(R)—C₃₋₈ cycloalkyl, —(R)—C₃₋₁₀ heterocyclyl, and —(R)₀₋₃C₅₋₁₀ heteroaryl, said alkyl, phenyl, cycloalkyl, heterocyclyl and heteroaryl optionally substituted with 1 to 4 groups selected from R^{a};
R is a bond or straight or branched C₁₋₆ alkylenyl, R² is selected from hydrogen, C₁₋₆ alkyl, and halogen; R³ is selected from C₁₋₆ alkyl, spiropentanyl, cyclopropyl, cyclobutyl, cyclohexyl, and C-linked tetrahyropyranyl, C-linked piperidinyl, and C-linked oxabicycloheptanyl, said alkyl, spiropentanyl, cyclopropyl, cyclobutyl, cyclohexyl, tetrahyropyranyl, piperidinyl, and oxabicycloheptanyl are unsubstituted or substituted with 1 to 3 groups independently selected from 1 to 3 groups from R^{c}. R^{a} is independently selected from the group consisting of H, halogen, —OH, CN, —OC₁₋₆ alkyl, C₁₋₆ alkyl, C₃₋₆ cycloalkyl, C(O)CF₃, C(O)C₁₋₆ alkyl, C₁₋₃ haloalkyl, C₁₋₃ haloalkoxy, NH(C₁₋₆alkyl), N(C₁₋₆alkyl)₂, —OC₁₋₃ haloalkyl, (CH₂)ₙC₅₋₆ heteroaryl, (CH₂)C₃₋₁₀ heterocyclyl, —NH2, said alkyl, heterocyclyl, and heteroaryl optionally substituted with 1 to 3 groups of R^{b}; R^{b} is independently selected from the group consisting of C₁₋₆ alkyl, —OC₁₋₆ alkyl, halogen, OH, CN, and C₁₋₃ haloalkyl; R^{c} is independently selected from the group consisting of hydrogen, C₁₋₆ alkyl, OC₁₋₆ alkyl, C₃₋₆ cycloalkyl, CN, OH, halogen, azetidinyl, pyrrolidinyl, oxetanyl, tetrahydrofuranyl, tetrahydropyranyl, and dioxidothietanyl, wherein said alkyl, cycloalkyl, azetidinyl, pyrrolidinyl, oxetanyl, tetrahydrofuranyl, tetrahydropyranyl, and dioxidothietanyl are optionally substituted with 1 to 3 groups independently selected from C₁₋₆ alkyl, halogen, CN, OC₁₋₆ alkyl, C₃₋₆ cycloalkyl and OH; and n is selected from 0 to 3.

An embodiment of this invention is realized when n is 0. Another embodiment of this invention is realized when n is 1. Another embodiment of this invention is realized when n is 2. Still another embodiment of this invention is realized when n is 3.

Another embodiment of this invention is realized when R is a bond. Still another embodiment of this invention is realized when R is a straight or branched C₁₋₆ alkylenyl.

Another embodiment of this invention is realized when R¹ is C₁₋₆ alkyl, said alkyl unsubstituted or substituted with 1 to 3 groups selected from R^{a}. A subembodiment of this aspect of the invention is realized when the C₁₋₆ alkyl is selected from the group consisting of CH₃, CH(CH₃)₂, CH₂CH(OH)CF₃, and CHF₂.

An embodiment of this invention is realized when R¹ is —(R)phenyl, said phenyl unsubstituted or substituted with 1 to 3 groups selected from R^{a}. A subembodiment of this aspect of the invention is realized when R is a bond. Another subembodiment of this aspect of the invention is realized when R is a straight or branched C₁₋₆ alkylenyl.

An embodiment of this invention is realized when R¹ is monocyclic or bicyclic —(R)C₃-8cycloalkyl, said cycloalkyl unsubstituted or substituted with 1 to 3 groups selected from R^{a}. A subembodiment of this aspect of the invention is realized when R is a bond. A subembodiment of this aspect of the invention is realized when R is a straight or branched C₁₋₆ alkylenyl. An embodiment of this invention is realized when the C₃₋₈ cycloalkyl is selected from cyclopropyl, cyclobutyl, cyclopentyl, cyclohexyl, bicyclopentanyl, bicyclohexanyl, spirohexanyl, spiropentanyl, and spirooctanyl unsubstituted or substituted with 1 to 3 groups selected from R^{a}. Another embodiment of this invention is realized when the C₃₋₈cycloalkyl is unsubstituted or substituted cyclopropyl. Another embodiment of this invention is realized when the C₃₋₈cycloalkyl is unsubstituted or substituted cyclobutyl. Another embodiment of this invention is realized when the C₃₋₈cycloalkyl is unsubstituted or substituted cyclopentyl. Another embodiment of this invention is realized when the C₃₋₈cycloalkyl is unsubstituted or substituted cyclohexyl. Another embodiment of this invention is realized when the C₃₋₈cycloalkyl is unsubstituted or substituted bicyclopentanyl. Another embodiment of this invention is realized when the C₃₋₈cycloalkyl is unsubstituted or substituted bicyclohexanyl. Another embodiment of this invention is realized when the C₃₋₈cycloalkyl is unsubstituted or substituted spirohexanyl.

Another embodiment of this invention is realized when the C₃₋₈cycloalkyl is unsubstituted or substituted spiropentanyl. Another embodiment of this invention is realized when the C₃₋₈cycloalkyl is unsubstituted or substituted spirooctanyl.

An embodiment of this invention is realized when R¹ is —(R)₀₋₃C₃₋₁₀ heterocyclyl, said heterocyclyl optionally substituted with 1 to 4 groups selected from R^{a}. A subembodiment of this aspect of the invention is realized when R is a bond. A subembodiment of this aspect of the invention is realized when R is a straight or branched C₁₋₆ alkylenyl. A subembodiment of this aspect of the invention is realized when the heterocylyl is selected from the group consisting of piperidinyl, morpholinyl, pyrrolidinyl, azetidinyl, oxaspirooctanyl, oxabicyclohexanyl, oxabicycloheptanyl, oxaspiroheptanyl, oxaspirononanyl, dioxaspirodecanyl, tetrahydrofuranyl, and tetrahydropyranyl unsubstituted or substituted with 1 to 3 groups selected from R^{a}. Another embodiment of this invention is realized when the heterocyclyl is unsubstituted or substituted piperidinyl. Another embodiment of this invention is realized when the heterocyclyl is unsubstituted or substituted morpholinyl. Another embodiment of this invention is realized when the heterocyclyl is unsubstituted or substituted pyrrolidinyl. Another embodiment of this invention is realized when the heterocyclyl is unsubstituted or substituted azetidinyl. Another embodiment of this invention is realized when the heterocyclyl is unsubstituted or substituted oxaspirooctanyl. Another embodiment of this invention is realized when the heterocyclyl is unsubstituted or substituted oxabicyclohexanyl. Another embodiment of this invention is realized when the heterocyclyl is unsubstituted or substituted oxabicycloheptanyl. Another embodiment of this invention is realized when the heterocyclyl is unsubstituted or substituted oxaspiroheptanyl. Another embodiment of this invention is realized when the heterocyclyl is unsubstituted or substituted oxaspirononanyl. Another embodiment of this invention is realized when the heterocyclyl is unsubstituted or substituted dioxaspirodecanyl. Another embodiment of this invention is realized when the heterocyclyl is unsubstituted or substituted tetrahydrofuranyl. Another embodiment of this invention is realized when the heterocyclyl is unsubstituted or substituted tetrahydropyranyl.

An embodiment of this invention is realized when R¹ is —(R)₀₋₃C₅₋₁₀ heteroaryl, said heteroaryl optionally substituted with 1 to 4 groups selected from R^{a}. A subembodiment of this aspect of the invention is realized when R is a bond. A subembodiment of this aspect of the invention is realized when R is is straight or branched C₁₋₆ alkylenyl. A subembodiment of this aspect of the invention is realized when the heteroaryl is selected from the group consisting of pyrazolyl, oxazolyl, pyridyl, and cyclopropafuropyridyl unsubstituted or substituted with 1 to 3 groups selected from R^{a}. Another embodiment of this invention is realized when the heteroaryl is unsubstituted or substituted pyrazolyl. Another embodiment of this invention is realized when the heteroaryl is unsubstituted or substituted oxazolyl. Another embodiment of this invention is realized when the heteroaryl is unsubstituted or substituted pyridyl. Another embodiment of this invention is realized when the heteroaryl is unsubstituted or substituted cyclopropafuropyridyl.

An embodiment of this invention is realized when R¹ is selected from the group consisting of C₁₋₆ alkyl, —(R)₀₋₁phenyl, —(R)₀₋₁pyrazolyl, —(R)₀₋₁oxazolyl, —(R)₀₋₁pyridyl, cyclopropafuropyridyl, —(R)₀₋₁cyclopropyl, —(R)₀₋₁cyclobutyl, —(R)₀₋₁cyclopentyl, —(R)₀₋₁cyclohexyl, —(R)₀₋₁bicyclopentanyl, —(R)₀₋₁bicyclohexanyl, —(R)₀₋₁spirohexanyl, —(R)₀₋₁spiropentanyl, —(R)₀₋₁spirooctanyl, —(R)₀₋₁piperidinyl, —(R)₀₋₁morpholinyl, —(R)₀₋₁pyrrolidinyl, —(R)₀₋₁azetidinyl, —(R)₀₋₁oxaspirooctanyl, —(R)₀₋₁oxabicyclohexanyl, —(R)₀₋₁oxabicycloheptanyl, —(R)₀₋₁oxaspiroheptanyl, —(R)₀₋₁oxaspirononanyl, —(R)₀₋₁tetrahydrofuranyl, and —(R)₀₋₁tetrahydropyranyl said alkyl, phenyl, pyrazolyl, oxazolyl, pyridyl, cyclopropafuropyridyl, cyclopropyl, cyclobutyl, cyclopentyl, cyclohexyl, bicyclopentanyl, bicyclohexanyl, spirohexanyl, spiropentanyl, spirooctanyl, piperidinyl, -morpholinyl, pyrrolidinyl, azetidinyl, oxaspirooctanyl, oxabicyclohexanyl, oxabicycloheptanyl, oxaspiroheptanyl, oxaspirononanyl, tetrahydrofuranyl, and tetrahydropyranyl unsubstituted or substituted with 1 to 3 groups selected from R.

An embodiment of this invention is realized when R² is selected from the group consisting of hydrogen, chlorine and methyl. Another embodiment of this invention is realized when R² is hydrogen. Another embodiment of this invention is realized when R² is chlorine.

Another embodiment of this invention is realized when R² is methyl.

An embodiment of this invention is realized when R³ is C₁₋₆ alkyl with 1 to 3 groups independently selected from 1 to 3 groups from R^{c}. A subembodiment of this aspect of the invention is realized when R³ is selected from —CH(CH₃)CH₂—OC₁₋₆ alkyl and —CH(CH₃)CH₂—CN.

Another embodiment of this invention is realized when R³ is spiropentanyl optionally substituted with 1 to 3 groups independently selected from R^{c}.

Another embodiment of this invention is realized when R³ is cyclopropyl optionally substituted with 1 to 3 groups independently selected from R^{c}.

Another embodiment of this invention is realized when R³ is cyclobutyl optionally substituted with 1 to 3 groups independently selected from R^{c}.

Another embodiment of this invention is realized when R³ is cyclohexyl optionally substituted with 1 to 3 groups independently selected from R^{c}.

Another embodiment of this invention is realized when R³ is C-linked tetrahyropyranyl optionally substituted with 1 to 3 groups independently selected from R^{c}.

Another embodiment of this invention is realized when R³ is C-linked piperidinyl optionally substituted with 1 to 3 groups independently selected from R^{c}.

Another embodiment of this invention is realized when R³ is C-linked oxabicycloheptanyl optionally substituted with 1 to 3 groups independently selected from R^{c}.

An embodiment of the invention is realized when R^{a} is independently selected from the group consisting of H, halogen, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, N(C₁₋₆alkyl)₂, —NH₂, (CH₂)ₙpyrazolyl, (CH₂)ₙpyridyl, (CH₂)ₙfuranyl, (CH₂)ₙtriazolyl, (CH₂)ₙoxadizolyl, (CH₂)ₙpyrimidinyl, (CH₂)ₙthiophenyl, (CH₂)ₙtetrahydropyranyl, (CH₂)ₙtetrahydrofuranyl, (CH₂)ₙcyclopropyl, (CH₂)ₙbicyclopentanyl, said (CH₂)ₙpyrazolyl, (CH₂)ₙpyridyl, (CH₂)ₙfuranyl, (CH₂)ₙtriazolyl, (CH₂)ₙoxadizolyl, (CH₂)ₙpyrimidinyl, (CH₂)ₙthiophenyl, (CH₂)ₙtetrahydropyranyl, (CH₂)ₙtetrahydrofuranyl, (CH₂)ₙcyclopropyl, and (CH₂)ₙbicyclopentanyl, said alkyl, pyrazolyl, pyridyl, furanyl, triazolyl, oxadizolyl, pyrimidinyl, thiophenyl, tetrahydropyranyl, tetrahydrofuranyl, cyclopropyl, bicyclopentanyl, optionally substituted with 1 to 3 groups of R^{b}.

A subembodiment of this aspect of the invention is realized when R^{a} is independently selected from the group consisting of H, halogen, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, and C(O)CF₃. A subembodiment of this aspect of the invention is realized when R^{a} is independently selected from the group consisting of NH(C₁₋₆alkyl), N(C₁₋₆alkyl)₂, and —NH₂. Another subembodiment of this aspect of the invention is realized when R^{a} is independently selected from the group consisting of (CH₂)ₙpyrazolyl, (CH₂)ₙpyridyl, (CH₂)ₙfuranyl, (CH₂)ₙtriazolyl, (CH₂)ₙoxadizolyl, (CH₂)ₙpyrimidinyl, (CH₂)ₙthiophenyl, (CH₂)ₙtetrahydropyranyl, (CH₂)ₙtetrahydrofuranyl, (CH₂)ₙcyclopropyl, (CH₂)ₙbicyclopentanyl, said (CH₂)ₙpyrazolyl, (CH₂)ₙpyridyl, (CH₂)ₙfuranyl, (CH₂)ₙtriazolyl, (CH₂)ₙoxadizolyl, (CH₂)ₙpyrimidinyl, (CH₂)ₙthiophenyl, (CH₂)ₙtetrahydropyranyl, (CH₂)ₙtetrahydrofuranyl, (CH₂)ₙcyclopropyl, and (CH₂)ₙbicyclopentanyl, said group optionally substituted with 1 to 3 groups of R^{b}. Still another embodiment of this aspect of the invention is realized when R^{a} is independently selected from the group consisting of H, fluorine, chlorine, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, (CH₂)ₙpyrazolyl, and (CH₂)ₙpyridyl, said pyrazolyl and pyridyl unsubstituted or substituted with 1 to 3 groups of R^{b}.

An embodiment of this invention is realized when R^{b} is C₁₋₆ alkyl.

Another embodiment of the invention is realized when R^{c} is selected from the group consisting of CN, and unsubstituted or substituted oxetanyl, tetrahydrofuranyl, azetidinyl, pyrrolidinyl, and tetrahydropyranyl.

Another embodiment of the invention is realized when the substituents on R^{c} is selected from 1 to 3 groups consisting of C₁₋₆ alkyl, fluorine, chlorine, cyclopropyl, cyclobutyl, OH, OCH₃, OCH₂CH₃, and CN.

Still another embodiment of the invention of Formula I is represented by structural Formula II:

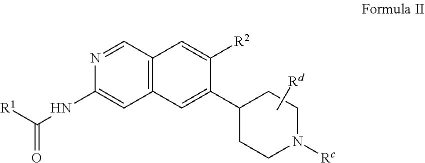

or a pharmaceutically acceptable salt thereof, wherein R¹, R² and R^{c} are as originally described and R^{d} is hydrogen or halogen. A subembodiment of the invention of Formula II is realized when R^{d} is hydrogen. Another subembodient of the invention of Formula II is realized when R^{d} is fluorine. Another subembodiment of the invention of Formula II is realized when R² is selected from the group consisting of hydrogen, chlorine, and CH₃. Another subembodiment of the invention of Formula II is realized when R^{c} is selected from the group consisting of oxetanyl, tetrahydrofuranyl, tetrahydropyranyl and cyclopentyl, said group unsubstituted or substituted with 1 to 3 groups of halogen, C₁₋₆ alkyl, —OC₁₋₆ alky, and OH. Another subembodiment of the invention of Formula II is realized when R^{c} is unsubstituted or substituted oxetanyl. Another subembodiment of the invention of Formula II is realized when R^{c} is unsubstituted or substituted tetrahydrofuranyl. Another subembodiment of the invention of Formula II is realized when R^{c} is unsubstituted or substituted tetrahydropyranyl. Another subembodiment of the invention of Formula II is realized when R^{c} is unsubstituted or substituted cyclopentyl.

Yet another embodiment of the invention Formula II is realized when R¹ is selected from the group consisting of C₁₋₆ alkyl, —(R)phenyl, —(R)pyrazolyl, —(R)oxazolyl, —(R)pyridyl, cyclopropafuropyridyl, —(R)cyclopropyl, —(R)cyclobutyl, —(R)cyclopentyl, —(R)cyclohexyl, —(R)bicyclopentanyl, —(R)bicyclohexanyl, —(R)spirohexanyl, —(R)spiropentanyl, —(R)spirooctanyl, —(R)piperidinyl, —(R)morpholinyl, —(R)pyrrolidinyl, —(R)azetidinyl, —(R)oxaspirooctanyl, —(R)oxabicyclohexanyl, —(R)oxabicycloheptanyl, —(R)oxaspiroheptanyl, —(R)oxaspirononanyl, —(R)tetrahydrofuranyl, and —(R)tetrahydropyranyl said alkyl, phenyl, pyrazolyl, oxazolyl, pyridyl, cyclopropafuropyridyl, cyclopropyl, cyclobutyl, cyclopentyl, cyclohexyl, bicyclopentanyl, bicyclohexanyl, spirohexanyl, spiropentanyl, spirooctanyl, piperidinyl, -morpholinyl, pyrrolidinyl, azetidinyl, oxaspirooctanyl, oxabicyclohexanyl, oxabicycloheptanyl, oxaspiroheptanyl, oxaspirononanyl, tetrahydrofuranyl, and tetrahydropyranyl unsubstituted or substituted with 1 to 3 groups selected from R^{a}. Still another embodiment of the invention of Formula II is realized when R¹ is unsubstituted or substituted —(R)cyclopropyl. Still another embodiment of the invention of Formula II is realized when R¹ is unsubstituted or substituted —(R)cyclobutyl. Still another embodiment of the invention of Formula II is realized when R¹ is unsubstituted or substituted —(R)tetrahydropyranyl. Still another embodiment of the invention of Formula II is realized when R¹ is unsubstituted or substituted —(R)pyrazolyl. Still another embodiment of the invention of Formula II is realized when R¹ is unsubstituted or substituted —(R)oxabicycloheptanyl. Still another embodiment of the invention of Formula II is realized when R¹ is unsubstituted or substituted —(R)pyridyl. Still another embodiment of the invention of Formula II is realized when R¹ is unsubstituted or substituted —(R)oxaspiroheptanyl. Still another embodiment of the invention of Formula II is realized when R¹ is unsubstituted or substituted —(R)spirohexanyl. Still another embodiment of the invention of Formula II is realized when R¹ is unsubstituted or substituted —(R)oxaspirooctanyl. Still another embodiment of the invention of Formula II is realized when R¹ is unsubstituted or substituted —(R)spiropentanyl. Still another embodiment of the invention of Formula II is realized when R¹ is unsubstituted or substituted —(R)oxabicyclohexanyl.

Still another aspect of the invention of Formula II is realized when R^{a} is independently selected from the group consisting of H, fluorine, chlorine, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, (CH₂)ₙpyrazolyl, and (CH₂)ₙpyridyl, said pyrazolyl and pyridyl unsubstituted or substituted with 1 to 3 groups of R^{b}.

Still another embodiment of the invention of Formula I is represented by structural Formula III:

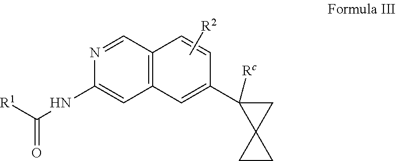

or a pharmaceutically acceptable salt thereof, wherein R¹, R² and R are as originally described. Another subembodiment of the invention of Formula III is realized when R² is selected from the group consisting of hydrogen, chlorine, and CH₃. Another subembodiment of the invention of Formula III is realized when R^{c} is selected from the group consisting of hydrogen, CN, halogen, and C₁₋₆ alkyl.

Yet another embodiment of the invention Formula III is realized when R¹ is selected from the group consisting of C₁₋₆ alkyl, —(R)₀₋₁phenyl, —(R)pyrazolyl, —(R)oxazolyl, —(R)pyridyl, cyclopropafuropyridyl, —(R)cyclopropyl, —(R)cyclobutyl, —(R)cyclopentyl, —(R)cyclohexyl, —(R)bicyclopentanyl, —(R)bicyclohexanyl, —(R)spirohexanyl, —(R)spiropentanyl, —(R)spirooctanyl, —(R)piperidinyl, —(R)morpholinyl, —(R)pyrrolidinyl, —(R)azetidinyl, —(R)oxaspirooctanyl, —(R)oxabicyclohexanyl, —(R)oxabicycloheptanyl, —(R)oxaspiroheptanyl, —(R)oxaspirononanyl, —(R)tetrahydrofuranyl, and —(R)tetrahydropyranyl said alkyl, phenyl, pyrazolyl, oxazolyl, pyridyl, cyclopropafuropyridyl, cyclopropyl, cyclobutyl, cyclopentyl, cyclohexyl, bicyclopentanyl, bicyclohexanyl, spirohexanyl, spiropentanyl, spirooctanyl, piperidinyl, -morpholinyl, pyrrolidinyl, azetidinyl, oxaspirooctanyl, oxabicyclohexanyl, oxabicycloheptanyl, oxaspiroheptanyl, oxaspirononanyl, tetrahydrofuranyl, and tetrahydropyranyl unsubstituted or substituted with 1 to 3 groups selected from R^{a}. Still another embodiment of the invention of Formula III is realized when R¹ is unsubstituted or substituted —(R)cyclopropyl. Still another embodiment of the invention of Formula III is realized when R¹ is unsubstituted or substituted —(R)cyclobutyl. Still another embodiment of the invention of Formula III is realized when R¹ is unsubstituted or substituted —(R)tetrahydropyranyl. Still another embodiment of the invention of Formula III is realized when R¹ is unsubstituted or substituted —(R)pyrazolyl. Still another embodiment of the invention of Formula III is realized when R¹ is unsubstituted or substituted —(R)oxabicycloheptanyl. Still another embodiment of the invention of Formula III is realized when R¹ is unsubstituted or substituted —(R)pyridyl. Still another embodiment of the invention of Formula III is realized when R¹ is unsubstituted or substituted —(R)oxaspiroheptanyl. Still another embodiment of the invention of Formula III is realized when R¹ is unsubstituted or substituted —(R)spirohexanyl. Still another embodiment of the invention of Formula III is realized when R¹ is unsubstituted or substituted —(R)oxaspirooctanyl. Still another embodiment of the invention of Formula III is realized when R¹ is unsubstituted or substituted —(R)spiropentanyl. Still another embodiment of the invention of Formula III is realized when R¹ is unsubstituted or substituted —(R)oxabicyclohexanyl.

Still another aspect of the invention of Formula III is realized when R^{a} is independently selected from the group consisting of H, fluorine, chlorine, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, (CH₂)ₙpyrazolyl, and (CH₂)ₙpyridyl, said pyrazolyl and pyridyl unsubstituted or substituted with 1 to 3 groups of R^{b}.

Still another embodiment of the invention of Formula I is represented by structural Formula IV:

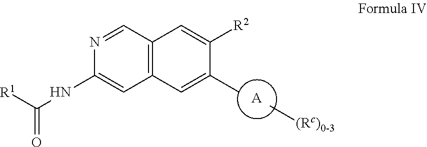

or a pharmaceutically acceptable salt thereof, wherein R¹, R² and R^{c} are as originally described and A is selected from the group consisting of cyclohexyl, cyclopropyl, cyclobutyl, oxabicycloheptanyl and pyranyl. A subembodiment of the invention of Formula IV is realized when A is cyclohexyl. Another subembodiment of the invention of Formula IV is realized when R² is selected from the group consisting of hydrogen, chlorine, and CH₃. Another subembodiment of the invention of Formula IV is realized when R^{c} is selected from the group consisting of CN, CH₃, azetidinyl and pyrrolidinyl, said group unsubstituted or substituted with 1 to 3 groups of halogen, C₁₋₆ alkyl, —OC₁₋₆ alky, and OH. Another subembodiment of the invention of Formula II is realized when R^{c} is unsubstituted or substituted azetidinyl. Another subembodiment of the invention of Formula II is realized when R^{c} is unsubstituted or substituted pyrrolidinyl.

Yet another embodiment of the invention Formula IV is realized when R¹ is selected from the group consisting of C₁₋₆ alkyl, —(R)phenyl, —(R)pyrazolyl, —(R)oxazolyl, —(R)pyridyl, cyclopropafuropyridyl, —(R)cyclopropyl, —(R)cyclobutyl, —(R)cyclopentyl, —(R)cyclohexyl, —(R)bicyclopentanyl, —(R)bicyclohexanyl, —(R)spirohexanyl, —(R)spiropentanyl, —(R)spirooctanyl, —(R)piperidinyl, —(R)morpholinyl, —(R)pyrrolidinyl, —(R)azetidinyl, —(R)oxaspirooctanyl, —(R)oxabicyclohexanyl, —(R)oxabicycloheptanyl, —(R)oxaspiroheptanyl, —(R)oxaspirononanyl, —(R)tetrahydrofuranyl, and —(R)tetrahydropyranyl said alkyl, phenyl, pyrazolyl, oxazolyl, pyridyl, cyclopropafuropyridyl, cyclopropyl, cyclobutyl, cyclopentyl, cyclohexyl, bicyclopentanyl, bicyclohexanyl, spirohexanyl, spiropentanyl, spirooctanyl, piperidinyl, -morpholinyl, pyrrolidinyl, azetidinyl, oxaspirooctanyl, oxabicyclohexanyl, oxabicycloheptanyl, oxaspiroheptanyl, oxaspirononanyl, tetrahydrofuranyl, and tetrahydropyranyl unsubstituted or substituted with 1 to 3 groups selected from R^{a}. Still another embodiment of the invention of Formula IV is realized when R¹ is unsubstituted or substituted —(R)cyclopropyl. Still another embodiment of the invention of Formula IV is realized when R¹ is unsubstituted or substituted —(R)cyclobutyl. Still another embodiment of the invention of Formula IV is realized when R¹ is unsubstituted or substituted —(R)tetrahydropyranyl. Still another embodiment of the invention of Formula IV is realized when R¹ is unsubstituted or substituted —(R)pyrazolyl. Still another embodiment of the invention of Formula IV is realized when R¹ is unsubstituted or substituted —(R)oxabicycloheptanyl. Still another embodiment of the invention of Formula IV is realized when R¹ is unsubstituted or substituted —(R)pyridyl. Still another embodiment of the invention of Formula IV is realized when R¹ is unsubstituted or substituted —(R)oxaspiroheptanyl. Still another embodiment of the invention of Formula IV is realized when R¹ is unsubstituted or substituted —(R)spirohexanyl. Still another embodiment of the invention of Formula IV is realized when R¹ is unsubstituted or substituted —(R)oxaspirooctanyl. Still another embodiment of the invention of Formula IV is realized when R¹ is unsubstituted or substituted —(R)spiropentanyl. Still another embodiment of the invention of Formula IV is realized when R¹ is unsubstituted or substituted —(R)oxabicyclohexanyl.

Still another aspect of the invention of Formula IV is realized when R^{a} is independently selected from the group consisting of H, fluorine, chlorine, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, (CH₂)ₙpyrazolyl, and (CH₂)ₙpyridyl, said pyrazolyl and pyridyl unsubstituted or substituted with 1 to 3 groups of R^{b}.

In another embodiment, the compounds of the invention include those identified herein as Examples in the tables below, and pharmaceutically acceptable salts thereof.

In another embodiment, the present invention provides pharmaceutical compositions comprising a pharmaceutically acceptable carrier and a compound of the invention or a pharmaceutically acceptable salt thereof.

In another embodiment, the present invention provides a method of treating a disease or disorder in which the LRRK2 kinase is involved, or one or more symptoms or conditions associated with said diseases or disorders, said method comprising administering to a subject (e.g., mammal, person, or patient) in need of such treatment an effective amount of a compound of the invention, or a pharmaceutically acceptable salt thereof, or pharmaceutically acceptable composition thereof. Non-limiting examples of such diseases or disorders, and symptoms associated with such diseases or disorders, each of which comprise additional independent embodiments of the invention, are described below.

Another embodiment provides the use of a compound of the invention, or a pharmaceutically acceptable salt thereof, and a pharmaceutically acceptable carrier, for the manufacture of a medicament for the treatment of Parkinson's Disease. The invention may also encompass the use of a compound of the invention, or a pharmaceutically acceptable salt thereof, and a pharmaceutically acceptable carrier, in therapy.

Another embodiment provides for medicaments or pharmaceutical compositions which may be useful for treating diseases or disorders in which LRRK2 is involved, such as Parkinson's Disease, which comprise a compound of the invention, or a pharmaceutically acceptable salt thereof, and a pharmaceutically acceptable carrier.

Another embodiment provides for the use of a compound of the invention which may be useful for treating diseases or disorders in which LRRK2 is involved, such as Parkinson's Disease.

Another embodiment provides a method for the manufacture of a medicament or a composition which may be useful for treating diseases or disorders in which LRRK2 is involved, such as Parkinson's Disease, comprising combining a compound of the invention with one or more pharmaceutically acceptable carriers.

### DETAILED DESCRIPTION OF THE INVENTION

The compounds of the invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Unless a specific stereochemistry is indicated, the present invention is meant to encompass all such isomeric forms of these compounds.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formulae I, II, III, and IV the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formulae I, II, III, and IV. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formulae I, II, III, and IV can be prepared without undue experimentation by conventional techniques well known to those skilled in the art or by processes analogous to those described in the Schemes and Examples herein using appropriate isotopically-enriched reagents and/or intermediates.

When a compound of the invention is capable of forming tautomers, all such tautomeric forms are also included within the scope of the present invention. For example, compounds including carbonyl —CH₂C(O)— groups (keto forms) may undergo tautomerism to form hydroxyl —CH═C(OH)— groups (enol forms). Both keto and enol forms, where present, are included within the scope of the present invention.

When any variable (e.g. R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-II. Accordingly, the methyl group of Illus-I is emerging from the page of the paper and the ethyl group in Illus-II is descending into the page, where the cyclohexene ring resides within the plane of the paper. It is assumed that the hydrogen on the same carbon as the methyl group of Illus-I descends into the page and the hydrogen on the same carbon as the ethyl group of Illus-II emerges from the page. The convention is the same where both a hashed and solid rectangle are appended to the same carbon as in Illus-III, the methyl group is emerging from the plane of the paper and the ethyl group is descending into the plane of the paper with the cyclohexene ring in the plane of the paper.

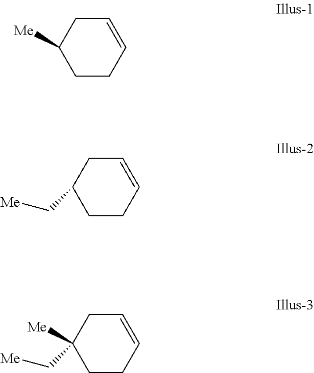 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present.

Components of a composition are typically aliquots of isolated pure material added to the composition, where the purity level of the isolated material added into the composition is the normally accepted purity level for a reagent of the type.

“at least one” used in reference to substituents appended to a compound substrate, for example, a halogen or a moiety appended to a portion of a structure replacing a hydrogen, means that one substituent of the group of substituents specified is present, and more than one of said substituents may be bonded to any of the defined or chemically accessible bonding points of the substrate.

Whether used in reference to a substituent on a compound or a component of a pharmaceutical composition the phrase “one or more”, means the same as “at least one”;
“concurrently” and “contemporaneously” both include in their meaning (1) simultaneously in time (e.g., at the same time); and (2) at different times but within the course of a common treatment schedule;
“consecutively” means one following the other;
“sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component;
“effective amount” or “therapeutically effective amount” is meant to describe the provision of an amount of at least one compound of the invention or of a composition comprising at least one compound of the invention which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of at least one compound of Formula I, Formula II, Formula III, or Formula IV that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;
“patient” and “subject” means an animal, such as a mammal (e.g., a human being) and is preferably a human being;
“prodrug” means compounds that are rapidly transformed, for example, by hydrolysis in blood, in vivo to the parent compound, e.g., conversion of a prodrug of Formula I through Formula IV to a compound of Formula I, Formula II, Formula III, or Formula IV or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention;

The term “substituted” means that one or more of the enumerated substituents can occupy one or more of the bonding positions on the substrate typically occupied by “—H”, provided that such substitution does not exceed the normal valency rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution of a moiety is described (e.g. “optionally substituted”) the term means that if substituents are present, one or more of the enumerated substituents for the specified substrate can be present on the substrate in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the invention or a substituent appended to a variable portion of a structural representation of a group of compounds of the invention have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

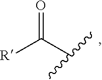

equally, with no differentiation implied using any or all of these representations;

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀-alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates an alkyl moiety connecting a hydroxyl moiety to a substrate.

The term “cycloalkyl” means a moiety having a main hydrocarbon chain forming a mono- or bicyclo-cyclic aliphatic moiety comprising at least 3 carbon atoms (the minimum number necessary to provide a monocyclic moiety) up to the maximum number of specified carbon atoms, generally 8 for a monocyclic moiety and 10 for a bicyclic moiety, inclusive of spirocyclic moieties. Examples of cycloalkyl moieties include, but are not limited to, cyclopropyl, cyclobutyl, cyclopentyl or cyclohexyl. The term “cycloalkyl” also includes non-aromatic, fused multicyclic ring system comprising up to 20 carbon atoms which may optionally be substituted as defined herein for “alkyl” generally. Suitable multicyclic cycloalkyls are, for example, but are not limited to: 1-decalin; norbornyl; adamantly; and the like;

As used herein, when the term “alkyl” is modified by “substituted” or “optionally substituted”, it means that one or more C—H bonds in the alkyl moiety group is substituted, or optionally may be substituted, by a substituent bonded to the alkyl substrate which is called out in defining the moiety.

Where a structural formula represents bonding between a moiety and a substrate using a bonding line that terminates in the middle of the structure, for example the following representations:

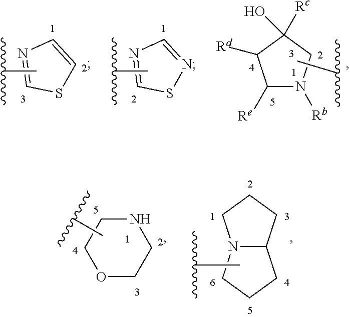

whether or not numbered the structure indicates that unless otherwise defined the moiety may be bonded to the substrate through any of available ring atom, for example, the numbered atoms of the example moieties.

The term “oxo” refers to an oxygen atom, which forms a carbonyl when attached to carbon, an N-oxide when attached to nitrogen, and a sulfoxide or sulfone when attached to sulfur.

The term “heteroaryl” refers to an aromatic 5-8 membered monocyclic, 8-12 membered bicyclic, or 11-14 membered tricyclic ring system having 1-3 heteroatoms for monocyclic, 1-6 heteroatoms for bicyclic, or 1-9 heteroatoms for tricyclic, said heteroatoms selected from O, N, or S (e.g., carbon atoms and 1-3, 1-6, or 1-9 heteroatoms of N, O, or S for monocyclic, bicyclic, or tricyclic, respectively). Non-limiting examples of heteroaryls are pyridyl, pyrazolyl, pyrimidinyl, furanyl, oxazolyl, triazolyl, oxadiazolyl, and thiophenyl. The heteroaryl groups herein described may also contain fused rings that share a common carbon-carbon bond.

The term “heterocyclyl” (or heterocycloalkyl) means a non-aromatic saturated monocyclic or multicyclic ring system comprising 3 to 10 ring atoms, preferably 5 to 10 ring atoms, in which one or more of the atoms in the ring system is an element other than carbon, for example nitrogen (e.g. piperidyl- or pyrrolidinyl), oxygen (e.g. furanyl and tetrahydropyranyl) or sulfur (e.g. tetrahydrothiopheneyl and tetrahydrothiopyranyl); and wherein the heteroatoms can be alone or in combination provided that the moiety does not contain adjacent oxygen and/or sulfur atoms present in the ring system; preferred heterocyclyl moieties contain 5 to 6 ring atoms; the prefix aza, oxa or thia before the heterocyclyl root name means that at least one nitrogen, oxygen or sulfur atom, respectively, is present as a ring atom; the heterocyclyl can be optionally substituted by one or more independently selected substituents;

The nitrogen or sulfur atom of the heterocyclyl can be optionally oxidized to the corresponding N-oxide, S-oxide or S,S-dioxide (SO₂); non-limiting examples of suitable monocyclic heterocyclyl rings include piperidyl, pyrrolidinyl, piperazinyl, morpholinyl-

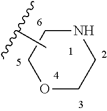

(where unless otherwise noted the moiety is bonded to the substrate through any of ring carbon atoms C2, C3, C5, or C6), thiomorpholinyl, thiazolidinyl, 1,3-dioxolanyl, 1,4-dioxanyl, tetrahydrofuranyl, tetrahydrothiophenyl, tetrahydrothiopyranyl, and the like; and polycyclicheterocyclvl compounds, for example, moieties of the structure:

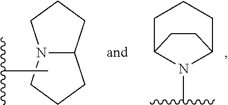

and the like.

The term “halogen” means fluorine, chlorine, bromine, or iodine; preferred halogens, unless specified otherwise where the term is used, are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group, perhaloalkyl (or “fully halogenated” alkyl) means that all bonding positions not participating in bonding the alkyl substituent to a substrate are occupied by a halogen, for example, where the alkyl is selected to be methyl, the term perfluoroalkyl means —CF₃;

The term “hydroxyl” and “hydroxy” means an HO— group, “hydroxyalkyl” means a substituent of the formula: “HO-alkyl-”, wherein the alkyl group is bonded to the substrate and may be substituted or unsubstituted as defined above; preferred hydroxyalkyl moieties comprise a lower alkyl; Non-limiting examples of suitable hydroxyalkyl groups include hydroxymethyl and 2-hydroxyethyl; and

The bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

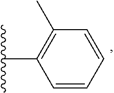 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of an atom to which it is bonded indicates that the moiety may be bonded to a substrate via any of the atoms in the moiety which are available for bonding as described in the examples above.

The line -, as a bond generally indicates a mixture of, or either of, the possible isomers, e.g., containing (R)- and (S)-stereochemical configuration. For example:

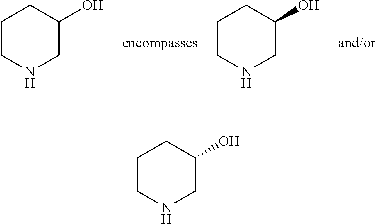 [SMILES: OC1CCCNC1 ; O[C@@H]1CCCNC1 ; O[C@H]1CCCNC1]

Furthermore, unwedged-bolded or unwedged-hashed lines are used in structures containing multiple stereocenters in order to depict relative configuration where it is known. For example:

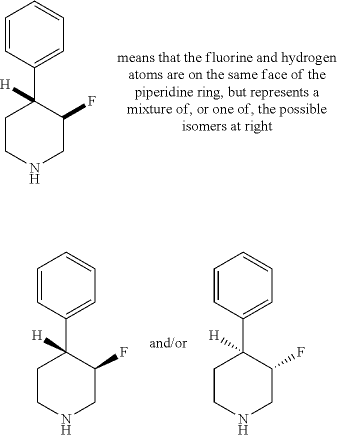 [SMILES: F[C@H]1CNCC[C@@H]1c1ccccc1 ; F[C@H]1CNCC[C@@H]1c1ccccc1 ; F[C@@H]1CNCC[C@H]1c1ccccc1]

whereas:

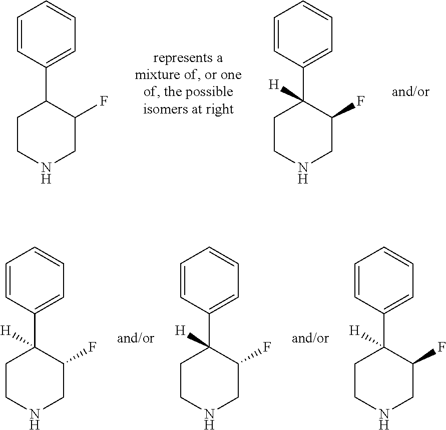 [SMILES: FC1CNCCC1c1ccccc1 ; F[C@H]1CNCC[C@@H]1c1ccccc1 ; F[C@@H]1CNCC[C@H]1c1ccccc1 ; F[C@@H]1CNCC[C@@H]1c1ccccc1 ; F[C@H]1CNCC[C@H]1c1ccccc1]

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the present invention is meant to encompass all such stereoisomeric forms of these compounds.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

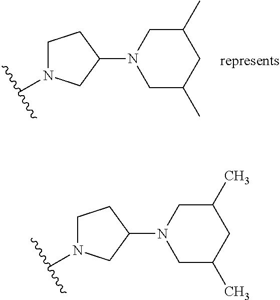 [SMILES: *N1CCC(N2CC(C)CC(C)C2)C1 ; *N1CCC(N2CC(C)CC(C)C2)C1]

Unsatisfied valences in the text, schemes, examples, structural formulae, and any Tables herein is assumed to have a hydrogen atom or atoms of sufficient number to satisfy the valences.

One or more compounds of the invention may also exist as, or optionally be converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This invention also includes the compounds of this invention in isolated and purified form obtained by routine techniques. Polymorphic forms of the compounds of Formula I, Formula II, Formula III, and Formula IV and of the salts, solvates and prodrugs of the compounds of Formula I, Formula II, Formula III, and Formula IV are intended to be included in the present invention. Certain compounds of the invention may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the invention, their salts, and solvates and prodrugs thereof, may exist in different tautomeric forms or in equilibrium among such forms, all such forms of the compound are embraced by, and included within the scope of the invention. Examples of such tautomers include, but are not limited to, ketone/enol tautomeric forms, imine-enamine tautomeric forms, and for example heteroaromatic forms such as the following moieties;

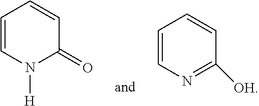 [SMILES: O=c1cccc[nH]1 ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the invention the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which LRRK2 is involved. The terms “preventing” or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals), in whom the inhibition of LRRK2 kinase activity is indicated or desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the invention or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the invention or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present invention are each directed to a method for the treatment a disease, disorder, or condition, or one or more symptoms thereof (“indications”) in which the LRRK2 kinase is involved and for which the inhibition of LRRK2 kinase is desired, which method comprises administering to a subject in need of such treatment a therapeutically effective amount of a compound of the invention, or a pharmaceutically acceptable salt thereof, or a pharmaceutical composition comprising said compound or salt thereof.

In another embodiment, the present invention is directed to a method for the manufacture of a medicament for inhibition of LRRK2 receptor activity in a subject comprising combining a compound of the present invention, or a pharmaceutically acceptable salt thereof, with a pharmaceutical carrier or diluent.

One such embodiment provides a method of treating Parkinson's disease in a subject in need thereof, said method comprising administering to a subject in need of such treatment a therapeutically effective amount of a compound of the invention, or a pharmaceutically acceptable salt thereof, or a pharmaceutical composition comprising said compound or salt thereof. In one such embodiment, the subject is a human.

Another embodiment provides a method for the treatment or prophylaxis of neurologic damage associated with Parkinson's disease in a subject in need thereof. Another embodiment provides a method of treating or improving dopaminergic tone to provide symptomatic relief in a subject in need thereof, for example, in treating, alleviating, ameliorating, or managing motor and non-motor symptoms of Parkinson's disease.

Another embodiment provides a method for the treatment or prophylaxis of abnormal motor symptoms associated with Parkinson's disease (including but not limited to bradykinesia, rigidity and resting tremor). Another embodiment provides a method for the treatment or prophylaxis of abnormal non-motor symptoms associated with Parkinson's disease (including but not limited to cognitive dysfunction, autonomic dysfunction, emotional changes and sleep disruption); Lewy body dementia; and L-Dopa induced dyskinesias. Each said method independently comprises administering to a patient in need of such treatment an effective amount of a compound of the invention, or a pharmaceutically acceptable salt thereof, or pharmaceutically acceptable composition thereof.

Non-limiting examples of additional indications in which LRRK2 is involved and in which the treatment or prophylaxis of said indications in a subject in need thereof are contemplated include the following, each of which, alone or in combination, comprise additional embodiments of the invention: Alzheimer's disease, mild cognitive impairment, the transition from mild cognitive impairment to Alzheimer's disease, tauopathy disorders characterized by hyperphosphorylation of tau such as argyrophilic grain disease, Picks disease, corticobasal degeneration, progressive supranuclear palsy, inherited frontotemporal dementia, and Parkinson's disease linked to chromosome 17.

Additional indications include neuroinflammation, including neuroinflammation associated with of microglial inflammatory responses associated with multiple sclerosis, HIV-induced dementia, ALS, ischemic stroke, traumatic brain injury and spinal cord injury.

Additional indications include diseases of the immune system including lymphomas, leukemias, multiple sclerosis, rheumatoid arthritis, systemic lupus erythematosus, autoimmune hemolytic anemia, pure red cell aplasia, idiopathic thrombocytopenic pupura (ITP), Evans Syndrome, vasculitis, bullous skin disorder, type I diabetes mellitus, Sjorgen's syndrome, Delvic's disease, inflammatory myopathies, and ankylosing spondylitis.

Additional indications include renal cancer, breast cancer, lung cancer, prostate cancer, and acute myelogenous leukemia (AML) in subjects expressing the LRRK2 G2019S mutation.

Additional indications include papillary renal and thyroid carcinomas in a subject in whom LRRK2 is amplified or overexpressed.

Additional indications include chronic autoimmune diseases including Crohn's disease and leprosy.

The present invention includes within its scope prodrugs of the compounds of this invention. In general, such prodrugs will be functional derivatives of the compounds of this invention which are readily convertible in vivo into the required compound. Thus, in the methods of treatment of the present invention, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The compounds of the present invention may be used in combination with one or more other drugs in the treatment, prevention, control, amelioration, or reduction of risk of diseases or conditions for which compounds of Formula I, Formula II, Formula III, and Formula IV, or the other drugs may have utility, where the combination of the drugs together are safer or more effective than either drug alone. Such other drug(s) may be administered, by a route and in an amount commonly used therefore, contemporaneously or sequentially with a compound of Formula I. When a compound of Formula I, Formula II, Formula III, and Formula IV is used contemporaneously with one or more other drugs, a pharmaceutical composition in unit dosage form containing such other drugs and the compound of Formula I, Formula II, Formula III, or Formula IV is preferred. However, the combination therapy may also include therapies in which the compound of Formula I, Formula II, Formula III, or Formula IV, and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I, Formula II, Formula III, or Formula IV.

For example, the present compounds may be used in conjunction with one or more additional therapeutic agents, for example: L-DOPA; dopaminergic agonists such as quinpirole, ropinirole, pramipexole, pergolide and bromocriptine; MAO-B inhibitors such as rasagiline, deprenyl and selegiline; DOPA decarboxylase inhibitors such as carbidopa and benserazide; and COMT inhibitors such as tolcapone and entacapone; or potential therapies such as an adenosine A2a antagonists, metabotropic glutamate receptor 4 modulators, or growth factors such as brain derived neurotrophic factor (BDNF), and a pharmaceutically acceptable carrier.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present invention are employed. Similarly, transdermal patches may also be used for topical administration.

The pharmaceutical composition and method of the present invention may further comprise other therapeutically active compounds as noted herein which are usually applied in the treatment of the above-mentioned pathological conditions.

In the treatment, prevention, control, amelioration, or reduction of risk of conditions which require inhibition of LRRK2 kinase activity an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this invention are illustrated in the following Schemes and Examples. Starting materials are made according to procedures known in the art or as illustrated herein.

### Preparative Examples

The compounds of the present invention can be prepared according to the following schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this invention can be readily understood by one skilled in the art from viewing the following schemes and descriptions.

Abbreviations used in the experimentals may include, but are not limited to the following.

| | |
|---|---|
| 2-MeTHF | 2-Methyltetrahydrofuran |
| AcOH | Acetic Acid |
| aq | Aqueous |
| BHT | 3,5-Di-tert-4-butylhydroxytoluene |
| BINAP | (2,2'-bis(diphenylphosphino)-1,1'-binaphthyl) |
| C₂Cl₆ | Hexachloroethane |
| CPME | Cyclopentyl methyl ether |
| DAST | Diethylaminosulfur trifluoride |
| DCE | Dichloroethane |
| DCM | Dichloromethane |
| DIPEA | N,N-Diisopropylethylamine |
| DMA | Dimethylacetamide |
| DMAP | 4-Dimethylaminopyridine |
| DMCDA | trans-N,N′-dimethylcyclohexane-1,2-diamine |
| DMF | Dimethylformamide |
| DMSO | Dimethyl sulfoxide |
| EtOAc | EtOAc |
| Et₃N | Triethylamine |
| ESI | Electrospray ionization |
| h | Hours |
| ¹H-NMR | Proton nuclear magnetic resonance |
| HPLC | High performance liquid chromatography |
| IPA | Isopropyl alcohol |
| Josiphos-SL-J009-1-Pd- | {(R)-1-[(Sp)-2-(Dicyclohexylphosphino)ferrocenyl]ethyldi-tert-butylphosphine} |
| G3 | [2-(2′-amino-1,1′-biphenyl)]palladium(II) methanesulfonate (MFCD27978424) |
| | 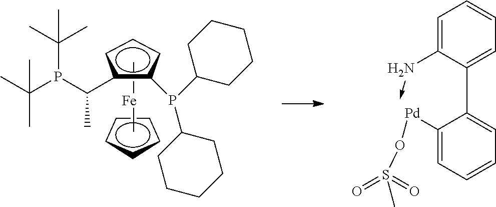 Josiphos-SL-J009-1-Pd-G3 |
| LCMS | Liquid chromatography-mass spectrometry |
| LiHMDS | Lithium bis(trimethylsilyl)amide |
| MeCN | Acetonitrile |
| MeOH | Methanol |
| MS | Mass spectrometry |
| m/z | Mass to charge ratio |
| NBS | N-bromosuccinimide |
| NMP | N-Methyl-2-pyrrolidone |
| Pd/C | Palladium on Carbon |
| PE | Petroleum ether |
| psi | Pounds per square inch |
| RT | Room temperature |
| SFC | Supercritical Fluid Chromatography |
| TFA | Trifluoroacetic acid |
| THF | Tetrahydrofuran |
| TLC | Thin Layer Chromatography |
| t_{R} | Retention time |
| TBAF | Tetra-n-butylammonium fluoride |
| TBAI | Tetrabutylammonium Iodide |
| TBDPS | Tert-butyldiphenylsilyl |
| Tf2O | Trifluoromethanesulfonic anhydride |
| TEA | triethylamine |
| MeONa | Sodium methanolate methanol |
| TBDPSCl | tert-Butyl(chloro)diphenylsilane |
| DEA | diethanolamine |
| DMP | Dess-Martin periodinane |
| TMS/TMS-CN | Trimethylsilyl/Trimethylsilyl cyanide |
| CAN | Cerric ammonium nitrate |
| 2-MeTHF | 2-Methyl Tetrahydrofuran |
| Celite | Trademark for diatomaceous earth |
| HATU | 1-[Bis(dimethylamino)methylene]-1H-1,2,3-triazolo[4,5-b] pyridinium |
| | 3-oxid hexafluorophosphate |
| PCC | Pyridinium Chlorochromate |
| NiCl2.dme | Nickel(II) chloride ethylene glycol dimethyl ether complex |
| RuPhos PdG3 | (2-Dicyclohexylphosphino-2′,6′-diisopropoxy-1,1′-biphenyl) |
| | [2-(2′-amino-1,1′-biphenyl)]palladium(II) methanesulfonate |
| | 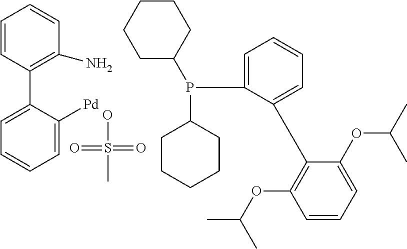 |
| RuPhos | 2-Dicyclohexylphosphino-2′,6′-diisopropoxybiphenyl |
| | 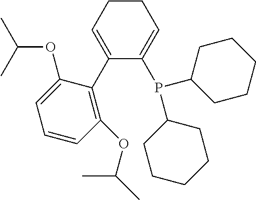 [SMILES: CC(C)Oc1cccc(OC(C)C)c1C1=CCCC=C1P(C1CCCCC1)C1CCCCC1] |
| Xantphos Pd G3 | [(4,5-Bis(diphenylphosphino)-9,9-dimethylxanthene)-2-(2′- |
| | amino-1,1′-biphenyl)]palladium(II) methanesulfonate |
| | 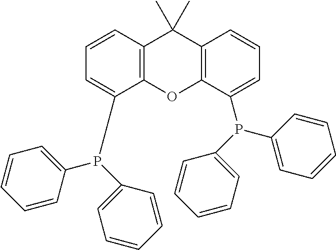 [SMILES: CC1(C)c2cccc(CP(c3ccccc3)c3ccccc3)c2Oc2c(P(c3ccccc3)c3ccccc3)cccc21] |
| | 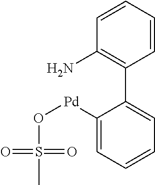 |
| Cataxium Pd G3 | Mesylate[(di(1-adamantyl)-n-butylphosphine)-2-(2′-amino-1,1′-biphenyl)]palladium(II), |
| | [(Di(1-adamantyl)- |
| | butylphosphine)-2-(2-amino-1,1′-biphenyl)]palladium(II) methanesulfonate |
| RuPhos Pd G4 | Methansulfonato(2-dicyclohexylphosphino-2′,6′-di-i-propoxy-1,1′-biphenyl) |
| | (2′-methylamino-1,1-biphenyl-2-yl) Palladium (II) |
| DIEA | Diisopropyl ethyl amine |
| XPhos PD G3 | (2-Dicyclohexylphosphino-2′,4′,6′-triisopropyl-1,1′-biphenyl) |
| | [2-(2′-amino-1,1-biphenyl)]palladium(II) methanesulfonate |
| RockPhos Pd G3 | [(2-Di-tert-butylphosphino-3-methoxy-6-methyl-2′,4′,6-triisopropyl-1,1-biphenyl)-2-(2- |
| | aminobiphenyl)]palladium(II) methanesulfonate |
| Si-DMT | SiliaMetS DMT (Dimercaptotriazine) |
| Rac-BINAP Pd G3 | Racemic methanesulfonato[2,2′-bis(diphenylphosphino)-1,1′-binaphthyl] |
| | (2′-amino-1,1′-biphenyl-2-yl)palladium(II) |
| Ni-Xantphos Pd G4 | 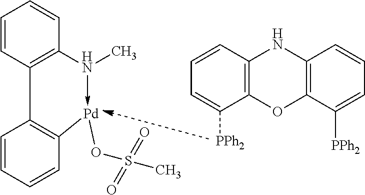 |

### General Experimental Information

Unless otherwise noted, all reactions are magnetically stirred. Unless otherwise noted, when diethyl ether is used in the experiments described below, it is Fisher ACS certified material and is stabilized with BHT. Unless otherwise noted, “concentrated” and/or “solvent removed under reduced pressure” means evaporating the solvent from a solution or mixture using a rotary evaporator or vacuum pump. Unless otherwise noted, flash chromatography is carried out on a Teledyne Isco (Lincoln, NE), Analogix (Burlington, WI), or Biotage (Stockholm, SWE) automated chromatography system using a commercially available cartridge as the column. Columns may be purchased from Teledyne Isco, Analogix, Biotage, Varian (Palo Alto, CA), or Supelco (Bellefonte, PA) and are usually filled with silica gel as the stationary phase. Reverse phase prep-HPLC conditions, where used, can be found at the end of each experimental section. Aqueous solutions were concentrated on a Genevac (Ipswich, ENG) or by freeze-drying/lyophilization. Unless otherwise noted, all LRRK2 pIC₅₀ data presented in tables refers to the LRRK2 G2019S Km ATP LanthaScreen™ assay (Life Technologies Corp., Carlsbad, CA) that is described in the Biological Assay section.

### Synthesis of Common Intermediates

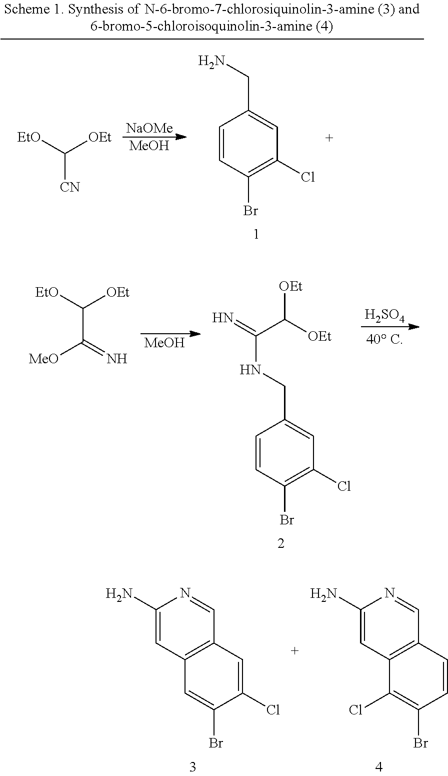

### N-(4-bromo-3-chlorobenzyl)-2,2-diethoxyacetimidamide (1)

A 5 L round-bottom flask was charged with 2,2-diethoxyacetonitrile (250 g, 1.94 mol, 1.00 eq.), and MeOH (1.50 L). Sodium methoxide (24.4 g, 135 mmol, 30% purity) was added to the mixture dropwise. The flask was evacuated and purged with N2 three times. The resulting mixture was stirred for 6 hours at 25° C. The crude reaction mixture was adjusted to a pH of 8-9 using dry CO₂. The reaction was concentrated and then diluted with water. The organic material was extracted from the aqueous solution using EtOAc. Four reactions of the same scale were combined for the following workup. The organic layers were combined and washed with water, dried over sodium sulfate. The solution was then concentrated in vacuo, to afford the title compound 1.

### N-(4-bromo-3-chlorobenzyl)-2,2-diethoxyacetimidamide (2)

A 500 mL round-bottom flask was charged with (4-bromo-3-chlorophenyl)methanamine 1 (1.00 kg, 4.56 mol) into MeOH (150 mL). Methyl 2,2-diethoxyacetimidate (918 g, 5.69 mol) was added to the mixture and stirred at 15° C. for 16 hours. The crude material concentrated in vacuo to afford the title compound 2 which was used directly in a subsequent reaction without further purification.

### 6-bromo-7-chloroisoquinolin-3-amine (3) and 6-bromo-5-chloroisoquinolin-3-amine (4)

A 5 L round-bottom flask was charged with N-(4-bromo-3-chlorobenzyl)-2,2-diethoxyacetimidamide 2 (280 g, 800 mmol). Sulfuric acid (1.4 L) was added, and the reaction was stirred overnight at 40° C. The pH of the mixture was adjusted to pH 9 with ammonium hydroxide (3.50 L) to precipitate out the product. The precipitate was collected by filtration and washed with water, affording a mixture of two isomers (1.3 kg, crude). The crude product was purified by pre-HPLC (column: phenomenex luna C18 250 mm*100 mm*0 mm; mobile phase: [water (0.1% TFA)-ACN]; b %: 10%-40%, 30 min). Ammonium hydroxide was used to adjust to a pH=7-8. The solution was filtered and washed with water. The organic layer was concentrated in vacuo to afford the title compound 3. MS (ESI): m/z calc'd for C₉H₇BrClN₂ [M+H]⁺: 257, found 257. ¹H NMR (400 MHz, DMSO-d₆, 25° C.) δ 8.79 (s, 1H), 8.06 (s, 1H), 8.02 (s, 1H), 6.55 (s, 1H), 6.23 (s, 2H). Compound 4 was not used.

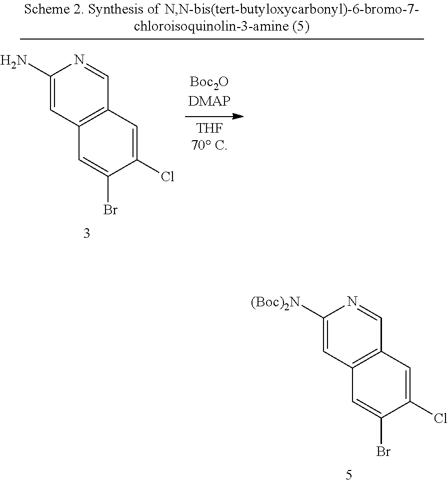

A 5 L round-bottom flask was charged with 6-bromo-7-chloroisoquinolin-3-amine 3 (54.0 g, 209 mmol) and THF (1.00 L) at 25° C. The mixture was heated to 70° C. for 30 minutes after which the solution turned clear. Di-tert-butyl dicarbonate (160 g, 733 mmol) and DMAP (2.56 g, 20.9 mmol) were added to the solution. The reaction was stirred at 70° C. for 1 hour. Solvent was removed under reduced pressure and the crude residue was purified by flash chromatography. The mixture was filtered with IPA, and the filtrate was concentrated in vacuo. The crude product was recrystallized from n-heptane (150 mL, 25° C.) to afford the title compound 5. MS (ESI): m z calc'd for C₁₉H₂₃BrClN₂O₂ [M+H]⁺: 457, found 457. ¹H NMR (400 MHz, DMSO-d₆, 25° C.) δ 9.24 (s, 1H), 8.58 (s, 1H), 8.52 (s, 1H), 7.85 (s, 1H), 1.39 (s, 18H).

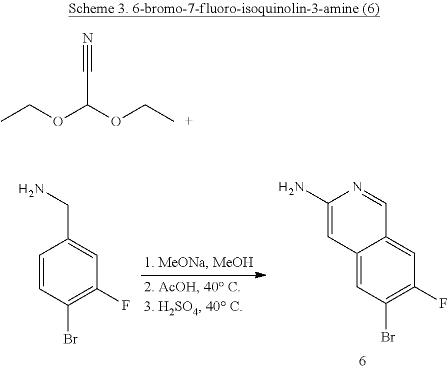

### 6-bromo-7-fluoro-isoquinolin-3-amine (6)

A solution of MeONa/MeOH (0.18 mL, 0.77 mmol) was added dropwise to a solution of 2,2-diethoxyacetonitrile (1.0 g, 7.74 mmol) in MeOH (7.74 mL). The resulting mixture was stirred for 20 hours at room temperature. AcOH (44.3 μL, 0.77 mmol) was added to adjust the pH to 7-8 (using pH strips). 4-Bromo-3-fluoro-phenyl)methanamine hydrochloride (1.86 g, 7.74 mmol) was added and the resulting mixture was stirred at 40° C. for 4 hours. The reaction mixture was concentrated under reduced pressure. Sulfuric acid (12.6 mL, 232.3 mmol) was added, and the resulting mixture was stirred at 40° C. for 16 hours. NH₄OH (30.8 mL, 240.0 mmol) was added dropwise at 0° C. The solvent was removed under reduced pressure, and the residue was purified by C18 silica gel to afford the title compound 6. MS (ESI): m/z calc'd for C₉H₇BrFN₂ [M+H]⁺: 241, found 241. ¹H NMR (499 MHz, DMSO-d6) 6.07 (s, 2H), 6.61 (s, 1H), 7.76 (m, 1H), 8.01 (m, 1H), 8.80 (1H, s).

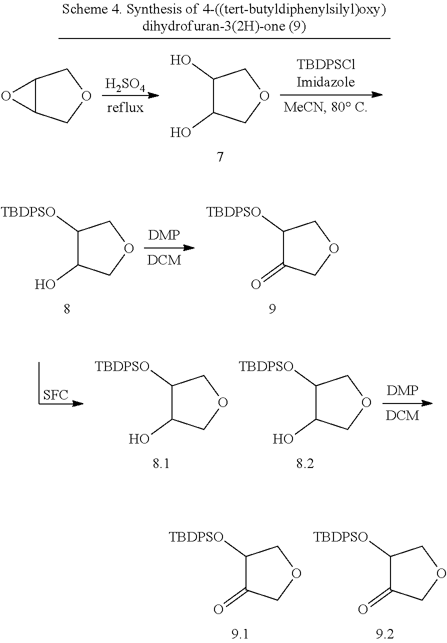

### Oxolane-3,4-diol (7)

A 10-L 4-necked round-bottom flask was charged with 3,6-dioxabicyclo[3.1.0]hexane (409 g, 4750 mmol) and H₂SO₄ (4 L, 1.5 mol/L). The resulting solution was stirred for 6 h at reflux. The reaction mixture was cooled to room temperature. The pH value of the solution was adjusted to 8 with Na₂CO₃. The resulting mixture was concentrated under vacuum, then washed with 5 L of THF, and concentrated under vacuum, affording the title compound 7.

### 4-[(tert-butyldiphenylsilyl)oxy]oxolan-3-ol (8)

A 3-L 4-necked round-bottom flask was purged and maintained with an inert atmosphere of nitrogen, and charged with oxolane-3,4-diol 7 (52.0 g, 499 mmol), ACN (1.5 L), imidazole (51.0 g, 749 mmol), and TBDPSCl (137 g, 498 mmol). The resulting solution was stirred for 4 h at 80° C., concentrated under vacuum, diluted with 1 L of EtOAc, washed with water (500 mL×2), dried over anhydrous sodium sulfate and concentrated under vacuum. The residue was purified on a silica gel column with EtOAc/PE (1:100-1:30), affording the title compound 8. (3S,4R)-4-[(tert-butyldiphenylsilyl)oxy]oxolan-3-ol or (3R,4S)-4-[(tert-butyldiphenylsilyl)oxy]oxolan-3-ol (8.1) and (8.2) Crude product 8 was purified by Prep-SFC using: Column, CHIRALPAK AS-H, 5*25 cm, 5 um; mobile phase, CO₂ (46%) and IPA (0.2% DEA) (54%); Detector, UV, affording title compounds 8.1 (tR=0.92 min) and 8.2 (tR=1.63 min).

### 4-[(tert-butyldiphenylsilyl)oxy]oxolan-3-one (9)

A 2-L 3-necked round-bottom flask was purged and maintained with an inert atmosphere of nitrogen, and charged with DMP (93 g, 219 mmol), DCM (1.1 L), 4-[(tert-butyldiphenylsilyl) oxy]oxolan-3-ol 8 (71 g, 207 mmol). The resulting solution was stirred for 3 h at 25-30° C. The resulting solution was diluted with 2 L of PE. The resulting mixture was washed with aq. NaHCO₃ and 1 L of brine. The mixture was dried over anhydrous sodium sulfate and concentrated under vacuum. The residue was applied onto a silica gel column with EtOAc/PE (1:100-1:30), affording the title compound 9. ¹H NMR (400 MHz, CDCl₃) δ: 7.85-7.76 (m, 2H), 7.72-7.64 (m, 2H), 7.53-7.38 (m, 6H), 4.30 (m, 1H), 4.11-4.02 (m, 2H), 3.93 (d, J=17.5 Hz, 1H), 3.80-3.70 (m, 1H), 1.12 (s, 9H).

### (R)-4-[(tert-butyldiphenylsilyl)oxy]oxolan-3-one or (S)-4-[(tert-butyldiphenylsilyl)oxy]oxolan-3-one (9.1)

Into a 5 L 4-necked round-bottom flask purged and maintained with an inert atmosphere of nitrogen, was placed (3S,4R or 3R,4S)-4-[(tert-butyldiphenylsilyl)oxy]oxolan-3-ol 8.1 (85 g, 249 mmol), DCM (1.700 L). This was followed by the addition of Dess-Martin periodinane (116 g, 274 mmol), in portions at room temperature. The resulting solution was stirred for 3 h at 30° C. The reaction was then quenched by the addition of 1.5 L of NaHCO₃/Na₂S₂O₃ (1:1). The resulting solution was stirred for 30 min. The resulting solution was extracted with DCM. The organic phase was washed with brine, dried over anhydrous sodium sulfate and concentrated under vacuum. The residue was applied onto a silica gel column affording the title compound 9.1. MS (ESI): m/z calc'd for C₂₀H₂₅O₃Si [M+H]⁺: 341, found 341. ¹H NMR (300 MHz, DMSO-d₆, 25° C.) δ 7.66 (m, 4H), 7.56-7.36 (m, 6H), 4.35 (m, 1H), 4.18-3.85 (m, 3H), 3.71 (t, 1H), 1.03 (s, 9H). Compound 9.2 was not used further.

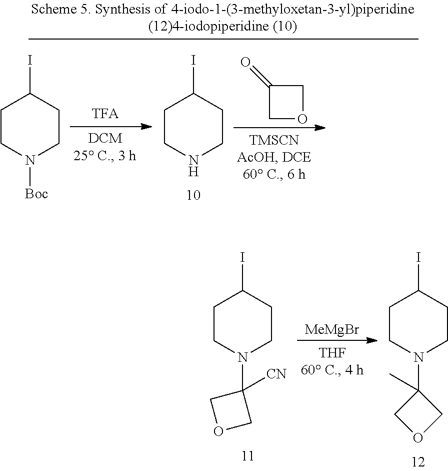

A round-bottom flask was charged with tert-butyl 4-iodopiperidine-1-carboxylate (100 g, 321 mmol) and DCM (500 mL). TFA (100 mL) was added to the reaction, and the reaction was stirred for 3 hours. The mixture was concentrated under reduced pressure to give the title compound which was used in a subsequent step without purification.

### 3-(4-iodopiperidin-1-yl)oxetane-3-carbonitrile (11)

A round-bottom flask was charged with 4-iodopiperidine (122 g, 578 mmol) and DCE (1.22 L). Oxetan-3-one (167 g, 2.31 mmol) and acetic acid (347 g, 5.78 mmol, 330 mL) were added to the mixture and were heated to 60° C. for 1 h. TMSCN (537 g, 5.78 mmol, 723 mL) was added, and the reaction was heated to 60° C. for 5 h. Water was added to the reaction mixture, then the mixture was extracted with DCM. The combined organic layers were washed with brine and dried over sodium sulfate. The solution was filtered and concentrated under reduced pressure to give a crude residue. The crude residue was subjected to purification by iterative flash chromatography over silica gel to afford the title compound 11.

### 4-iodo-1-(3-methyloxetan-3-yl)piperidine (12)

A round-bottom flask was charged with 3-(4-iodopiperidin-1-yl)oxetane-3-carbonitrile 11 (162 g, 556 mmol) and THF (1950 mL). The reaction was heated to 60° C. under an inert atmosphere. Methyl magnesium bromide (3M, 556 mL) was added to the reaction at 60° C., and the reaction was heated for 4 h. The reaction was quenched with saturated aqueous ammonium chloride andextracted with ethyl acetate. The combined organic layers were washed with brine and dried over sodium sulfate. The solution was filtered and concentrated under reduced pressure. The mixture was further purification by pre-HPLC. (column: Phenomenex luna C18 250*150 mm*um; mobile phase: [Water-ACN]; B %: 30%-55%, 25 min) affording title compound 12. MS (ESI): m/z calc'd for C₉H₁₆INO [M+H]⁺: 282, found 282. ¹H NMR (400 MHz, CDCl₃-d, 25° C.) δ4.53 (m, 2H), 4.26-4.39 (m, 1H), 4.19 (m, 2H), 2.36-2.49 (m, 2H), 2.07-2.25 (m, 6H), 1.35 (s, 3H).

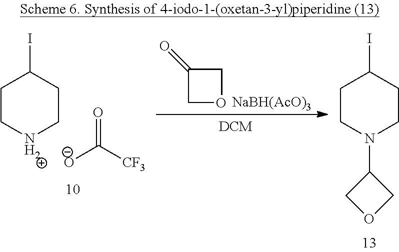 [SMILES: IC1CC[NH2+]CC1 ; O=C([O-])C(F)(F)F ; O=C1COC1 ; IC1CCN(C2COC2)CC1 ; "NaBH(AcO)3"]

### 4-iodo-1-(oxetan-3-yl)piperidine (13)

Into a 10-L 4-necked round-bottom flask purged and maintained with an inert atmosphere of nitrogen, were placed 4-iodopiperidine trifluoroacetic acid (420.00 g, 1 eq.), DCM (4.2 L), and 3-oxetanone (138.71 g, 1.50 eq.). This was followed by the addition of NaBH(AcO)₃ (816.00 g, 3.00 eq.) in several batches at RT in 20 min. The resulting solution was stirred for 15 h at room temperature. The reaction was then quenched by the addition of 5 L of Na₂CO₃. The resulting solution was extracted with 2×5 L of dichloromethane, and the organic layers combined. The resulting mixture was washed with 1×3 L of brine. The mixture was dried over anhydrous sodium sulfate and concentrated under vacuum. The residue was applied onto a silica gel column to afford title compound 13. MS (ESI): m/z calc'd for C₈H₁₄INO [M+H]⁺: 268, found 268. ¹H NMR (400 MHz, CDCl₃-d, 25° C.) δ 4.61 (m, 4H), 4.46-4.17 (m, 1H), 3.49 (m, 1H), 2.61-2.35 (m, 2H), 2.16 (m, 6H).

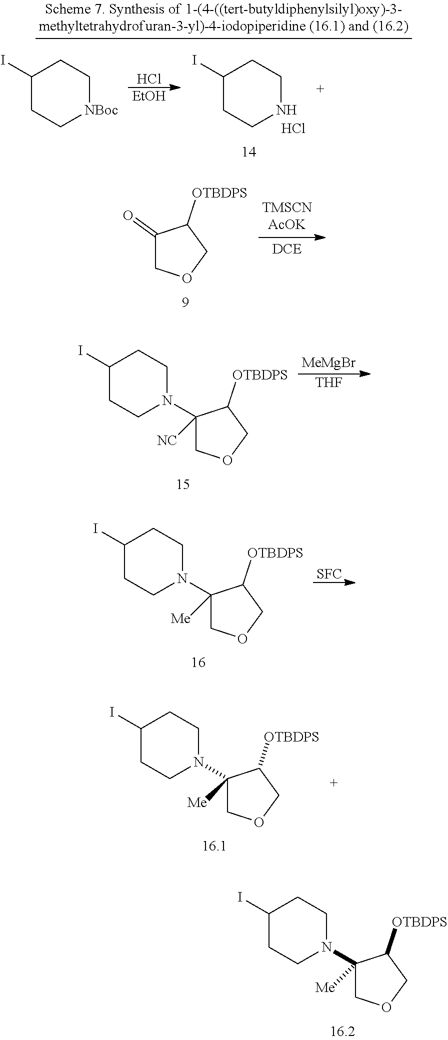

### 4-iodopiperidine hydrochloride (14)

Into a 10-L 4-necked round-bottom flask was placed tert-butyl 4-iodopiperidine-1-carboxylate (400.00 g, 1 eq.), EtOH (3.2 L). This was followed by the addition of HCl (gas) in 1,4-dioxane (1.6 L) dropwise with stirring at room temperature. The resulting solution was stirred for 16 h at room temperature. The resulting mixture was concentrated under vacuum. This resulted in the title compound 14.

### 4-((tert-butyldiphenylsilyl)oxy)-3-(4-iodopiperidin-1-yl)tetrahydrofuran-3-carbonitrile (15)

Into a 3-L 4-necked round-bottom flask purged and maintained with an inert atmosphere of nitrogen, was placed 4-iodopiperidine hydrochloride (250.00 g, 1010.101 mmol, 1.00 eq.), DCE (1250.00 mL), CH₃COOK (110.00 g, 1120.822 mmol, 1.11 eq.). The resulting solution was stirred for 1 h at 50° C., tert-butyldiphenylsilyl)oxy]oxolan-3-one, 9 (370.00 g, 1086.656 mmol, 1.08 eq.) was added at RT. The resulting solution was stirred for 1 h at 50° C. This was followed by the addition of TMSCN (150.00 g, 1511.998 mmol, 1.50 eq.) dropwise with stirring at 50° C. The resulting solution was stirred for 16 h at 50° C. The reaction was then quenched by the addition of aqueous sodium bicarbonate. The resulting solution was extracted with dichloromethane. The organic layers were combined and dried over anhydrous sodium sulfate and concentrated under vacuum. This resulted in the title compound 15.

### 1-((3S,4S)-4-((tert-butyldiphenylsilyl)oxy)-3-methyltetrahydrofuran-3-yl)-4-iodopiperidine and 1-((3R,4R)-4-((tert-butyldiphenylsilyl)oxy)-3-methyltetrahydrofuran-3-yl)-4-iodopiperidine (16.1) and (16.2)

Into a 5-L 4-necked round-bottom flask purged and maintained with an inert atmosphere of nitrogen, was placed 4-[(tert-butyldiphenylsilyl)oxy]-3-(4-iodopiperidin-1-yl)oxolane-3-carbonitrile (700.00 g, 1.249 mol, 1.00 eq., crude), THF (2 L). This was followed by the addition of MeMgBr (1200.00 mL) (3 mol/L) dropwise with stirring at <10° C. The resulting solution was stirred for 3 h at 50° C. The reaction was then quenched by the addition of aqueous sodium bicarbonate. The resulting solution was extracted with ethyl acetate and the organic layers combined and dried over anhydrous sodium sulfate and concentrated under vacuum. The crude product was purified by Flash-Prep-HPLC to afford title compound 16.

The crude product was purified by Prep-SFC with the following conditions (Prep SFC350-1): Column, CHIRALPAK AD-H SFC, 5*25 cm, 5 um; mobile phase, CO₂ (70%) and IPA (2 mM NH₃-MEOH) (30%); Detector, UV affording the title compounds 16.1 and 16.2. MS (ESI): m z calc'd for C₂₆H₃₆INO₂Si [M+H]⁺: 550, found 550. ¹H NMR (499 MHz, DMSO-d6) δ 7.80 (m, 2H), 7.71 (m, 2H), 7.53-7.35 (m, 6H), 4.28 (s, 1H), 4.09-3.96 (m, 2H), 3.90-3.76 (m, 2H), 3.65 (m, 1H), 2.62-2.52 (m, 1H), 2.42 (s, 1H), 2.24 (m, 1H), 2.06 (m, 4H), 1.11 (s, 9H), 0.94 (s, 3H).

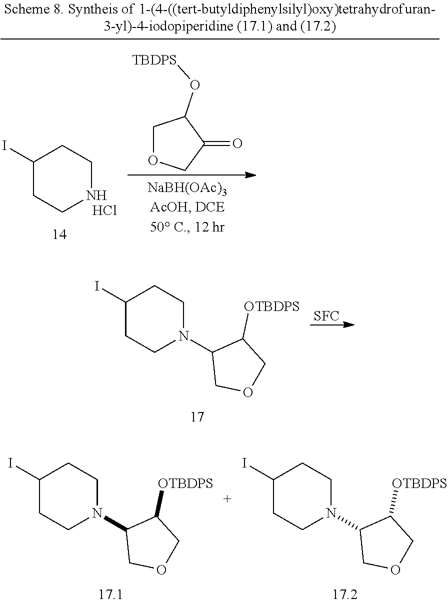

### 1-(4-((tert-butyldiphenylsilyl)oxy)tetrahydrofuran-3-yl)-4-iodopiperidine (17)

In a 5 L flask was added 4-iodopiperidine hydrochloride (121 g, 488.8 mmol, 1.0 eq, HCl), tert-butyldiphenylsilyl)oxy]oxolan-3-one (199.7 g, 586.6 mmol, 1.2 eq), molecular sieves (480 g) and DCE (2.50 L) forming a white suspension. The reaction mixture was stirred for 15 minutes at room temperature. Acetic acid (35.2 g, 586.6 mmol, 33.55 mL, 1.2 eq), and NaBH(OAc)₃ (259.0 g, 1.22 mol, 2.5 eq) were added to the reaction vessel. The reaction was then heated to 65° C. The reaction mixture was diluted with DCM and extracted with saturated 1M ammonium chloride. The organic layers were combined, dried over magnesium sulfate and concentrated under reduced pressure. The residue was purified by column chromatography to afford the title compound 17.

1-(4-((tert-butyldiphenylsilyl)oxy)tetrahydrofuran-3-yl)-4-iodopiperidine (111 g, 95.93 mmol) was separated by SFC (column: DAICEL CHIRALCEL OJ (250 mm*50 mm, 10 um); mobile phase: [0.1% NH₃H₂O EtOH]; B %: 30%-30%, 7 min) affording the title compounds 17.1 and 17.2. MS (ESI): m/z calc'd for C₂₆H₃₆INO₂Si [M+H]⁺: 536, found 536. ¹H NMR (499 MHz, DMSO-d6) δ 7.77-7.79 (m, 2H), 7.66-7.68 (m, 2H), 7.38-7.45 (m, 6H), 4.24-4.25 (m, 2H), 3.90-3.98 (m, 2H), 3.68-3.80 (m, 2H), 2.57-2.63 (m, 3H), 2.05-2.10 (m, 6H), 1.09 (s, 9H).

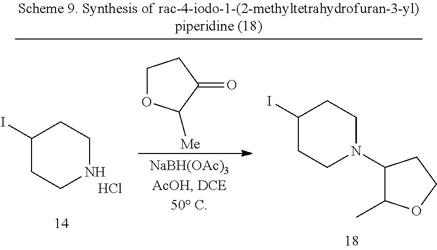

### Rac-4-iodo-1-(2-methyltetrahydrofuran-3-yl)piperidine (18)

To a vial were added 4-iodopiperidine, HCl (410 mg, 1.657 mmol), 2-methyldihydrofuran-3(2H)-one (498 mg, 4.97 mmol) and molecular sieves. The reagents were dissolved in DCE (8283 μl), and DIPEA (868 μl, 4.97 mmol) was added. The reaction was stirred for 1 hour at room temperature. Acetic acid (284 μl, 4.97 mmol) and sodium triacetoxyborohydride (1053 mg, 4.97 mmol) were added and heated to 50° C. overnight. The reaction was quenched with ammonium chloride, extracted with DCM, and then the combined organic layers were concentrated in vacuo. The residue was purified by column chromatography on silica gel to afford title compound 18. ¹H NMR (499 MHz, CDCl₃) δ ¹H NMR (499 MHz, Chloroform-d) δ 4.27 (m, 1H), 4.05 (t, J=6.3 Hz, 1H), 3.94 (m, 1H), 3.75 (m, 1H), 2.80 (m, 1H), 2.68 (m, 1H), 2.49 (m, 1H), 2.24 (m, 1H), 2.12 (s, 4H), 1.92 (m, 2H), 1.23 (m, 1H), 1.09 (d, J=6.4 Hz, 3H).

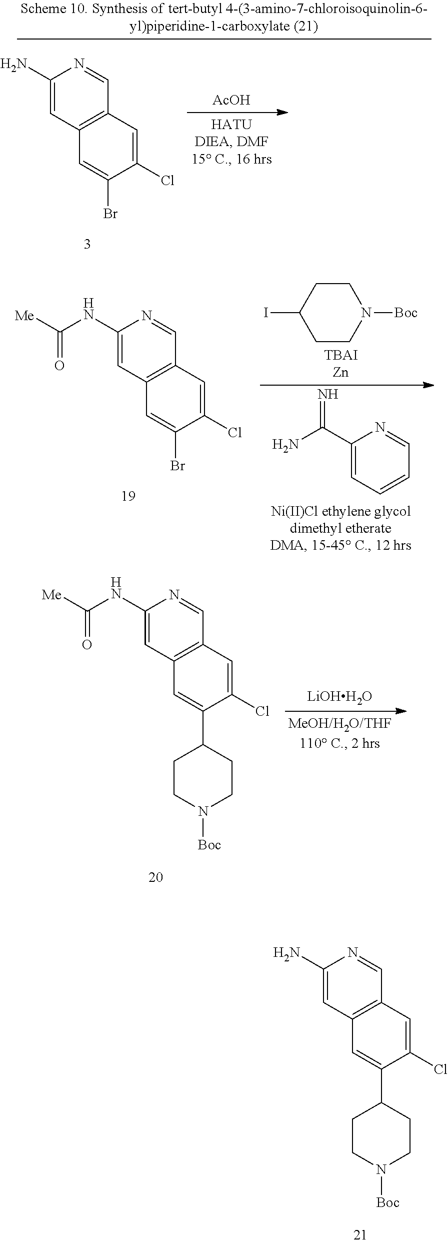

### N-(6-bromo-7-chloroisoquinolin-3-yl)acetamide (19)

Into a 1 L flask was added DMF (610 mL), followed by acetic acid (61.8 g, 1.03 mol, 58.8 mL 2.5 eq) and diisopropyl ethyl amine (212 g, 1.65 mol, 286 mL, 4 eq). HATU (391 g, 1.03 mol, 2.5 eq) and 6-bromo-7-chloroisoquinolin-3-amine (106 g, 411 mol, 1 eq) were added to the mixture. The reaction was stirred at 15° C. for 16 hours. The mixture was poured into water to form a precipitate. The solid was filtered out and washed with MTBE. The title compound was used without further purification.

tert-butyl 4-(3-acetamido-7-chloroisoquinolin-6-yl)piperidine-1-carboxylate (20) Into a 500 mL flask were added N-(6-bromo-7-chloroisoquinolin-3-yl)acetamide (5 g, 16.6 mmol, 1 eq), tert-butyl 4-iodopiperidine-1-carboxylate (7.79 g, 25 mmol, 1.5 eq), zinc (2.18 g, 33.3 mmol, 2 eq), and TBAI (1.23 g, 3.34 mmol, 0.2 eq). DMA (240 mL) was added at 40° C. The flask was evacuated with nitrogen. In a separate flask was added pyridine-2-carboxamidine hydrochloride (526 mg, 3.34 mmol, 0.2 eq) and dichloronickel 1,2-dimethoxyethane (733 mg, 3.34 mmol, 0.2 eq) in DMA (10 ml). The flask was evacuated and back filled with nitrogen 3 times and stirred for 10 minutes at 15° C. The nickel complex solution was added to the main reaction vessel and heated to 40° C. for 15 hours. The reaction was diluted with ethyl acetate and water. The reaction was filtered. The aqueous layers were extracted with ethyl acetate, washed with brine and concentrated under reduced pressure. Twenty-three identical reactions were combined to afford the title compound 20.

### tert-butyl 4-(3-amino-7-chloroisoquinolin-6-yl)piperidine-1-carboxylate (21)

Into a 100 mL flask, lithium hydroxide (42.2 g, 1.01 mol, 10 eq) was added into water (100 mL). In a 1 L flask was added tert-butyl 4-(3-acetamido-7-chloroisoquinolin-6-yl)piperidine-1-carboxylate (51 g, 100 mmol, 1 eq) dissolved in methanol (350 mL) and THF (100 mL). The saturated lithium hydroxide solution was added to the main reaction vessel. The reaction was evacuated, back filled with nitrogen 3 times and then heated to 110° C. for 2 hours. Two identical reactions were combined and concentrated under reduced pressure. The residue was filtered and washed with water (50 mL) and concentrated under vacuum. Tert-butyl 4-(3-amino-7-chloroisoquinolin-6-yl)piperidine-1-carboxylate was purified by prep-HPLC (column: Agela DuraShell C18 250*80 mm*10 um; mobile phase: [water (10 mM NH₄HCO₃)-ACN]; B %: 40%-70%, 20 min). The solution was concentrated under reduced pressure to afford the title compound 21. MS (ESI): m/z calc'd for C₁₉H₂₄ClIN₃O₂ [M+H]⁺: 362, found 362. ¹H NMR (400 MHz, CDCl₃-d, 25° C.) δ 8.75 (s, 1H), 7.91 (s, 1H), 7.60-7.43 (m, 1H), 6.59 (s, 1H), 6.02 (s, 2H), 4.13 (m, 2H), 3.11 (m, 1H), 2.87 (m, 2H), 1.91-1.76 (m, 2H), 1.63-1.49 (m, 2H), 1.43 (s, 9H).

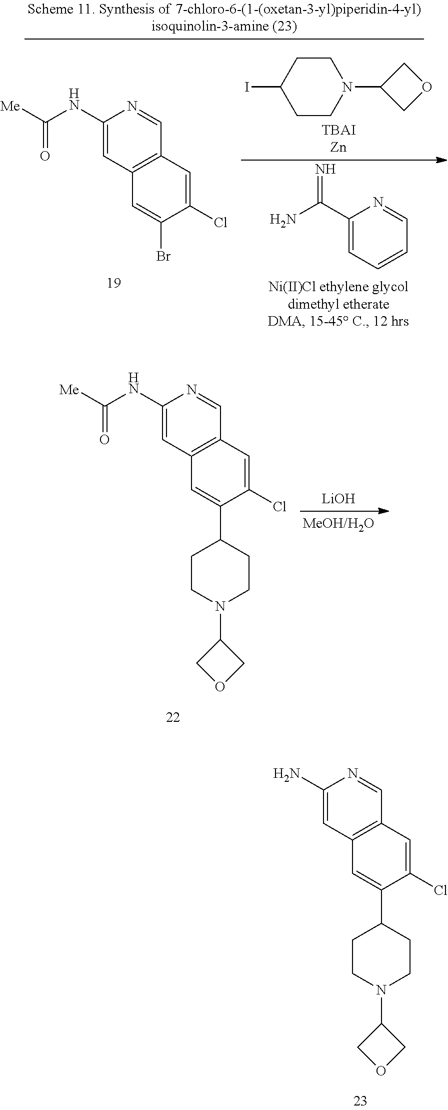

### N-(7-chloro-6-(1-(oxetan-3-yl)piperidin-4-yl)isoquinolin-3-yl)acetamide (22)

A vial was charged with nickel(II) chloride ethylene glycol dimethyl ether complex (12.91 mg, 0.059 mmol) and picolinimidamide (7.12 mg, 0.059 mmol), then sealed and its contents were placed under an inert atmosphere. DMA (1469 μl) was added, and the resulting mixture was stirred for 5 minutes at room temperature. In a separate vial, N-(6-bromo-7-chloroisoquinolin-3-yl)acetamide 19 (88 mg, 0.294 mmol), 4-iodo-1-(oxetan-3-yl)piperidine (157 mg, 0.588 mmol), TBAI (21.70 mg, 0.059 mmol), and zinc (57.6 mg, 0.881 mmol) were added. The vial was sealed and its contents were placed under an inert atmosphere. The nickel complex was added through the septum and the resulting mixture was stirred at 75° C. for 2 hours. The reaction mixture was diluted with ethyl acetate and washed twice with water and once with brine. The combined organic fractions were dried over anhydrous sodium sulfate, filtered and concentrated under reduced pressure. The reaction mixture was filtered, purified by HPLC, eluting acetonitrile/water gradient with 0.1% TFA modifier, linear gradient and lyophilized to afford title compound 22.

### 7-chloro-6-(1-(oxetan-3-yl)piperidin-4-yl)isoquinolin-3-amine (23)

LiOH (100 mg, 4.18 mmol) was added to a solution of N-(7-chloro-6-(1-(oxetan-3-yl)piperidin-4-yl)isoquinolin-3-yl)acetamide 22 (80 mg, 0.222 mmol) in methanol (3 ml) and water. The resulting mixture was stirred for 9 hours at 50° C., then concentrated under reduced pressure, diluted with DCM and washed twice with water and once with brine. The combined organic fractions were dried over anhydrous sodium sulfate, filtered and concentrated under reduced pressure to afford title compound 23. MS (ESI): m/z calc'd for C₁₇H₂₀ClN₃O [M+H]⁺: 318, found 318.

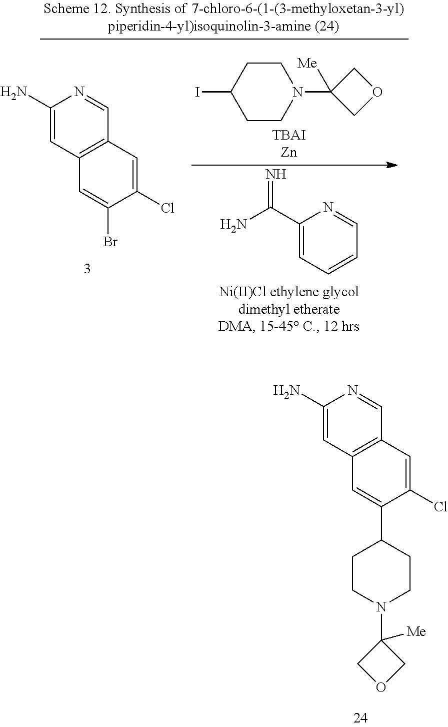 [SMILES: Nc1cc2cc(Br)c(Cl)cc2cn1 ; CC1(N2CCC(I)CC2)COC1 ; N=C(N)c1ccccn1 ; CC1(N2CCC(c3cc4cc(N)ncc4cc3Cl)CC2)COC1]

### 7-chloro-6-(1-(3-methyloxetan-3-yl)piperidin-4-yl)isoquinolin-3-amine (24)

Around bottom flask was charged with 6-bromo-7-chloroisoquinolin-3-amine 3 (1.0 g, 3.88 mmol), 4-iodo-1-(3-methyloxetan-3-yl)piperidine (1.638 g, 5.82 mmol), picolinimidamide hydrochloride (0.367 g, 2.330 mmol), NiCl₂·dme (0.427 g, 1.942 mmol), manganese (0.640 g, 11.65 mmol) and TBAI (1.434 g, 3.88 mmol). The solids were dissolved in DMA (30 ml), and the solution was stirred at 70° C. for 3 h, then added dropwise to a solution of EtOAc (100 mL) and pet. ether (100 mL) with vigorous stirring. The solution was filtered and the solid residue was collected. The residue was purified by flash column chromatography to give title compound 24. MS (ESI): m/z calc'd for C₁₈H₂₂ClN₃O [M+H]⁺: 332, found 332.

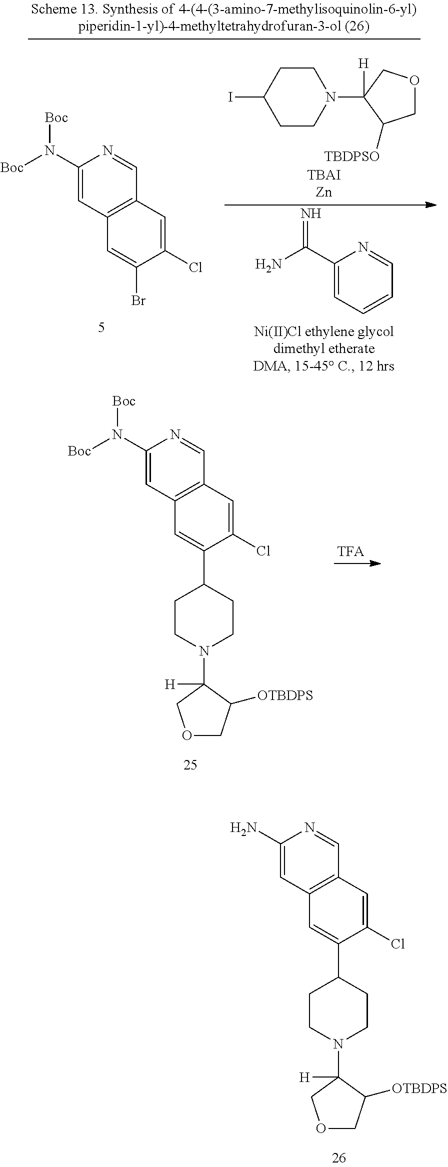

### N-(6-(1-(4-((tert-butyldiphenylsilyl)oxy)-3-methyltetrahydrofuran-3-yl)piperidin-4-yl)-7-fluoroisoquinolin-3-yl)-6-oxaspiro[2.5]octane-1-carboxamide (25)

A vial was charged with nickel (II) chloride ethylene glycol dimethyl ether complex (96 mg, 0.437 mmol) and picolinimidamide hydrochloride (68.9 mg, 0.437 mmol). The vial was sealed, and its contents were placed under an inert atmosphere. DMA (5 mL) was added, and the resulting mixture was stirred for 5 minutes at room temperature. In a separate vial, tert-butyl (6-bromo-7-chloroisoquinolin-3-yl)(tert-butoxycarbonyl)carbamate 5 (1000 mg, 2.185 mmol), TBAI (161 mg, 0.437 mmol), and zinc (428 mg, 6.55 mmol) were added. The vial was sealed and its contents were placed under an inert atmosphere. A solution of 1-(4-((tert-butyldiphenylsilyl)oxy)tetrahydrofuran-3-yl)-4-iodopiperidine 17 (2340 mg, 4.37 mmol) in DMA (5.00 mL) was added through the septum. The previously mentioned nickel complex was added through the septum, and the resulting mixture was stirred for 2 hours at 75° C. The reaction mixture was filtered through a pad of celite and concentrated under reduced pressure. The residue was purified by column chromatography on silica to afford title compound 25.

### 4-(4-(3-amino-7-methylisoquinolin-6-yl)piperidin-1-yl)-4-methyltetrahydrofuran-3-ol (26)

TFA (1500 μl, 19.47 mmol) was added to a solution of tert-butyl (tert-butoxycarbonyl)(6-(1-(4-((tert-butyldiphenylsilyl)oxy)tetrahydrofuran-3-yl)piperidin-4-yl)-7-chloroisoquinolin-3-yl)carbamate (815 mg, 1.036 mmol) in DCM (2.5 mL). The resulting mixture was stirred at room temperature for 2 hours. The reaction mixture was quenched with a saturated aqueous sodium bicarbonate solution, extracted with DCM, washed with brine. The combined organic fractions were dried over anhydrous sodium sulfate, filtered and concentrated under reduced pressure to afford title compound 26. MS (ESI): m/z calc'd for C₃₄H₄₁ClN₃O₂Si [M+H]⁺: 586, found 586.

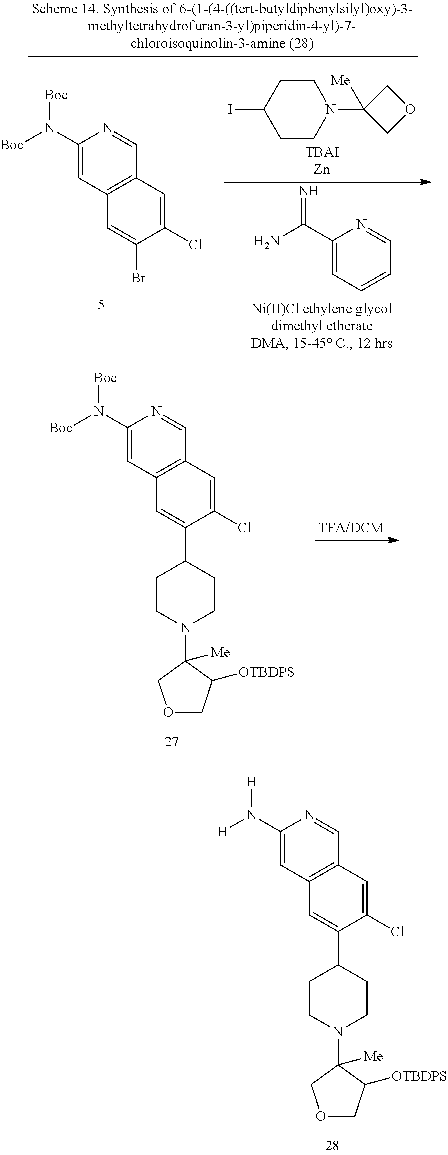

### Tert-butyl (tert-butoxycarbonyl)(6-(1-(4-((tert-butyldiphenylsilyl)oxy)-3-methyltetrahydrofuran-3-yl)piperidin-4-yl)-7-chloroisoquinolin-3-yl)carbamate (27)

A vial was charged with pyridine-2-carboximidamide hydrochloride (4.74 mg, 0.030 mmol) and nickel(II) chloride ethylene glycol dimethyl ether complex (6.61 mg, 0.030 mmol). The flask was evacuated and back filled with nitrogen 3 times and DMA (752 μl) was added and stirred for 10 minutes. In a separate vial N-(6-bromo-7-chloroisoquinolin-3-yl)-1-methyl-1H-pyrazole-4-carboxamide (55 mg, 0.150 mmol), 1-(4-((tert-butyldiphenylsilyl)oxy)-3-methyltetrahydrofuran-3-yl)-4-iodopiperidine 12 (124 mg, 0.226 mmol), TBAI (11.11 mg, 0.030 mmol), and zinc (29.5 mg, 0.451 mmol). The flask was evacuated and back filled with nitrogen 3 times. The nickel solution was added to the second vial and heated to 80° C. The reaction was heated for 1 hour. The residue was purified by column chromatography on silica gel to afford the title compound 27.

### 6-(1-(4-((tert-butyldiphenylsilyl)oxy)-3-methyltetrahydrofuran-3-yl)piperidin-4-yl)-7-chloroisoquinolin-3-amine (28)

TFA (3061 μl) was added to a solution of tert-butyl(tert-butoxycarbonyl)(6-(1-(4-((tert-butyldiphenylsilyl)oxy)-3-methyltetrahydrofuran-3-yl)piperidin-4-yl)-7-chloroisoquinolin-3-yl)carbamate (980 mg, 1.224 mmol) in DCM (9.1 mL). The resulting mixture was stirred for 1 hour at room temperature. The mixture was concentrated under reduced to pressure to afford title compound 28. MS (ESI): m/z calc'd for C₃₅H₄₂ClN₃O₂Si [M+H]⁺: 600, found 600.

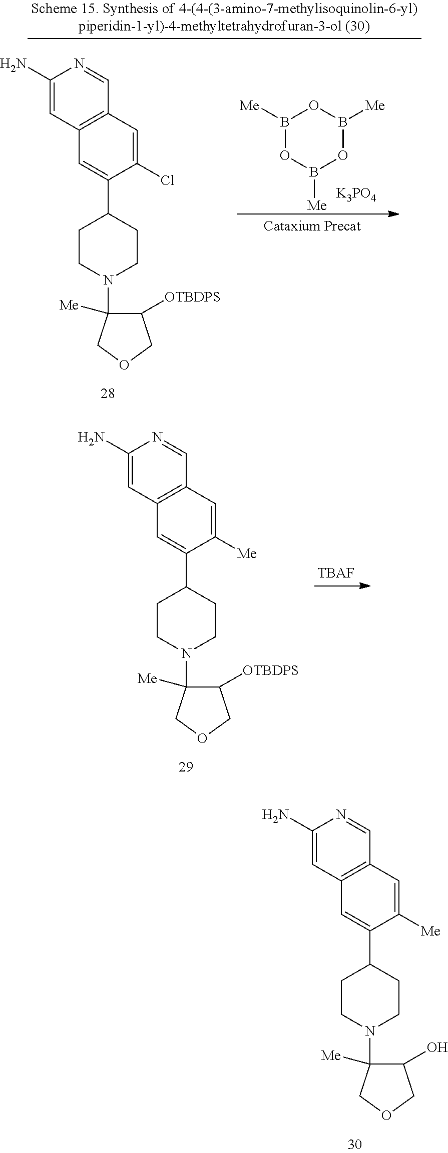

### 6-(1-(4-((tert-butyldiphenylsilyl)oxy)-3-methyltetrahydrofuran-3-yl)piperidin-4-yl)-7-methylisoquinolin-3-amine (29)

A vial was charged with 6-(1-(4-((tert-butyldiphenylsilyl)oxy)-3-methyltetrahydrofuran-3-yl)piperidin-4-yl)-7-chloroisoquinolin-3-amine, 2HCl 27 (500 mg, 0.743 mmol), 2,4,6-trimethyl-1,3,5,2,4,6-trioxatriborinane (416 μl, 2.97 mmol), potassium phosphate, tribasic (473 mg, 2.228 mmol), and Cataxium Pd G3 (54.1 mg, 0.074 mmol). The vial was sealed and its contents were placed under an inert atmosphere. Dioxane (3342 μl) and water (371 μl) were added through the septum, and the resulting mixture was stirred for 72 hours at 80° C. The reaction mixture was diluted with ethyl acetate and washed twice with saturated aqueous ammonium chloride and once with brine. The combined organic fractions were dried over anhydrous magnesium sulfate, filtered and concentrated under reduced pressure. The residue was purified by column chromatography on silica to afford title compound 29.

### 4-(4-(3-amino-7-methylisoquinolin-6-yl)piperidin-1-yl)-4-methyltetrahydrofuran-3-ol (30)

TBAF (1M in THF) (1121 μl, 1.121 mmol) was added to a solution of 29 (325 mg, 0.560 mmol) in THF (2500 μl). The resulting mixture was stirred overnight at room temperature. A precipitate formed overnight and was collected by vacuum filtration and washed with iPAc to afford title compound 30. MS (ESI): m/z calc'd for C₂₀H₂₈N₃O₂Si [M+H]⁺: 342, found 342.

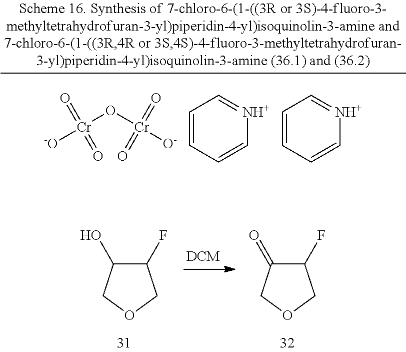

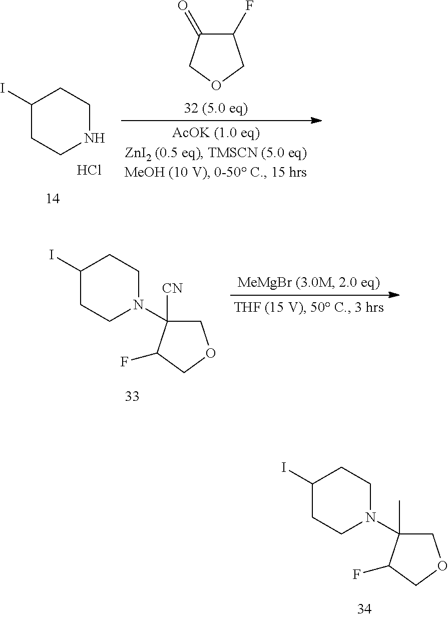

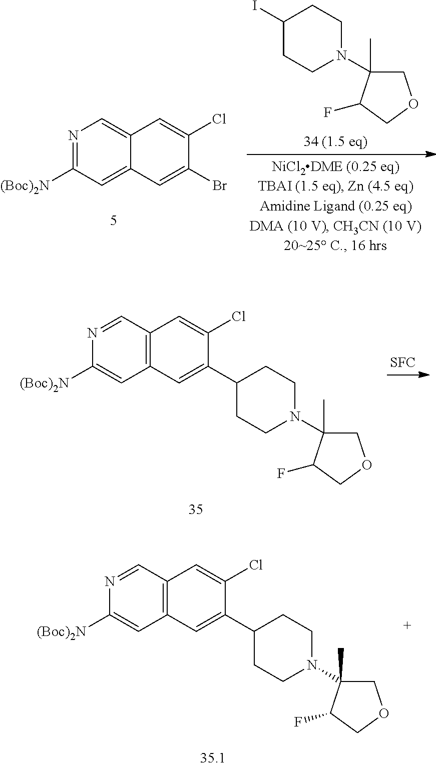

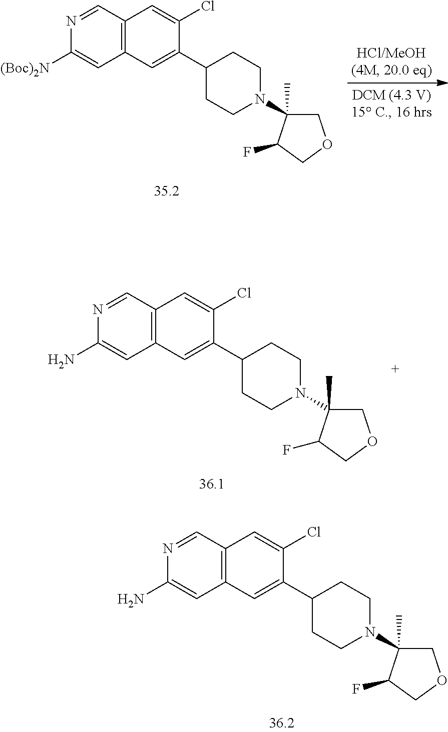

### 4-fluorodihydrofuran-3(2H)-one (32)

A vial was charged with 4-fluorotetrahydrofuran-3-ol 31 (1.5 g, 14.14 mmol), pyridinium dichromate (10.64 g, 28.3 mmol) and DCM (47.1 ml). The vial was sealed, and its contents were placed under an inert atmosphere by performing 3 vacuum/nitrogen cycles and the resulting mixture was stirred at room temperature overnight for 16 hours. The reaction mixture was filtered through a celite pad and the eluant was concentrated. The resultant crude residue was subjected to purification by silica gel chromatography (Hexanes in 3:1 EtOAc/EtOH, 0-100%) to afford title compound 32. ¹HNMR (400 MHz, CDCl₃) δ 4.89-5.05 (m, 1H), 4.40-4.42 (m, 1H), 3.88-4.04 (m, 3H).

### 4-fluoro-3-(4-iodopiperidin-1-yl)tetrahydrofuran-3-carbonitrile (33)

A vial was charged with 4-iodopiperidine, HCl (100 g, 404 mmol), AcOK (39.6 g, 404 mmol, 1.0 eq) and MeOH (1 L). The vial was sealed, and its contents were placed under an inert atmosphere by performing 3 vacuum/nitrogen cycles and the resulting mixture was stirred at 50° C. for 1 hr. 4-fluorodihydrofuran-3(2H)-one (3.50 kg, 2.02 mol) and ZnI₂ (64.5 g, 202 mmol) were added through the rubber septum, and the resulting mixture was stirred at room temperature for 2 hrs. At this point TMSCN (200 g, 2.02 mol) was added to the reaction mixture dropwise at 0° C. The resulting mixture was stirred at room temperature for 12 hrs. At 12 hrs, the reaction was diluted with DCM (1 L) and quenched by dropwise addition of 1M sodium hydroxide (1 L mL). The combined organic phases were washed with H₂O (1 L), dried over Na₂SO₄, and the solvent removed under reduced pressure. The crude residue was triturated with MTBE (40.0 mL) and petroleum ether (100 mL) at 15° C. for 12 hrs. The solid was filtered to afford title compound 33. MS (ESI) m/z calc'd for C₁₀H₁₄FIN₂O [M+H]⁺: 325, found 325.

### 1-(4-fluoro-3-methyltetrahydrofuran-3-yl)-4-iodopiperidine (34)

A vial was charged with 4-fluoro-3-(4-iodopiperidin-1-yl)tetrahydrofuran-3-carbonitrile (30.0 g, 92.6 mmol) and THF (450 mL). The vial was sealed, and its contents were placed under an inert atmosphere by performing 3 vacuum/nitrogen cycles. Methylmagnesium Bromide (61.7 mL, 3M in THF) was added through the septum, and the resulting mixture was allowed to stir overnight at 50° C. At 16 hrs, the reaction was diluted with DCM (500 mL) and quenched by dropwise addition of saturated ammonium chloride (500 mL). The combined organic phases were washed with H₂O (500 mL), dried over Na₂SO₄, and the solvent removed under reduced pressure. The resultant crude residue was subjected to purification by silica gel chromatography (0-100% EtOAc in hexanes) to afford title compound 34. MS (ESI) m/z calc'd for C₁₀H₁₇FINO [M+H]⁺: 314, found 314.

### Ditert-butyl (7-chloro-6-(1-((3R,4R and 3S,4S)-4-fluoro-3-methyltetrahydrofuran-3-yl)piperidin-4-yl)isoquinolin-3-yl)carbamate (35.1) and (35.2)

A vial was charged with ditert-butyl (6-bromo-7-chloroisoquinolin-3-yl)carbamate (40.0 g, 87.4 mmol), Zn (25.7 g, 393 mmol), 1-(4-fluoro-3-methyltetrahydrofuran-3-yl)-4-iodopiperidine (41.0 g, 131 mmol), and TBAI (48.4 g, 131 mmol). The vial was sealed, and its contents were placed under an inert atmosphere by performing 3 vacuum/nitrogen cycles. DMA (400 mL), NiCl₂·DME (4.80 g, 21.8 mmol), pyridine-2-carboxamidine; hydrochloride (3.44 g, 21.8 mmol) in CH₃CN (400 mL) were added through the rubber septum. The resulting mixture was stirred at room temperature overnight. At 16 hrs the crude mixture was filtered through a Celite pad and concentrated. The concentrated mixture was poured into an Erlenmeyer flask containing water (2 L) to precipitate the desired product. The crude residue filtered and subjected to purification by reversed phase HPLC, eluting with water (0.1% NH₄OH)-ACN to afford 35. The racemic material could be resolved to its component enantiomers by chiral preparative SFC (Column & dimensions: DAICEL CHIRALPAK AD (250 mm×50 mm, 10 μm); Mobile phase: [0.1% NH₃H₂O EtOH]; B %: 20%-20%, 8.7 min) to afford title compounds 35.1 and 35.2. MS (ESI) m/z calc'd for C₂₉H₃₉ClFN₃O₅ [M+H]⁺: 564, found 564. ¹HNMR: (400 MHz, DMSO-d6) δ 9.16 (s, 1H), 8.30 (s, 1H), 8.11 (s, 1H), 7.80 (s, 1H), 4.86-5.01 (m, 1H), 4.06-4.20 (m, 1H), 3.82-3.92 (m, 1H), 3.74 (d, J=7.6 Hz, 1H), 3.66 (d, J=7.6 Hz, 1H), 2.98-3.08 (m, 2H), 2.52-2.55 (m, 2H), 2.45-2.48 (m, 1H), 1.71-1.90 (m, 4H), 1.40 (s, 18H), 1.02 (s, 3H). MS (ESI) m/z calc'd for C₂₉H₃₉ClFN₃O₅ [M+H]⁺: 564, found 564. ¹HNMR: (400 MHz, DMSO-d6) δ 9.16 (s, 1H), 8.30 (s, 1H), 8.11 (s, 1H), 7.80 (s, 1H), 4.86-5.00 (m, 1H), 4.06-4.20 (m, 1H), 3.82-3.92 (m, 1H), 3.74 (d, J=7.6 Hz, 1H), 3.66 (d, J=7.6 Hz, 1H), 2.98-3.08 (m, 2H), 2.52-2.55 (m, 2H), 2.45-2.48 (m, 1H), 1.73-1.90 (m, 4H), 1.40 (s, 18H), 1.01 (s, 3H).

### 7-chloro-6-(1-((3S,4S or 3R,4R)-4-fluoro-3-methyltetrahydrofuran-3-yl)piperidin-4-yl)isoquinolin-3-amine (36.1) or (36.2)

A vial was charged with ditert-butyl (7-chloro-6-(1-((3S,4S)-4-fluoro-3-methyltetrahydrofuran-3-yl)piperidin-4-yl)isoquinolin-3-yl)carbamate 35.1 or 35.2 (3.00 g, 5.32 mmol) and DCM (15.0 mL). The vial was sealed, and its contents were placed under an inert atmosphere by performing 3 vacuum/nitrogen cycles. HCl/MeOH (4 M, 26.6 mL) was added dropwise through the septum, and the resulting reaction mixture was stirred at room temperature for 16 hrs. At that time, the crude mixture was concentrated to give a residue. The pH of the residue was adjusted to pH=7 by adding saturated sodium bicarbonate. The desired product was extracted with DCM (2×100 mL). The combined organic phases were washed with H₂O (500 mL), dried over Na₂SO₄, and the solvent removed under reduced pressure which afforded title compound 36.1 or 36.2. MS (ESI) m/z calc'd for C₁₉H₂₃ClFN₃O [M+H]⁺: 364, found 364. ¹HNMR: (400 MHz, DMSO-d6) δ 8.73 (s, 1H), 7.88 (s, 1H), 7.53 (s, 1H), 6.60 (s, 1H), 5.99 (s, 2H), 4.85-4.99 (m, 1H), 4.06-4.19 (m, 1H), 3.82-3.92 (m, 1H), 3.74 (d, J=7.6 Hz, 1H), 3.66 (d, J=7.6 Hz, 1H), 2.88-2.98 (m, 2H), 2.52-2.55 (m, 2H), 2.41-2.45 (m, 1H), 1.68-1.84 (m, 4H), 1.00 (s, 3H).

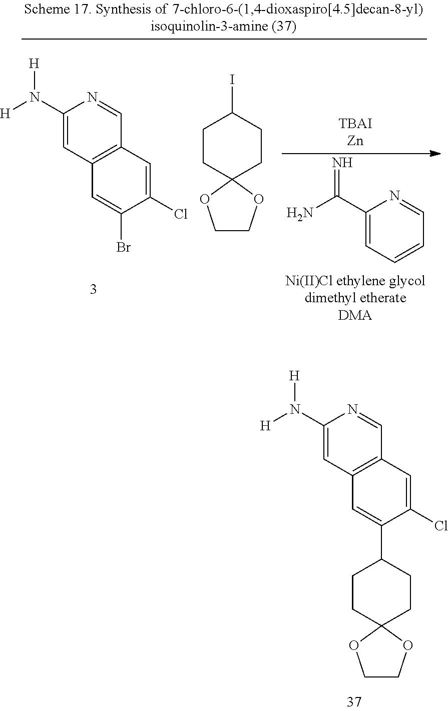

### 7-chloro-6-(1,4-dioxaspiro[4.5]decan-8-yl)isoquinolin-3-amine (37)

A vial was charged with 6-bromo-7-chloroisoquinolin-3-amine 3 (200 mg, 0.777 mmol), 8-iodo-1,4-dioxaspiro[4.5]decane (625 mg, 2.33 mmol), TBAI (1148 mg, 3.11 mmol) and zinc (203 mg, 3.11 mmol). In a separate vial, nickel (II) chloride ethylene glycol dimethyl ether complex (188 mg, 0.854 mmol) and pyridine-2-carboximidamide hydrochloride (184 mg, 1.165 mmol) were added. The vial was evacuated and back filled with nitrogen 3 times, and the solids were dissolved in DMA (7.8 mL) and stirred for 10 minutes to complex. The solution was added to the main reaction vial and heated to 80° C. overnight. The reaction was poured into water, and a solid formed and was collected. The residue was purified by column chromatography on silica gel to afford title compound 37. MS (ESI): m/z calc'd for C₁₇H₂₀ClN₂O₂ [M+H]⁺: 319, found 319.

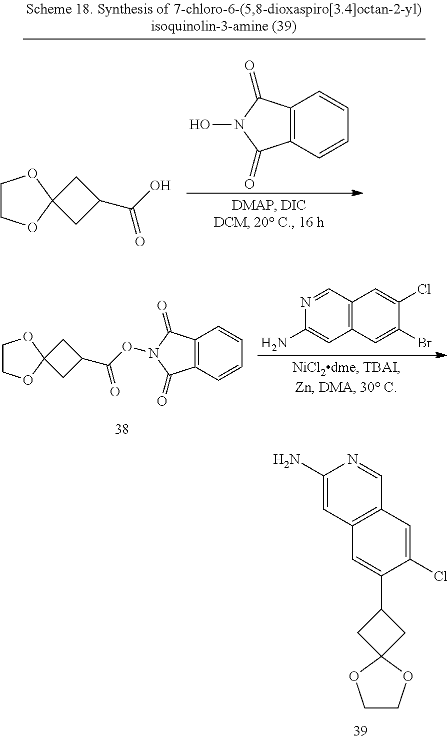

### 1,3-dioxoisoindolin-2-yl 5,8-dioxaspiro[3.4]octane-2-carboxylate (38)

To a mixture of 5,8-dioxaspiro[3.4]octane-2-carboxylic acid (1.2 g, 7.59 mmol), 2-hydroxyisoindoline-1,3-dione (1.362 g, 8.35 mmol) and DMAP (0.093 g, 0.759 mmol) in anhydrous DCM (20 mL) was added DIC (1.300 mL, 8.35 mmol), and the resulting mixture was stirred at 20° C. for 16 h. The reaction mixture was filtered through a pad of Celite and was washed with DCM. The filtrate was concentrated in vacuo to give the residue which was purified by flash silica gel chromatography to afford the title compound 38. ¹H NMR (500 MHz, CDCl₃) δ=7.91-7.77 (m, 4H), 3.96-3.89 (m, 4H), 3.27 (m, 1H), 2.91-2.81 (m, 2H), 2.77-2.66 (m, 2H)

### 7-chloro-6-(5,8-dioxaspiro[3.4]octan-2-yl)isoquinolin-3-amine (39)

To a solution of 6-bromo-7-chloroisoquinolin-3-amine 3 (200 mg, 0.777 mmol) and zinc (203 mg, 3.11 mmol) in DMA (6 mL) were added tetrabutylammonium iodide (230 mg, 0.621 mmol), nickel (II) chloride ethylene glycol dimethyl ether complex (188 mg, 0.854 mmol), 1,3-dioxoisoindolin-2-yl 5,8-dioxaspiro[3.4]octane-2-carboxylate (471 mg, 1.553 mmol) and N-cyano-4-methoxypicolinimidamide (205 mg, 1.165 mmol). The mixture was stirred for 16 h at 30° C. After filtration and concentration, EtOAc and brine were added into the mixture. The organic layer was separated. The organic phase was washed with brine and the organic layer was dried over anhydrous Na₂SO₄, filtered and concentrated in vacuo. The crude product was purified by prep-TLC to afford title compound 39. MS (ESI): m/z calc'd for C₁₅H₁₆ClN₂O₂ [M+H]⁺: 291, found 291.

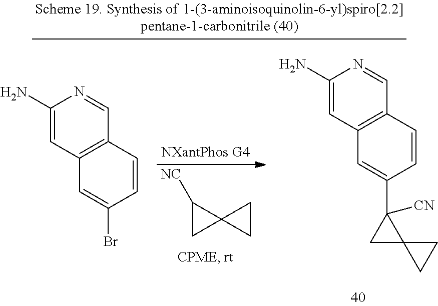

A round bottom was charged with NXanthPhos G4 (363 mg, 0.388 mmol) and 6-bromo-8-chloroisoquinolin-3-amine (500 mg, 1.942 mmol). The flask was evacuated and back filled with nitrogen 3 times. CPME (9708 μl) was added to the vessel followed by spiro[2.2]pentane-1-carbonitrile (199 mg, 2.136 mmol). LiHMDS (7767 μl, 7.77 mmol) was added to the reaction and stirred overnight at room temperature. The residue was purified by column chromatography on silica gel, eluting with hexanes/3:1 ethyl acetate:ethanol to afford title compound 40. MS (ESI): m/z calc'd for C₁₅H₁₃ClN₃ [M+H]⁺: 270, found 270.

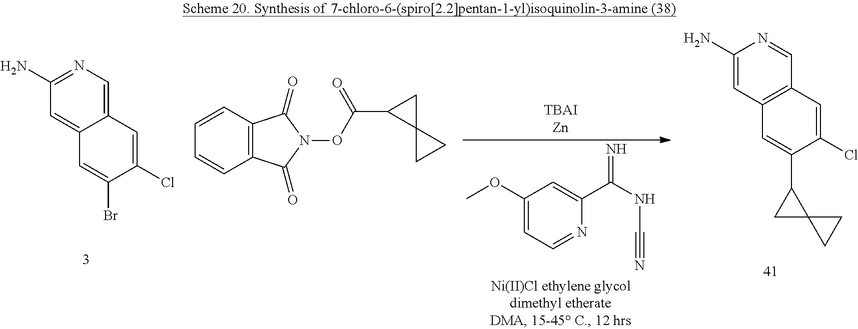

To a solution of 6-bromo-7-chloroisoquinolin-3-amine 3 (300 mg, 1.165 mmol) and 1,3-dioxoisoindolin-2-yl spiro[2.2]pentane-1-carboxylate (345 mg, 1.340 mmol) in DMA (10 ml) were added tetrabutylammonium iodide (344 mg, 0.932 mmol), nickel (II) chloride ethylene glycol dimethyl ether complex (282 mg, 1.281 mmol), N-cyano-4-methoxypicolinimidamide (308 mg, 1.747 mmol) and zinc (305 mg, 4.66 mmol). The mixture was stirred for 16 h at 30° C. The reaction was filtered and concentrated in vacuo, extracted with ethyl acetate and washed with brine. The organic layer was dried over anhydrous Na₂SO₄, filtered and concentrated in vacuo. The crude product as purified by prep-TLC(SiO₂, Pet.ether:EtOAc=1:1) to afford title compound 38. MS (ESI): m/z calc'd for C₁₄H₁₄ClN₂ [M+H]⁺: 246, found 246.

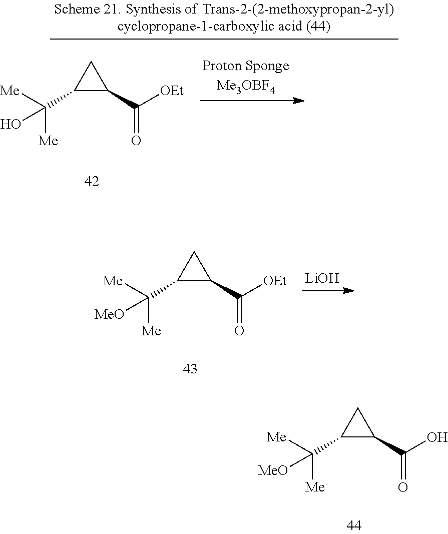

### ethyl-2-(2-methoxypropan-2-yl)cyclopropane-1-carboxylate (43)

To a vial was added ethyl-2-(2-hydroxypropan-2-yl)cyclopropane-1-carboxylate 42 (500 mg, 2.90 mmol), and 1,8-bis(dimethylamino)napthalene (1244 mg, 5.81 mmol). DCM (1.45E+04 μl) were added followed by trimethyloxonium tetrafluoroborate (859 mg, 5.81 mmol) in portions. The reaction was stirred overnight. The reaction was washed with ammonium chloride, extracted with DCM and concentrated in vacuo. The residue was purified by column chromatography on silica gel to afford title compound 43. ¹H NMR (499 MHz, CDCl₃) δ 4.23-4.10 (m, 2H), 3.23 (s, 3H), 1.28 (m, 3H), 1.15 (m, 6H), 0.98 (m, 2H), 0.90 (m, 2H).

### Trans-2-(2-methoxypropan-2-yl)cyclopropane-1-carboxylic acid (44)

To a vial was added trans-ethyl-2-(2-methoxypropan-2-yl)cyclopropane-1-carboxylate (260 mg, 1.396 mmol) which was dissolved in THF (3490 μl). Lithium hydroxide (334 mg, 13.96 mmol) in water (3490 μl) was added, and the reaction stirred at room temperature overnight, then extracted with ethyl acetate, acidified with 1M HCl, extracted with DCM and concentrated in vacuo. The residue was purified by column chromatography on silica gel to afford title compound 44. ¹H NMR (499 MHz, CDCl₃) δ 3.23 (s, 3H), 1.75-1.58 (m, 2H), 1.36-1.27 (m, 1H), 1.17 (m, 6H), 1.06 (m, 1H).

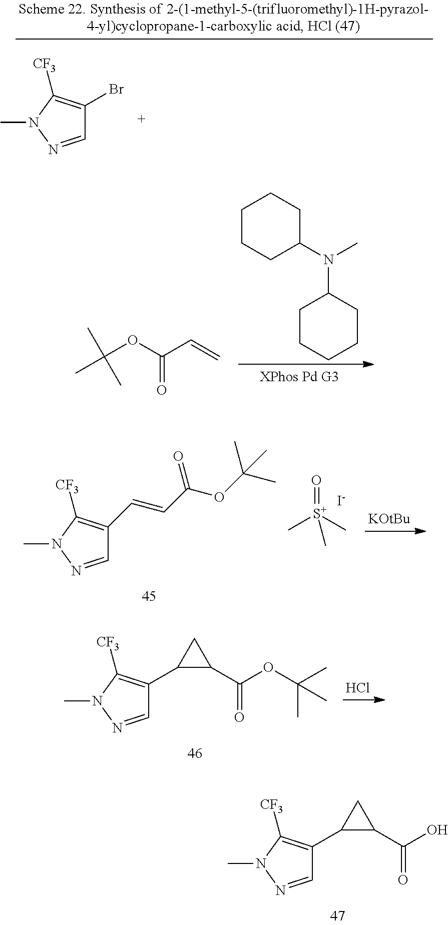

### tert-butyl (E)-3-(1-methyl-5-(trifluoromethyl)-1H-pyrazol-4-yl)acrylate (45)

In a ventilated balance enclosure, a 20 mL microwave vial equipped with a magnetic stirrer was charged with 4-bromo-1-methyl-5-(trifluoromethyl)-1H-pyrazole (1.00 g, 4.37 mmol), tertbutyl acrylate (1.919 ml, 13.10 mmol), and XPHOS PD G3 (0.370 g, 0.437 mmol). The vial was then sealed with a microwave cap, evacuated and backfilled with N₂ three times. Then, under a positive flow of argon anhydrous DMF (6 mL) was added. Finally, N,N-dicyclohexylmethylamine (2.81 ml, 13.10 mmol) was added. The reaction was heated to 90° C. overnight. The mixture was diluted with EtOAc and transferred to a separatory funnel containing sat. aq. NH₄Cl. The phases were separated, and the aqueous phase was extracted once more with EtOAc. The combined organic layers were washed with brine, dried over Na₂SO₄, filtered, and the collected filtrate concentrated to dryness in vacuo. The crude residue was purified by column chromatography on silica gel to afford title compound 45.

### tert-butyl 2-(1-methyl-5-(trifluoromethyl)-1H-pyrazol-4-yl)cyclopropane-1-carboxylate (46)

In a ventilated balance enclosure, a 30 mL scintillation vial equipped with a magnetic stirrer was charged with potassium tert-butoxide (944 mg, 8.42 mmol) and trimethylsulfoxonium iodide (1852 mg, 8.42 mmol). Then, under a positive flow of argon anhydrous DMSO (5 mL) was added. The mixture was stirred at RT for 30 minutes. Meanwhile, a separate solution of tert-butyl (E)-3-(1-methyl-5-(trifluoromethyl)-1H-pyrazol-4-yl)acrylate (775 mg, 2.81 mmol) in DMSO (10 mL) was prepared. This solution was transferred into the mixture containing the sulfur ylid, and the resultant reaction was stirred at RT overnight. The reaction mixture was carefully quenched by addition of saturated aqueous NH₄Cl. EtOAc was added, and the biphasic mixture was then transferred to a separatory funnel where the phases were separated. The combined organic layers were dried over Na₂SO₄, filtered, and concentrated to dryness in vacuo. The crude residue was purified by column chromatography on silica gel to afford title compound 46. MS (ESI): m/z calc'd for C₁₃H₁₈F₃N₂O₂ [M+H]⁺: 291, found 291.

### 2-(1-methyl-5-(trifluoromethyl)-1H-pyrazol-4-yl)cyclopropane-1-carboxylic acid, HC (47)

In a ventilated balance enclosure, a 30 mL scintillation vial equipped with a magnetic stirrer was charged with tert-butyl 2-(1-methyl-5-(trifluoromethyl)-1H-pyrazol-4-yl)cyclopropane-1-carboxylate (317 mg, 1.092 mmol). Then, under a positive flow of argon anhydrous dioxane (5.46 mL) was added, followed by HCl in dioxane (2.730 mL, 10.92 mmol). Conversion at RT was very slow even after 2 days. A another 10 equiv of HCl was added, and the reaction mixture was and heated to 60° C. The mixture was co-evaporated with MeCN in order to remove excess HCl, and the crude residue was carried on directly without further purification. MS (ESI): m z calc'd for C₉H₁₀F₃N₂O₂ [M+H]⁺: 234, found 234.

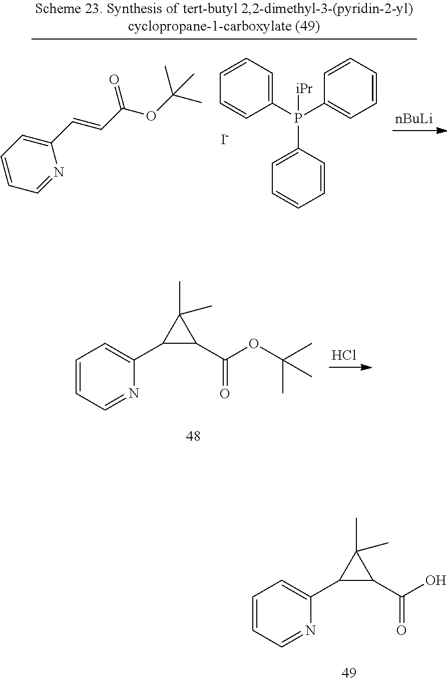

### tert-butyl 2,2-dimethyl-3-(pyridin-2-yl)cyclopropane-1-carboxylate (48)

In a ventilated balance enclosure, a 40 mL scintillation vial equipped with a magnetic stirrer was charged with isopropyltriphenyl phosphonium iodide (853 mg, 1.973 mmol). Then, under a positive flow of argon, anhydrous THF (2.5 mL) was added. The suspension was cooled to 0° C., and to the stirring suspension under inert atmosphere was added 1.00 equiv of n-BuLi. At 10 minutes another 1 equiv of n-BuLi was added. After 15 minutes or so, a solution of tert-butyl (E)-3-(pyridin-2-yl)acrylate (135 mg, 0.658 mmol) in THF (4 mL) was added to the stirring ylide at 0° C. An additional 500 μL of THF was used to wash the source vial and syringe, and added to the reaction mixture. The mixture was stirred overnight, allowing to warm to RT. The reaction was quenched carefully with sat. aq. NH₄Cl, diluted with EtOAc and additional H₂O, and transferred to a separatory funnel where the phases were separated. The aqueous phase was extracted once more with EtOAc. The combined extracts were washed with brine, dried over anhydrous Na₂SO₄, filtered, and the collected filtrate concentrated to dryness in vacuo. The crude residue was purified by column chromatography on silica gel (chromatogram attached) to afford the title compound 48. MS (ESI): m/z calc'd for C₁₅H₂₂NO₂ [M+H]⁺: 248, found 248.

### tert-butyl 2,2-dimethyl-3-(pyridin-2-yl)cyclopropane-1-carboxylate (49)

In a ventilated balance enclosure, a 30 mL scintillation vial equipped with a magnetic stirrer was charged with tert-butyl 2,2-dimethyl-3-(pyridin-2-yl)cyclopropane-1-carboxylate (93 mg, 0.376 mmol). Then, under a positive flow of argon anhydrous dioxane (3.760 mL) was added. Finally, hydrochloric acid (1.880 mL, 7.52 mmol) (4 M in dioxane) was added, and the reaction was warmed to 45° C. and stirred at this temperature for 3 hrs. The reaction was removed from heat, and volatiles were removed. The residue was re-dissolved in MeCN and co-evaporated. This process was repeated once more, and the resultant residue was carried forward without further purification. MS (ESI): m/z calc'd for C₁₁H₁₄NO₂ [M+H]⁺: 192, found 192. Compounds in Table 1 below were prepared in accordance with the synthetic sequences illustrated in Scheme 22 and Scheme 23 using the corresponding starting materials.

TABLE 1
Scheme 24. Synthesis of 1-(1-methyl-1H-pyrazol-4-yl)pyrrolidine-3-carboxylic acid (51)

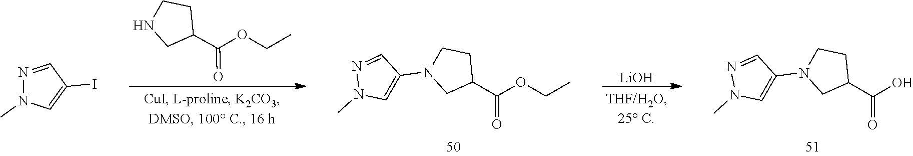

| | | | |
|---|---|---|---|
| Int-1.1 | 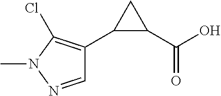 | 2-(5-chloro-1-methyl-1H-pyrazol-4-yl) cyclopropane-1-carboxylic acid | Cal'c: 201 Found: 201 |
| Int-1.2 | 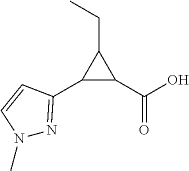 | 2-ethyl-3-(1-methyl-1H-pyrazol-3-yl) cyclopropane-1-carboxylic acid | Cal'c: 195 Found 195 |
| Int-1.3 | 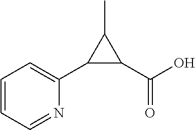 | 2-methyl-3-(pyridin-2- yl)cyclopropane-1-carboxylic acid | Cal'c: 178 Found: 178 |
| Int-1.4 | 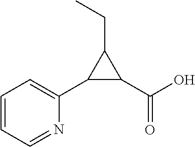 | 2-ethyl-3-(pyridin-2- yl)cyclopropane-1-carboxylic acid | Cal'c: 192 Found: 192 |

### ethyl 1-(1-methyl-1H-pyrazol-4-yl)pyrrolidine-3-carboxylate (50)

To a solution of 4-iodo-1-methyl-1H-pyrazole (1 g, 4.81 mmol), ethyl pyrrolidine-3-carboxylate hydrochloride (2.59 g, 14.42 mmol) and L-proline (0.554 g, 4.81 mmol) in anhydrous DMSO (20 mL) was added K₂CO₃ (2.66 g, 19.23 mmol) and copper(I) iodide (0.458 g, 2.404 mmol). The resulting mixture was stirred at 100° C. for 16 hours, poured into water and extracted with EtOAc. The organic layer was washed with water, dried over Na₂SO₄. After filtration and concentration, the crude product was purified by flash silica gel chromatography to afford title compound 50. MS (ESI): m/z calc'd for C₁₁H₁₈N₃O₂ [M+H]⁺: 224, found 224.

### 1-(1-methyl-1H-pyrazol-4-yl)pyrrolidine-3-carboxylic acid (51)

To a solution of ethyl 1-(1-methyl-1H-pyrazol-4-yl)pyrrolidine-3-carboxylate (350 mg, 1.568 mmol) in THF (5 mL) and H₂O (1 mL) was added lithium hydroxide monohydrate (197 mg, 4.70 mmol), and the resulting mixture was stirred at 25° C. for 16 hours. After filtration and concentration, the crude product was purified by prep-HPLC (base) to afford title compound 51. MS (ESI): m/z calc'd for C₉H₁₄N₃O₂ [M+H]⁺: 196, found 196.

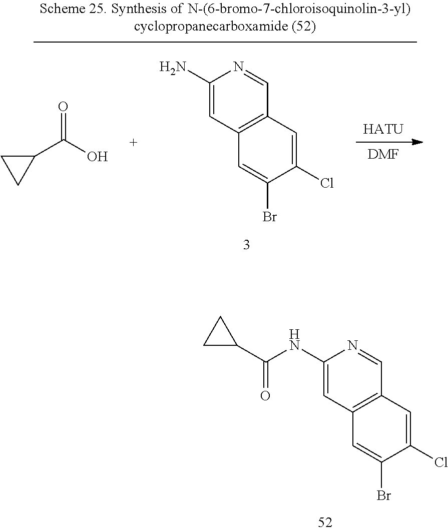

A 20 mL vial was charged with 6-bromo-7-chloroisoquinolin-3-amine 3 (400 mg, 1.553 mmol), cyclopropanecarboxylic acid (247 μl, 3.11 mmol), HATU (1181 mg, 3.11 mmol), DMF (4000 μl), and DIEA (1356 μl, 7.77 mmol). The mixture was allowed to stir overnight at room temperature, diluted with EtOAc and washed twice with water and once with brine. The combined organic fractions were dried over anhydrous sodium sulfate, filtered and concentrated under reduced pressure. The residue was purified by column chromatography on silica (0-100% EtOAc/hexanes). The desired fractions were pooled and concentrated under reduced pressure to afford the title compound 52. MS (ESI): m/z calc'd for C₁₃H₁₁BrClN₂O [M+H]⁺: 327, found 327. Compounds in Table 2 below were prepared in accordance with the synthetic sequences illustrated in Scheme 25 using the corresponding starting materials.

TABLE 2

| | | | |
|---|---|---|---|
| Int-2.2 | 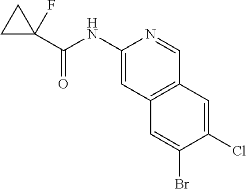 [SMILES: O=C(Nc1cc2cc(Br)c(Cl)cc2cn1)C1(F)CC1] | N-(6-bromo-7- chloroisoquinolin-3-yl)-1- fluorocyclopropane-1- carboxamide | Cal'c: 343 Found: 343 |
| Int-2.3 | 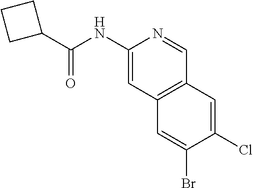 [SMILES: O=C(Nc1cc2cc(Br)c(Cl)cc2cn1)C1CCC1] | N-(6-bromo-7- chloroisoquinolin-3- yl)cyclobutanecarboxamide | Cal'c: 338 Found: 338 |
| Int-2.4 | 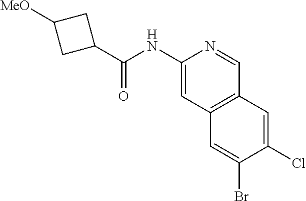 [SMILES: COC1CC(C(=O)Nc2cc3cc(Br)c(Cl)cc3cn2)C1] | cis or trans-N-(6-bromo-7- chloroisoquinolin-3-yl)-3- methoxycyclobutane-1- carboxamide | Cal'c: 369 Found: 369 |
| Int-2.5 | 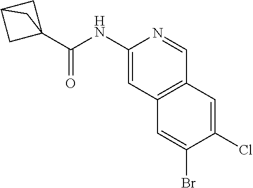 [SMILES: O=C(Nc1cc2cc(Br)c(Cl)cc2cn1)C12CC(C1)C2] | N-(6-bromo-7- chloroisoquinolin-3- yl)bicyclo[1.1.1]pentane-1- carboxamide | Cal'c: 352 Found: 352 |
| Int-2.6 | 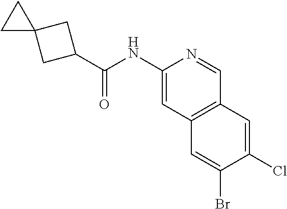 [SMILES: O=C(Nc1cc2cc(Br)c(Cl)cc2cn1)C1CC2(CC2)C1] | N-(6-bromo-7- chloroisoquinolin-3- yl)spiro[2.3]hexane-5- carboxamide | Cal'c: 365 Found: 365 |
| Int-2.7 | 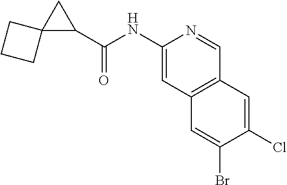 [SMILES: O=C(Nc1cc2cc(Br)c(Cl)cc2cn1)C1CC12CCC2] | Rac-N-(6-bromo-7- chloroisoquinolin-3- yl)spiro[2.3]hexane-1- carboxamide | Cal'c: 365 Found: 365 |
| Int-2.8 | 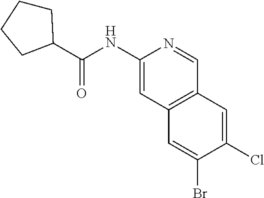 [SMILES: O=C(Nc1cc2cc(Br)c(Cl)cc2cn1)C1CCCC1] | N-(6-bromo-7- chloroisoquinolin-3- yl)cyclopentanecarboxamide | Cal'c: 353 Found: 353 |
| Int-2.9 | 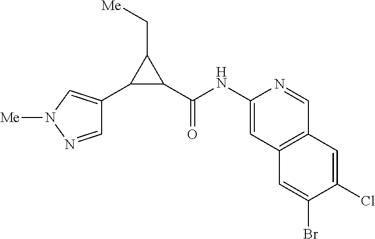 [SMILES: CCC1C(C(=O)Nc2cc3cc(Br)c(Cl)cc3cn2)C1c1cnn(C)c1] | trans-N-(6-bromo-7- chloroisoquinolin-3-yl)-2- ethyl-3-(1-methyl-1H- pyrazol-4-yl)cyclopropane- 1-carboxamide | Cal'c: 433 Found: 433 |
| Int-2.10 | 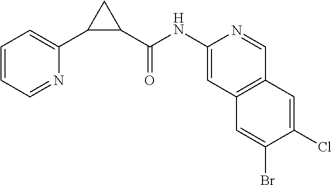 [SMILES: O=C(Nc1cc2cc(Br)c(Cl)cc2cn1)C1CC1c1ccccn1] | trans-N-(6-bromo-7- chloroisoquinolin-3-yl)-2- (pyridin-2-yl)cyclopropane- 1-carboxamide | Cal'c: 401 Found: 401 |
| Int-2.11 | 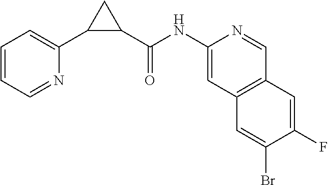 [SMILES: O=C(Nc1cc2cc(Br)c(F)cc2cn1)C1CC1c1ccccn1] | trans-N-(6-bromo-7- fluoroisoquinolin-3-yl)-2- (pyridin-2-yl)cyclopropane- 1-carboxamide | Cal'c: 386 Found: 386 |
| Int-2.12 | 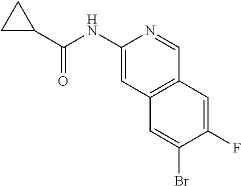 [SMILES: O=C(Nc1cc2cc(Br)c(F)cc2cn1)C1CC1] | N-(6-bromo-7- fluoroisoquinolin-3- yl)cyclopropanecarboxamide | Cal'c: 309 Found: 309 |
| Int-2.13 | 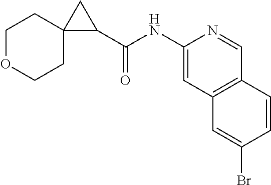 [SMILES: O=C(Nc1cc2cc(Br)ccc2cn1)C1CC12CCOCC2] | N-(6-bromoisoquinolin-3- yl)-6-oxaspiro[2.5]octane-1- carboxamide | Cal'c: 363 Found: 363 |

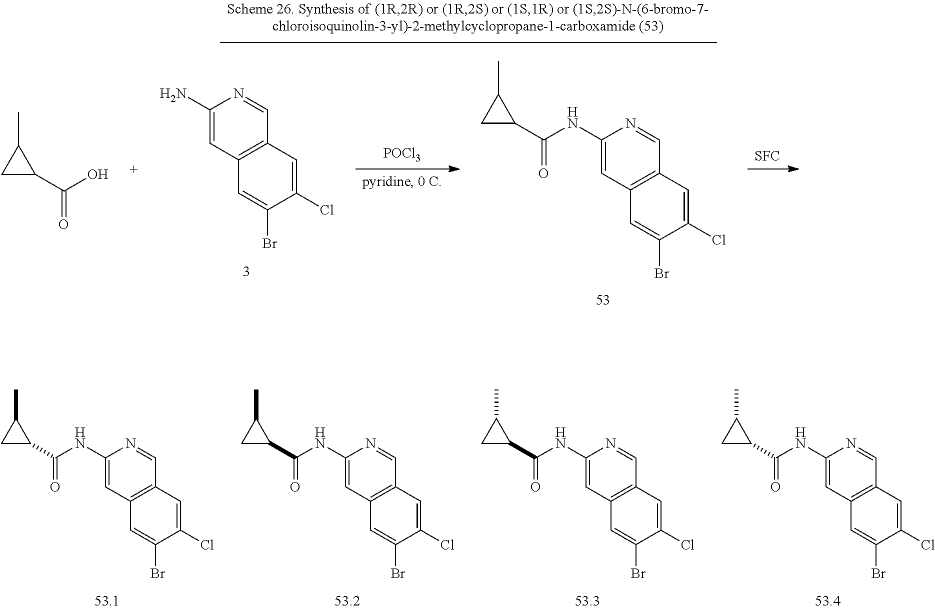

A vial was charged with a solution of 6-bromo-7-chloroisoquinolin-3-amine (100 mg, 0.388 mmol) and 2-methylcyclopropanecarboxylic acid (46.7 mg, 0.466 mmol) in pyridine (1 ml) at 0° C. to give a brown mixture. POCl₃ (80 μl, 0.858 mmol) was added to the mixture and stirred at 0° C. for 2 h. The reaction mixture was quenched with sat. NH₄Cl and extracted with EtOAc. The combined organic phases were washed with brine (50 mL), dried over anhydrous Na₂SO₄, filtered and concentrated under reduced pressure. The residue was purified by Pre-HPLC (Column YMC-Actus Triart C18 150*30 mm*5 um Condition water (0.2 mM HFBA+0.1% TFA)-ACN) to give a mixture of enantiomers. The mixture of four stereoisomers was purified by chiral SFC (IG-3, 100×4.6 (mm), Mobile phase: 40% iPrOH (0.05% DEA) in CO₂ afford the title compounds 53.1 (tR=1.9 min), 53.2 (tR=2.1 min), 53.3 (tR=2.3 min) and 53.4 (tR=4.1 min). MS (ESI): m/z calc'd for C₁₄H₁₄BrClN₂O [M+2H]⁺: 340, found 340.

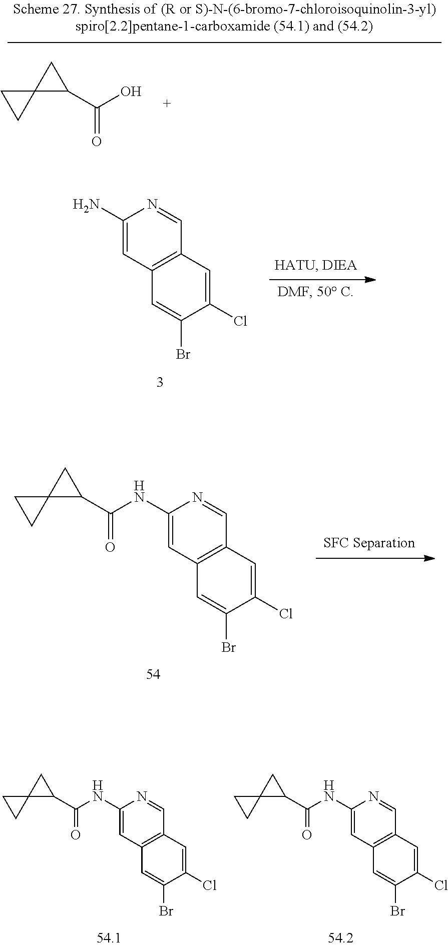

A vial was charged with 6-bromo-7-chloroisoquinolin-3-amine 3 (250 mg, 0.971 mmol), spiro[2.2]pentane-1-carboxylic acid (169 mg, 1.507 mmol), and HATU (517 mg, 1.359 mmol). DMF (5000 μl), and DIEA (1000 μl, 5.73 mmol) were added. The reaction was allowed to stir overnight at 50° C., then cooled to room temperature. Water was added to form a precipitate. The solids were collected by vacuum filtration and dried. The residue was purified by column chromatography on silica (0-100% EtOAc/hexanes). The mixture of two stereoisomers was purified by chiral SFC (OJ-H, 21×250 (mm), Mobile phase A: CO₂: Mobile phase B: MeOH 0.1% NH₄OH) to afford the title compounds 54.1 (tR=4.0 min) and 54.2 (tR=5.4 min). 54.1: MS (ESI): m/z calc'd for C₁₅H₁₃BrClN₂O [M+H]⁺: 351, found 351. 54.2: MS (ESI): m/z calc'd for C₁₅H₁₃BrClN₂O [M+H]⁺: 351, found 351.

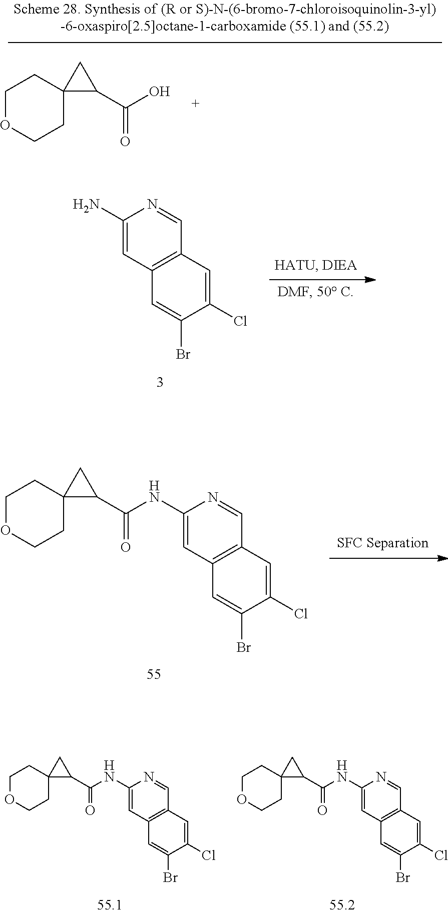

A round bottom flask was charged with 6-bromo-7-chloroisoquinolin-3-amine 3 (10 g, 38.8 mmol), 6-oxaspiro[2.5]octane-1-carboxylic acid (12.13 g, 78 mmol), and HATU (29.5 g, 78 mmol). DMF (97 ml), and DIEA (33.9 ml, 194 mmol) were added. The reaction was allowed to stir 72 h. at 50° C., then was cooled to room temperature and added to 1.6 L of water to form a precipitate. The solids were collected by vacuum filtration and dried under reduced pressure. The residue was purified by column chromatography on silica (20-100% EtOAc:hexanes). The mixture of two stereoisomers was purified by chiral SFC (OJ-H, 21×250 (mm), Mobile phase A: 25% CO₂ Mobile phase B: 75% MeOH 0.1% NH₄OH) and concentrated to afford the title compounds 55.1 (tR=4.0 min) and 55.2 (tR=5.4 min). 55.1: MS (ESI): m/z calc'd for C₁₇H₁₇BrClN₂O₂ [M+H]⁺: 395, found 395. 55.2: MS (ESI): m/z calc'd for C₁₇H₁₇BrClN₂O₂ [M+H]⁺: 395, found 395. Final stereochemistry was not determined.

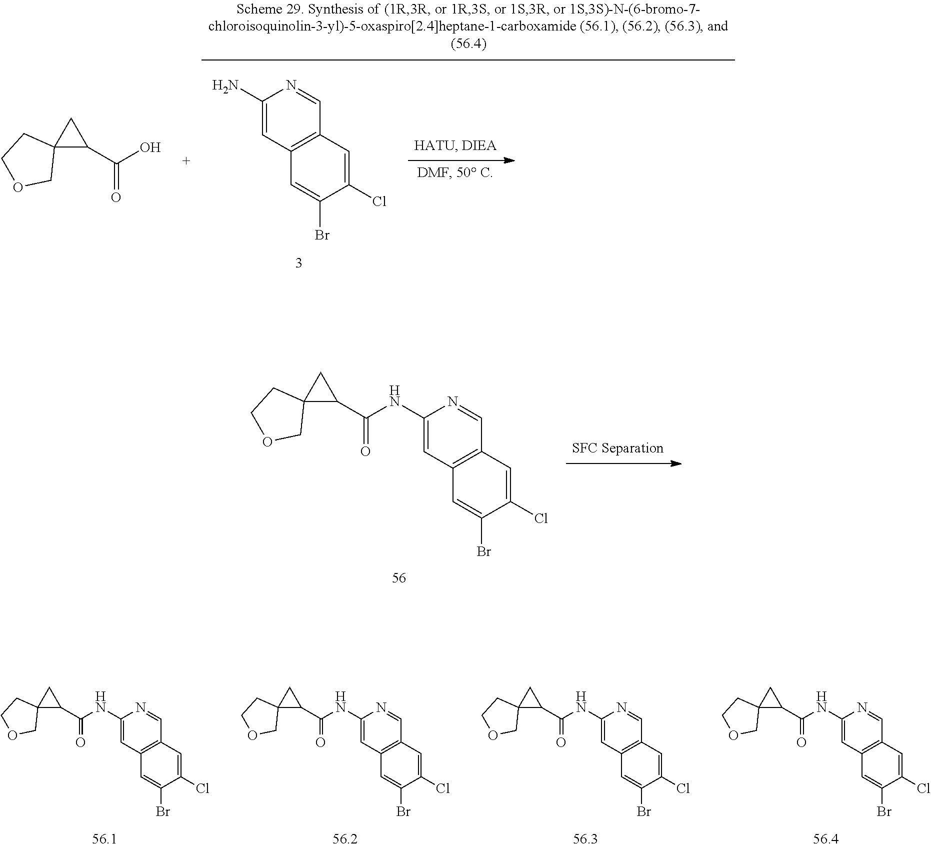

A vial was charged with 5-oxaspiro[2.4]heptane-1-carboxylic acid (331 mg, 2.330 mmol), 6-bromo-7-chloroisoquinolin-3-amine 3 (500 mg, 1.942 mmol) and HATU (1477 mg, 3.88 mmol). DMF (6472 μl) and DIPEA (678 μl, 3.88 mmol) were added. The reaction was heated to 50° C. overnight, then cooled to room temperature and added to 30 mL of water. The solid was filtered, collected, and purified through a silica gel column 0-100% 3:1 EtOAc:EtOH/hexanes. The mixture of two stereoisomers was purified by chiral SFC (Lux-3, 21×250 (mm), Mobile phase A: 15% CO₂ Mobile phase B: 85% MeOH 0.1% NH₄OH) and concentrated to afford the title compounds 56.1 (tR=6.0 min), 56.2 (tR=6.9 min), 56.3 (tR=7.3 min) and 56.4 (tR=8.9 min). 56.1: MS (ESI): m/z calc'd for C₁₆H₁₅BrClN₂O₂ [M+H]⁺: 381, found 381. 56.2: MS (ESI): m z calc'd for C₁₆H₁₅BrClN₂O₂ [M+H]⁺: 381, found 381. 56.3: MS (ESI): m/z calc'd for C₁₆H₅BrClN₂O₂ [M+H]⁺: 381, found 381. 56.4: MS (ESI): m/z calc'd for C₁₆H₁₅BrClN₂O₂ [M+H]⁺: 381, found 381. Final stereochemistry was not determined.

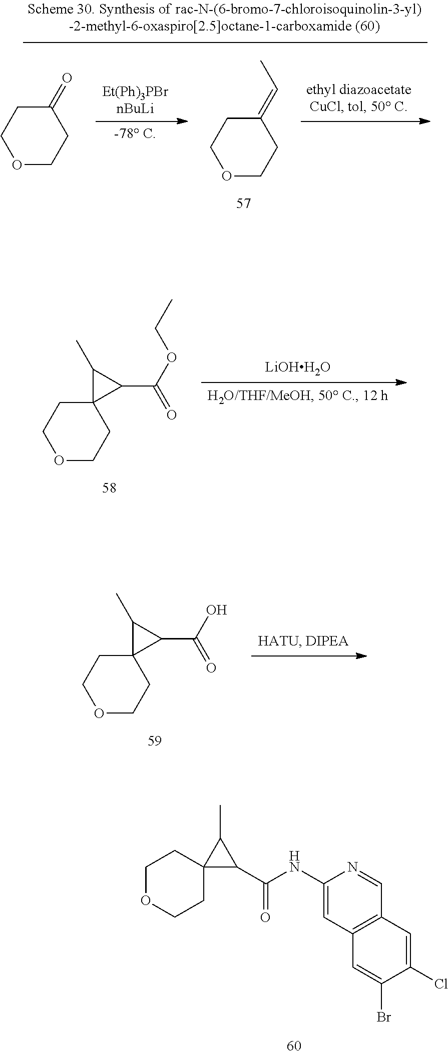

### 4-ethylidenetetrahydro-2H-pyran (57)

N-butyllithium (43.9 mL, 110 mmol) was added dropwise at 0° C. to a solution of ethyltriphenyl phosphonium bromide (40.8 g, 110 mmol) in diethyl ether (400 mL). The reaction mixture was stirred for 20 minutes, then tetrahydro-4H-pyran-4-one (10 g, 100 mmol) in diethyl ether (50 mL) was added. The mixture was warmed to room temperature and stirred for 2 hours. The crude reaction mixture was diluted with diethyl ether and washed with water. The layers were separated and the aqueous layer was re-extracted with diethyl ether. The combined organic layers were dried over sodium sulfate, filtered, and carefully concentrated under reduced pressure at low temperature. The resulting solid was diluted with hexanes and filtered. The filtrate was concentrated under reduced pressure to provide the title compound 57. ¹H NMR (400 MHz, CDCl₃) δ 5.28-5.19 (m, 1H), 3.69-3.61 (m, 4H), 2.26 (t, J=5.5 Hz, 2H), 2.21-2.16 (m, 2H), 1.58 (d, J=6.7 Hz, 3H).

### ethyl 2-methyl-6-oxaspiro[2.5]octane-1-carboxylate (58)

A suspension of copper (I) chloride (2.118 g, 21.40 mmol) and 4-ethylidenetetrahydro-2H-pyran (4.8 g, 42.8 mmol) in toluene (100 mL) was stirred for 20 min under N₂ atmosphere. The suspension was heated to 50° C. and stirred for 10 min. Ethyl diazoacetate (22.60 mL, 214 mmol) was added to the solution dropwise over 4.5 h at 50° C. The reaction was stirred for 16 h at 50° C., then filtered, and the filtrate was concentrated in vacuo to give a residue, which was purified by flash silica gel chromatography to afford the title compound 58. MS (ESI): m/z calc'd for C₁₇H₁₇BrClN₂O₂ [M+H]⁺: 395, found 199.

### Rac-2-methyl-6-oxaspiro[2.5]octane-1-carboxylic acid (59)

A suspension of ethyl 2-methyl-6-oxaspiro[2.5]octane-1-carboxylate (500 mg, 2.52 mmol) and lithium hydroxide monohydrate (317 mg, 7.57 mmol) in MeOH (2.5 mL), H₂O (5 mL) and THF (2.5 mL) was heated to 50° C. and stirred for 12 h. Water (20 mL) was added to the mixture, and the mixture was adjusted with HCl (4M) to pH ˜2, then extracted with EtOAc. The combined organic layers were washed with brine, dried over anhydrous Na₂SO₄ and filtered. The filtrate was concentrated in vacuo to afford title compound 59. ¹H NMR (400 MHz, CDCl₃) δ 3.73-3.59 (m, 3H), 3.59-3.46 (m, 1H), 1.92-1.78 (m, 1H), 1.77-1.71 (m, 1H), 1.67-1.39 (m, 2H), 1.39-1.26 (m, 1H), 1.22-1.14 (m, 2H), 1.09 (d, J=6.4 Hz, 1H)

### Rac-N-(6-bromo-7-chloroisoquinolin-3-yl)-2-methyl-6-oxaspiro[2.5]octane-1-carboxamide (60)

A solution of 6-bromo-7-chloroisoquinolin-3-amine (400 mg, 1.553 mmol), 2-methyl-6-oxaspiro[2.5]octane-1-carboxylic acid (344 mg, 2.019 mmol) in DCM (10 mL) and pyridine (0.5 mL) was cooled to 0° C. under N₂ atmosphere. Then POCl₃ (0.290 mL, 3.11 mmol) in DCM (1.0 mL) was added to the solution dropwise. The reaction was stirred for 12 h at 25° C. under N2 atmosphere. The mixture was poured into ice-water and extracted with DCM. The combined organic layers were washed with brine, dried over anhydrous Na₂SO₄ and filtered. The filtrate was concentrated in vacuo to give a residue, which was purified by flash silica gel chromatography to afford title compound 60. MS (ESI): m/z calc'd for C₁₇H₁₇BrClN₂O₂ [M+H]⁺: 395, found 411.

### General Synthetic Schemes and Preparative Examples

The compounds of the invention may be prepared by methods known in the art of organic synthesis as set forth in part by the following general synthetic schemes and specific preparative examples. Starting materials are available commercially or may be prepared by known methods. In Tables 1 through 18, generally the racemic compounds were not tested, unless otherwise indicated or is an example number having a single listed compound. Example numbers are assigned only to the isolated resolved compounds.

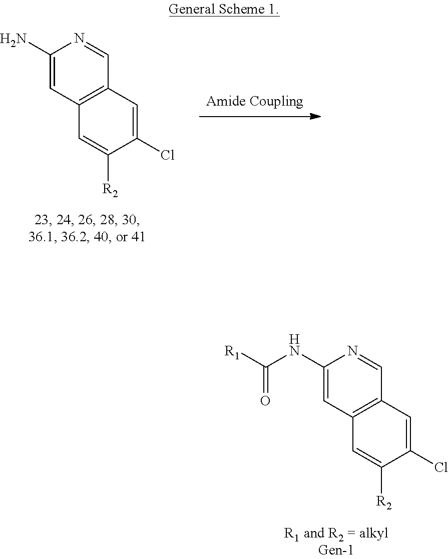

In General Scheme 1, commercially available carboxylic acids or acid chlorides were coupled with intermediates 23, 24, 26, 28, 30, 36.1, 36.2, 40 or 41 through amide coupling conditions to provide fully elaborated compounds in the form of Gen-1. The representative compounds are described in more detail below.

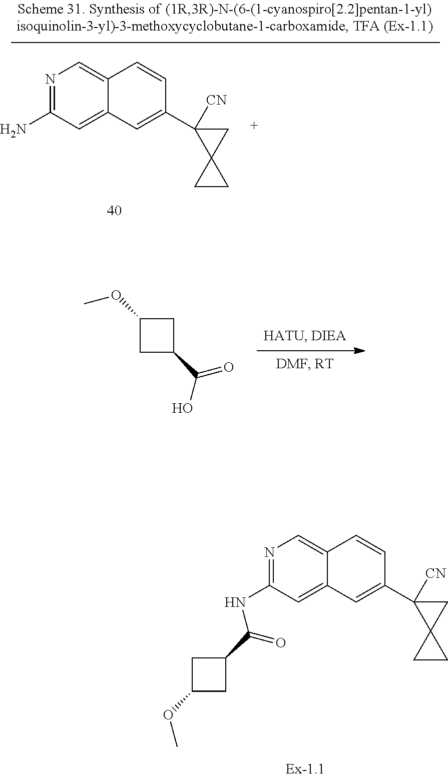

1-(3-aminoisoquinolin-6-yl)spiro[2.2]pentane-1-carbonitrile 40 (30 mg, 0.128 mmol), (1R,3R)-3-methoxycyclobutane-1-carboxylic acid (24.89 mg, 0.191 mmol), HATU (97 mg, 0.255 mmol), DIEA (89 μl, 0.510 mmol), and DMF (1275 μl) were added to a vial. The resulting mixture was allowed to stir overnight at room temperature. The reaction mixture was filtered, (purified by HPLC, eluting acetonitrile/water gradient with 0.1% TFA modifier, linear gradient, and lyophilized to afford the title compound as a TFA salt. MS (ESI): m/z calc'd for C₂₁H₂₁N₃O₂·TFA [M+H]⁺: 348, found 348. ¹H NMR (499 MHz, DMSO-d6) δ 10.59 (s, 1H), 9.12 (s, 1H), 8.51 (s, 1H), 8.07 (d, J=8.6 Hz, 1H), 7.79 (s, 1H), 7.39 (dd, J=8.6, 1.8 Hz, 1H), 4.05 (p, J=6.3 Hz, 1H), 3.37-3.29 (m, 1H), 3.16 (s, 3H), 2.46-2.40 (m, 2H), 2.39 (d, J=5.2 Hz, 1H), 2.19 (d, J=5.2 Hz, 1H), 2.17-2.10 (m, 2H), 1.31-1.21 (m, 3H), 1.01-0.95 (m, 1H). Compounds in Table 1 below were prepared in accordance with the synthetic sequence illustrated in General Scheme 1 and Scheme 31 using the corresponding starting materials.

TABLE 1

| | Structure | Observed |
|---|---|---|
| Example | Name | m/z [M + H]⁺ |
| 1.2-1.4 | 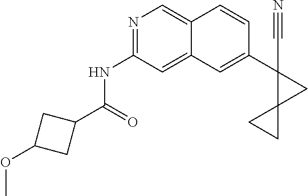 [SMILES: COC1CC(C(=O)Nc2cc3cc(C4(C#N)CC45CC5)ccc3cn2)C1] | Cal'c: 348 Found: 348 |
| | Rac-N-(6-(1-cyanospiro[2.2]pentan-1-yl)isoquinolin-3-yl)-3- | |
| | methoxycyclobutane-1-carboxamide, (1R,3R or 1S,3S)-N- | |
| | (6-((R or S)-1-cyanospiro[2.2]pentan-1-yl)isoquinolin-3-yl)-3- | |
| | methoxycyclobutane-1-carboxamide | |
| 1.5 | 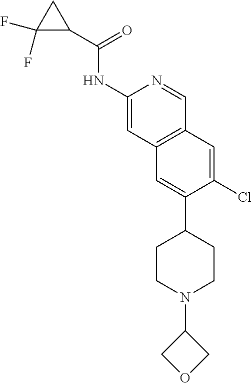 [SMILES: O=C(Nc1cc2cc(C3CCN(C4COC4)CC3)c(Cl)cc2cn1)C1CC1(F)F] | Cal'c: 422 Found: 422 |
| | Rac-N-(7-chloro-6-(1-(oxetan-3-yl)piperidin-4-yl)isoquinolin-3- | |
| | yl)-2,2-difluorocyclopropane-1-carboxamide, (R or S)-N- | |
| | (7-chloro-6-(1-(oxetan-3-yl)piperidin-4-yl)isoquinolin-3-yl)- | |
| | 2,2-difluorocyclopropane-1-carboxamide | |
| 1.6 | 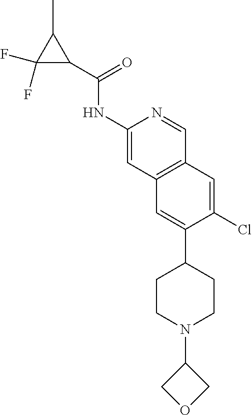 [SMILES: CC1C(C(=O)Nc2cc3cc(C4CCN(C5COC5)CC4)c(Cl)cc3cn2)C1(F)F] | Cal'c: 436 Found: 436 |
| | Rac-N-(7-chloro-6-(1-(oxetan-3-yl)piperidin-4-yl)isoquinolin-3- | |
| | yl)-2,2-difluoro-3-methylcyclopropane-1-carboxamide (tested), | |
| | (1R,3R, 1R,3S, 1S,3R, or 1S3S,)-N-(7-chloro-6-(1-(oxetan-3- | |
| | yl)piperidin-4-yl)isoquinolin-3-yl)-2,2-difluoro-3- | |
| | methylcyclopropane-1-carboxamide, | |
| 1.7 | 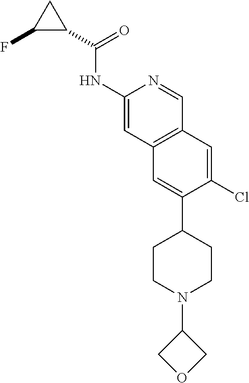 [SMILES: O=C(Nc1cc2cc(C3CCN(C4COC4)CC3)c(Cl)cc2cn1)[C@H]1C[C@@H]1F] | Cal'c: 404 Found: 404 |
| | Rac-trans-N-(7-chloro-6-(1-(oxetan-3-yl)piperidin-4- | |
| | yl)isoquinolin-3-yl)-2-fluorocyclopropane-1-carboxamide, (1R,3S | |
| | or 1S,3R)-N-(7-chloro-6-(1-(oxetan-3-yl)piperidin-4-yl)isoquinolin- | |
| | 3-yl)-2-fluorocyclopropane-1-carboxamide | |
| 1.8 | 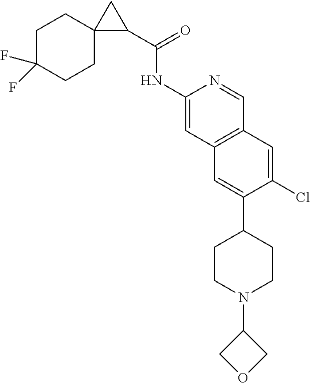 [SMILES: O=C(Nc1cc2cc(C3CCN(C4COC4)CC3)c(Cl)cc2cn1)C1CC12CCC(F)(F)CC2] | Cal'c: 490 Found: 490 |
| | Rac-N-(7-chloro-6-(1-(oxetan-3-yl)piperidin-4-yl)isoquinolin-3- | |
| | yl)-6,6-difluorospiro[2.5]octane-1-carboxamide, (R or S)-N-(7- | |
| | chloro-6-(1-(oxetan-3-yl)piperidin-4-yl)isoquinolin-3-yl)-6,6- | |
| | difluorospiro[2.5]octane-1-carboxamide | |
| 1.9 | 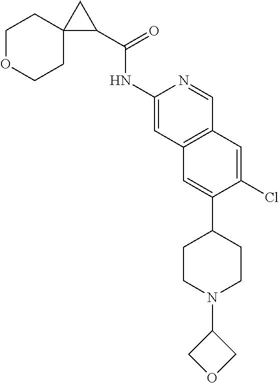 [SMILES: O=C(Nc1cc2cc(C3CCN(C4COC4)CC3)c(Cl)cc2cn1)C1CC12CCOCC2] | Cal'c: 456 Found: 456 |
| | Rac-N-(7-chloro-6-(1-(oxetan-3-yl)piperidin-4-yl)isoquinolin- | |
| | 3-yl)-6-oxaspiro[2.5]octane-1-carboxamide | |
| 1.10 | 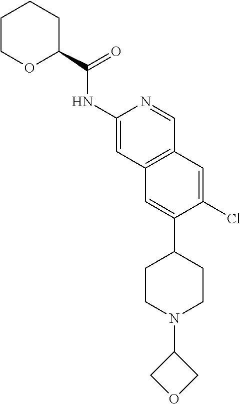 [SMILES: O=C(Nc1cc2cc(C3CCN(C4COC4)CC3)c(Cl)cc2cn1)[C@@H]1CCCCO1] | Cal'c: 430 Found: 430 |
| | (S)-N-(7-chloro-6-(1-(oxetan-3-yl)piperidin-4-yl)isoquinolin- | |
| | 3-yl)tetrahydro-2H-pyran-2-carboxamide | |
| 1.11 | 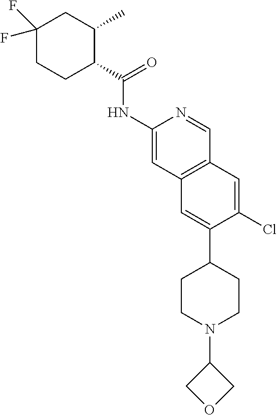 [SMILES: C[C@H]1CC(F)(F)CC[C@H]1C(=O)Nc1cc2cc(C3CCN(C4COC4)CC3)c(Cl)cc2cn1] | Cal'c: 478 Found: 478 |
| | (1R,2S)-N-(7-chloro-6-(1-(oxetan-3-yl)piperidin-4-yl)isoquinolin- | |
| | 3-yl)-4,4-difluoro-2-methylcyclohexane-1-carboxamide | |
| 1.12 | 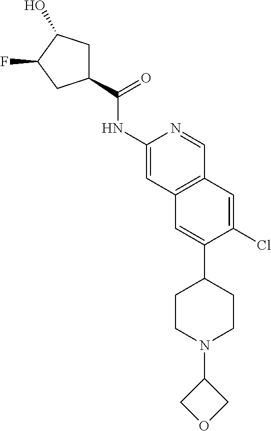 [SMILES: O=C(Nc1cc2cc(C3CCN(C4COC4)CC3)c(Cl)cc2cn1)[C@@H]1C[C@@H](O)[C@H](F)C1] | Cal'c: 448 Found: 448 |
| | (1R,3R,4R)-N-(7-chloro-6-(1-(oxetan-3-yl)piperidin-4- | |
| | yl)isoquinolin-3-yl)-3-fluoro-4-hydroxycyclopentane-1- | |
| | carboxamide | |
| 1.13 | 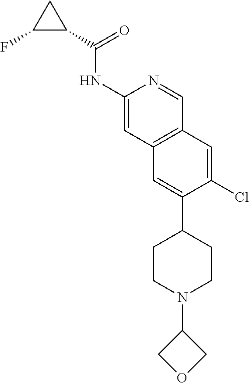 [SMILES: O=C(Nc1cc2cc(C3CCN(C4COC4)CC3)c(Cl)cc2cn1)[C@H]1C[C@H]1F] | Cal'c: 404 Found: 404 |
| | Rac-Cis-N-(7-chloro-6-(1-(oxetan-3-yl)piperidin-4-yl)isoquinolin- | |
| | 3-yl)-2-fluorocyclopropane-1-carboxamide, (1R,2R)-N-(7-chloro- | |
| | 6-(1-(oxetan-3-yl)piperidin-4-yl)isoquinolin-3-yl)-2- | |
| | fluorocyclopropane-1-carboxamide | |
| 1.14 | 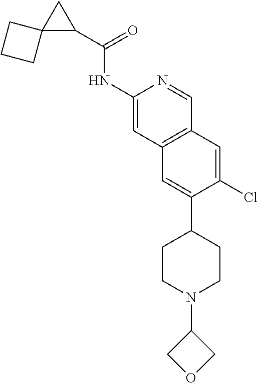 [SMILES: O=C(Nc1cc2cc(C3CCN(C4COC4)CC3)c(Cl)cc2cn1)C1CC12CCC2] | Cal'c: 426 Found: 426 |
| | Rac-N-(7-chloro-6-(1-(oxetan-3-yl)piperidin-4-yl)isoquinolin-3- | |
| | yl)spiro[2.3]hexane-1-carboxamide | |
| 1.15 | 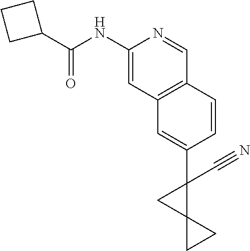 [SMILES: N#CC1(c2ccc3cnc(NC(=O)C4CCC4)cc3c2)CC12CC2] | Cal'c: 318 Found: 318 |
| | Rac-N-(6-(1-cyanospiro[2.2]pentan-1-yl)isoquinolin- | |
| | 3-yl)cyclobutanecarboxamide, (R or S)-N-(6-(1- | |
| | cyanospiro[2.2]pentan-1-yl)isoquinolin-3- | |
| | yl)cyclobutanecarboxamide | |
| 1.16 | 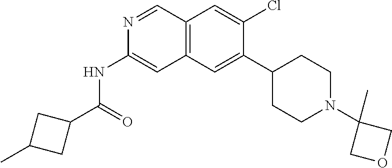 [SMILES: CC1CC(C(=O)Nc2cc3cc(C4CCN(C5(C)COC5)CC4)c(Cl)cc3cn2)C1] | Cal'c: 428 Found: 428 |
| | Rac-N-(7-chloro-6-(1-(3-methyloxetan-3-yl)piperidin-4- | |
| | yl)isoquinolin-3-yl)-3-methylcyclobutane-1-carboxamide | |
| 1.17 | 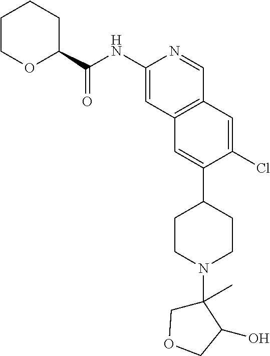 [SMILES: CC1(N2CCC(c3cc4cc(NC(=O)[C@@H]5CCCCO5)ncc4cc3Cl)CC2)COCC1O] | Cal'c: 474 Found: 474 |
| | (2S)-N-(7-chloro-6-(1-(4-hydroxy-3-methyltetrahydrofuran-3- | |
| | yl)piperidin-4-yl)isoquinolin-3-yl)tetrahydro-2H-pyran-2- | |
| | carboxamide, (S)-N-(7-chloro-6-(1-((3R,4R or 3S,4S)-4-hydroxy-3- | |
| | methyltetrahydrofuran-3-yl)piperidin-4-yl)isoquinolin-3- | |
| | yl)tetrahydro-2H-pyran-2-carboxamide | |
| 1.18 | 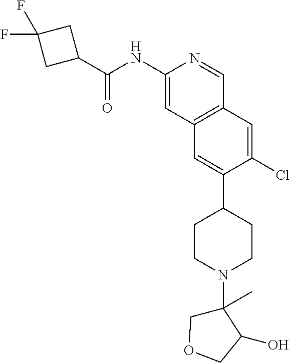 [SMILES: CC1(N2CCC(c3cc4cc(NC(=O)C5CC(F)(F)C5)ncc4cc3Cl)CC2)COCC1O] | Cal'c: 480 Found: 480 |
| | Rac-N-(7-chloro-6-(1-(4-hydroxy-3-methyltetrahydrofuran-3- | |
| | yl)piperidin-4-yl)isoquinolin-3-yl)-3,3-difluorocyclobutane-1- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3-yl)-3,3- | |
| | difluorocyclobutane-1-carboxamide | |
| 1.19 | 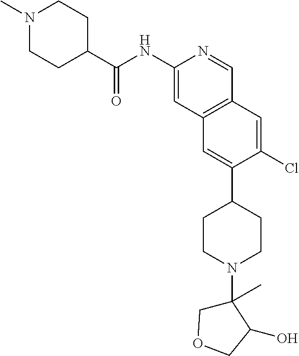 [SMILES: CN1CCC(C(=O)Nc2cc3cc(C4CCN(C5(C)COCC5O)CC4)c(Cl)cc3cn2)CC1] | Cal'c: 487 Found: 487 |
| | Rac-N-(7-chloro-6-(1-(4-hydroxy-3-methyltetrahydrofuran-3- | |
| | yl)piperidin-4-yl)isoquinolin-3-yl)-1-methylpiperidine-4- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3-yl)-1- | |
| | methylpiperidine-4-carboxamide | |
| 1.20 | 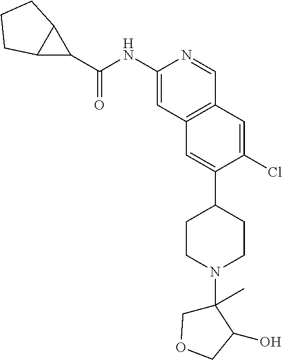 [SMILES: CC1(N2CCC(c3cc4cc(NC(=O)C5C6CCCC65)ncc4cc3Cl)CC2)COCC1O] | Cal'c: 470 Found: 470 |
| | Rac-N-(7-chloro-6-(1-(4-hydroxy-3-methyltetrahydrofuran-3- | |
| | yl)piperidin-4-yl)isoquinolin-3-yl)bicyclo[3.1.0]hexane-6- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3- | |
| | yl)bicyclo[3.1.0]hexane-6-carboxamide | |
| 1.21 | 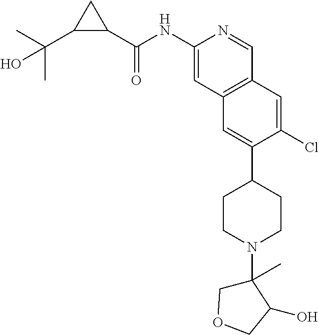 [SMILES: CC(C)(O)C1CC1C(=O)Nc1cc2cc(C3CCN(C4(C)COCC4O)CC3)c(Cl)cc2cn1] | Cal'c: 488 Found: 488 |
| | Rac-N-(7-chloro-6-(1-(4-hydroxy-3-methyltetrahydrofuran-3- | |
| | yl)piperidin-4-yl)isoquinolin-3-yl)-2-(2-hydroxypropan-2- | |
| | yl)cyclopropane-1-carboxamide, (1R,2R or 1S,2S)-N-(7-chloro-6- | |
| | (1-((3R,4R or 3S,4S)-4-hydroxy-3-methyltetrahydrofuran-3- | |
| | yl)piperidin-4-yl)isoquinolin-3-yl)-2-(2-hydroxypropan-2- | |
| | yl)cyclopropane-1-carboxamide | |
| 1.22 | 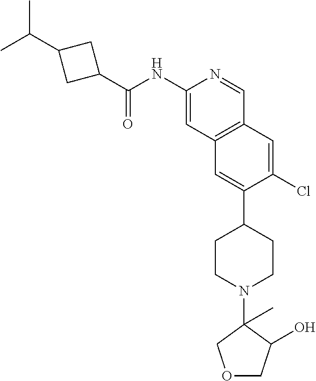 [SMILES: CC(C)C1CC(C(=O)Nc2cc3cc(C4CCN(C5(C)COCC5O)CC4)c(Cl)cc3cn2)C1] | Cal'c: 486 Found: 486 |
| | Rac-N-(7-chloro-6-(1-(4-hydroxy-3-methyltetrahydrofuran-3- | |
| | yl)piperidin-4-yl)isoquinolin-3-yl)-3-isopropylcyclobutane-1- | |
| | carboxamide | |
| 1.23-1.24 | 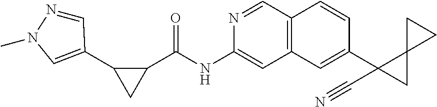 [SMILES: Cn1cc(C2CC2C(=O)Nc2cc3cc(C4(C#N)CC45CC5)ccc3cn2)cn1] | Cal'c: 311 Found: 311 |
| | Rac-N-(6-(1-cyanospiro[2.2]pentan-1-yl)isoquinolin-3-yl)-2-(1- | |
| | methyl-1H-pyrazol-4-yl)cyclopropane-1-carboxamide, (1R,2R or | |
| | 1S,2S)-N-(6-((R or S)-1-cyanospiro[2.2]pentan-1-yl)isoquinolin-3- | |
| | yl)-2-(1-methyl-1H-pyrazol-4-yl)cyclopropane-1-carboxamide | |
| 1.25 | 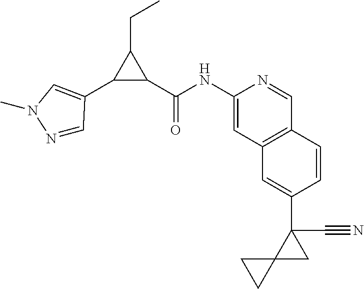 [SMILES: CCC1C(C(=O)Nc2cc3cc(C4(C#N)CC45CC5)ccc3cn2)C1c1cnn(C)c1] | Cal'c: 412 Found: 412 |
| | Rac-N-(6-(1-cyanospiro[2.2]pentan-1-yl)isoquinolin-3-yl)-2-ethyl- | |
| | 3-(1-methyl-1H-pyrazol-4-yl)cyclopropane-1-carboxamide, (1R,3R | |
| | or 1S,3S)-N-(6-((R or S)-1-cyanospiro[2.2]pentan-1-yl)isoquinolin- | |
| | 3-yl)-2-ethyl-3-(1-methyl-1H-pyrazol-4-yl)cyclopropane-1- | |
| | carboxamide | |
| 1.26 | 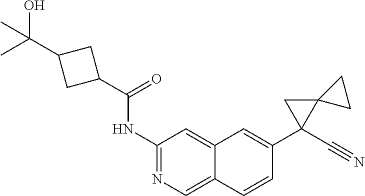 [SMILES: CC(C)(O)C1CC(C(=O)Nc2cc3cc(C4(C#N)CC45CC5)ccc3cn2)C1] | Cal'c: 376 Found: 376 |
| | Rac-N-(6-(1-cyanospiro[2.2]pentan-1-yl)isoquinolin-3-yl)-3-(2- | |
| | hydroxypropan-2-yl)cyclobutane-1-carboxamide, (1R,3R or 1S,3S)- | |
| | N-(6-((R or S)-1-cyanospiro[2.2]pentan-1-yl)isoquinolin-3-yl)-3-(2- | |
| | hydroxypropan-2-yl)cyclobutane-1-carboxamide | |
| 1.27 | 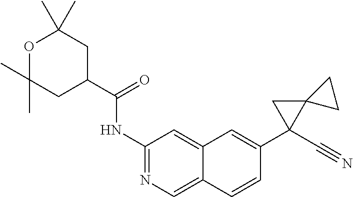 [SMILES: CC1(C)CC(C(=O)Nc2cc3cc(C4(C#N)CC45CC5)ccc3cn2)CC(C)(C)O1] | Cal'c: 405 Found: 405 |
| | Rac-N-(6-(1-cyanospiro[2.2]pentan-1-yl)isoquinolin-3-yl)- | |
| | 2,2,6,6-tetramethyltetrahydro-2H-pyran-4-carboxamide, N- | |
| | (6-((R or S)-1-cyanospiro[2.2]pentan-1-yl)isoquinolin-3-yl)- | |
| | 2,2,6,6-tetramethyltetrahydro-2H-pyran-4-carboxamide | |
| 1.28 | 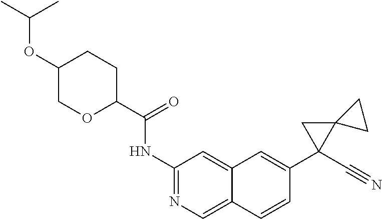 [SMILES: CC(C)OC1CCC(C(=O)Nc2cc3cc(C4(C#N)CC45CC5)ccc3cn2)OC1] | Cal'c: 406 Found: 406 |
| | Rac-N-(6-(1-cyanospiro[2.2]pentan-1-yl)isoquinolin-3-yl)-5- | |
| | isopropoxytetrahydro-2H-pyran-2-carboxamide, N-(6-((R or S)- | |
| | 1-cyanospiro[2.2]pentan-1-yl)isoquinolin-3-yl)-5R or 5S- | |
| | isopropoxytetrahydro-2H-pyran-2R-carboxamide | |
| 1.29 | 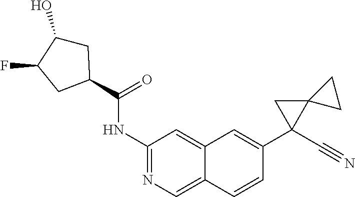 [SMILES: N#CC1(c2ccc3cnc(NC(=O)[C@@H]4C[C@@H](O)[C@H](F)C4)cc3c2)CC12CC2] | Cal'c: 366 Found: 366 |
| | (1R,3R,4R)-N-(6-(1-cyanospiro[2.2]pentan-1-yl)isoquinolin-3-yl)- | |
| | 3-fluoro-4-hydroxycyclopentane-1-carboxamide, (1R,3R,4R)-N-(6- | |
| | ((R or S)-1-cyanospiro[2.2]pentan-1-yl)isoquinolin-3-yl)-3-fluoro- | |
| | 4-hydroxycyclopentane-1-carboxamide | |
| 1.30 | 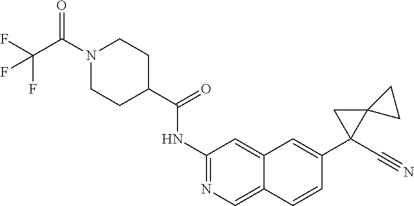 [SMILES: N#CC1(c2ccc3cnc(NC(=O)C4CCN(C(=O)C(F)(F)F)CC4)cc3c2)CC12CC2] | Cal'c: 443 Found: 443 |
| | Rac-N-(6-(1-cyanospiro[2.2]pentan-1-yl)isoquinolin-3-yl)-1-(2,2,2- | |
| | trifluoroacetyl)piperidine-4-carboxamide, N-(6-((R or S)-1- | |
| | cyanospiro[2.2]pentan-1-yl)isoquinolin-3-yl)-1-(2,2,2- | |
| | trifluoroacetyl)piperidine-4-carboxamide | |
| 1.31 | 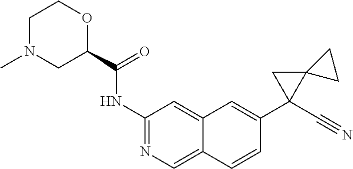 [SMILES: CN1CCO[C@@H](C(=O)Nc2cc3cc(C4(C#N)CC45CC5)ccc3cn2)C1] | Cal'c: 363 Found: 363 |
| | (2R)-N-(6-(1-cyanospiro[2.2]pentan-1-yl)isoquinolin-3-yl)-4- | |
| | methylmorpholine-2-carboxamide, (2R)-N-(6-((R or S)-1- | |
| | cyanospiro[2.2]pentan-1-yl)isoquinolin-3-yl)-4-methylmorpholine- | |
| | 2-carboxamide | |
| 1.32 | 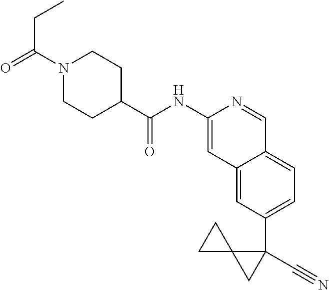 [SMILES: CCC(=O)N1CCC(C(=O)Nc2cc3cc(C4(C#N)CC45CC5)ccc3cn2)CC1] | Cal'c: 403 Found: 403 |
| | Rac-N-(6-(1-cyanospiro[2.2]pentan-1-yl)isoquinolin-3-yl)- | |
| | 1-propionylpiperidine-4-carboxamide, N-(6-((R or S)-1- | |
| | cyanospiro[2.2]pentan-1-yl)isoquinolin-3-yl)-1- | |
| | propionylpiperidine-4-carboxamide | |
| 1.33 | 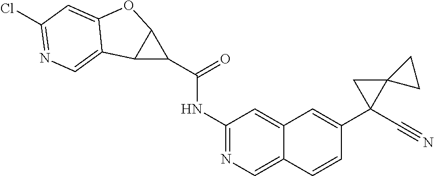 [SMILES: N#CC1(c2ccc3cnc(NC(=O)C4C5Oc6cc(Cl)ncc6C54)cc3c2)CC12CC2] | Cal'c: 429 Found: 429 |
| | Rac-4-chloro-N-(6-(1-cyanospiro[2.2]pentan-1-yl)isoquinolin-3- | |
| | yl)-1a,6b-dihydro-1H-cyclopropa[4,5]furo[3,2-c]pyridine-1- | |
| | carboxamide, (1S,1aR,6bS or 1R,1aS,6bR)-4-chloro-N-(6-((R or S)- | |
| | 1-cyanospiro[2.2]pentan-1-yl)isoquinolin-3-yl)-1a,6b-dihydro-1H- | |
| | cyclopropa[4,5]furo[3,2-c]pyridine-1-carboxamide | |
| 1.34 | 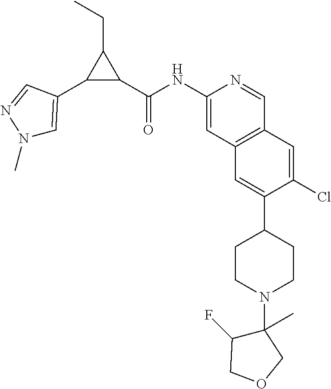 [SMILES: CCC1C(C(=O)Nc2cc3cc(C4CCN(C5(C)COCC5F)CC4)c(Cl)cc3cn2)C1c1cnn(C)c1] | Cal'c: 540 Found: 540 |
| | Rac-N-(7-chloro-6-(1-(4-fluoro-3-methyltetrahydrofuran-3- | |
| | yl)piperidin-4-yl)isoquinolin-3-yl)-2-ethyl-3-(1-methyl-1H-pyrazol- | |
| | 4-yl)cyclopropane-1-carboxamide, (1R,2R,3R or 1R,2S,3R or | |
| | 1S,2R,3S, or 1S,2S,3S)-N-(7-chloro-6-(1-((3R,4R or 3S,4S)-4- | |
| | fluoro-3-methyltetrahydrofuran-3-yl)piperidin-4-yl)isoquinolin-3- | |
| | yl)-2-ethyl-3-(1-methyl-1H-pyrazol-4-yl)cyclopropane-1- | |
| | carboxamide | |
| 1.35 | 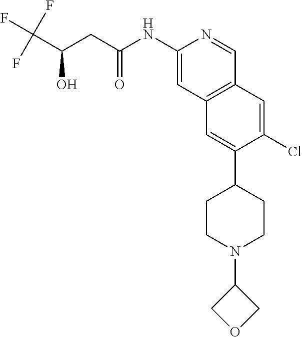 [SMILES: O=C(C[C@@H](O)C(F)(F)F)Nc1cc2cc(C3CCN(C4COC4)CC3)c(Cl)cc2cn1] | Cal'c: 458 Found: 458 |
| | (R)-N-(7-chloro-6-(1-(oxetan-3-yl)piperidin-4-yl)isoquinolin- | |
| | 3-yl)-4,4,4-trifluoro-3-hydroxybutanamide | |
| 1.36 | 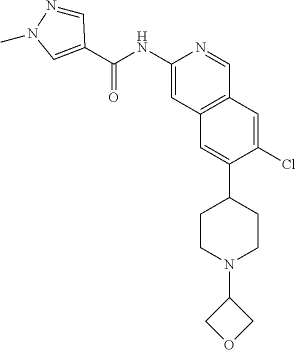 [SMILES: Cn1cc(C(=O)Nc2cc3cc(C4CCN(C5COC5)CC4)c(Cl)cc3cn2)cn1] | Cal'c: 426 Found: 426 |
| | N-(7-chloro-6-(1-(oxetan-3-yl)piperidin-4-yl)isoquinolin- | |
| | 3-yl)-1-methyl-1H-pyrazole-4-carboxamide | |
| 1.37 | 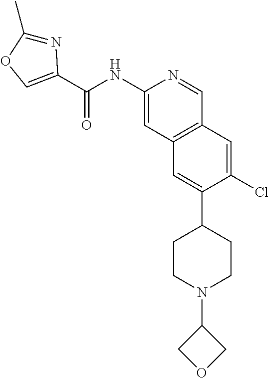 [SMILES: Cc1nc(C(=O)Nc2cc3cc(C4CCN(C5COC5)CC4)c(Cl)cc3cn2)co1] | Cal'c: 427 Found: 427 |
| | N-(7-chloro-6-(1-(oxetan-3-yl)piperidin-4-yl)isoquinolin- | |
| | 3-yl)-2-methyloxazole-4-carboxamide | |
| 1.38 | 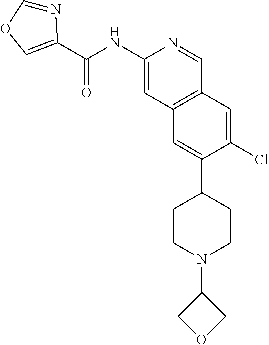 [SMILES: O=C(Nc1cc2cc(C3CCN(C4COC4)CC3)c(Cl)cc2cn1)c1cocn1] | Cal'c: 413 Found: 413 |
| | N-(7-chloro-6-(1-(oxetan-3-yl)piperidin-4-yl)isoquinolin- | |
| | 3-yl)oxazole-4-carboxamide | |
| 1.39 | 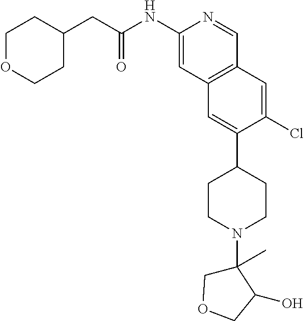 [SMILES: CC1(N2CCC(c3cc4cc(NC(=O)CC5CCOCC5)ncc4cc3Cl)CC2)COCC1O] | Cal'c: 488 Found: 488 |
| | Rac-N-(7-chloro-6-(1-(4-hydroxy-3-methyltetrahydrofuran-3- | |
| | yl)piperidin-4-yl)isoquinolin-3-yl)-2-(tetrahydro-2H-pyran-4- | |
| | yl)acetamide, N-(7-chloro-6-(1-((3R,4R or 3S,4S)-4-hydroxy- | |
| | 3-methyltetrahydrofuran-3-yl)piperidin-4-yl)isoquinolin- | |
| | 3-yl)-2-(tetrahydro-2H-pyran-4-yl)acetamide | |
| 1.40 | 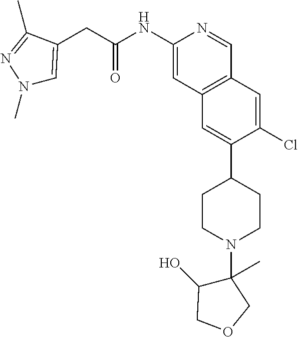 [SMILES: Cc1nn(C)cc1CC(=O)Nc1cc2cc(C3CCN(C4(C)COCC4O)CC3)c(Cl)cc2cn1] | Cal'c: 498 Found: 498 |
| | Rac-N-(7-chloro-6-(1-(4-hydroxy-3-methyltetrahydrofuran-3- | |
| | yl)piperidin-4-yl)isoquinolin-3-yl)-2-(1,3-dimethyl-1H-pyrazol- | |
| | 4-yl)acetamide, N-(7-chloro-6-(1-((3R,4R or 3S,4S)-4-hydroxy- | |
| | 3-methyltetrahydrofuran-3-yl)piperidin-4-yl)isoquinolin- | |
| | 3-yl)-2-(1,3-dimethyl-1H-pyrazol-4-yl)acetamide | |
| 1.41 | 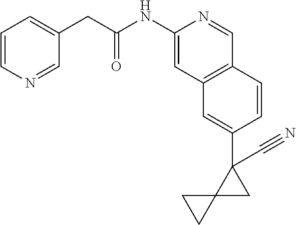 [SMILES: N#CC1(c2ccc3cnc(NC(=O)Cc4cccnc4)cc3c2)CC12CC2] | Cal'c: 355 Found: 355 |
| | Rac-N-(6-(1-cyanospiro[2.2]pentan-1-yl)isoquinolin-3-yl)-2- | |
| | (pyridin-3-yl)acetamide, (R or S)-N-(6-(1-cyanospiro[2.2] | |
| | pentan-1-yl)isoquinolin-3-yl)-2-(pyridin-3-yl)acetamide | |
| 1.42 | 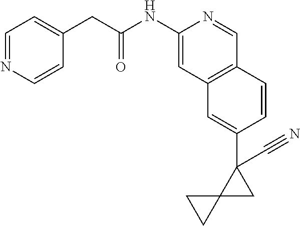 [SMILES: N#CC1(c2ccc3cnc(NC(=O)Cc4ccncc4)cc3c2)CC12CC2] | Cal'c: 355 Found: 355 |
| | Rac-N-(6-(1-cyanospiro[2.2]pentan-1-yl)isoquinolin-3-yl)-2- | |
| | (pyridin-3-yl)acetamide, (R or S)-N-(6-(1-cyanospiro[2.2] | |
| | pentan-1-yl)isoquinolin-3-yl)-2-(pyridin-3-yl)acetamide | |
| 1.43 | 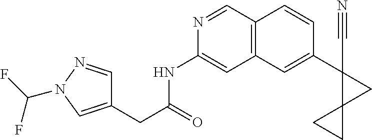 [SMILES: N#CC1(c2ccc3cnc(NC(=O)Cc4cnn(C(F)F)c4)cc3c2)CC12CC2] | Cal'c: 394 Found: 394 |
| | Rac-N-(6-(1-cyanospiro[2.2]pentan-1-yl)isoquinolin-3-yl)-2-(1- | |
| | (difluoromethyl)-1H-pyrazol-4-yl)acetamide, (R or S)-N-(6-(1- | |
| | cyanospiro[2.2]pentan-1-yl)isoquinolin-3-yl)-2- | |
| | (1-(difluoromethyl)-1H-pyrazol-4-yl)acetamide | |
| 1.44 | 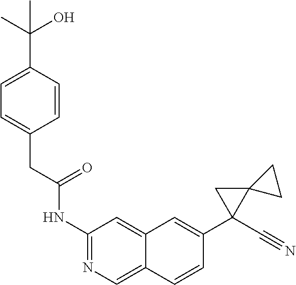 [SMILES: CC(C)(O)c1ccc(CC(=O)Nc2cc3cc(C4(C#N)CC45CC5)ccc3cn2)cc1] | Cal'c: 412 Found: 412 |
| | Rac-N-(6-(1-cyanospiro[2.2]pentan-1-yl)isoquinolin-3-yl)- | |
| | 2-(4-(2-hydroxypropan-2-yl)phenyl)acetamide, (R or S)- | |
| | N-(6-(1-cyanospiro[2.2]pentan-1-yl)isoquinolin-3-yl)- | |
| | 2-(4-(2-hydroxypropan-2-yl)phenyl)acetamide | |
| 1.45 | 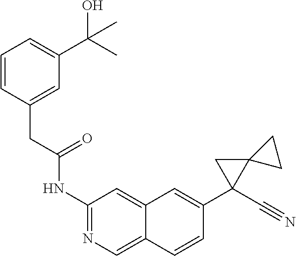 [SMILES: CC(C)(O)c1cccc(CC(=O)Nc2cc3cc(C4(C#N)CC45CC5)ccc3cn2)c1] | Cal'c: 412 Found: 412 |
| | Rac-N-(6-(1-cyanospiro[2.2]pentan-1-yl)isoquinolin-3-yl)- | |
| | 2-(3-(2-hydroxypropan-2-yl)phenyl)acetamide, (R or S)- | |
| | N-(6-(1-cyanospiro[2.2]pentan-1-yl)isoquinolin-3-yl)- | |
| | 2-(3-(2-hydroxypropan-2-yl)phenyl)acetamide | |
| 1.46 | 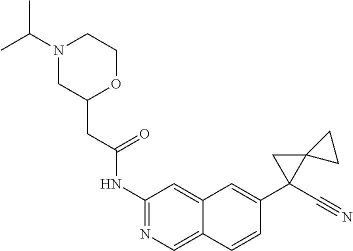 [SMILES: CC(C)N1CCOC(CC(=O)Nc2cc3cc(C4(C#N)CC45CC5)ccc3cn2)C1] | Cal'c: 405 Found: 405 |
| | Rac-N-(6-(1-cyanospiro[2.2]pentan-1-yl)isoquinolin-3-yl)- | |
| | 2-(4-isopropylmorpholin-2-yl)acetamide, (R or S)-N-(6-(1- | |
| | cyanospiro[2.2]pentan-1-yl)isoquinolin-3-yl)-2-(4- | |
| | isopropylmorpholin-2-yl)acetamide | |
| 1.47 | 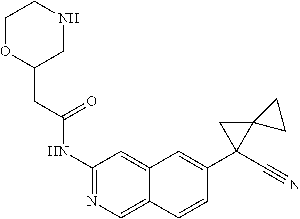 [SMILES: N#CC1(c2ccc3cnc(NC(=O)CC4CNCCO4)cc3c2)CC12CC2] | Cal'c: 377 Found: 377 |
| | Rac-N-(6-(1-cyanospiro[2.2]pentan-1-yl)isoquinolin-3- | |
| | yl)-2-(4-methylmorpholin-2-yl)acetamide, (R or S)- | |
| | N-(6-(1-cyanospiro[2.2]pentan-1-yl)isoquinolin-3-yl)- | |
| | 2-(4-methylmorpholin-2-yl)acetamide | |
| 1.48 | 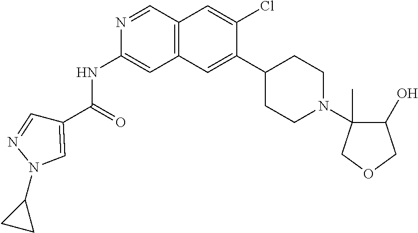 [SMILES: CC1(N2CCC(c3cc4cc(NC(=O)c5cnn(C6CC6)c5)ncc4cc3Cl)CC2)COCC1O] | Cal'c: 496 Found: 496 |
| | Rac-N-(7-chloro-6-(1-(4-hydroxy-3-methyltetrahydrofuran-3- | |
| | yl)piperidin-4-yl)isoquinolin-3-yl)-1-cyclopropyl-1H-pyrazole-4- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3-yl)-1- | |
| | cyclopropyl-1H-pyrazole-4-carboxamide | |
| 1.49 | 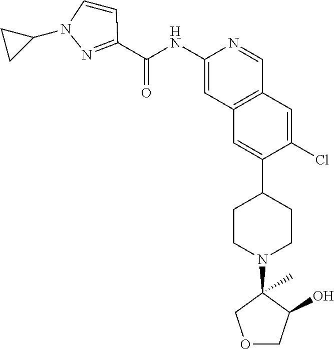 [SMILES: C[C@@]1(N2CCC(c3cc4cc(NC(=O)c5ccn(C6CC6)n5)ncc4cc3Cl)CC2)COC[C@@H]1O] | Cal'c: 496 Found: 496 |
| | Rac-N-(7-chloro-6-(1-(4-hydroxy-3-methyltetrahydrofuran-3- | |
| | yl)piperidin-4-yl)isoquinolin-3-yl)-1-cyclopropyl-1H-pyrazole-3- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3-yl)-1- | |
| | cyclopropyl-1H-pyrazole-3-carboxamide | |
| 1.50 | 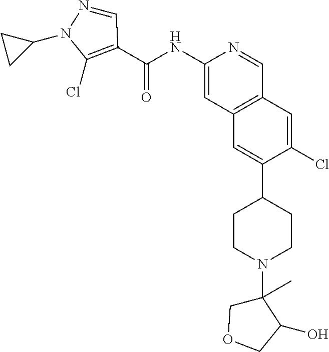 [SMILES: CC1(N2CCC(c3cc4cc(NC(=O)c5cnn(C6CC6)c5Cl)ncc4cc3Cl)CC2)COCC1O] | Cal'c: 530 Found: 530 |
| | Rac-5-chloro-N-(7-chloro-6-(1-(4-hydroxy-3- | |
| | methyltetrahydrofuran-3-yl)piperidin-4-yl)isoquinolin-3-yl)-1- | |
| | cyclopropyl-1H-pyrazole-4-carboxamide, 5-chloro-N-(7-chloro-6- | |
| | (1-((3R,4R or 3S,4S)-4-hydroxy-3-methyltetrahydrofuran-3- | |
| | yl)piperidin-4-yl)isoquinolin-3-yl)-1-cyclopropyl-1H-pyrazole-4- | |
| | carboxamide | |
| 1.51 | 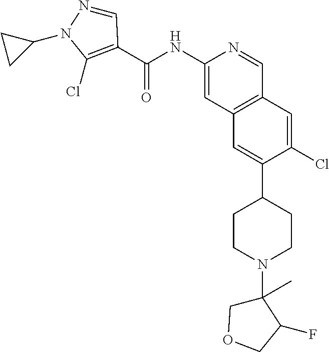 [SMILES: CC1(N2CCC(c3cc4cc(NC(=O)c5cnn(C6CC6)c5Cl)ncc4cc3Cl)CC2)COCC1F] | Cal'c: 532 Found: 532 |
| | Rac-5-chloro-N-(7-chloro-6-(1-(4-fluoro-3-methyltetrahydrofuran- | |
| | 3-yl)piperidin-4-yl)isoquinolin-3-y1)-1-cyclopropyl-1H-pyrazole-4- | |
| | carboxamide, 5-chloro-N-(7-chloro-6-(1-((3R,4R or 3S,4S)-4- | |
| | fluoro-3-methyltetrahydrofuran-3-yl)piperidin-4-yl)isoquinolin-3- | |
| | yl)-1-cyclopropyl-1H-pyrazole-4-carboxamide | |
| 1.52 | 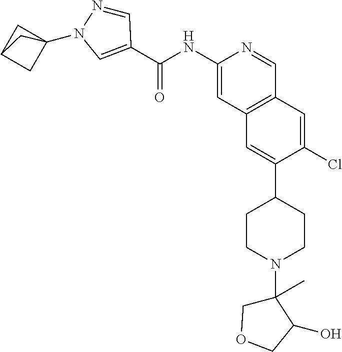 [SMILES: CC1(N2CCC(c3cc4cc(NC(=O)c5cnn(C67CC(C6)C7)c5)ncc4cc3Cl)CC2)COCC1O] | Cal'c: 522 Found: 522 |
| | Rac-1-(bicyclo[1.1.1]pentan-1-yl)-N-(7-chloro-6-(1-(4-hydroxy-3- | |
| | methyltetrahydrofuran-3-yl)piperidin-4-yl)isoquinolin-3-yl)-1H- | |
| | pyrazole-4-carboxamide, 1-(bicyclo[1.1.1]pentan-1-yl)-N-(7- | |
| | chloro-6-(1-((3R,4R or 3S,4S)-4-hydroxy-3-methyltetrahydrofuran- | |
| | 3-yl)piperidin-4-yl)isoquinolin-3-yl)-1H-pyrazole-4-carboxamide | |
| 1.53 | 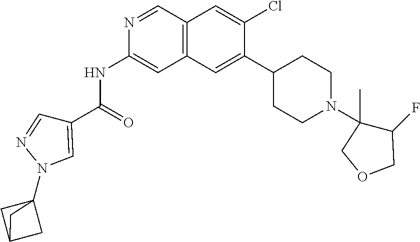 [SMILES: CC1(N2CCC(c3cc4cc(NC(=O)c5cnn(C67CC(C6)C7)c5)ncc4cc3Cl)CC2)COCC1F] | Cal'c: 524 Found: 524 |
| | Rac-1-(bicyclo[1.1.1]pentan-1-yl)-N-(7-chloro-6-(1-(4-fluoro-3- | |
| | methyltetrahydrofuran-3-yl)piperidin-4-yl)isoquinolin-3-yl)-1H- | |
| | pyrazole-4-carboxamide, 1-(bicyclo[1.1.1]pentan-1-yl)-N-(7- | |
| | chloro-6-(1-((3R,4R or 3S,4S)-4-fluoro-3-methyltetrahydrofuran-3- | |
| | yl)piperidin-4-yl)isoquinolin-3-yl)-1H-pyrazole-4-carboxamide | |

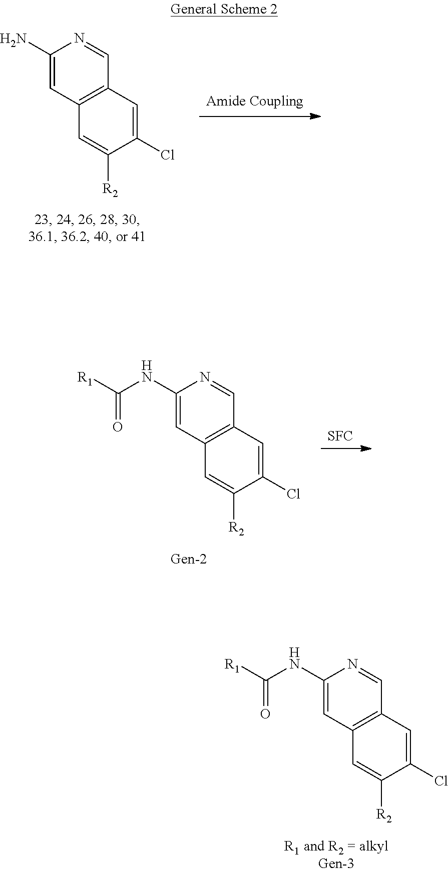

In General Scheme 2, commercially available carboxylic acids or acid chlorides were coupled with intermediate 23, 24, 26, 28, 30, 36.1, 36.2, 40, or 41 through amide coupling conditions to provide fully elaborated compounds in the form of Gen-2 and the stereoisomers could then be separated by chiral SFC to provide fully elaborated products in the form of Gen-3. The representative compounds are described in more detail below.

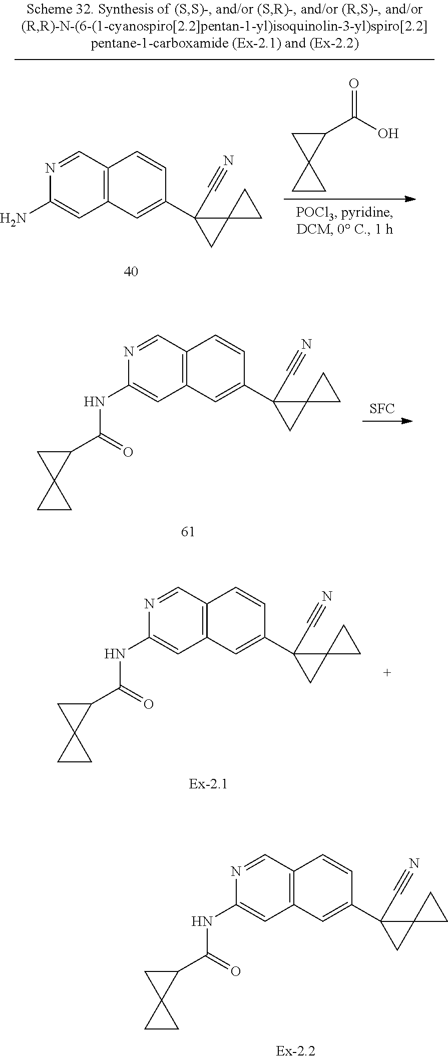

### (S,S)-, and/or (S,R)-, and/or (R,S)-, and/or (R,R)-N-(6-(1-cyanospiro[2.2]pentan-1-yl)isoquinolin-3-yl)spiro[2.2]pentane-1-carboxamide (Ex-2.1) and (Ex-2.1)

To a solution of 1-(3-aminoisoquinolin-6-yl)spiro[2.2]pentane-1-carbonitrile 40 (60 mg, 0.255 mmol) and spiro[2.2]pentane-1-carboxylic acid (34.3 mg, 0.306 mmol) in anhydrous DCM (5 mL) was added pyridine (170 μL, 2.102 mmol), and the resulting mixture was stirred at 0° C. To this mixture was then added POCl₃ (48 μL, 0.510 mmol), and the reaction mixture was stirred at 0° C. for 1 h. The reaction mixture was carefully quenched by pouring into water (10 mL). The biphasic mixture was transferred to a separatory funnel and extracted with DCM (3×10 mL). The combined organic layers were dried over anhydrous Na₂SO₄. After filtration and concentration, the crude product was purified by HPLC (MeCN/H₂O/TFA) to give N-(6-(1-cyanospiro[2.2]pentan-1-yl)isoquinolin-3-yl)spiro[2.2]pentane-1-carboxamide 61 as a mixture of diastereomers.

The mixture of two stereoisomers was purified by chiral SFC (Chiralpak C2; 250 mm*30 mm, 10 um; 50% EtOH/CO₂) and lyophilized to afford the chiral resolved stereoisomers of the title compound Ex-2.1 (tR=3.85 min) and Ex-2.2 (tR=5.69 min). Ex-2.1: MS (ESI): m/z calc'd for C₂₁H₂₀N₃O [M+H]⁺: 330.1, found 330.1. ¹H NMR (400 MHz, CDCl₃) δ: 8.92 (s, 1H), 8.55 (s, 1H), 8.17 (s, 1H), 7.85 (d, J=8.61 Hz, 1H), 7.67 (s, 1H), 7.39 (dd, J=8.61, 1.57, Hz, 1H), 2.35 (d, J=5.09 Hz, 1H), 2.03 (dd, J=7.63, 4.11 Hz, 1H), 1.88 (d, J=5.09 Hz, 1H), 1.62 (t, J=4.11 Hz, 1H), 1.49 (dd, J=7.63, 4.11 Hz, 1H), 1.35 (dd, J=10.56, 3.52 Hz, 1H), 1.23-1.30 (m, 2H), 1.04-1.15 (m, 2H), 0.94-1.03 (m, 3H). Ex-2.2: MS (ESI): m/z calc'd for C₂₁H₂₀N₃O [M+H]⁺: 330.1, found 330.1. ¹H NMR (400 MHz, CDCl₃) δ: 8.92 (s, 1H), 8.57 (s, 1H), 8.38 (br s, 1H), 7.86 (d, J=8.61 Hz, 1H), 7.68 (s, 1H), 7.40 (dd, J=8.61, 1.96 Hz, 1H), 2.36 (d, J=5.09 Hz, 1H), 2.05 (dd, J=7.63, 4.11 Hz, 1H), 1.90 (d, J=5.09 Hz, 1H), 1.62 (t, J=4.11 Hz, 1H), 1.49 (dd, J=7.63, 4.11 Hz, 1H), 1.33-1.39 (m, 1H), 1.24-1.31 (m, 2H), 1.04-1.15 (m, 2H), 0.96-1.03 (m, 3H). Final stereochemistry was not defined.

Compounds in Table 2 below were prepared in accordance with the synthetic sequence illustrated in General Scheme 2 and Scheme 32 using the corresponding starting materials.

TABLE 2

| | | |
|---|---|---|
| 2.3-2.4 | 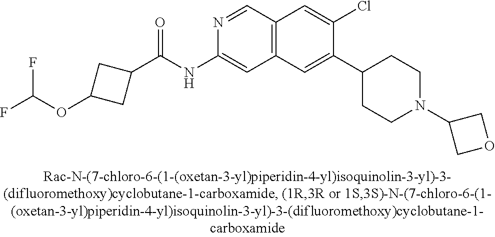 [SMILES: O=C(Nc1cc2cc(C3CCN(C4COC4)CC3)c(Cl)cc2cn1)C1CC(OC(F)F)C1] | Cal'c: 466 Found: 466 |
| 2.5-2.7 | 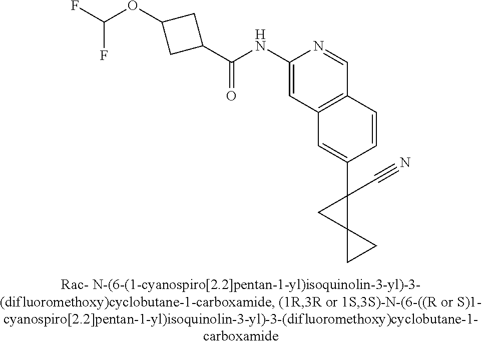 [SMILES: N#CC1(c2ccc3cnc(NC(=O)C4CC(OC(F)F)C4)cc3c2)CC12CC2] | Cal'c: 384 Found: 384 |
| 2.8-2.9 | 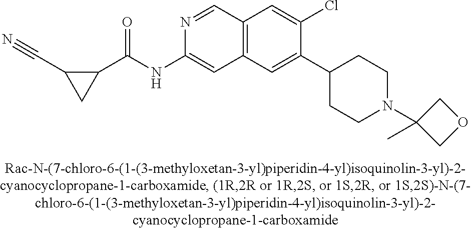 [SMILES: CC1(N2CCC(c3cc4cc(NC(=O)C5CC5C#N)ncc4cc3Cl)CC2)COC1] | Cal'c: 425 Found: 425 |
| 2.10 | 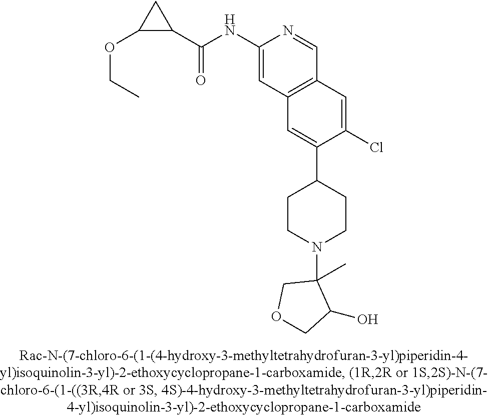 [SMILES: CCOC1CC1C(=O)Nc1cc2cc(C3CCN(C4(C)COCC4O)CC3)c(Cl)cc2cn1] | Cal'c: 474 Found: 474 |
| 2.11-2.12 | 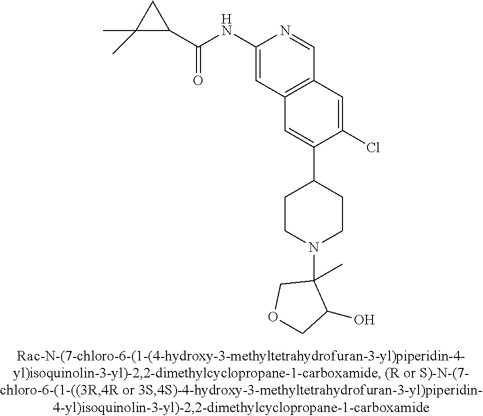 [SMILES: CC1(C)CC1C(=O)Nc1cc2cc(C3CCN(C4(C)COCC4O)CC3)c(Cl)cc2cn1] | Cal'c: 458 Found: 458 |
| 2.13-2.14 | 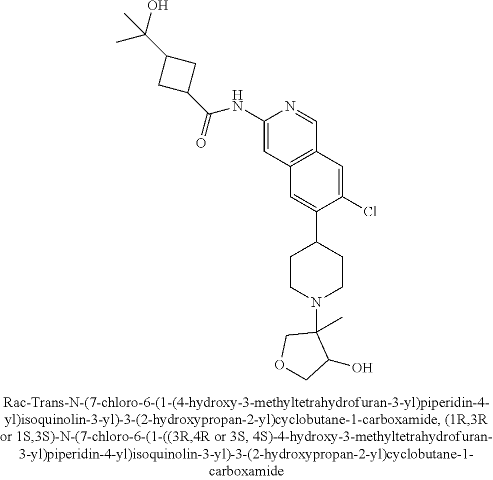 [SMILES: CC(C)(O)C1CC(C(=O)Nc2cc3cc(C4CCN(C5(C)COCC5O)CC4)c(Cl)cc3cn2)C1] | Cal'c: 502 Found: 502 |
| 2.15 | 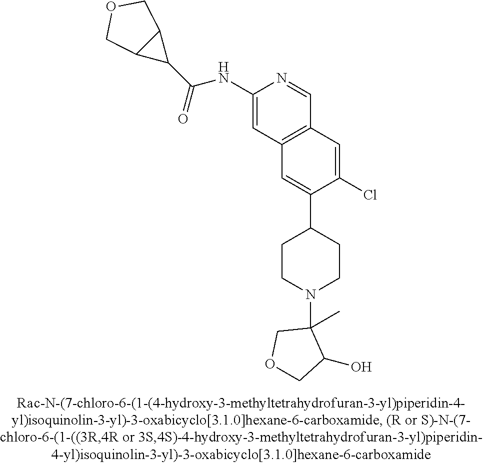 [SMILES: CC1(N2CCC(c3cc4cc(NC(=O)C5C6COCC65)ncc4cc3Cl)CC2)COCC1O] | Cal'c: 472 Found: 472 |
| 2.16-2.17 | 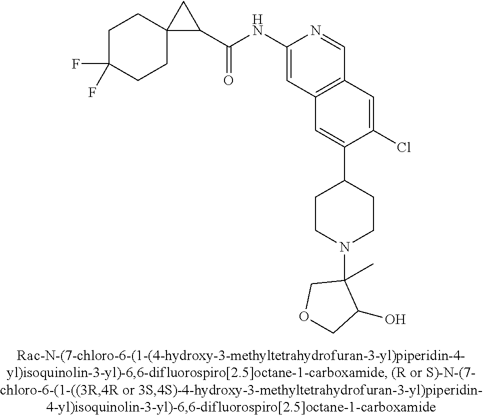 [SMILES: CC1(N2CCC(c3cc4cc(NC(=O)C5CC56CCC(F)(F)CC6)ncc4cc3Cl)CC2)COCC1O] | Cal'c: 534 Found: 534 |
| 2.18-2.19 | 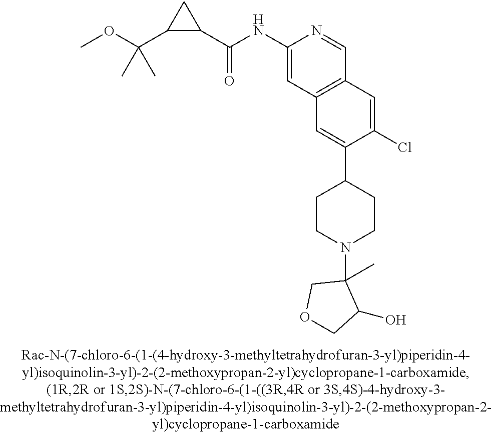 [SMILES: COC(C)(C)C1CC1C(=O)Nc1cc2cc(C3CCN(C4(C)COCC4O)CC3)c(Cl)cc2cn1] | Cal'c: 502 Found: 502 |
| 2.20-2.21 | 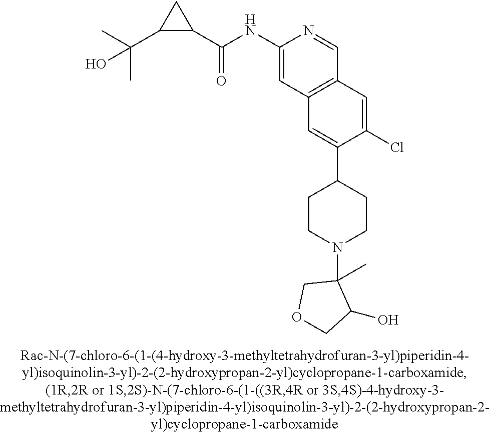 [SMILES: CC(C)(O)C1CC1C(=O)Nc1cc2cc(C3CCN(C4(C)COCC4O)CC3)c(Cl)cc2cn1] | Cal'c: 488 Found: 488 |
| 2.22-2.23 | 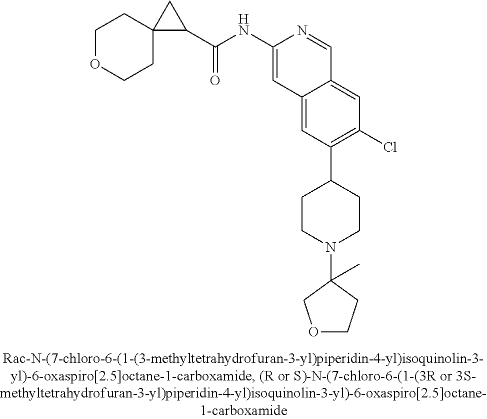 [SMILES: CC1(N2CCC(c3cc4cc(NC(=O)C5CC56CCOCC6)ncc4cc3Cl)CC2)CCOC1] | Cal'c: 484 Found: 484 |
| 2.24 | 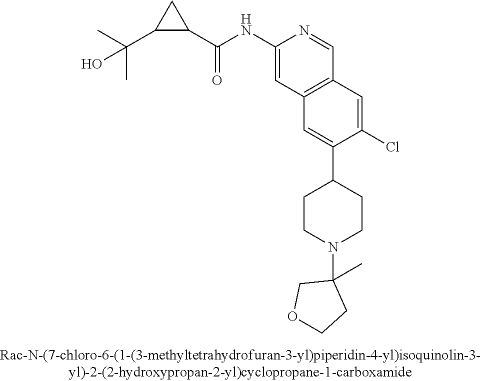 [SMILES: CC(C)(O)C1CC1C(=O)Nc1cc2cc(C3CCN(C4(C)CCOC4)CC3)c(Cl)cc2cn1] | Cal'c: 472 Found: 472 |
| 2.25-2.26 | 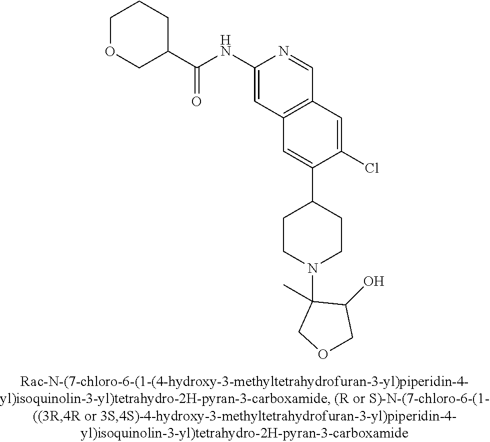 [SMILES: CC1(N2CCC(c3cc4cc(NC(=O)C5CCCOC5)ncc4cc3Cl)CC2)COCC1O] | Cal'c: 474 Found: 474 |
| 2.27 | 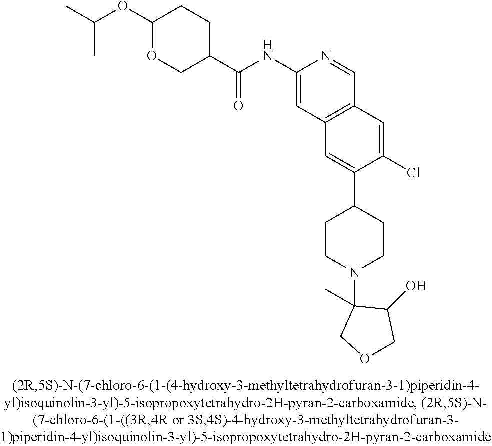 [SMILES: CC(C)OC1CCC(C(=O)Nc2cc3cc(C4CCN(C5(C)COCC5O)CC4)c(Cl)cc3cn2)CO1] | Cal'c: 532 Found: 532 |
| 2.28 | 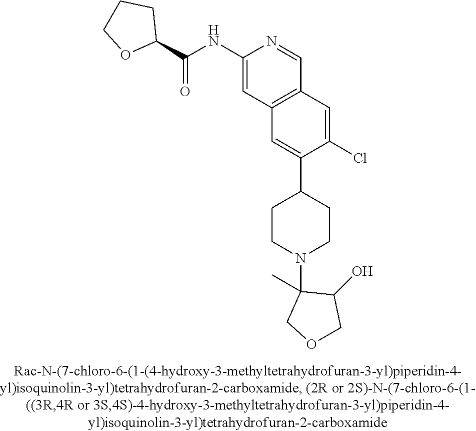 [SMILES: CC1(N2CCC(c3cc4cc(NC(=O)[C@@H]5CCCO5)ncc4cc3Cl)CC2)COCC1O] | Cal'c: 460 Found: 460 |
| 2.29-2.30 | 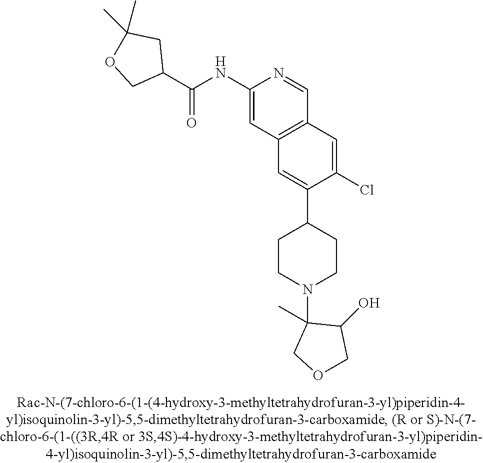 [SMILES: CC1(C)CC(C(=O)Nc2cc3cc(C4CCN(C5(C)COCC5O)CC4)c(Cl)cc3cn2)CO1] | Cal'c: 488 Found: 488 |
| 2.31 | 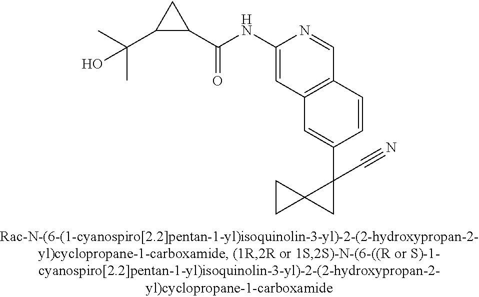 [SMILES: CC(C)(O)C1CC1C(=O)Nc1cc2cc(C3(C#N)CC34CC4)ccc2cn1] | Cal'c: 362 Found: 362 |
| 2.32-2.33 | 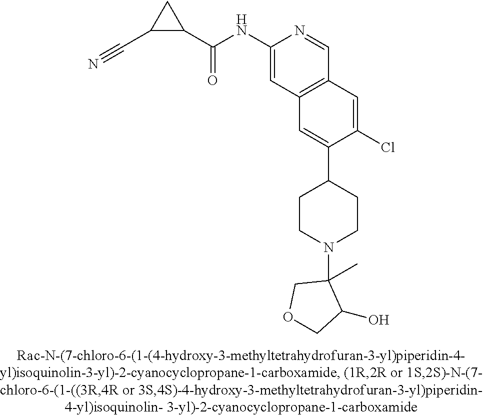 [SMILES: CC1(N2CCC(c3cc4cc(NC(=O)C5CC5C#N)ncc4cc3Cl)CC2)COCC1O] | Cal'c: 455 Found: 455 |
| 2.34-2.35 | 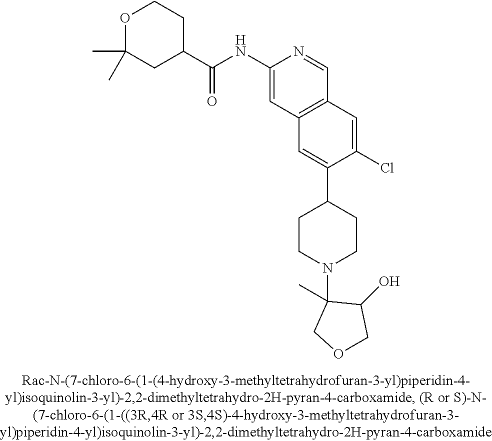 [SMILES: CC1(C)CC(C(=O)Nc2cc3cc(C4CCN(C5(C)COCC5O)CC4)c(Cl)cc3cn2)CCO1] | Cal'c: 502 Found: 502 |
| 2.36-2.37 | 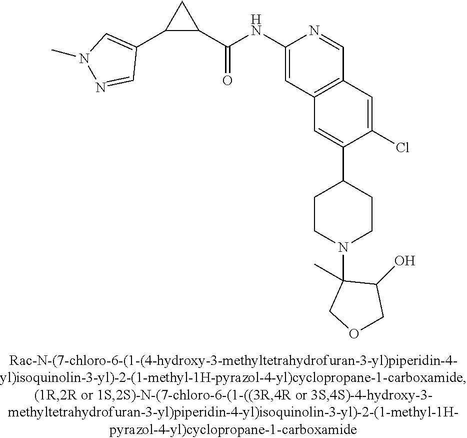 [SMILES: Cn1cc(C2CC2C(=O)Nc2cc3cc(C4CCN(C5(C)COCC5O)CC4)c(Cl)cc3cn2)cn1] | Cal'c: 510 Found: 510 |
| 2.38-2.40 | 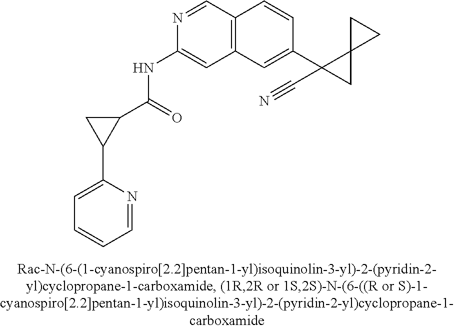 [SMILES: N#CC1(c2ccc3cnc(NC(=O)C4CC4c4ccccn4)cc3c2)CC12CC2] | Cal'c: 381 Found: 381 |
| 2.41-2.42 | 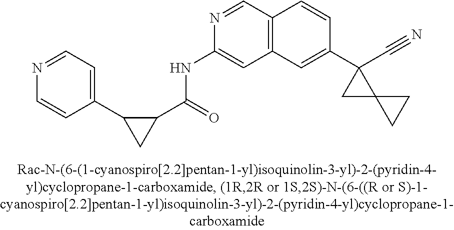 [SMILES: N#CC1(c2ccc3cnc(NC(=O)C4CC4c4ccncc4)cc3c2)CC12CC2] | Cal'c: 381 Found: 381 |
| 2.43-2.44 | 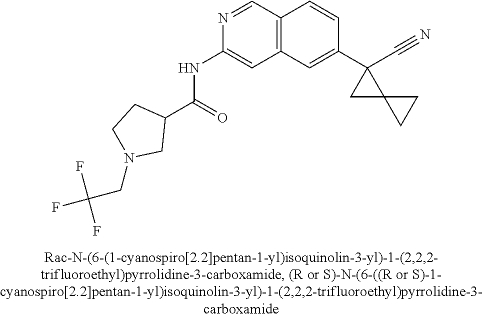 [SMILES: N#CC1(c2ccc3cnc(NC(=O)C4CCN(CC(F)(F)F)C4)cc3c2)CC12CC2] | Cal'c: 415 Found: 415 |
| 2.45-2.46 | 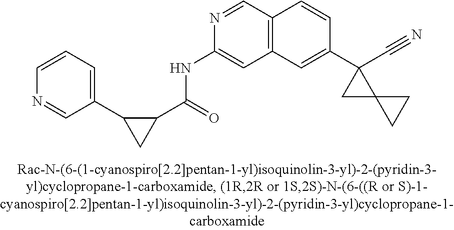 [SMILES: N#CC1(c2ccc3cnc(NC(=O)C4CC4c4cccnc4)cc3c2)CC12CC2] | Cal'c: 381 Found: 381 |
| 2.47-2.48 | 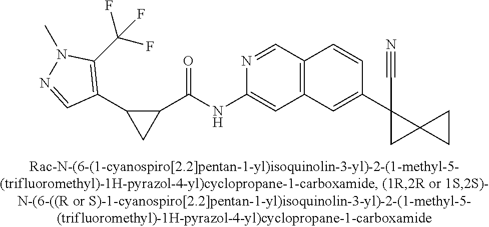 [SMILES: Cn1ncc(C2CC2C(=O)Nc2cc3cc(C4(C#N)CC45CC5)ccc3cn2)c1C(F)(F)F] | Cal'c: 452 Found: 452 |
| 2.49-2.50 | 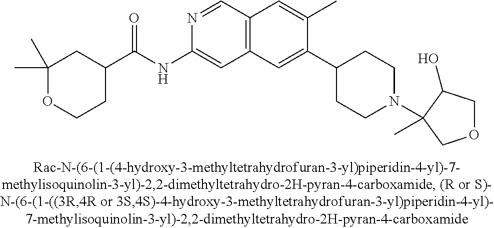 [SMILES: Cc1cc2cnc(NC(=O)C3CCOC(C)(C)C3)cc2cc1C1CCN(C2(C)COCC2O)CC1] | Cal'c: 482 Found: 482 |
| 2.51-2.52 | 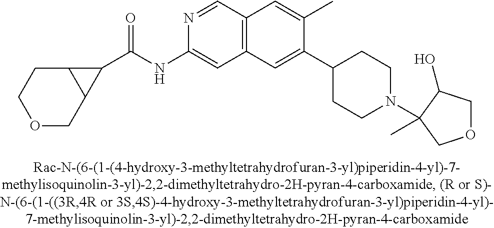 [SMILES: Cc1cc2cnc(NC(=O)C3C4CCOCC43)cc2cc1C1CCN(C2(C)COCC2O)CC1] | Cal'c: 466 Found: 466 |
| 2.53-2.56 | 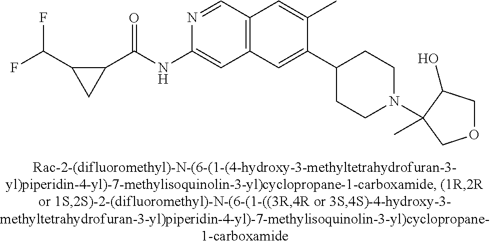 [SMILES: Cc1cc2cnc(NC(=O)C3CC3C(F)F)cc2cc1C1CCN(C2(C)COCC2O)CC1] | Cal'c: 460 Found: 460 |
| 2.57-2.58 | 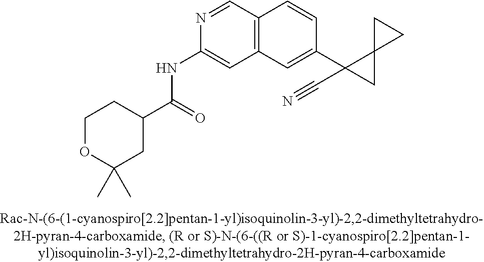 [SMILES: CC1(C)CC(C(=O)Nc2cc3cc(C4(C#N)CC45CC5)ccc3cn2)CCO1] | Cal'c: 376 Found: 376 |
| 2.59-2.62 | 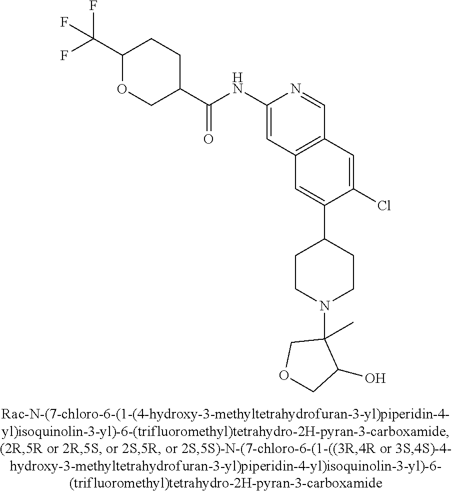 [SMILES: CC1(N2CCC(c3cc4cc(NC(=O)C5CCC(C(F)(F)F)OC5)ncc4cc3Cl)CC2)COCC1O] | Cal'c: 542 Found: 542 |
| 2.63-2.66 | 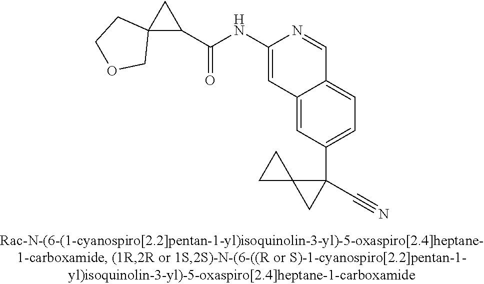 [SMILES: N#CC1(c2ccc3cnc(NC(=O)C4CC45CCOC5)cc3c2)CC12CC2] | Cal'c: 360 Found: 360 |
| 2.67-2.70 | 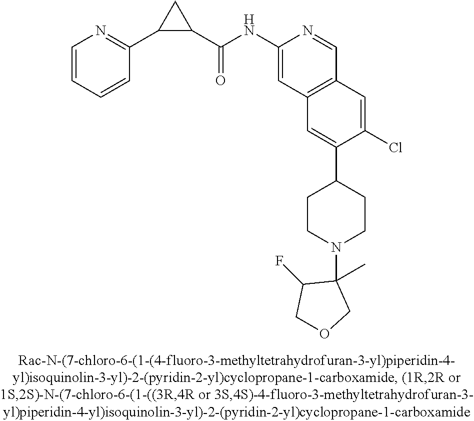 [SMILES: CC1(N2CCC(c3cc4cc(NC(=O)C5CC5c5ccccn5)ncc4cc3Cl)CC2)COCC1F] | Cal'c: 509 Found: 509 |
| 2.71-2.72 | 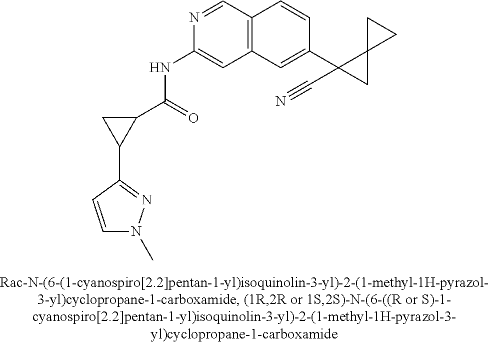 [SMILES: Cn1ccc(C2CC2C(=O)Nc2cc3cc(C4(C#N)CC45CC5)ccc3cn2)n1] | Cal'c: 442 Found: 442 |
| 2.73-2.75 | 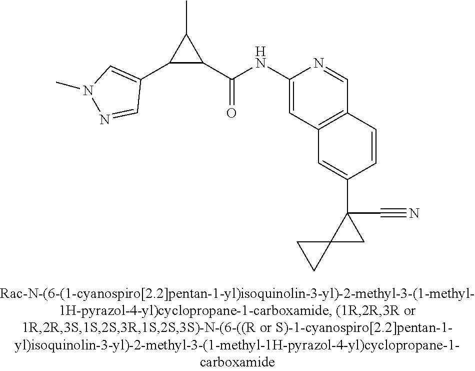 [SMILES: CC1C(C(=O)Nc2cc3cc(C4(C#N)CC45CC5)ccc3cn2)C1c1cnn(C)c1] | Cal'c: 398 Found: 398 |
| 2.76-2.77 | 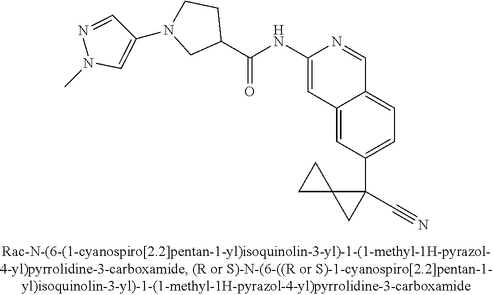 [SMILES: Cn1cc(N2CCC(C(=O)Nc3cc4cc(C5(C#N)CC56CC6)ccc4cn3)C2)cn1] | Cal'c: 413 Found: 413 |
| 2.78-2.79 | 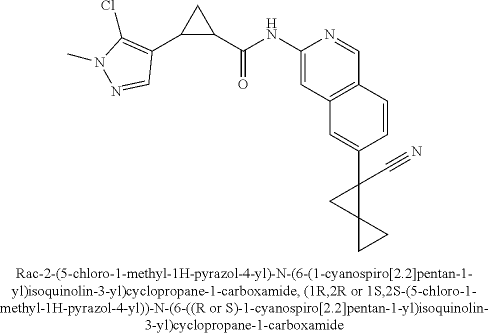 [SMILES: Cn1ncc(C2CC2C(=O)Nc2cc3cc(C4(C#N)CC45CC5)ccc3cn2)c1Cl] | Cal'c: 418 Found: 418 |
| 2.80-2.81 | 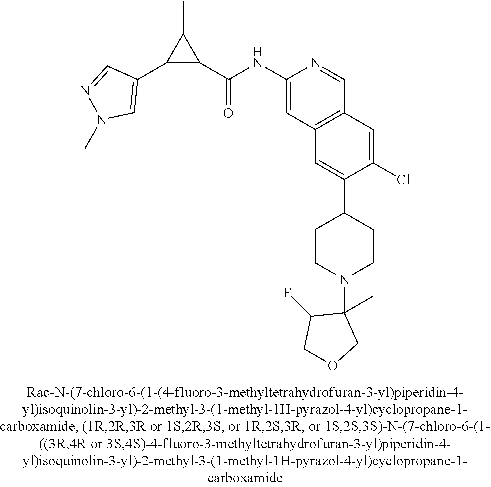 [SMILES: CC1C(C(=O)Nc2cc3cc(C4CCN(C5(C)COCC5F)CC4)c(Cl)cc3cn2)C1c1cnn(C)c1] | Cal'c: 526 Found: 526 |
| 2.82-2.83 | 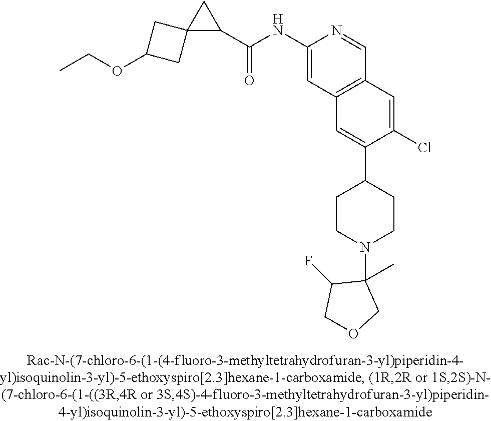 [SMILES: CCOC1CC2(C1)CC2C(=O)Nc1cc2cc(C3CCN(C4(C)COCC4F)CC3)c(Cl)cc2cn1] | Cal'c: 516 Found: 516 |
| 2.84-2.85 | 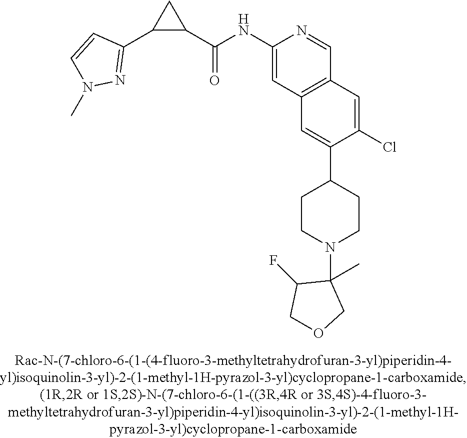 [SMILES: Cn1ccc(C2CC2C(=O)Nc2cc3cc(C4CCN(C5(C)COCC5F)CC4)c(Cl)cc3cn2)n1] | Cal'c: 512 Found: 512 |
| 2.86-2.89 | 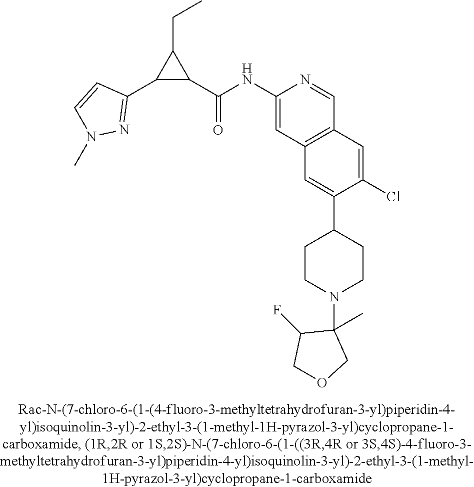 [SMILES: CCC1C(C(=O)Nc2cc3cc(C4CCN(C5(C)COCC5F)CC4)c(Cl)cc3cn2)C1c1ccn(C)n1] | Cal'c: 540 Found: 540 |
| 2.90-2.91 | 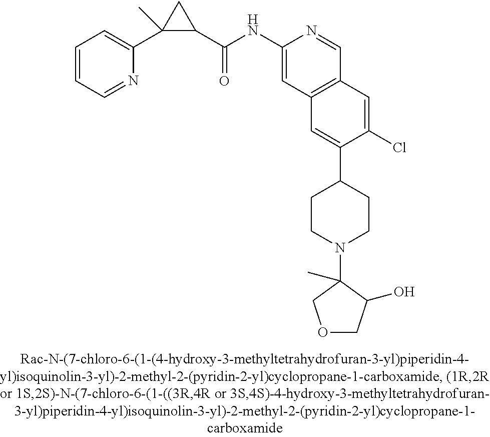 [SMILES: CC1(c2ccccn2)CC1C(=O)Nc1cc2cc(C3CCN(C4(C)COCC4O)CC3)c(Cl)cc2cn1] | Cal'c: 521 Found: 521 |
| 2.92-2.93 | 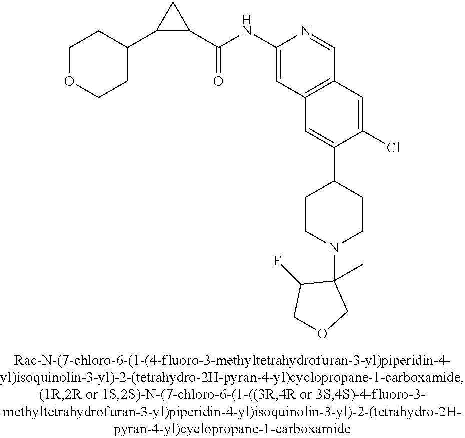 [SMILES: CC1(N2CCC(c3cc4cc(NC(=O)C5CC5C5CCOCC5)ncc4cc3Cl)CC2)COCC1F] | Cal'c: 516 Found: 516 |
| 2.94-2.95 | 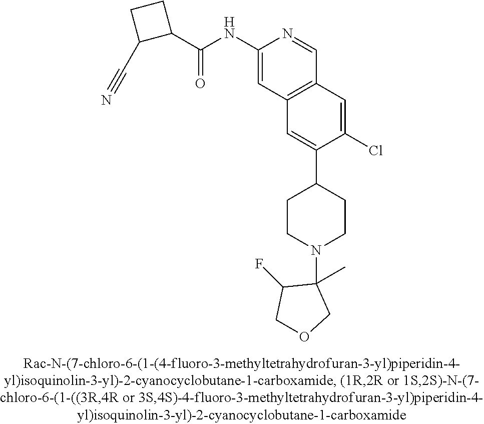 [SMILES: CC1(N2CCC(c3cc4cc(NC(=O)C5CCC5C#N)ncc4cc3Cl)CC2)COCC1F] | Cal'c: 516 Found: 516 |
| 2.96-2.97 | 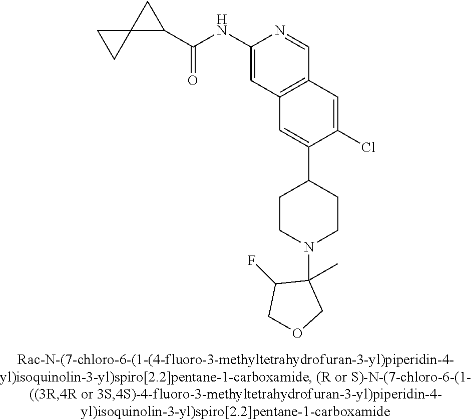 [SMILES: CC1(N2CCC(c3cc4cc(NC(=O)C5CC56CC6)ncc4cc3Cl)CC2)COCC1F] | Cal'c: 458 Found: 458 |
| 2.98-2.99 | 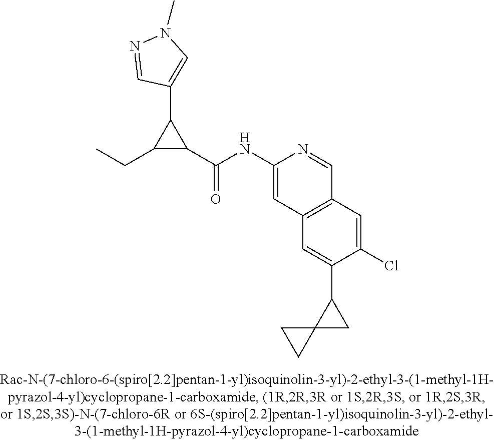 [SMILES: CCC1C(C(=O)Nc2cc3cc(C4CC45CC5)c(Cl)cc3cn2)C1c1cnn(C)c1] | Cal'c: 421 Found: 421 |
| 2.100-2.101 | 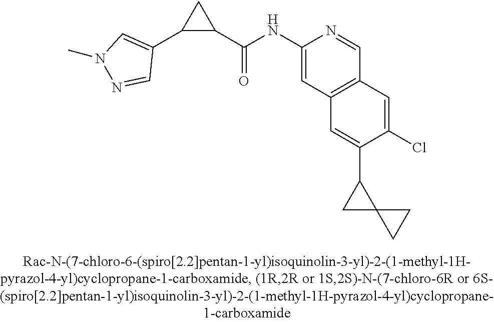 [SMILES: Cn1cc(C2CC2C(=O)Nc2cc3cc(C4CC45CC5)c(Cl)cc3cn2)cn1] | Cal'c: 393 Found: 393 |
| 2.102-2.103 | 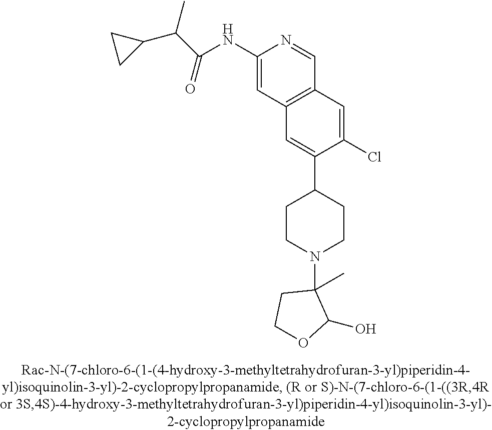 [SMILES: CC(C(=O)Nc1cc2cc(C3CCN(C4(C)CCOC4O)CC3)c(Cl)cc2cn1)C1CC1] | Cal'c: 458 Found: 458 |
| 2.104-2.105 | 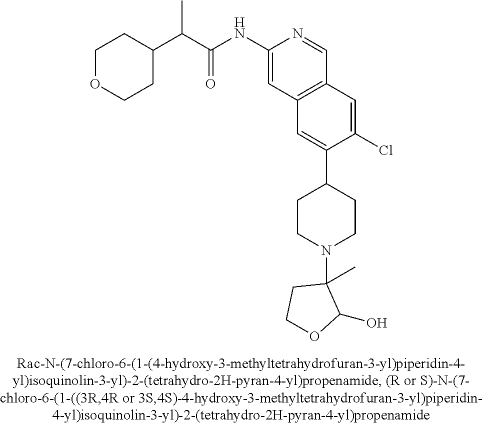 [SMILES: CC(C(=O)Nc1cc2cc(C3CCN(C4(C)CCOC4O)CC3)c(Cl)cc2cn1)C1CCOCC1] | Cal'c: 502 Found: 502 |
| 2.106-2.107 | 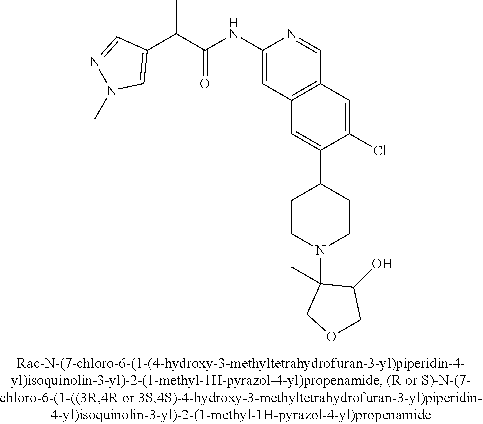 [SMILES: CC(C(=O)Nc1cc2cc(C3CCN(C4(C)COCC4O)CC3)c(Cl)cc2cn1)c1cnn(C)c1] | Cal'c: 498 Found: 498 |
| 2.108-2.111 | 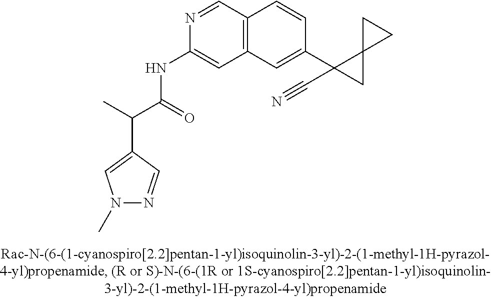 [SMILES: CC(C(=O)Nc1cc2cc(C3(C#N)CC34CC4)ccc2cn1)c1cnn(C)c1] | Cal'c: 372 Found: 372 |
| 2.112-2.113 | 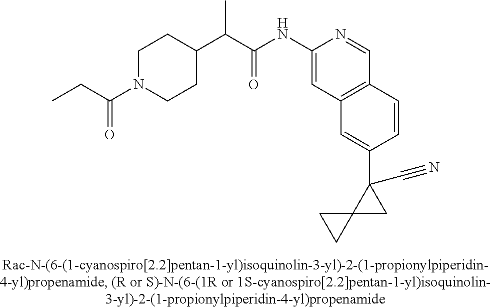 [SMILES: CCC(=O)N1CCC(C(C)C(=O)Nc2cc3cc(C4(C#N)CC45CC5)ccc3cn2)CC1] | Cal'c: 431 Found: 431 |

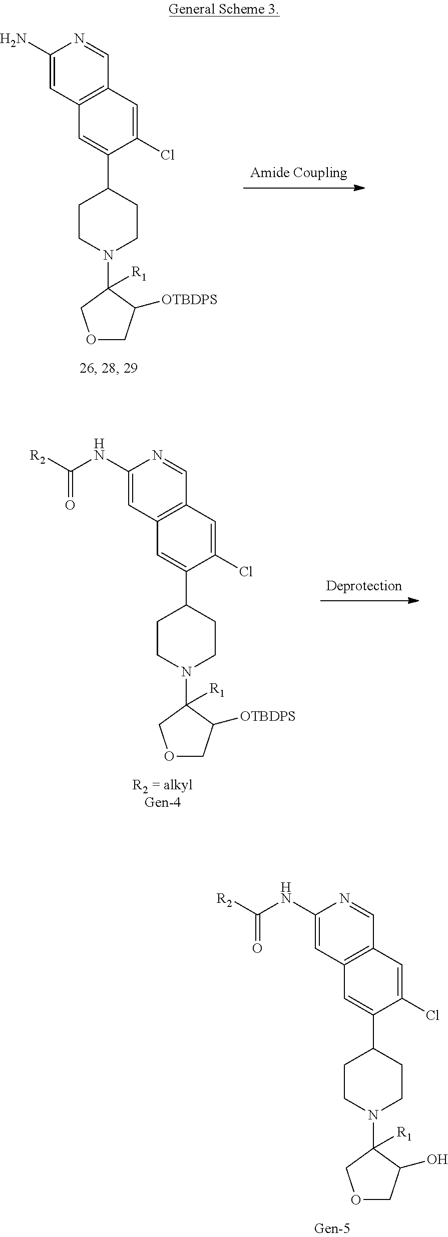

In General Scheme 3, commercially available carboxylic acids or acid chlorides were coupled with intermediates 26, 28 or 29 through amide coupling conditions to provide Gen-4. These compounds could in turn be deprotected to afford compounds that are fully elaborated in the form of Gen-5. The representative compounds are described in more detail below.

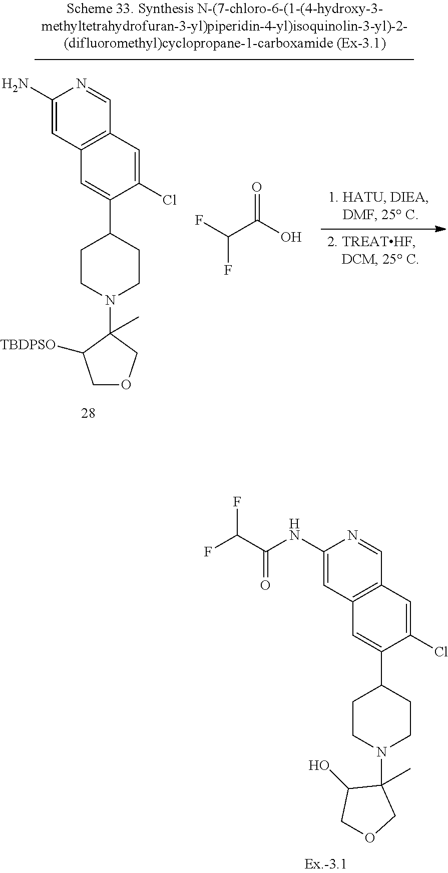

2-(difluoromethyl)cyclopropane-1-carboxylic acid (45.3 mg, 0.333 mmol), 6-(1-(4-((tert-butyldiphenylsilyl)oxy)-3-methyltetrahydrofuran-3-yl)piperidin-4-yl)-7-chloroisoquinolin-3-amine 28 (100 mg, 0.167 mmol), HATU (190 mg, 0.500 mmol), DMF (833 μl) and Hunig's Base (145 μl, 0.833 mmol). The reaction mixture was stirred at rt overnight. The reaction mixture was diluted in DCM and extracted with saturated ammonium chloride. Organic layers were combined, dried, and concentrated. The reaction mixture was taken up in THF (833 μl) and TBAF (500 μl, 0.500 mmol) was added. The reaction mixture was stirred at 40° C. for 3 hrs. Reaction mixture was extracted with saturated sodium thiosulfate and DCM. Organic layers were combined, dried, and concentrated. The crude reaction mixture was diluted in DCM and purified by column chromatography using 0-70% Hexanes in 3:1 Ethyl Acetate Ethanol to afford title compound Ex-3.1. MS (ESI) m/z calc'd for C₂₄H₂₉ClFN₃O₃ [M+H]⁺: 480, found 480. ¹H NMR (400 MHz, d-DMSO, 25° C.) δ ¹H NMR (499 MHz, DMSO-d₆) δ 11.10 (s, 1H), 9.09 (m, 1H), 8.45 (m, 1H), 8.19 (s, 1H), 7.97 (s, 1H), 3.96 (m, 1H), 3.79 (m, 1H), 3.70 (m, 1H), 3.62 (m, 1H), 3.55 (m, 1H), 3.08-2.96 (m, 1H), 2.86 (m, 1H), 2.57 (m, 1H), 2.55-2.48 (m, 2H), 2.46-2.35 (m, 2H), 1.91-1.85 (m 4H), 1.82-1.76 (m, 1H), 1.31 (m, 1H), 1.19 (m, 1H), 1.15-1.06 (m, 1H), 1.05 (s, 3H).

Compounds in Table 3 below were prepared in accordance with the synthetic sequence illustrated in General Scheme 3 and Scheme 33 using the corresponding starting materials.

TABLE 3

| | | |
|---|---|---|
| 3.2 | 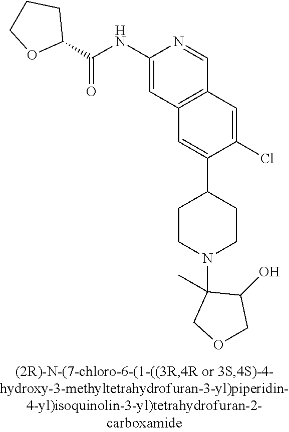 [SMILES: CC1(N2CCC(c3cc4cc(NC(=O)[C@H]5CCCO5)ncc4cc3Cl)CC2)COCC1O] | Cal'c: 460 Found: 460 |
| 3.3 | 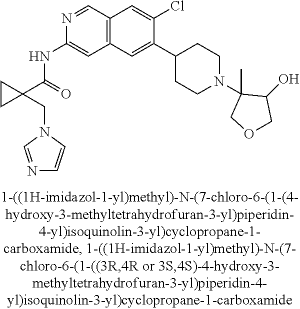 [SMILES: CC1(N2CCC(c3cc4cc(NC(=O)C5(Cn6ccnc6)CC5)ncc4cc3Cl)CC2)COCC1O] | Cal'c: 510 Found: 510 |
| 3.4 | 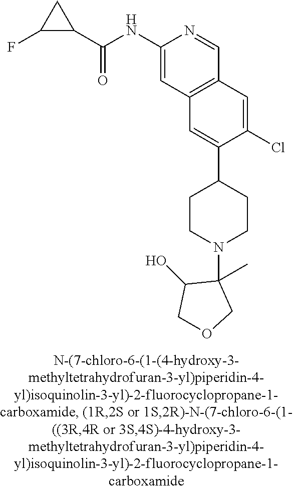 [SMILES: CC1(N2CCC(c3cc4cc(NC(=O)C5CC5F)ncc4cc3Cl)CC2)COCC1O] | Cal'c: 448 Found: 448 |
| 3.5 | 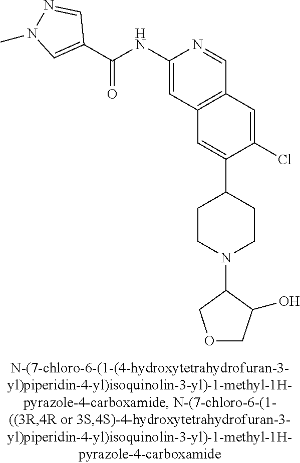 [SMILES: Cn1cc(C(=O)Nc2cc3cc(C4CCN(C5COCC5O)CC4)c(Cl)cc3cn2)cn1] | Cal'c: 456 Found: 456 |
| 3.6 | 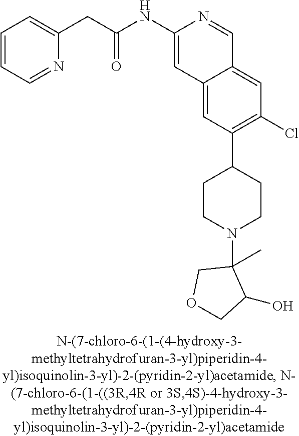 [SMILES: CC1(N2CCC(c3cc4cc(NC(=O)Cc5ccccn5)ncc4cc3Cl)CC2)COCC1O] | Cal'c: 481 Found: 481 |
| 3.7 | 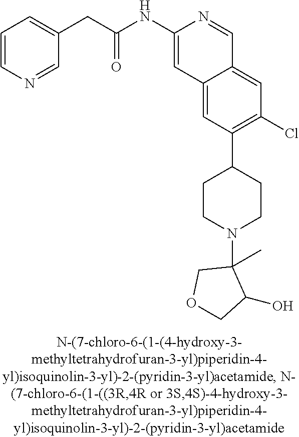 [SMILES: CC1(N2CCC(c3cc4cc(NC(=O)Cc5cccnc5)ncc4cc3Cl)CC2)COCC1O] | Cal'c: 481 Found: 481 |
| 3.8 | 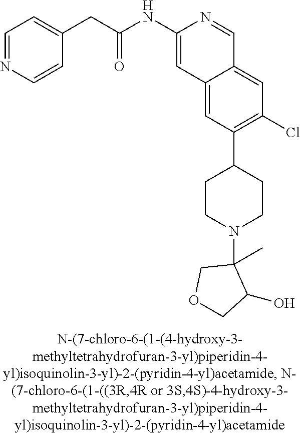 [SMILES: CC1(N2CCC(c3cc4cc(NC(=O)Cc5ccncc5)ncc4cc3Cl)CC2)COCC1O] | Cal'c: 481 Found: 481 |
| 3.9 | 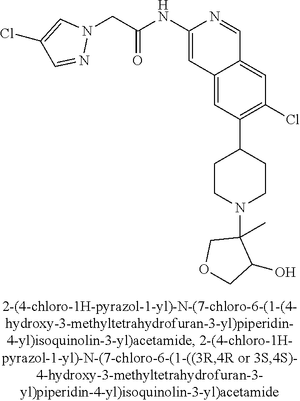 [SMILES: CC1(N2CCC(c3cc4cc(NC(=O)Cn5cc(Cl)cn5)ncc4cc3Cl)CC2)COCC1O] | Cal'c: 504 Found: 504 |
| 3.10 | 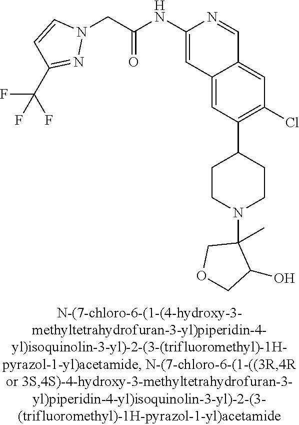 [SMILES: CC1(N2CCC(c3cc4cc(NC(=O)Cn5ccc(C(F)(F)F)n5)ncc4cc3Cl)CC2)COCC1O] | Cal'c: 538 Found: 538 |
| 3.11 | 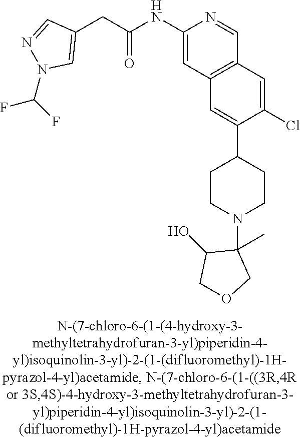 [SMILES: CC1(N2CCC(c3cc4cc(NC(=O)Cc5cnn(C(F)F)c5)ncc4cc3Cl)CC2)COCC1O] | Cal'c: 520 Found: 520 |
| 3.12 | 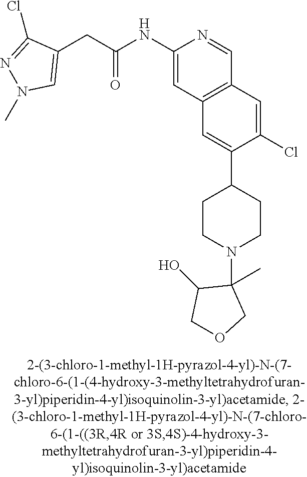 [SMILES: Cn1cc(CC(=O)Nc2cc3cc(C4CCN(C5(C)COCC5O)CC4)c(Cl)cc3cn2)c(Cl)n1] | Cal'c: 518 Found: 518 |

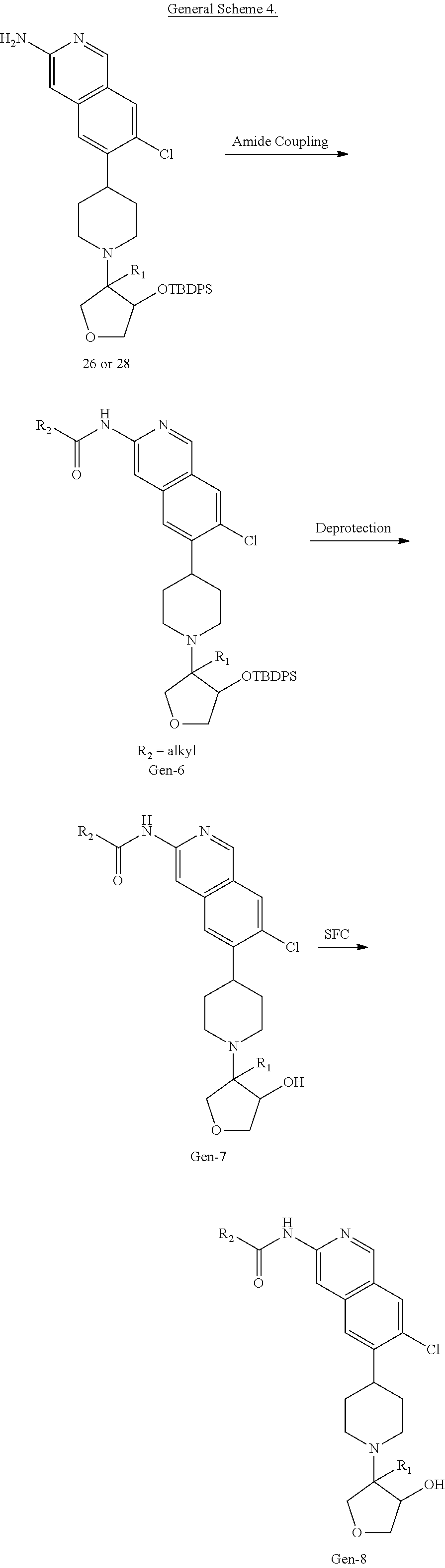

In General Scheme 4, commercially available carboxylic acids or acid chlorides were coupled with intermediates 26 or 28 through amide coupling conditions to provide Gen-6. These compounds could in turn be deprotected to afford compounds that are fully elaborated in the form of Gen-7 and the stereoisomers could then be separated by chiral SFC to provide fully elaborated products in the form of Gen-8. The representative compounds are described in more detail below.

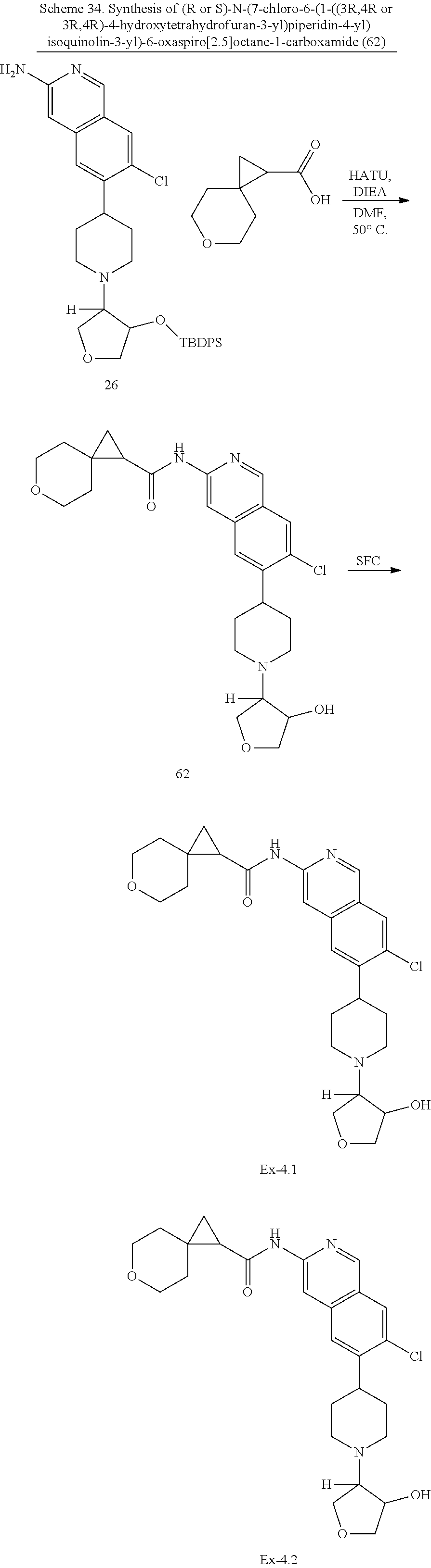

(R or S)-N-(7-chloro-6-(1-((3R,4R or 3R, 4R)-4-hydroxytetrahydrofuran-3-yl)piperidin-4-yl)isoquinolin-3-yl)-6-oxaspiro[2.5]octane-1-carboxamide (Ex-4.1) and (Ex-4.2) 6-(1-(4-((tert-butyldiphenylsilyl)oxy)tetrahydrofuran-3-yl)piperidin-4-yl)-7-chloroisoquinolin-3-amine 26 (382 mg, 0.652 mmol), 6-oxaspiro[2.5]octane-1-carboxylic acid (204 mg, 1.303 mmol), HATU (496 mg, 1.303 mmol), DMF (3258 μl), and DIEA (341 μl, 1.955 mmol) were added to a flask. The resulting mixture was allowed to stir overnight at 50° C. The reaction was cooled to room temperature and water was added to form a precipitate. The solids were collected by vacuum filtration and dried. The residue was dissolved in 4 mL of methanol and LiOH (1706 μl, 3.41 mmol) was added. The resulting mixture was stirred at 75° C. for 3 hours. The reaction mixture was concentrated under reduced pressure. The reaction mixture was diluted with DCM and washed twice with saturated sodium bicarbonate and once with brine. The combined organic fractions were dried over anhydrous magnesium sulfate, filtered and concentrated under reduced pressure. The residue was filtered and submitted directly for HPLC purification, eluting acetonitrile/water gradient with 0.10% TFA modifier, linear gradient and lyophilized to afford title compound 62 as a mixture of stereoisomers. The mixture of two stereoisomers was purified by chiral SFC (SJ, 21×250 (mm), 25%/75% Methanol/CO₂+0.1% NH₄OH) and lyophilized to afford each of the two stereoisomers of the title compound Ex-4.1 (tR=3.7 min) and Ex-4.2 (tR=4.7 min). MS (ESI): m/z calc'd for C₂₆H₃₂ClN₃O₄ [M+H]⁺: 486, found 486. ¹H NMR (499 MHz, DMSO-d6) δ 10.90 (s, 1H), 9.08 (s, 1H), 8.46 (s, 1H), 8.18 (s, 1H), 7.87 (s, 1H), 4.30 (s, 1H), 4.18 (s, 1H), 3.91-3.85 (m, 2H), 3.76-3.64 (m, 3H), 3.64-3.57 (m, 2H), 3.46-3.40 (m, 1H), 3.22 (m, 1H), 3.08-2.99 (m, 1H), 2.78 (m, 1H), 2.75-2.66 (m, 1H), 2.35-2.26 (m, 1H), 2.24-2.16 (m, 1H), 2.03 (m, 1H), 1.90-1.80 (m, 4H), 1.76-1.69 (m, 1H), 1.69-1.63 (m, 1H), 1.58-1.48 (m, 1H), 1.42-1.35 (m, 1H), 1.13 (t, 1H), 0.94 (m, 1H).

Compounds in Table 4 below were prepared in accordance with the synthetic sequence illustrated in General Scheme 4 and Scheme 34 using the corresponding starting materials.

TABLE 4

| | | |
|---|---|---|
| 4.3-4.4 | 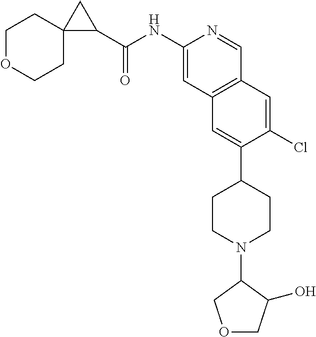 [SMILES: O=C(Nc1cc2cc(C3CCN(C4COCC4O)CC3)c(Cl)cc2cn1)C1CC12CCOCC2] | Cal'c: 486 Found: 486 |
| | N-(7-chloro-6-(1-(4-hydroxytetrahydrofuran-3-yl)piperidin-4- | |
| | yl)isoquinolin-3-yl)-6-oxaspiro[2.5]octane-1-carboxamide, (R or S)- | |
| | N-(7-chloro-6-(1-((3R,4R or 3S,4S)-4-hydroxytetrahydrofuran-3- | |
| | yl)piperidin-4-yl)isoquinolin-3-yl)-6-oxaspiro[2.5]octane-1- | |
| | carboxamide | |
| 4.5-4.6 | 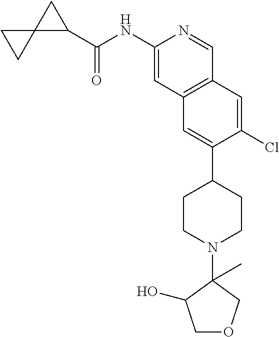 [SMILES: CC1(N2CCC(c3cc4cc(NC(=O)C5CC56CC6)ncc4cc3Cl)CC2)COCC1O] | Cal'c: 456 Found: 456 |
| | N-(7-chloro-6-(1-(4-hydroxy-3-methyltetrahydrofuran-3- | |
| | yl)piperidin-4-yl)isoquinolin-3-yl)spiro[2.2]pentane-1-carboxamide, | |
| | (R or S)-N-(7-chloro-6-(1-((3R,4R or 3S,4S)-4-hydroxy-3- | |
| | methyltetrahydrofuran-3-yl)piperidin-4-yl)isoquinolin-3- | |
| | yl)spiro[2.2]pentane-1-carboxamide | |
| 4.7-4.8 | 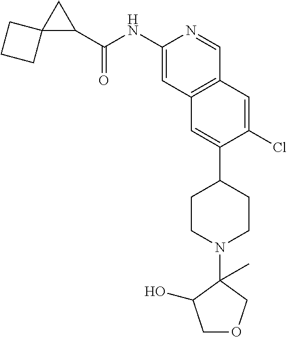 [SMILES: CC1(N2CCC(c3cc4cc(NC(=O)C5CC56CCC6)ncc4cc3Cl)CC2)COCC1O] | Cal'c: 470 Found: 470 |
| | N-(7-chloro-6-(1-(4-hydroxy-3-methyltetrahydrofuran-3- | |
| | yl)piperidin-4-yl)isoquinolin-3-yl)spiro[2.3]hexane-1-carboxamide, | |
| | (R or S)-N-(7-chloro-6-(1-((3R,4R or 3S,4S)-4-hydroxy-3- | |
| | methyltetrahydrofuran-3-yl)piperidin-4-yl)isoquinolin-3- | |
| | yl)spiro[2.3]hexane-1-carboxamide | |
| 4.9-4.10 | 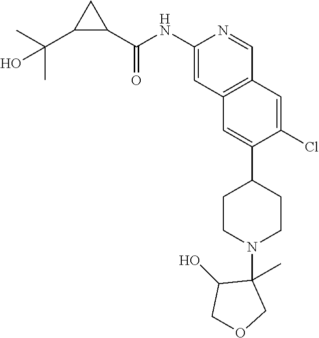 [SMILES: CC(C)(O)C1CC1C(=O)Nc1cc2cc(C3CCN(C4(C)COCC4O)CC3)c(Cl)cc2cn1] | Cal'c: 488 Found: 488 |
| | Trans-N-(7-chloro-6-(1-(4-hydroxy-3-methyltetrahydrofuran-3- | |
| | yl)piperidin-4-yl)isoquinolin-3-yl)-2-(2-hydroxypropan-2- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(2-hydroxypropan-2- | |
| | yl)cyclopropane-1-carboxamide | |
| 4.11-4.14 | 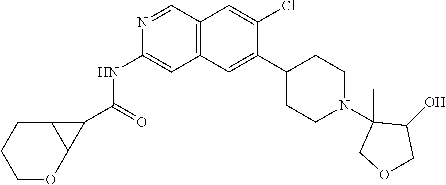 [SMILES: CC1(N2CCC(c3cc4cc(NC(=O)C5C6CCCOC65)ncc4cc3Cl)CC2)COCC1O] | Cal'c: 486 Found: 486 |
| | N-(7-chloro-6-(1-(4-hydroxy-3-methyltetrahydrofuran-3- | |
| | yl)piperidin-4-yl)isoquinolin-3-yl)-2-oxabicyclo[4.1.0]heptane-7- | |
| | carboxamide, (1S,6S,7R or 1R,6R,7R)-N-(7-chloro-6-(1-((3R,4R or | |
| | 3S,4S)-4-hydroxy-3-methyltetrahydrofuran-3-yl)piperidin-4- | |
| | yl)isoquinolin-3-yl)-2-oxabicyclo[4.1.0]heptane-7-carboxamide | |
| 4.15-4.16 | 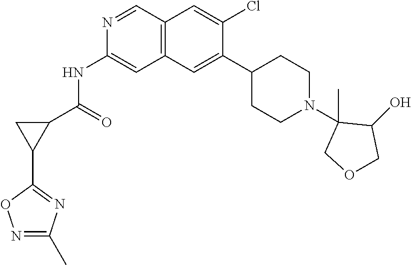 [SMILES: Cc1noc(C2CC2C(=O)Nc2cc3cc(C4CCN(C5(C)COCC5O)CC4)c(Cl)cc3cn2)n1] | Cal'c: 512 Found: 512 |
| | N-(7-chloro-6-(1-(4-hydroxy-3-methyltetrahydrofuran-3- | |
| | yl)piperidin-4-yl)isoquinolin-3-yl)-2-(3-methyl-1,2,4-oxadiazol-5- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(3-methyl-1,2,4-oxadiazol-5- | |
| | yl)cyclopropane-1-carboxamide | |
| 4.17-4.18 | 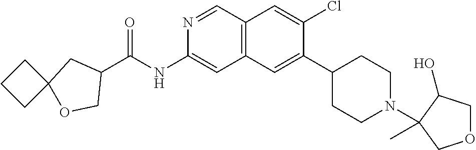 [SMILES: CC1(N2CCC(c3cc4cc(NC(=O)C5COC6(CCC6)C5)ncc4cc3Cl)CC2)COCC1O] | Cal'c: 500 Found: 500 |
| | N-(7-chloro-6-(1-(4-hydroxy-3-methyltetrahydrofuran-3- | |
| | yl)piperidin-4-yl)isoquinolin-3-yl)-5-oxaspiro[3.4]octane-7- | |
| | carboxamide, (7S or 7R)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-5-oxaspiro[3.4]octane-7-carboxamide | |
| 4.19-4.20 | 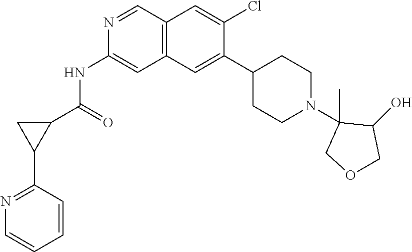 [SMILES: CC1(N2CCC(c3cc4cc(NC(=O)C5CC5c5ccccn5)ncc4cc3Cl)CC2)COCC1O] | Cal'c: 507 Found: 507 |
| | N-(7-chloro-6-(1-(4-hydroxy-3-methyltetrahydrofuran-3- | |
| | yl)piperidin-4-yl)isoquinolin-3-yl)-2-(pyridin-2-yl)cyclopropane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(pyridin-2-yl)cyclopropane-1-carboxamide | |
| 4.21-4.22 | 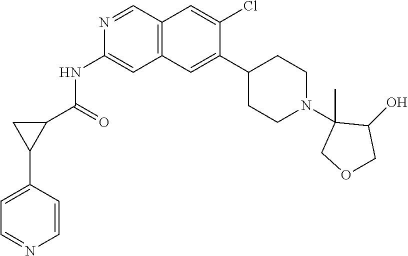 [SMILES: CC1(N2CCC(c3cc4cc(NC(=O)C5CC5c5ccncc5)ncc4cc3Cl)CC2)COCC1O] | Cal'c: 507 Found: 507 |
| | N-(7-chloro-6-(1-(4-hydroxy-3-methyltetrahydrofuran-3- | |
| | yl)piperidin-4-yl)isoquinolin-3-yl)-2-(pyridin-4-yl)cyclopropane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(pyridin-4-yl)cyclopropane-1-carboxamide | |
| 4.23-4.24 | 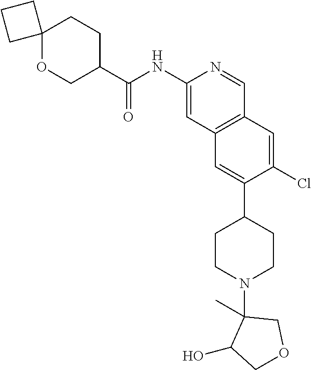 [SMILES: CC1(N2CCC(c3cc4cc(NC(=O)C5CCC6(CCC6)OC5)ncc4cc3Cl)CC2)COCC1O] | Cal'c: 514 Found: 514 |
| | N-(7-chloro-6-(1-(4-hydroxy-3-methyltetrahydrofuran-3- | |
| | yl)piperidin-4-yl)isoquinolin-3-yl)-5-oxaspiro[3.5]nonane-7- | |
| | carboxamide, (7S or 7R)-N-(7-chloro-6-(1-(3R,4R or 3S,4S)-4- | |
| | hydroxy-3-methyltetrahydrofuran-3-yl)piperidin-4-yl)isoquinolin-3- | |
| | yl)-5-oxaspiro[3.5]nonane-7-carboxamide | |
| 4.25-4.26 | 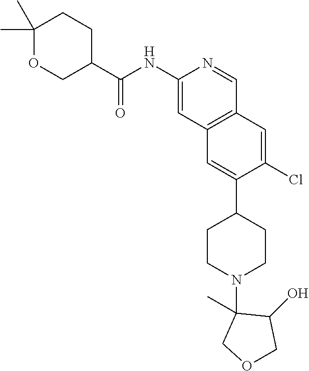 [SMILES: CC1(C)CCC(C(=O)Nc2cc3cc(C4CCN(C5(C)COCC5O)CC4)c(Cl)cc3cn2)CO1] | Cal'c: 502 Found: 502 |
| | N-(7-chloro-6-(1-(4-hydroxy-3-methyltetrahydrofuran-3- | |
| | yl)piperidin-4-yl)isoquinolin-3-yl)-6,6-dimethyltetrahydrofuran-2H-pyran- | |
| | 3-carboxamide, (3S or 3R)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-6,6-dimethyltetrahydro-2H-pyran-3-carboxamide | |
| 4.27-4.28 | 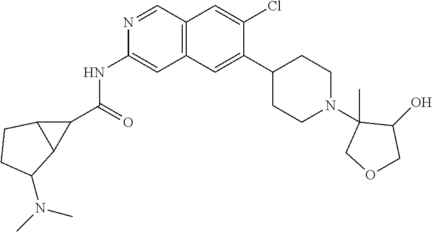 [SMILES: CN(C)C1CCC2C(C(=O)Nc3cc4cc(C5CCN(C6(C)COCC6O)CC5)c(Cl)cc4cn3)C21] | Cal'c: 513 Found: 513 |
| | N-(7-chloro-6-(1-(4-hydroxy-3-methyltetrahydrofuran-3- | |
| | yl)piperidin-4-yl)isoquinolin-3-yl)-2- | |
| | (dimethylamino)bicyclo[3.1.0]hexane-6-carboxamide, (1R,5S,6R or | |
| | 1S,5R,6R, 1R,5S,6S, or 1S,5R,6R)-N-(7-chloro-6-(1-((3R,4R or | |
| | 3S,4S)-4-hydroxy-3-methyltetrahydrofuran-3-yl)piperidin-4- | |
| | yl)isoquinolin-3-yl)-2-(dimethylamino)bicyclo[3.1.0]hexane-6- | |
| | carboxamide | |
| 4.29-4.32 | 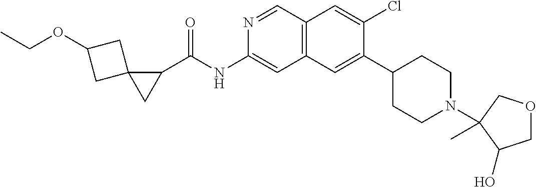 [SMILES: CCOC1CC2(C1)CC2C(=O)Nc1cc2cc(C3CCN(C4(C)COCC4O)CC3)c(Cl)cc2cn1] | Cal'c: 514 Found: 514 |
| | N-(7-chloro-6-(1-(4-hydroxy-3-methyltetrahydrofuran-3- | |
| | yl)piperidin-4-yl)isoquinolin-3-yl)-5-ethoxyspiro[2.3]hexane-1- | |
| | carboxamide, (1R or 1S)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-5-ethoxyspiro[2.3]hexane-1-carboxamide | |
| 4.33-4.34 | 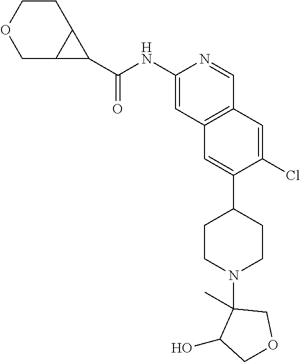 [SMILES: CC1(N2CCC(c3cc4cc(NC(=O)C5C6CCOCC65)ncc4cc3Cl)CC2)COCC1O] | Cal'c: 486 Found: 486 |
| | N-(7-chloro-6-(1-(4-hydroxy-3-methyltetrahydrofuran-3- | |
| | yl)piperidin-4-yl)isoquinolin-3-yl)-3-oxabicyclo[4.1.0]heptane-7- | |
| | carboxamide, (1R,6S,7R or 1R,6S,7S, or 1S,6R,7R, or 1S,6R,7S)-N- | |
| | (7-chloro-6-(1-((3R,4R or 3S,4S)-4-hydroxy-3- | |
| | methyltetrahydrofuran-3-yl)piperidin-4-yl)isoquinolin-3-yl)-3- | |
| | oxabicyclo[4.1.0]heptane-7-carboxamide | |
| 4.35-4.36 | 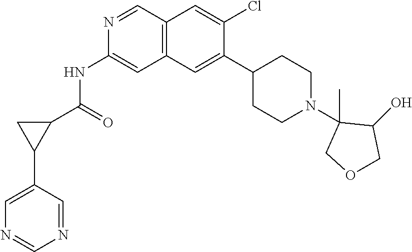 [SMILES: CC1(N2CCC(c3cc4cc(NC(=O)C5CC5c5cncnc5)ncc4cc3Cl)CC2)COCC1O] | Cal'c: 508 Found: 508 |
| | N-(7-chloro-6-(1-(4-hydroxy-3-methyltetrahydrofuran-3- | |
| | yl)piperidin-4-yl)isoquinolin-3-yl)-2-(pyrimidin-5-yl)cyclopropane- | |
| | 1-carboxamide, (1R,2R or 1S,2S)-N-(7-chloro-6-(1-((3R,4R or | |
| | 3S,4S)-4-hydroxy-3-methyltetrahydrofuran-3-yl)piperidin-4- | |
| | yl)isoquinolin-3-yl)-2-(pyrimidin-5-yl)cyclopropane-1-carboxamide | |
| 4.37-4.38 | 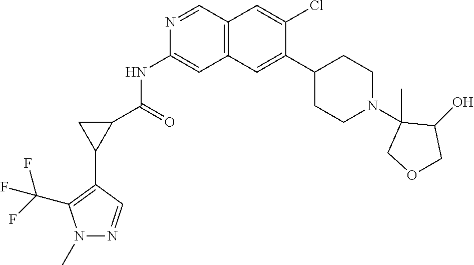 [SMILES: Cn1ncc(C2CC2C(=O)Nc2cc3cc(C4CCN(C5(C)COCC5O)CC4)c(Cl)cc3cn2)c1C(F)(F)F] | Cal'c: 578 Found: 578 |
| | N-(7-chloro-6-(1-(4-hydroxy-3-methyltetrahydrofuran-3- | |
| | yl)piperidin-4-yl)isoquinolin-3-yl)-2-(1-methyl-5-(trifluoromethyl)- | |
| | 1H-pyrazol-4-yl)cyclopropane-1-carboxamide, (1R,2R or 1S,2S)-N- | |
| | (7-chloro-6-(1-((3R,4R or 3S,4S)-4-hydroxy-3- | |
| | methyltetrahydrofuran-3-yl)piperidin-4-yl)isoquinolin-3-yl)-2-(1- | |
| | methyl-5-(trifluoromethyl)-1H-pyrazol-4-yl)cyclopropane-1- | |
| | carboxamide | |
| 4.39-4.40 | 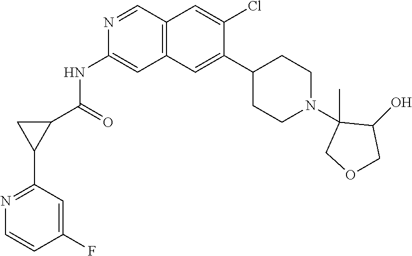 [SMILES: CC1(N2CCC(c3cc4cc(NC(=O)C5CC5c5cc(F)ccn5)ncc4cc3Cl)CC2)COCC1O] | Cal'c: 525 Found: 525 |
| | N-(7-chloro-6-(1-(4-hydroxy-3-methyltetrahydrofuran-3- | |
| | yl)piperidin-4-yl)isoquinolin-3-yl)-2-(4-fluoropyridin-2- | |
| | yl)cyclopropane-1-carboxamide, (1R,2R or 1S,2S)-N-(7-chloro-6-(1- | |
| | ((3R,4R or 4S,4S)-4-hydroxy-3-methyltetrahydrofuran-3- | |
| | yl)piperidin-4-yl)isoquinolin-3-yl)-2-(4-fluoropyridin-2- | |
| | yl)cyclopropane-1-carboxamide | |
| 4.41-4.42 | 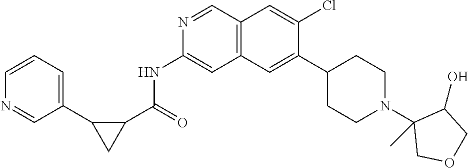 [SMILES: CC1(N2CCC(c3cc4cc(NC(=O)C5CC5c5cccnc5)ncc4cc3Cl)CC2)COCC1O] | Cal'c: 507 Found: 507 |
| | N-(7-chloro-6-(1-(4-hydroxy-3-methyltetrahydrofuran-3- | |
| | yl)piperidin-4-yl)isoquinolin-3-yl)-2-(pyridin-3-yl)cyclopropane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(pyridin-3-yl)cyclopropane-1-carboxamide | |
| 4.43-4.44 | 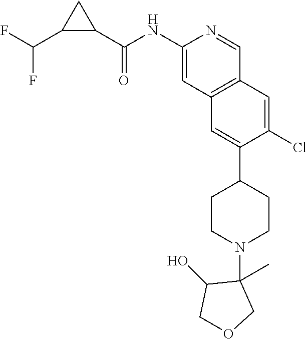 [SMILES: CC1(N2CCC(c3cc4cc(NC(=O)C5CC5C(F)F)ncc4cc3Cl)CC2)COCC1O] | Cal'c: 480 Found: 480 |
| | N-(7-chloro-6-(1-(4-hydroxy-3-methyltetrahydrofuran-3- | |
| | yl)piperidin-4-yl)isoquinolin-3-yl)-2-(difluoromethyl)cyclopropane- | |
| | 1-carboxamide, (1R,2R, or 1R,2S, or 1S,2R or 1S,2S)-N-(7-chloro-6- | |
| | (1-((3R,4R or 3S,4S)-4-hydroxy-3-methyltetrahydrofuran-3- | |
| | yl)piperidin-4-yl)isoquinolin-3-yl)-2-(difluoromethyl)cyclopropane- | |
| | 1-carboxamide | |
| 4.45-4.46 | 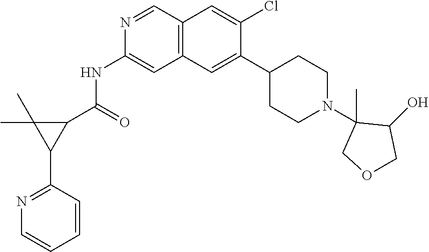 [SMILES: CC1(C)C(C(=O)Nc2cc3cc(C4CCN(C5(C)COCC5O)CC4)c(Cl)cc3cn2)C1c1ccccn1] | Cal'c: 535 Found: 535 |
| | N-(7-chloro-6-(1-(4-hydroxy-3-methyltetrahydrofuran-3- | |
| | yl)piperidin-4-yl)isoquinolin-3-yl)-2,2-dimethyl-3-(pyridin-2- | |
| | yl)cyclopropane-1-carboxamide, (1R,3R or 1S,3S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2,2-dimethyl-3-(pyridin-2- | |
| | yl)cyclopropane-1-carboxamide | |
| 4.47-4.48 | 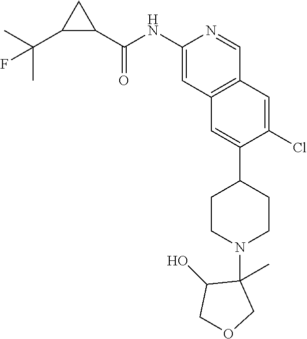 [SMILES: CC(C)(F)C1CC1C(=O)Nc1cc2cc(C3CCN(C4(C)COCC4O)CC3)c(Cl)cc2cn1] | Cal'c: 490 Found: 490 |
| | N-(7-chloro-6-(1-(4-hydroxy-3-methyltetrahydrofuran-3- | |
| | yl)piperidin-4-yl)isoquinolin-3-yl)-2-(2-fluoropropan-2- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(2-fluoropropan-2- | |
| | yl)cyclopropane-1-carboxamide | |
| 4.49-4.50 | 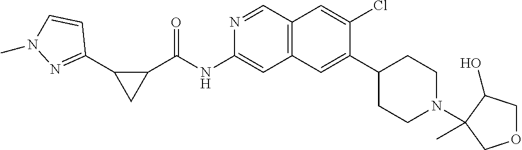 [SMILES: Cn1ccc(C2CC2C(=O)Nc2cc3cc(C4CCN(C5(C)COCC5O)CC4)c(Cl)cc3cn2)n1] | Cal'c: 510 Found: 510 |
| | N-(7-chloro-6-(1-(4-hydroxy-3-methyltetrahydrofuran-3- | |
| | yl)piperidin-4-yl)isoquinolin-3-yl)-2-(1-methyl-1H-pyrazol-3- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(1-methyl-1H-pyrazol-3- | |
| | yl)cyclopropane-1-carboxamide | |
| 4.51-4.54 | 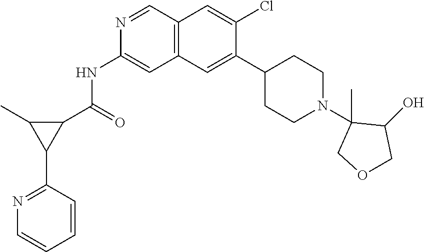 [SMILES: CC1C(C(=O)Nc2cc3cc(C4CCN(C5(C)COCC5O)CC4)c(Cl)cc3cn2)C1c1ccccn1] | Cal'c: 521 Found: 521 |
| | N-(7-chloro-6-(1-(4-hydroxy-3-methyltetrahydrofuran-3- | |
| | yl)piperidin-4-yl)isoquinolin-3-yl)-2-methyl-3-(pyridin-2- | |
| | yl)cyclopropane-1-carboxamide, (1S,2R,3R or 1S,2S,3R, or | |
| | 1R,2R,3S, or 1R,2S,3S)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-2-methyl-3-(pyridin-2-yl)cyclopropane-1-carboxamide | |
| 4.55-4.56 | 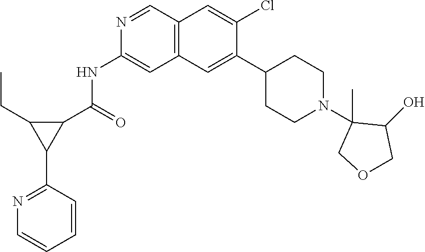 [SMILES: CCC1C(C(=O)Nc2cc3cc(C4CCN(C5(C)COCC5O)CC4)c(Cl)cc3cn2)C1c1ccccn1] | Cal'c: 535 Found: 535 |
| | N-(7-chloro-6-(1-(4-hydroxy-3-methyltetrahydrofuran-3- | |
| | yl)piperidin-4-yl)isoquinolin-3-yl)-2-ethyl-3-(pyridin-2- | |
| | yl)cyclopropane-1-carboxamide, (1S,2R,3R or 1S,2S,3R, or | |
| | 1R,2R,3S, or 1R,2S,3S)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-2-ethyl-3-(pyridin-2-yl)cyclopropane-1-carboxamide | |
| 4.57-4.58 | 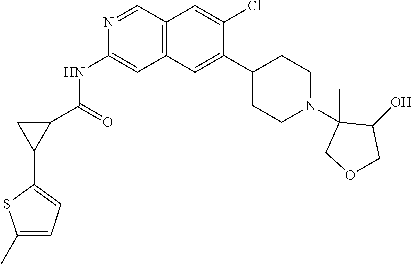 [SMILES: Cc1ccc(C2CC2C(=O)Nc2cc3cc(C4CCN(C5(C)COCC5O)CC4)c(Cl)cc3cn2)s1] | Cal'c: 527 Found: 527 |
| | N-(7-chloro-6-(1-(4-hydroxy-3-methyltetrahydrofuran-3- | |
| | yl)piperidin-4-yl)isoquinolin-3-yl)-2-(5-methylthiophen-2- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(5-methylthiophen-2- | |
| | yl)cyclopropane-1-carboxamide | |
| 4.59-4.60 | 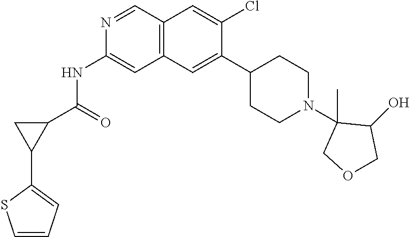 [SMILES: CC1(N2CCC(c3cc4cc(NC(=O)C5CC5c5cccs5)ncc4cc3Cl)CC2)COCC1O] | Cal'c: 512 Found: 512 |
| | N-(7-chloro-6-(1-(4-hydroxy-3-methyltetrahydrofuran-3- | |
| | yl)piperidin-4-yl)isoquinolin-3-yl)-2-(thiophen-2-yl)cyclopropane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(thiophen-2-yl)cyclopropane-1-carboxamide | |
| 4.61-4.62 | 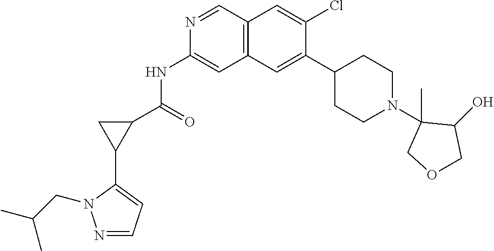 [SMILES: CC(C)Cn1nccc1C1CC1C(=O)Nc1cc2cc(C3CCN(C4(C)COCC4O)CC3)c(Cl)cc2cn1] | Cal'c: 552 Found: 552 |
| | N-(7-chloro-6-(1-(4-hydroxy-3-methyltetrahydrofuran-3- | |
| | yl)piperidin-4-yl)isoquinolin-3-yl)-2-(1-isobutyl-1H-pyrazol-5- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(1-isobutyl-1H-pyrazol-5- | |
| | yl)cyclopropane-1-carboxamide | |
| 4.63-4.64 | 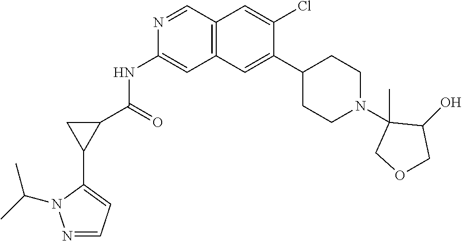 [SMILES: CC(C)n1nccc1C1CC1C(=O)Nc1cc2cc(C3CCN(C4(C)COCC4O)CC3)c(Cl)cc2cn1] | Cal'c: 538 Found: 538 |
| | N-(7-chloro-6-(1-(4-hydroxy-3-methyltetrahydrofuran-3- | |
| | yl)piperidin-4-yl)isoquinolin-3-yl)-2-(1-isopropyl-1H-pyrazol-5- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(1-isopropyl-1H-pyrazol-5- | |
| | yl)cyclopropane-1-carboxamide | |
| 4.65-4.66 | 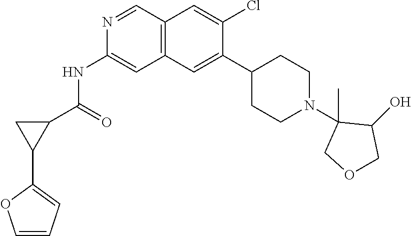 [SMILES: CC1(N2CCC(c3cc4cc(NC(=O)C5CC5c5ccco5)ncc4cc3Cl)CC2)COCC1O] | Cal'c: 496 Found: 496 |
| | N-(7-chloro-6-(1-(4-hydroxy-3-methyltetrahydrofuran-3- | |
| | yl)piperidin-4-yl)isoquinolin-3-yl)-2-(furan-2-yl)cyclopropane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(furan-2-yl)cyclopropane-1-carboxamide | |
| 4.67-4.68 | 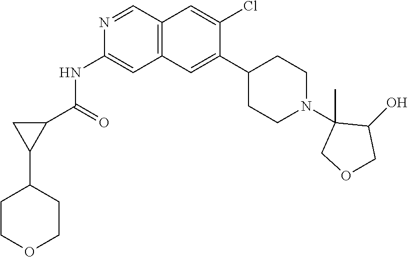 [SMILES: CC1(N2CCC(c3cc4cc(NC(=O)C5CC5C5CCOCC5)ncc4cc3Cl)CC2)COCC1O] | Cal'c: 514 Found: 514 |
| | N-(7-chloro-6-(1-(4-hydroxy-3-methyltetrahydrofuran-3- | |
| | yl)piperidin-4-yl)isoquinolin-3-yl)-2-(tetrahydro-2H-pyran-4- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(tetrahydrofuran-2H-pyran-4- | |
| | yl)cyclopropane-1-carboxamide | |
| 4.69-4.70 | 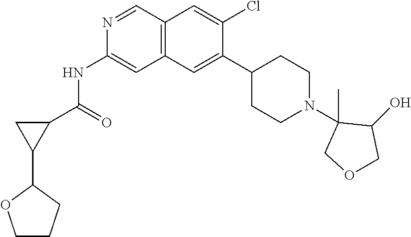 [SMILES: CC1(N2CCC(c3cc4cc(NC(=O)C5CC5C5CCCO5)ncc4cc3Cl)CC2)COCC1O] | Cal'c: 500 Found: 500 |
| | N-(7-chloro-6-(1-(4-hydroxy-3-methyltetrahydrofuran-3- | |
| | yl)piperidin-4-yl)isoquinolin-3-yl)-2-(tetrahydrofuran-2- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(tetrahydrofuran-2- | |
| | yl)cyclopropane-1-carboxamide | |
| 4.71-4.72 | 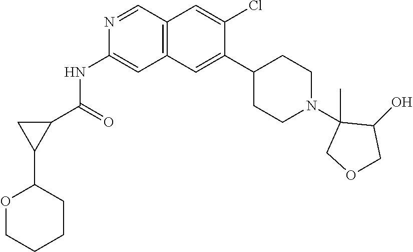 [SMILES: CC1(N2CCC(c3cc4cc(NC(=O)C5CC5C5CCCCO5)ncc4cc3Cl)CC2)COCC1O] | Cal'c: 514 Found: 514 |
| | N-(7-chloro-6-(1-(4-hydroxy-3-methyltetrahydrofuran-3- | |
| | yl)piperidin-4-yl)isoquinolin-3-yl)-2-(tetrahydro-2H-pyran-2- | |
| | yl)cyclopropane-1-carboxamide, (1R,2R or 1S,2S)-N-(7-chloro-6-(1- | |
| | ((3R,4R or 3S,4S)-4-hydroxy-3-methyltetrahydrofuran-3- | |
| | yl)piperidin-4-yl)isoquinolin-3-yl)-2-(tetrahydro-2H-pyran-2- | |
| | yl)cyclopropane-1-carboxamide | |
| 4.73-4.74 | 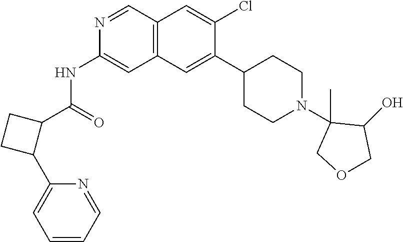 [SMILES: CC1(N2CCC(c3cc4cc(NC(=O)C5CCC5c5ccccn5)ncc4cc3Cl)CC2)COCC1O] | Cal'c: 521 Found: 521 |
| | N-(7-chloro-6-(1-(4-hydroxy-3-methyltetrahydrofuran-3- | |
| | yl)piperidin-4-yl)isoquinolin-3-yl)-2-(pyridin-2-yl)cyclobutane-1- | |
| | carboxamide, (1R,2R or 1S,2S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-2-(pyridin-2-yl)cyclobutane-1-carboxamide | |
| 4.75-4.76 | 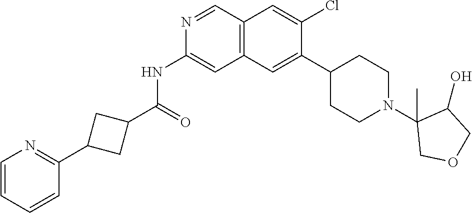 [SMILES: CC1(N2CCC(c3cc4cc(NC(=O)C5CC(c6ccccn6)C5)ncc4cc3Cl)CC2)COCC1O] | Cal'c: 521 Found: 521 |
| | N-(7-chloro-6-(1-(4-hydroxy-3-methyltetrahydrofuran-3- | |
| | yl)piperidin-4-yl)isoquinolin-3-yl)-3-(pyridin-2-yl)cyclobutane-1- | |
| | carboxamide, (1R,3R or 1S,3S)-N-(7-chloro-6-(1-((3R,4R or 3S,4S)- | |
| | 4-hydroxy-3-methyltetrahydrofuran-3-yl)piperidin-4-yl)isoquinolin- | |
| | 3-yl)-3-(pyridin-2-yl)cyclobutane-1-carboxamide | |
| 4.77-4.78 | 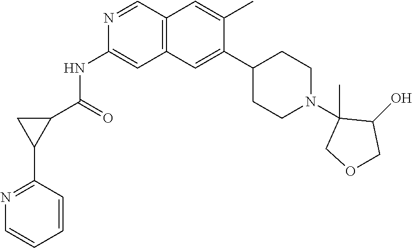 [SMILES: Cc1cc2cnc(NC(=O)C3CC3c3ccccn3)cc2cc1C1CCN(C2(C)COCC2O)CC1] | Cal'c: 487 Found: 487 |
| | N-(6-(1-(4-hydroxy-3-methyltetrahydrofuran-3-yl)piperidin-4-yl)-7- | |
| | methylisoquinolin-3-yl)-2-(pyridin-2-yl)cyclopropane-1- | |
| | carboxamide, (1R,2R or 1S,2S)-N-(6-(1-((3S,4S or 3R,4R)-4- | |
| | hydroxy-3-methyltetrahydrofuran-3-yl)piperidin-4-yl)-7- | |
| | methylisoquinolin-3-yl)-2-(pyridin-2-yl)cyclopropane-1-carboxamide | |

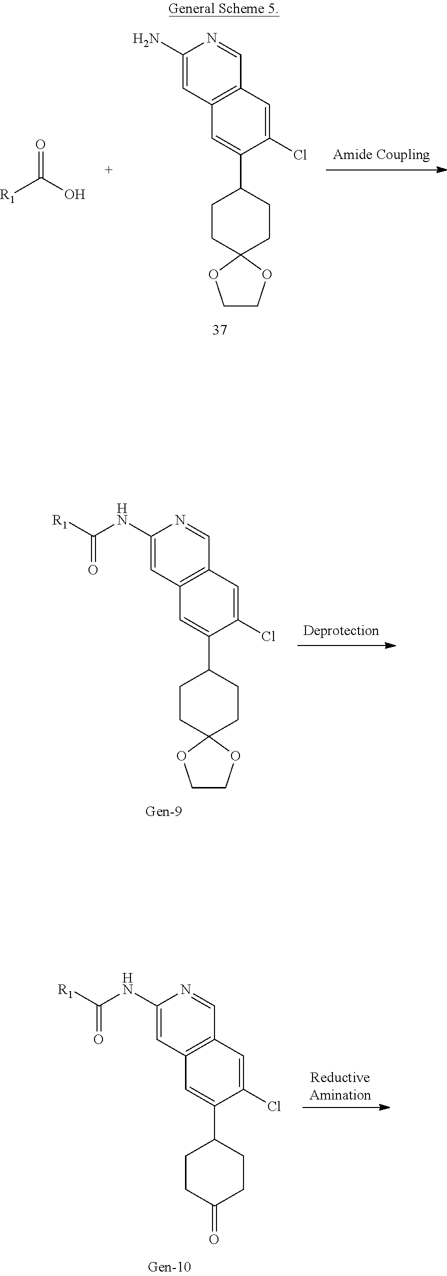

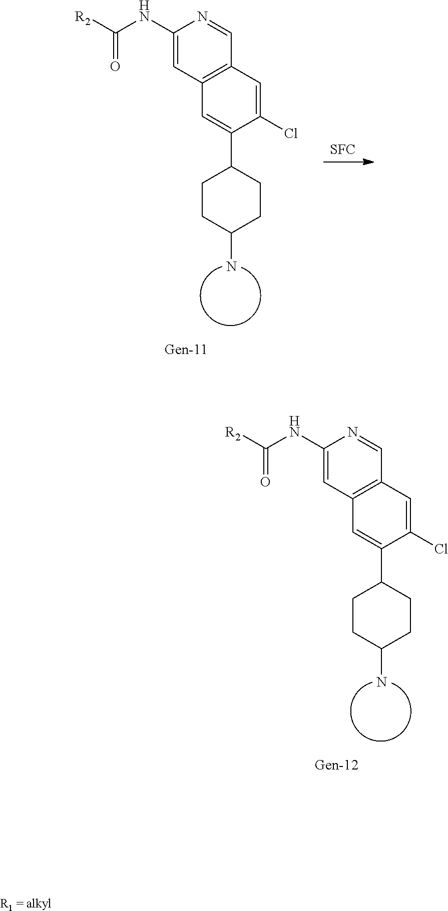

In General Scheme 5, commercially available carboxylic acids or acid chlorides were coupled with intermediate 37 through amide coupling conditions to provide Gen-9, which in turn could be deprotected to afford products in the form of Gen-10. A reductive amination with commercially available ketones was then performed to provide compounds that are fully elaborated in the form of Gen-11 and the stereoisomers could then be separated by chiral SFC to provide fully elaborated products in the form of Gen-12. The representative compounds are described in more detail below.

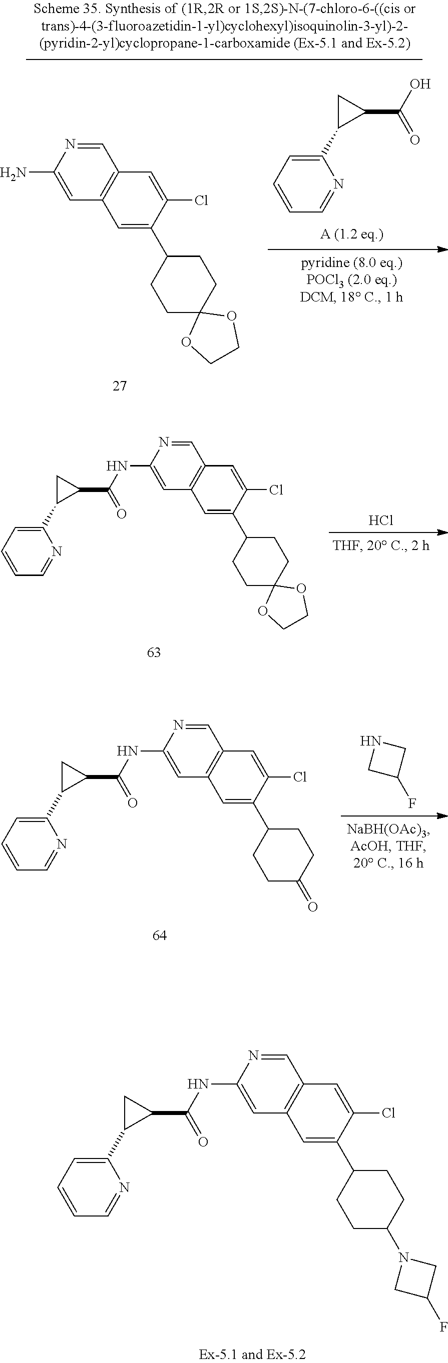

### (1R,2R)-N-(7-chloro-6-(1,4-dioxaspiro[4.5]decan-8-yl)isoquinolin-3-yl)-2-(pyridin-2-yl)cyclopropane-1-carboxamide (63)

To a solution of 7-chloro-6-(1,4-dioxaspiro[4.5]decan-8-yl)isoquinolin-3-amine 27 (80 mg, 0.251 mmol) and trans-2-(pyridin-2-yl)cyclopropane-1-carboxylic acid (49.1 mg, 0.301 mmol) in DCM (2 mL) was added pyridine (0.162 mL, 2.008 mmol) and phosphoryl trichloride (0.047 mL, 0.502 mmol) at 0° C. The mixture was stirred at 18° C. for 2 hours, then was added into ice water slowly. The resulting mixture was extracted with EtOAc. The combined organic layer was washed with water and dried over Na₂SO₄. After filtration and concentration, the residue was purified by prep-HPLC to afford title compound 63. MS (ESI): m/z calc'd for C₂₆H₂₇ClN₃O₃ [M+H]⁺: 464, found 464.

### (1R,2R)-N-(7-chloro-6-(4-oxocyclohexyl)isoquinolin-3-yl)-2-(pyridin-2-yl)cyclopropane-1-carboxamide (64)

To a solution of N-(7-chloro-6-(1,4-dioxaspiro[4.5]decan-8-yl)isoquinolin-3-yl)-2-(pyridin-2-yl)cyclopropane-1-carboxamide (70 mg, 0.151 mmol) in anhydrous THF (2 mL) was added HCl (0.2 mL, 2.4 mmol), and the resulting mixture was stirred at 20° C. for 2 hours. The reaction mixture was adjusted pH to 8-9 using aq. NaHCO₃, extracted with EtOAc, and dried over Na₂SO₄. Filtration and concentration afforded title compound 64. MS (ESI): m/z calc'd for C₂₄H₂₃ClN₃O₂ [M+H]⁺: 420, found 420.

### (1R,2R or 1S,2S)-N-(7-chloro-6-((cis or trans)-4-(3-fluoroazetidin-1-yl)cyclohexyl)isoquinolin-3-yl)-2-(pyridin-2-yl)cyclopropane-1-carboxamide (Ex-5.1 and Ex-5.2)

To a solution of (1R,2R)-N-(7-chloro-6-(4-oxocyclohexyl) isoquinolin-3-yl)-2-(pyridin-2-yl) cyclopropane-1-carboxamide (60 mg, 0.143 mmol) and 3-fluoroazetidine (16.09 mg, 0.214 mmol) in anhydrous THF (2 mL) was added AcOH (0.041 mL, 0.714 mmol). The resulting mixture was stirred at 20° C. for 0.5 hour, then sodium triacetoxyborohydride (60.6 mg, 0.286 mmol) was added, and the resulting mixture and stirred at 20° C. for 16 hours. The reaction mixture was adjusted pH to 8-9 using aq. NaHCO₃, extracted with EtOAc, and dried over Na₂SO₄. After filtration and concentration, the crude product was purified by Prep-HPLC (TFA) to afford title compounds Ex-5.1 and Ex-5.2.

Ex-5.1: MS (ESI): m/z calc'd for C₂₇H₂₉ClFN₄O [M+H]⁺: 479, found 479. ¹H NMR (400 MHz, MeOD-d₄) δ 8.96 (s, 1H), 8.60 (br d, J=4.3 Hz, 1H), 8.44 (s, 1H), 8.22 (m, 1H), 8.07 (s, 1H), 7.78 (s, 1H), 7.65 (m, 2H), 5.55-5.32 (m, 1H), 4.63 (m, 2H), 4.42 (m, 2H), 3.39 (br s, 1H), 3.18 (m, 1H), 2.89-2.81 (m, 1H), 2.65-2.57 (m, 1H), 2.25 (br s, 2H), 2.17 (m, 2H), 1.91-1.83 (m, 1H), 1.77 (m, 1H), 1.72-1.62 (m, 2H), 1.50 (q, J=11.5 Hz, 2H)

Ex-5.2: MS (ESI): m/z calc'd for C₂₇H₂₉ClFN₄O [M+H]⁺: 479, found 479. ¹H NMR (400 MHz, MeOD-d₄) δ 8.93 (s, 1H), 8.61 (d, J=5.5 Hz, 1H), 8.41 (s, 1H), 8.29 (m, 1H), 8.04 (s, 1H), 7.80 (s, 1H), 7.73-7.64 (m, 2H), 5.55-5.30 (m, 1H), 4.78-4.32 (m, 4H), 3.67 (br s, 1H), 3.27-3.20 (m, 1H), 2.93-2.83 (m, 1H), 2.68-2.55 (m, 1H), 2.09 (m, 2H), 2.02-1.92 (m, 2H), 1.92-1.86 (m, 3H), 1.77 (m, 3H)

Compounds in Table 5 below were prepared in accordance with the synthetic sequence illustrated in General Scheme 5 and Scheme 35 using the corresponding starting materials.

TABLE 5

| | | |
|---|---|---|
| 5.3-5.6 | 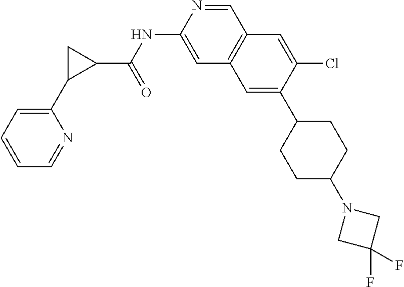 [SMILES: O=C(Nc1cc2cc(C3CCC(N4CC(F)(F)C4)CC3)c(Cl)cc2cn1)C1CC1c1ccccn1] | Cal'c: 497 Found: 497 |
| | N-(7-chloro-6-(4-(3,3-difluoroazetidin-1-yl)cyclohexyl)isoquinolin- | |
| | 3-yl)-2-(pyridin-2-yl)cyclopropane-1-carboxamide, (1R,2R or | |
| | 1S,2S)-N-(7-chloro-6-(cis-4-(3,3-difluoroazetidin-1- | |
| | yl)cyclohexyl)isoquinolin-3-yl)-2-(pyridin-2-yl)cyclopropane-1- | |
| | carboxamide | |
| 5.7-5.8 | 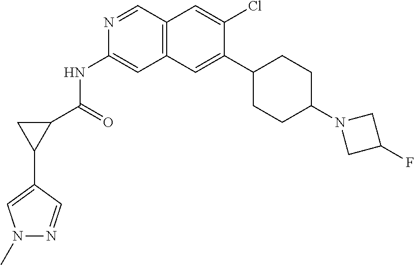 [SMILES: Cn1cc(C2CC2C(=O)Nc2cc3cc(C4CCC(N5CC(F)C5)CC4)c(Cl)cc3cn2)cn1] | Cal'c: 482 Found: 482 |
| | N-(7-cchloro-6-(4-(3-fluoroazetidin-1-yl)cyclohexyl)isoquinolin-3- | |
| | yl)-2-(1-methyl-1H-pyrazol-4-yl)cyclopropane-1-carboxamide, | |
| | (1S,2S or 1R,2R)-N-(7-chloro-6-(cis or trans-4-(3-fluoroazetidin-1- | |
| | yl)cyclohexyl)isoquinolin-3-yl)-2-(1-methyl-1H-pyrazol-4- | |
| | yl)cyclopropane-1-carboxamide | |
| 5.9-5.16 | 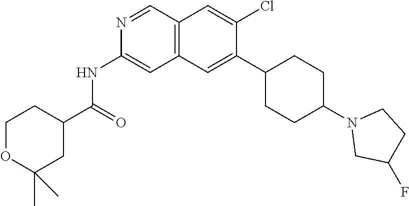 [SMILES: CC1(C)CC(C(=O)Nc2cc3cc(C4CCC(N5CCC(F)C5)CC4)c(Cl)cc3cn2)CCO1] | Cal'c: 488 Found: 488 |
| | N-(7-chloro-6-(4-(3-fluoropyrrolidin-1-yl)cyclohexyl)isoquinolin-3- | |
| | yl)-2,2-dimethyltetrahydro-2H-pyran-4-carboxamid, (4R or S)-N-(7- | |
| | chloro-6-(cis or trans-4-((R or S)-3-fluoropyrrolidin-1- | |
| | yl)cyclohexyl)isoquinolin-3-yl)-2,2-dimethyltetrahydro-2H-pyran-4- | |
| | carboxamide | |
| 5.17-5.24 | 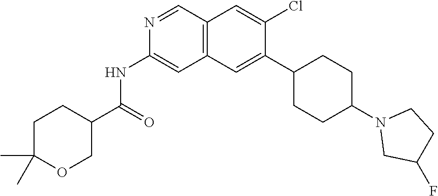 [SMILES: CC1(C)CCC(C(=O)Nc2cc3cc(C4CCC(N5CCC(F)C5)CC4)c(Cl)cc3cn2)CO1] | Cal'c: 488 Found: 488 |
| | N-(7-chloro-6-(4-(3-fluoropyrrolidin-1-yl)cyclohexyl)isoquinolin-3- | |
| | yl)-6,6-dimethyltetrahydro-2H-pyran-3-carboxamide, (5R or 5S)-N- | |
| | (7-chloro-6-(cis or trans-4-((R or S)-3-fluoropyrrolidin-1- | |
| | yl)cyclohexyl)isoquinolin-3-yl)-6,6-dimethyltetrahydro-2H-pyran-3- | |
| | carboxamide | |
| 5.25-5.26 | 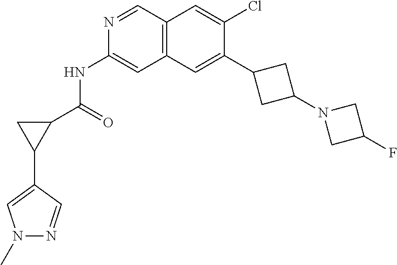 [SMILES: Cn1cc(C2CC2C(=O)Nc2cc3cc(C4CC(N5CC(F)C5)C4)c(Cl)cc3cn2)cn1] | Cal'c: 482 Found: 482 |
| | N-(7-chloro-6-(3-(3-fluoroazetidin-1-yl)cyclobutyl)isoquinolin-3- | |
| | yl)-2-(1-methyl-1H-pyrazol-4-yl)cyclopropane-1-carboxamide, | |
| | (1R,2R or 1S,2S)-N-(7-chloro-6-(cis or trans-3-(3-fluoroazetidin-1- | |
| | yl)cyclobutyl)isoquinolin-3-yl)-2-(1-methyl-1H-pyrazol-4- | |
| | yl)cyclopropane-1-carboxamide | |

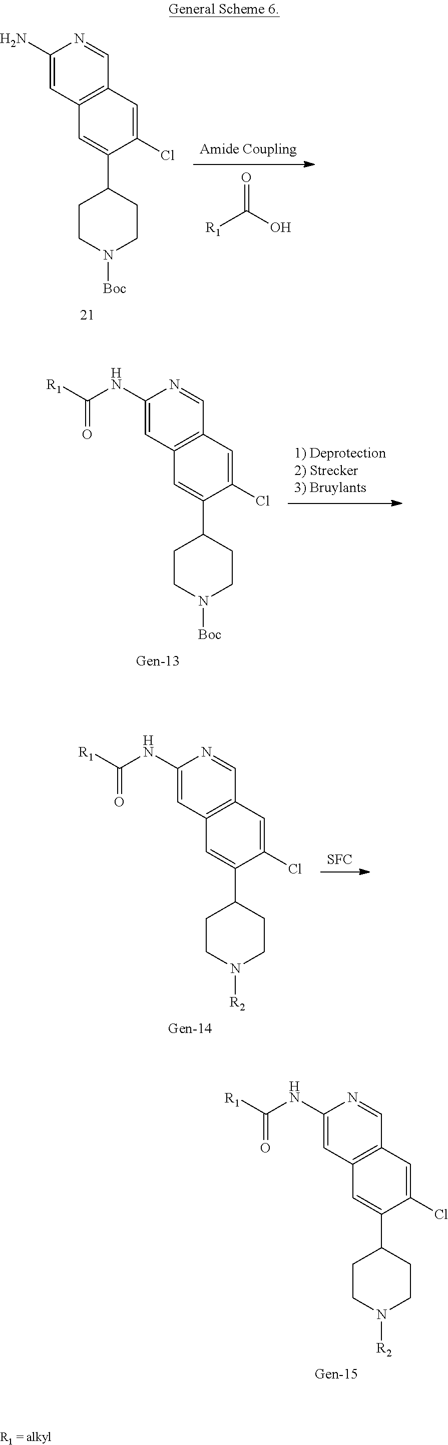

In General Scheme 6, amide coupling conditions with intermediate 21 commercially available carboxylic acids were performed to afford Gen-13. The corresponding Boc-protected piperidine was then deprotected followed by a Strecker reaction to afford Gen-14 and the stereoisomers could then be separated by chiral SFC to provide fully elaborated products in the form of Gen-15. The representative compounds are shown below.

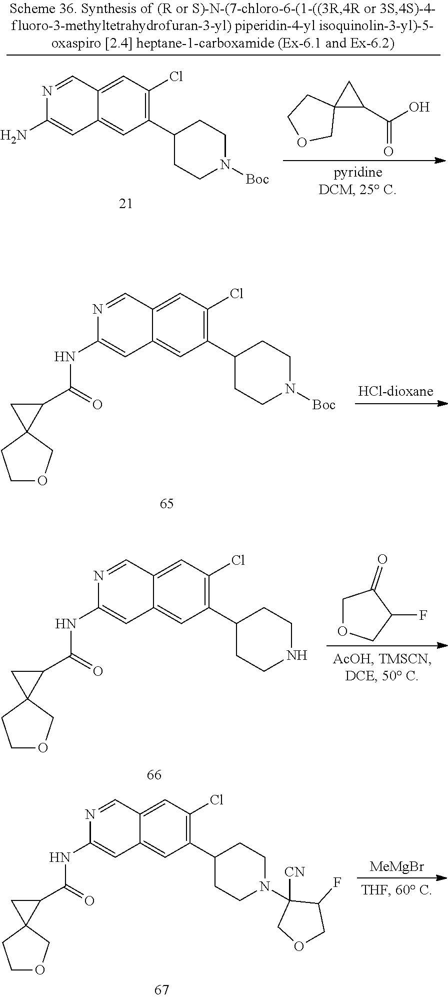

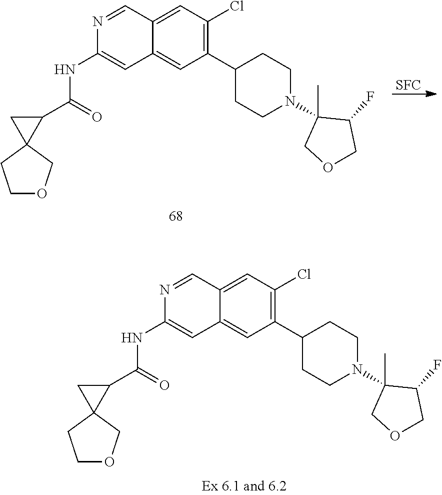

### tert-butyl 4-(7-chloro-3-(5-oxaspiro[2.4]heptane-1-carboxamido)isoquinolin-6-yl) piperidine-1-carboxylate (65)

To a solution of 5-oxaspiro[2.4]heptane-1-carboxylic acid (1.178 g, 4.15 mmol) and tert-butyl 4-(3-amino-7-chloroisoquinolin-6-yl)piperidine-1-carboxylate 21 (2 g, 2.76 mmol) in DCM (20 mL) was added pyridine (3.576 mL, 22.11 mmol) and phosphoryl trichloride (1.03 mL, 5.53 mmol) at 0° C. and the mixture was warmed to 18° C. The mixture was added into ice water slowly, then extracted with EA. The organic layer was washed with water, dried over Na₂SO₄. After filtration and concentration, the residue was purified by column chromatography to afford title compound 65. The stereoisomers were separated by SFC using column: Cellulose 2 100×4.6 mm I.D., 3 um, Mobile phase: A: CO₂ B: methanol (0.05% DEA), Isocratic: 40% B, Flow rate: 2.8 mL/min, Column temp 35° C. and dried by lyophilization to separate the stereoisomers.

### N-(7-chloro-6-(piperidin-4-yl)isoquinolin-3-yl)-5-oxaspiro[2.4]heptane-1-carboxamide (66)

To a solution of tert-butyl 4-(7-chloro-3-(5-oxaspiro[2.4] heptane-1-carboxamido) isoquinolin-6-yl) piperidine-O-carboxylate (560 mg, 1.152 mmol) in HCl-dioxane (5 mL) at 25° C. The mixture was stirred at 25° C. The reaction mixture was adjusted pH to 8˜9 using aq. KOH (3 M) extracted with EA. The organic layer was washed with water, dried over Na₂SO₄. The combined layers were concentrated to afford title compound 66. MS (ESI): m/z calc'd for C₂₁H₂₅CN₃O₂ [M+H]⁺: 386, found 386.

### N-(7-chloro-6-(1-(3-cyano-4-fluorotetrahydrofuran-3-yl)piperidin-4-yl) isoquinolin-3-yl)-5-oxaspiro 12.41 heptane-1-carboxamide (67)

To a solution of N-(7-chloro-6-(piperidin-4-yl)isoquinolin-3-yl)-5-oxaspiro[2.4]heptane-1-carboxamide (400 mg, 1.037 mmol) and 4-fluorodihydrofuran-3(2H)-one (863 mg, 8.29 mmol) in DCE (4 mL) was added AcOH (0.297 mL, 5.18 mmol). The mixture was stirred at 50° C. Trimethylsilanecarbonitrile (0.648 mL, 5.18 mmol) was added to the mixture. The resulting mixture was stirred for at 50° C. Water was added to the mixture, and the mixture was extracted with EtOAc. The combined organic layers were dried over Na₂SO₄ and filtered. The filtrate was concentrated in vacuo and the residue was purified by prep-HPLC (basic) to afford title compound 67. MS (ESI): m/z calc'd for C₂₆H₂₉ClFN₄O₃ [M+H]⁺: 499, found 499.

### N-(7-chloro-6-(1-(4-fluoro-3-methyltetrahydrofuran-3-yl) piperidin-4-yl) isoquinolin-3-yl)-5-oxaspiro [2.4] heptane-1-carboxamide (68)

To a solution of N-(7-chloro-6-(1-(3-cyano-4-fluorotetrahydrofuran-3-yl)piperidin-4-yl)isoquinolin-3-yl)-5-oxaspiro[2.4]heptane-1-carboxamide (140 mg, 0.281 mmol) in THF (2 mL) was added methylmagnesium bromide (0.935 mL, 2.81 mmol) at 0° C. The resulting solution was heated to 60° C. The reaction was quenched with saturated aqueous NH₄Cl and extracted with EtOAc (20 mL×3). The combined organic fractions were dried over Na₂SO₄ and filtered. The filtrate was concentrated in vacuo to give a crude product. The crude product was purified by prep-HPLC (TFA) to afford title compound 68.

### (R or S)-N-(7-chloro-6-(1-((3R,4R or 3S,4S)-4-fluoro-3-methyltetrahydrofuran-3-yl) piperidin-4-yl) isoquinolin-3-yl)-5-oxaspiro [2.4] heptane-1-carboxamide (Ex-6.1, Ex-6.2, Ex-6.3, Ex-6.4, Ex-6.5, Ex-6.6, Ex-6.7 or Ex-6.8)

A mixture of stereoisomers was separated by SFC column: Chiralpak IG-3 50×4.6 mm I.D., 3 um, Mobile phase: A: CO₂, B:iso-propanol (0.05% DEA), Isocratic: 40% B, Flow rate: 4 mL/min, Column temp.: 35° C. and dried by lyophilization to afford Ex-6.1 (Rt=1.270 min) and Ex-6.2 (Rt=1.964 min). A similar synthesis for all four isomers after step one were completed to afford 8 stereoisomeric compounds. Absolute stereoconfiguration was not determined for any of these compounds.

Ex-6.1: MS (ESI): m/z calc'd for C₂₆H₃₂ClFN₃O₃ [M+H]+: 488, found 488. ¹H NMR (400 MHz, CDCl₃-d) δ 8.84 (s, 1H), 8.61 (br s, 1H), 8.47 (s, 1H), 7.89 (s, 1H), 7.69 (s, 1H), 4.94-4.70 (m, 1H), 4.33-4.09 (m, 2H), 4.07 (m, 1H), 4.02-3.87 (m, 4H), 3.82 (d, J=7.8 Hz, 1H), 3.23 (m, 1H), 3.16-3.06 (m, 1H), 2.89-2.66 (m, 3H), 2.10-2.01 (m, 4H), 1.99-1.93 (m, 1H), 1.92-1.86 (m, 1H), 1.54 (t, J=4.9 Hz, 1H), 1.36-1.23 (m, 2H), 1.23-1.16 (m, 3H)

Ex-6.2: MS (ESI): m/z calc'd for C₂₆H₃₂ClFN₃O₃ [M+H]+: 488, found 488. ¹H NMR (400 MHz, CDCl₃-d) δ 8.85 (s, 1H), 8.44 (s, 1H), 8.37 (s, 1H), 7.89 (s, 1H), 7.69 (s, 1H), 4.86-4.67 (m, 1H), 4.32-4.13 (m, 1H), 4.13-4.02 (m, 1H), 4.00-3.94 (m, 2H), 3.93 (s, 3H), 3.80 (d, J=7.4 Hz, 1H), 3.13-3.00 (m, 2H), 2.69-2.54 (m, 3H), 2.10-2.02 (m, 1H), 2.00-1.91 (m, 3H), 1.86 (m, 2H), 1.83-1.78 (m, 1H), 1.55 (t, J=4.9 Hz, 1H), 1.31-1.25 (m, 1H), 1.12 (d, J=2.0 Hz, 3H)

Ex-6.3: MS (ESI): m/z calc'd for C₂₆H₃₂ClFN₃O₃ [M+H]+: 488, found 488. ¹H NMR (400 MHz, CDCl₃-d) δ 8.86 (s, 1H), 8.47 (s, 1H), 8.35 (s, 1H), 7.90 (s, 1H), 7.70 (s, 1H), 4.88-4.66 (m, 1H), 4.30-4.14 (m, 1H), 4.13-4.02 (m, 1H), 3.98 (t, J=6.8 Hz, 2H), 3.93 (br d, J=7.4 Hz, 1H), 3.81 (d, J=7.4 Hz, 1H), 3.76-3.67 (m, 2H), 3.13-2.96 (m, 2H), 2.71-2.53 (m, 3H), 2.24-2.09 (m, 2H), 2.05-1.95 (m, 2H), 1.93-1.74 (m, 3H), 1.62 (br d, J=5.1 Hz, 1H), 1.27-1.19 (m, 1H), 1.12 (d, J=2.0 Hz, 3H)

Ex-6.4: MS (ESI): m/z calc'd for C₂₆H₃₂ClFN₃O₃ [M+H]+: 488, found 488. ¹H NMR (400 MHz, CDCl₃-d) δ 8.87 (s, 1H), 8.60 (s, 1H), 8.48 (s, 1H), 7.90 (s, 1H), 7.70 (s, 1H), 4.89-4.66 (m, 1H), 4.31-4.13 (m, 1H), 4.13-4.04 (m, 1H), 3.99 (t, J=6.8 Hz, 2H), 3.92 (s, 1H), 3.80 (d, J=7.4 Hz, 1H), 3.72 (d, J=0.8 Hz, 1H), 3.15-3.01 (m, 2H), 2.71-2.54 (m, 3H), 2.25-2.08 (m, 2H), 2.05-1.96 (m, 2H), 1.94-1.85 (m, 2H), 1.80 (m, 1H), 1.61 (t, J=5.1 Hz, 1H), 1.33-1.17 (m, 2H), 1.13 (d, J=1.6 Hz, 3H)

Ex-6.5: MS (ESI): m/z calc'd for C₂₆H₃₂ClFN₃O₃ [M+H]+: 488, found 488. ¹H NMR (400 MHz, CDCl₃-d) δ 8.85 (s, 1H), 8.49-8.34 (m, 2H), 7.89 (s, 1H), 7.69 (s, 1H), 4.87-4.65 (m, 1H), 4.30-4.14 (m, 1H), 4.13-4.02 (m, 1H), 4.00-3.94 (m, 2H), 3.94-3.91 (m, 3H), 3.80 (d, J=7.4 Hz, 1H), 3.13-2.93 (m, 2H), 2.70-2.46 (m, 3H), 2.09-2.02 (m, 1H), 2.00-1.93 (m, 3H), 1.90-1.83 (m, 2H), 1.55 (t, J=4.9 Hz, 1H), 1.32-1.23 (m, 2H), 1.12 (d, J=2.0 Hz, 3H)

Ex-6.6: MS (ESI): m/z calc'd for C₂₆H₃₂ClFN₃O₃ [M+H]+: 488, found 488. ¹H NMR (400 MHz, CDCl₃-d) δ 8.84 (s, 1H), 8.46 (m, 2H), 7.88 (s, 1H), 7.69 (s, 1H), 4.86-4.67 (m, 1H), 4.30-4.13 (m, 1H), 4.00-3.90 (m, 5H), 3.79 (d, J=7.4 Hz, 1H), 3.13-3.00 (m, 2H), 2.69-2.51 (m, 3H), 2.10-2.02 (m, 1H), 2.02-1.92 (m, 4H), 1.91-1.82 (m, 3H), 1.54 (t, J=4.9 Hz, 1H), 1.28 (dd, J=4.7, 8.2 Hz, 1H), 1.11 (d, J=1.6 Hz, 3H)

Ex-6.7: MS (ESI): m/z calc'd for C₂₆H₃₂ClFN₃O₃ [M+H]+: 488, found 488. ¹H NMR (400 MHz, CDCl₃-d) δ 8.86 (s, 1H), 8.45 (m, 2H), 7.90 (s, 1H), 7.70 (s, 1H), 4.89-4.66 (m, 1H), 4.30-4.13 (m, 1H), 3.98 (t, J=7.0 Hz, 2H), 3.93 (br d, J=7.4 Hz, 1H), 3.80 (d, J=7.4 Hz, 1H), 3.76-3.68 (m, 2H), 3.16-3.03 (m, 2H), 2.70-2.52 (m, 3H), 2.25-2.08 (m, 2H), 2.04-1.96 (m, 2H), 1.94-1.78 (m, 3H), 1.61 (t, J=5.1 Hz, 1H), 1.30-1.18 (m, 2H), 1.12 (d, J=2.0 Hz, 3H)

Ex-6.8: MS (ESI): m/z calc'd for C₂₆H₃₂ClFN₃O₃ [M+H]+: 488, found 488. ¹H NMR (400 MHz, CDCl₃-d) δ 8.92-8.82 (m, 2H), 8.53 (s, 1H), 7.92 (s, 1H), 7.71 (s, 1H), 5.00-4.76 (m, 1H), 4.32-4.27 (m, 1H), 4.23-4.15 (m, 1H), 3.98 (t, J=7.0 Hz, 2H), 3.87 (d, J=8.2 Hz, 1H), 3.75-3.68 (m, 2H), 3.25-3.12 (m, 3H), 2.96 (q, J=9.9 Hz, 2H), 2.23 (br d, J=13.3 Hz, 2H), 2.20-2.13 (m, 2H), 2.12-2.05 (m, 2H), 1.84 (m, 1H), 1.63-1.56 (m, 1H), 1.34 (s, 3H), 1.29-1.20 (m, 2H)

Compounds in Table 6 below were prepared in accordance with the synthetic sequence illustrated in General Scheme 6 and Scheme 36 using the corresponding starting materials.

TABLE 6

| | | |
|---|---|---|
| 6.9-6.10 | 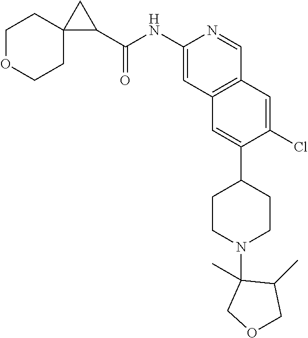 [SMILES: CC1COCC1(C)N1CCC(c2cc3cc(NC(=O)C4CC45CCOCC5)ncc3cc2Cl)CC1] | Cal'c: 498 Found: 498 |
| | N-(7-chloro-6-(1-(3,4-dimethyltetrahydrofuran-3-yl)piperidin-4- | |
| | yl)isoquinolin-3-yl)-6-oxaspiro[2.5]octane-1-carboxamide, (1S or | |
| | 1R)-N-(7-chloro-6-(1-(3R,4R or 3S,4S-dimethyltetrahydrofuran-3- | |
| | yl)piperidin-4-yl)isoquinolin-3-yl)-6-oxaspiro[2.5]octane-1- | |
| | carboxamide | |
| 6.11-6.12 | 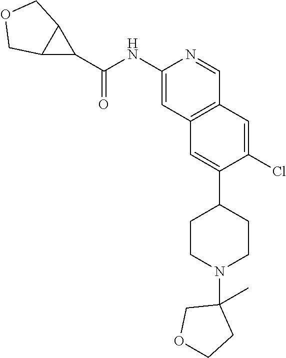 [SMILES: CC1(N2CCC(c3cc4cc(NC(=O)C5C6COCC65)ncc4cc3Cl)CC2)CCOC1] | Cal'c: 456 Found: 456 |
| | N-(7-chloro-6-(1-(3-methyltetrahydrofuran-3-yl)piperidin-4- | |
| | yl)isoquinolin-3-yl)-3-oxabicyclo[3.1.0]hexane-6-carboxamide, (1R | |
| | or 1S)-N-(7-chloro-6-(1-(3R or 3S-methyltetrahydrofuran-3- | |
| | yl)piperidin-4-yl)isoquinolin-3-yl)-3-oxabicyclo[3.1.0]hexane-6- | |
| | carboxamide | |
| 6.13-6.15 | 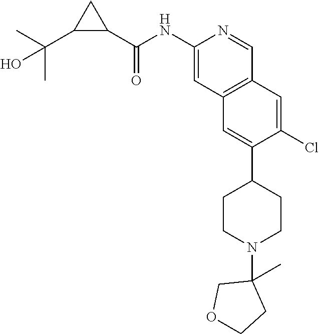 [SMILES: CC(C)(O)C1CC1C(=O)Nc1cc2cc(C3CCN(C4(C)CCOC4)CC3)c(Cl)cc2cn1] | Cal'c: 472 Found: 472 |
| | N-(7-chloro-6-(1-(3-methyltetrahydrofuran-3-yl)piperidin-4- | |
| | yl)isoquinolin-3-yl)-2-(2-hydroxypropan-2-yl)cyclopropane-1- | |
| | carboxamide, (1R,2R or 1S,2S)-N-(7-chloro-6-(1-(3R or 3S- | |
| | methyltetrahydrofuran-3-yl)piperidin-4-yl)isoquinolin-3-yl)-2-(2- | |
| | hydroxypropan-2-yl)cyclopropane-1-carboxamide | |
| 6.16-6.17 | 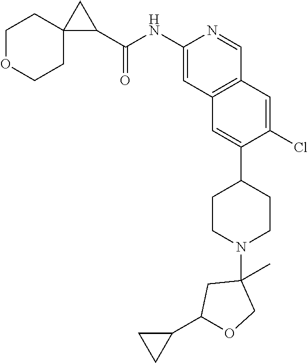 [SMILES: CC1(N2CCC(c3cc4cc(NC(=O)C5CC56CCOCC6)ncc4cc3Cl)CC2)COC(C2CC2)C1] | Cal'c: 524 Found: 524 |
| | N-(7-chloro-6-(1-(5-cyclopropyl-3-methyltetrahydrofuran-3- | |
| | yl)piperidin-4-yl)isoquinolin-3-yl)-6-oxaspiro[2.5]octane-1- | |
| | carboxamide, (1R or 1S)-N-(7-chloro-6-(1-(5R or 5S-cyclopropyl- | |
| | 3R or 3S-methyltetrahydrofuran-3-yl)piperidin-4-yl)isoquinolin-3- | |
| | yl)-6-oxaspiro[2.5]octane-1-carboxamide | |
| 6.18-6.19 | 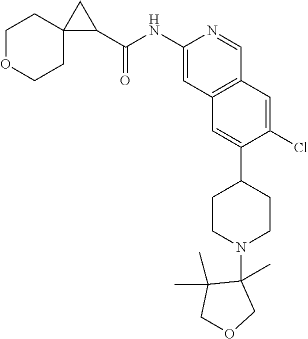 [SMILES: CC1(C)COCC1(C)N1CCC(c2cc3cc(NC(=O)C4CC45CCOCC5)ncc3cc2Cl)CC1] | Cal'c: 512 Found: 512 |
| | N-(7-chloro-6-(1-(3,4,4-trimethyltetrahydrofuran-3-yl)piperidin-4- | |
| | yl)isoquinolin-3-yl)-6-oxaspiro[2.5]octane-1-carboxamide, (1R or | |
| | 1S)-N-(7-chloro-6-(1-((S or R)-3,4,4-trimethyltetrahydrofuran-3- | |
| | yl)piperidin-4-yl)isoquinolin-3-yl)-6-oxaspiro[2.5]octane-1- | |
| | carboxamide | |
| 6.20-6.21 | 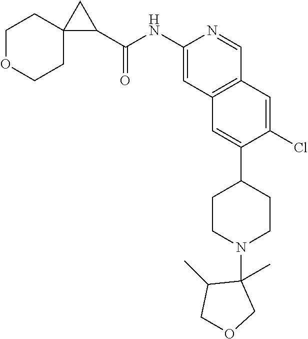 [SMILES: CC1COCC1(C)N1CCC(c2cc3cc(NC(=O)C4CC45CCOCC5)ncc3cc2Cl)CC1] | Cal'c: 496 Found: 496 |
| | N-(7-chloro-6-(1-(1,2-dimethylcyclopentyl)piperidin-4- | |
| | yl)isoquinolin-3-yl)-6-oxaspiro[2.5]octane-1-carboxamide, (1R or | |
| | 1S)-N-(7-chloro-6-(1-((R or S)-1,2-dimethylcyclopentyl)piperidin- | |
| | 4-yl)isoquinolin-3-yl)-6-oxaspiro[2.5]octane-1-carboxamide | |
| 6.22-6.23 | 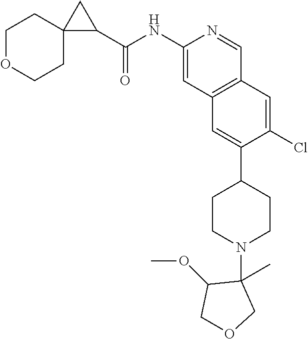 [SMILES: COC1COCC1(C)N1CCC(c2cc3cc(NC(=O)C4CC45CCOCC5)ncc3cc2Cl)CC1] | Cal'c: 514 Found: 514 |
| | N-(7-chloro-6-(1-(4-methoxy-3-methyltetrahydrofuran-3- | |
| | yl)piperidin-4-yl)isoquinolin-3-yl)-6-oxaspiro[2.5]octane-1- | |
| | carboxamide, (1R or 1S)-N-(7-chloro-6-(1-((3R,4R or 3S,4S)-4- | |
| | methoxy-3-methyltetrahydrofuran-3-yl)piperidin-4-yl)isoquinolin-3- | |
| | yl)-6-oxaspiro[2.5]octane-1-carboxamide | |
| 6.24-6.25 | 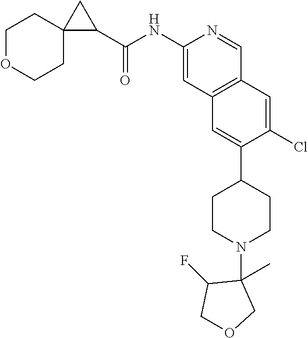 [SMILES: CC1(N2CCC(c3cc4cc(NC(=O)C5CC56CCOCC6)ncc4cc3Cl)CC2)COCC1F] | Cal'c: 502 Found: 502 |
| | N-(7-chloro-6-(1-(4-fluoro-3-methyltetrahydrofuran-3-yl)piperidin- | |
| | 4-yl)isoquinolin-3-yl)-6-oxaspiro[2.5]octane-1-carboxamide, (1R or | |
| | 1S)-N-(7-chloro-6-(1-((3R,4R or 3S,4S)-4-fluoro-3- | |
| | methyltetrahydrofuran-3-yl)piperidin-4-yl)isoquinolin-3-yl)-6- | |
| | oxaspiro[2.5]octane-1-carboxamide | |
| 6.26-6.29 | 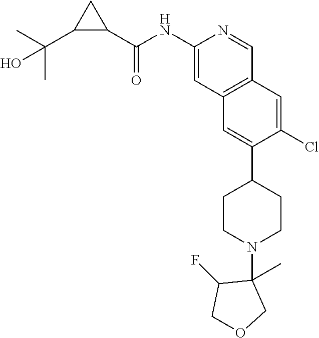 [SMILES: CC(C)(O)C1CC1C(=O)Nc1cc2cc(C3CCN(C4(C)COCC4F)CC3)c(Cl)cc2cn1] | Cal'c: 490 Found: 490 |
| | N-(7-chloro-6-(1-(4-fluoro-3-methyltetrahydrofuran-3-yl)piperidin- | |
| | 4-yl)isoquinolin-3-yl)-2-(2-hydroxypropan-2-yl)cyclopropane-1- | |
| | carboxamide, (1R or 1S)-N-(7-chloro-6-(1-((3R,4R or 3S,4S)-4- | |
| | fluoro-3-methyltetrahydrofuran-3-yl)piperidin-4-yl)isoquinolin-3- | |
| | yl)-2-(2-hydroxypropan-2-yl)cyclopropane-1-carboxamide | |
| 6.30-6.31 | 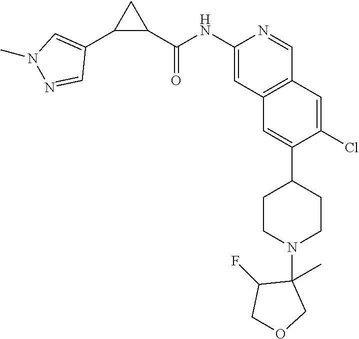 [SMILES: Cn1cc(C2CC2C(=O)Nc2cc3cc(C4CCN(C5(C)COCC5F)CC4)c(Cl)cc3cn2)cn1] | Cal'c: 512 Found: 512 |
| | N-(7-chloro-6-(1-(4-fluoro-3-methyltetrahydrofuran-3-yl)piperidin- | |
| | 4-yl)isoquinolin-3-yl)-2-(1-methyl-1H-pyrazol-4-yl)cyclopropane-1- | |
| | carboxamide, (1R,2R or 1S,2S)-N-(7-chloro-6-(1-((3R,4R or | |
| | 3S,4S)-4-fluoro-3-methyltetrahydrofuran-3-yl)piperidin-4- | |
| | yl)isoquinolin-3-yl)-2-(1-methyl-1H-pyrazol-4-yl)cyclopropane-1- | |
| | carboxamide | |
| 6.32-6.35 | 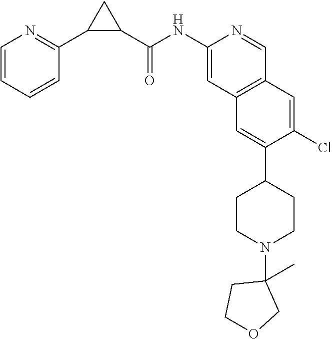 [SMILES: CC1(N2CCC(c3cc4cc(NC(=O)C5CC5c5ccccn5)ncc4cc3Cl)CC2)CCOC1] | Cal'c: 491 Found: 491 |
| | N-(7-chloro-6-(1-(3-methyltetrahydrofuran-3-yl)piperidin-4- | |
| | yl)isoquinolin-3-yl)-2-(pyridin-2-yl)cyclopropane-1-carboxamide, | |
| | (1R,2R or 1S,2S)-N-(7-chloro-6-(1-(3R or 3S- | |
| | methyltetrahydrofuran-3-yl)piperidin-4-yl)isoquinolin-3-yl)-2- | |
| | (pyridin-2-yl)cyclopropane-1-carboxamide | |
| 6.36-6.39 | 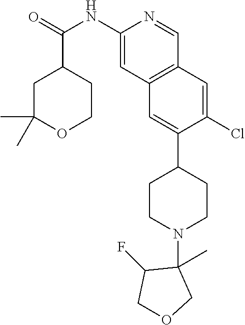 [SMILES: CC1(C)CC(C(=O)Nc2cc3cc(C4CCN(C5(C)COCC5F)CC4)c(Cl)cc3cn2)CCO1] | Cal'c: 504 Found: 504 |
| | N-(7-chloro-6-(1-(4-fluoro-3-methyltetrahydrofuran-3-yl)piperidin- | |
| | 4-yl)isoquinolin-3-yl)-2,2-dimethyltetrahydro-2H-pyran-4- | |
| | carboxamide, (4R or 4S)-N-(7-chloro-6-(1-((3R,4R or 3S,4S)-4- | |
| | fluoro-3-methyltetrahydrofuran-3-yl)piperidin-4-yl)isoquinolin-3- | |
| | yl)-2,2-dimethyltetrahydro-2H-pyran-4-carboxamide | |
| 6.40-6.43 | 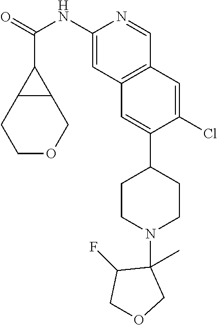 [SMILES: CC1(N2CCC(c3cc4cc(NC(=O)C5C6CCOCC65)ncc4cc3Cl)CC2)COCC1F] | Cal'c: 488 Found: 488 |
| | N-(7-chloro-6-(1-(4-fluoro-3-methyltetrahydrofuran-3-yl)piperidin- | |
| | 4-yl)isoquinolin-3-yl)-3-oxabicyclo[4.1.0]heptane-7-carboxamide, | |
| | (1R,6R or 1S,6S)-N-(7-chloro-6-(1-((3R,4R or 3S,4S)-4-fluoro-3- | |
| | methyltetrahydrofuran-3-yl)piperidin-4-yl)isoquinolin-3-yl)-3- | |
| | oxabicyclo[4.1.0]heptane-7-carboxamide | |
| 6.44-6.47 | 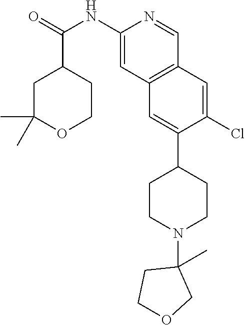 [SMILES: CC1(C)CC(C(=O)Nc2cc3cc(C4CCN(C5(C)CCOC5)CC4)c(Cl)cc3cn2)CCO1] | Cal'c: 486 Found: 486 |
| | N-(7-chloro-6-(1-(3-methyltetrahydrofuran-3-yl)piperidin-4- | |
| | yl)isoquinolin-3-yl)-2,2-dimethyltetrahydro-2H-pyran-4- | |
| | carboxamide, (4R or 4S)-N-(7-chloro-6-(1-(3R or 3S- | |
| | methyltetrahydrofuran-3-yl)piperidin-4-yl)isoquinolin-3-yl)-2,2- | |
| | dimethyltetrahydro-2H-pyran-4-carboxamide | |
| 6.48-6.51 | 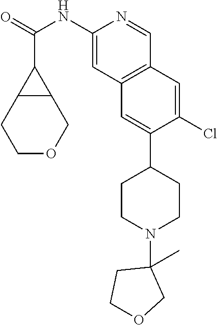 [SMILES: CC1(N2CCC(c3cc4cc(NC(=O)C5C6CCOCC65)ncc4cc3Cl)CC2)CCOC1] | Cal'c: 470 Found: 470 |
| | N-(7-chloro-6-(1-(3-methyltetrahydrofuran-3-yl)piperidin-4- | |
| | yl)isoquinolin-3-yl)-2-oxabicyclo[4.1.0]heptane-7-carboxamide, | |
| | (1R,6R or 1S,6S)-N-(7-chloro-6-(1-(3R or 3S- | |
| | methyltetrahydrofuran-3-yl)piperidin-4-yl)isoquinolin-3-yl)-3- | |
| | oxabicyclo[4.1.0]heptane-7-carboxamide | |
| 6.52-6.53 | 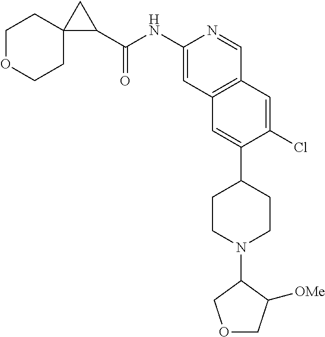 [SMILES: COC1COCC1N1CCC(c2cc3cc(NC(=O)C4CC45CCOCC5)ncc3cc2Cl)CC1] | Cal'c: 400 Found: 400 |
| | N-(7-chloro-6-(1-(4-methoxytetrahydrofuran-3-yl)piperidin-4- | |
| | yl)isoquinolin-3-yl)-6-oxaspiro[2.5]octane-1-carboxamide, (R or S)- | |
| | N-(7-chloro-6-(1-((3R,4R or 3S,4S)-4-methoxytetrahydrofuran-3- | |
| | yl)piperidin-4-yl)isoquinolin-3-yl)-6-oxaspiro[2.5]octane-1- | |
| | carboxamide | |
| 6.54-6.55 | 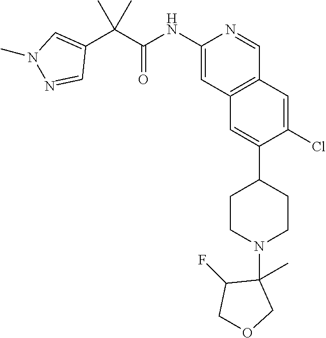 [SMILES: Cn1cc(C(C)(C)C(=O)Nc2cc3cc(C4CCN(C5(C)COCC5F)CC4)c(Cl)cc3cn2)cn1] | Cal'c: 514 Found: 514 |
| | N-(7-chloro-6-(1-(4-fluoro-3-methyltetrahydrofuran-3-yl)piperidin- | |
| | 4-yl)isoquinolin-3-yl)-2-methyl-2-(1-methyl-1H-pyrazol-4- | |
| | yl)propenamide, N-(7-chloro-6-(1-((3R,4R or 3S,4S)-4-fluoro-3- | |
| | methyltetrahydrofuran-3-yl)piperidin-4-yl)isoquinolin-3-yl)-2- | |
| | methyl-2-(1-methyl-1H-pyrazol-4-yl)propenamide | |

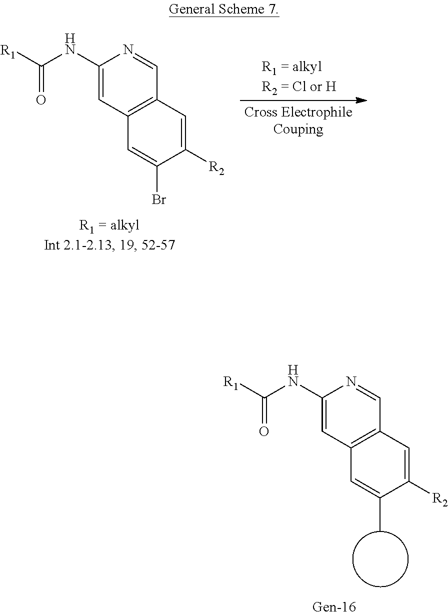

In General Scheme 7, reductive nickel C—C coupling conditions with commercially available or synthetically prepared intermediates Int 2.1-2.13, 19 or 52-57 afforded elaborated compounds of the form Gen-16. The representative compounds are described in more detail below.

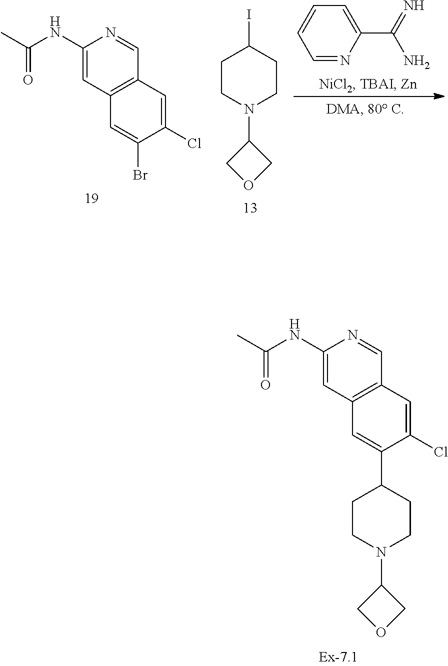

N-(7-chloro-6-(1-(oxetan-3-yl)piperidin-4-yl)isoquinolin-3-yl)acetamide (Ex-7.1) Nickel (II) chloride ethylene glycol dimethyl ether complex (12.91 mg, 0.059 mmol) and picolinimidamide (7.12 mg, 0.059 mmol) were added to a vial. The vial was sealed, and its contents were placed under an inert atmosphere by performing 3 vacuum/nitrogen cycles. DMA (1469 μl) was added, and the resulting mixture was stirred for 5 minutes at room temperature. In a separate vial, N-(6-bromo-7-chloroisoquinolin-3-yl)acetamide 19 (88 mg, 0.294 mmol), 4-iodo-1-(oxetan-3-yl)piperidine 13 (157 mg, 0.588 mmol), TBAI (21.70 mg, 0.059 mmol), and zinc (57.6 mg, 0.881 mmol) were added. The vial was sealed, and its contents were placed under an inert atmosphere by performing 3 vacuum/nitrogen cycles. The aforementioned nickel complex was added through the septum, and the resulting mixture was allowed to stir at 75° C. for 2 hours. The reaction mixture was diluted with ethyl acetate and washed twice with water and once with brine. The combined organic fractions were dried over anhydrous sodium sulfate, filtered and concentrated under reduced pressure. The residue was filtered (purified by HPLC, eluting acetonitrile/water gradient with 0.10% TFA modifier, linear gradient) and lyophilized to afford the title compound. MS (ESI): m/z calc'd for C₁₉H₂₂ClN₃O₂ [M+H]⁺: 360, found 360. ¹H NMR (499 MHz, DMSO-d6) δ 10.68 (s, 1H), 9.11 (s, 1H), 8.51 (s, 1H), 8.24 (s, 1H), 7.86 (s, 1H), 4.85-4.74 (m, 4H), 4.42 (s, 1H), 3.81 (m, 1H), 3.56 (m, 2H), 3.37 (m, 1H), 3.10 (s, 2H), 2.19 (s, 1H), 2.16 (s, 3H), 2.00 (m, 2H).

Compounds in Table 7 below were prepared in accordance with the synthetic sequence illustrated in General Scheme 7 and Scheme 37 using the corresponding starting materials.

TABLE 7

| | | |
|---|---|---|
| 7.2 | 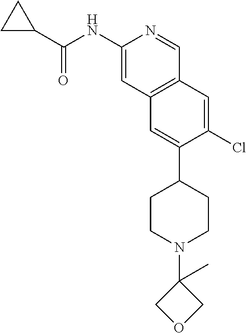 [SMILES: CC1(N2CCC(c3cc4cc(NC(=O)C5CC5)ncc4cc3Cl)CC2)COC1] | Cal'c: 399 Found: 399 |
| | N-(7-chloro-6-(1-(3-methyloxetan-3-yl)piperidin-4-yl)isoquinolin-3-yl) | |
| | cyclopropanecarboxamide | |
| 7.3 | 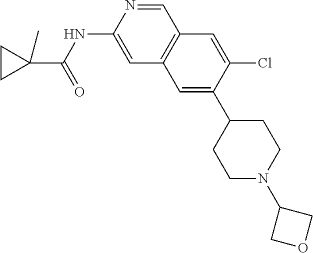 [SMILES: CC1(C(=O)Nc2cc3cc(C4CCN(C5COC5)CC4)c(Cl)cc3cn2)CC1] | Cal'c: 400 Found: 400 |
| | N-(7-chloro-6-(1-(oxetan-3-yl)piperidin-4-yl)isoquinolin-3-yl)-1- | |
| | methylcyclopropane-1-carboxamide | |
| 7.4 | 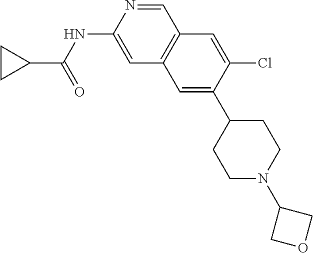 [SMILES: O=C(Nc1cc2cc(C3CCN(C4COC4)CC3)c(Cl)cc2cn1)C1CC1] | Cal'c: 400 Found: 400 |
| | N-(7-chloro-6-(1-(oxetan-3-yl)piperidin-4-yl)isoquinolin-3- | |
| | yl)cyclobutanecarboxamide | |
| 7.5 | 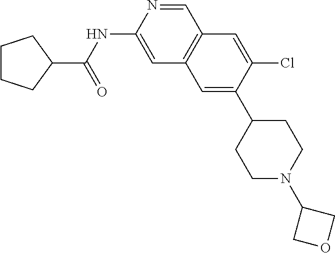 [SMILES: O=C(Nc1cc2cc(C3CCN(C4COC4)CC3)c(Cl)cc2cn1)C1CCCC1] | Cal'c: 414 Found: 414 |
| | N-(7-chloro-6-(1-(oxetan-3-yl)piperidin-4-yl)isoquinolin-3- | |
| | yl)cyclopentanecarboxamide | |
| 7.6 | 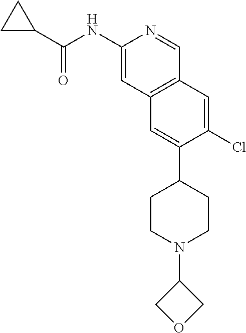 [SMILES: O=C(Nc1cc2cc(C3CCN(C4COC4)CC3)c(Cl)cc2cn1)C1CC1] | Cal'c: 385 Found: 385 |
| | N-(7-chloro-6-(1-(oxetan-3-yl)piperidin-4-yl)isoquinolin-3- | |
| | yl)cyclopropanecarboxamide | |
| 7.7 | 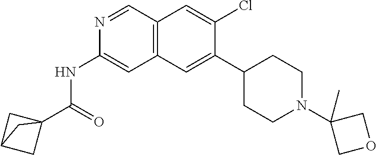 [SMILES: CC1(N2CCC(c3cc4cc(NC(=O)C56CC(C5)C6)ncc4cc3Cl)CC2)COC1] | Cal'c: 426 Found: 426 |
| | N-(7-chloro-6-(1-(3-methyloxetan-3-yl)piperidin-4-yl)isoquinolin-3- | |
| | yl)bicyclo[1.1.1]pentane-1-carboxamide | |
| 7.8 | 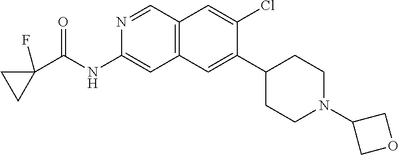 [SMILES: O=C(Nc1cc2cc(C3CCN(C4COC4)CC3)c(Cl)cc2cn1)C1(F)CC1] | Cal'c: 404 Found: 404 |
| | N-(7-chloro-6-(1-(oxetan-3-yl)piperidin-4-yl)isoquinolin-3-yl)-1- | |
| | fluorocyclopropane-1-carboxamide | |
| 7.9 | 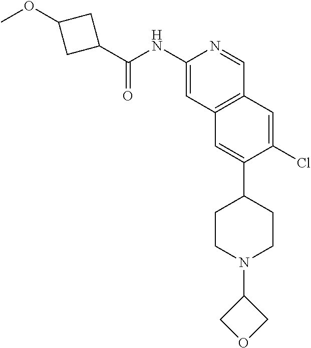 [SMILES: COC1CC(C(=O)Nc2cc3cc(C4CCN(C5COC5)CC4)c(Cl)cc3cn2)C1] | Cal'c: 430 Found: 430 |
| | Rac-N-(7-chloro-6-(1-(oxetan-3-yl)piperidin-4-yl)isoquinolin-3-yl)-3- | |
| | methoxycyclobutane-1-carboxamide | |
| 7.10 | 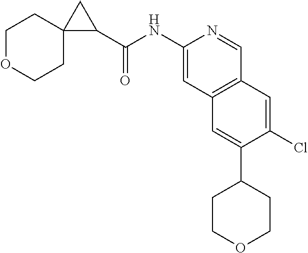 [SMILES: O=C(Nc1cc2cc(C3CCOCC3)c(Cl)cc2cn1)C1CC12CCOCC2] | Cal'c: 401 Found: 401 |
| | N-(7-chloro-6-(tetrahydro-2H-pyran-4-yl)isoquinolin-3-yl)-6- | |
| | oxaspiro[2.5]octane-1-carboxamide | |

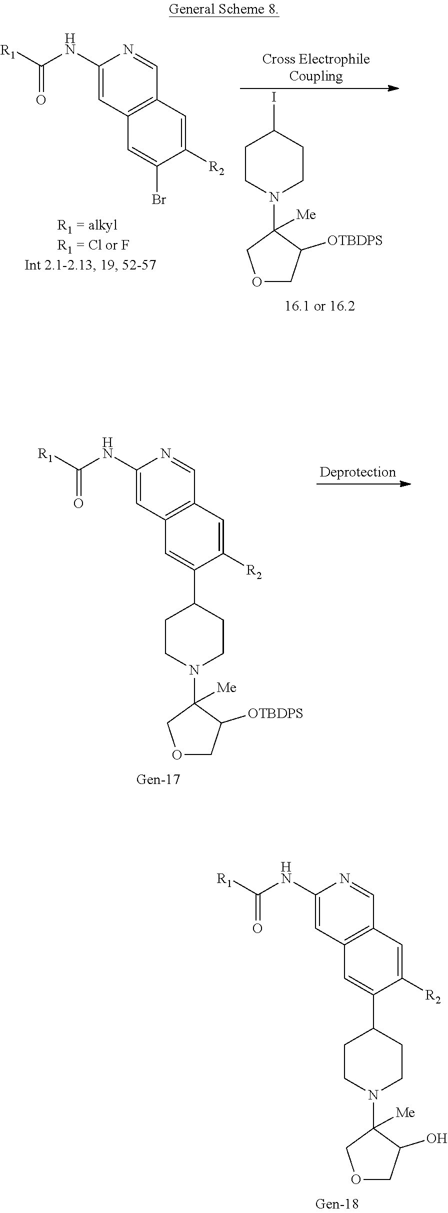

In General Scheme 8, reductive nickel C—C coupling conditions with intermediate 16.1 or 16.2 and iodopiperidine intermediates Int 2.1-2.13, 19 or 52-57 were performed to afford Gen-17. The corresponding TBDPS protected alcohol was then deprotected to access fully elaborated products in the form of Gen-18. The representative compounds are shown below.

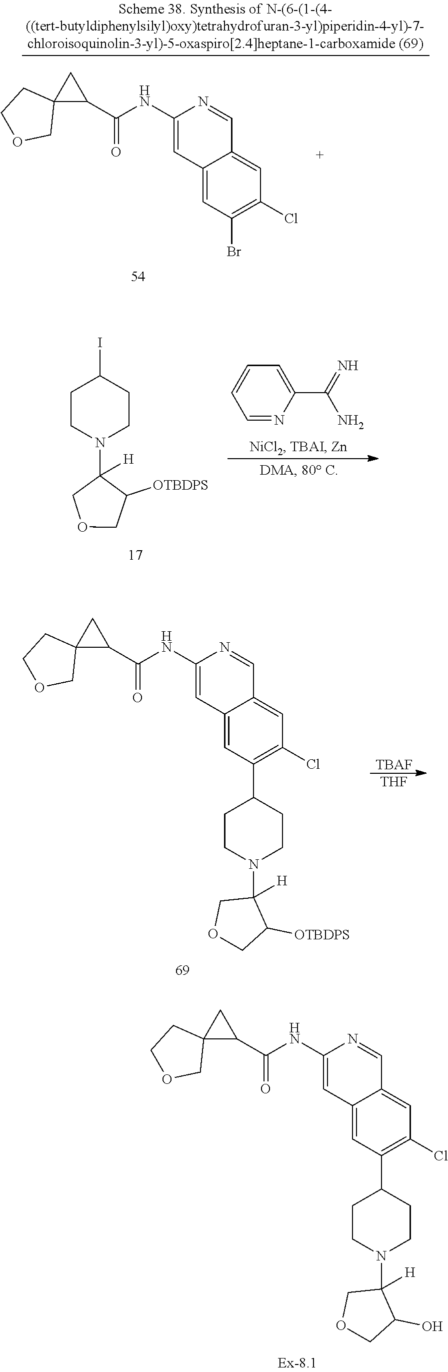

Pyridine-2-carboximidamide hydrochloride (8.26 mg, 0.052 mmol) and nickel (II) chloride ethylene glycol dimethyl ether complex (11.51 mg, 0.052 mmol) were added to a vial. The vial was evacuated and back filled with nitrogen 3 times. DMA (1310 μl) was added through the septum, and the resulting mixture was stirred for 10 minutes to fully complex the nickel and ligand. In a separate vial, N-(6-bromo-7-chloroisoquinolin-3-yl)-5-oxaspiro[2.4]heptane-1-carboxamide 64 (100 mg, 0.262 mmol), 11-(4-((tert-butyldiphenylsilyl)oxy)tetrahydrofuran-3-yl)-4-iodopiperidine 17 (210 mg, 0.393 mmol), TBAI (19.36 mg, 0.052 mmol) and zinc (51.4 mg, 0.786 mmol) were added. The vial was evacuated and back filled with nitrogen 3 times. The aforementioned nickel complex was added through the septum and the mixture was heated to 80° C. for 1 hour. The reaction was filtered over Celite, washed with EtOAc and concentrated under reduced pressure. The residue was purified by column chromatography on silica gel, eluting with 3:1 EtOAc:EtOH/hexanes (0-100%) to afford the title compound. MS (ESI): m/z calc'd for 20 C₄₁H₄₈ClN₃O₄Si [M+H]+: 710, found 710.

### N-(7-chloro-6-(1-((3R,4R or 3S,4S)-4-hydroxytetrahydrofuran-3-yl)piperidin-4-yl)isoquinolin-3-yl)-5-oxaspiro[2.4]heptane-1-carboxamide (EX 8.1)

TBAF (541 μl, 0.541 mmol) was added to a solution of N-(6-(1-(4-((tert-butyldiphenylsilyl)oxy)tetrahydrofuran-3-yl)piperidin-4-yl)-7-chloroisoquinolin-3-yl)-5-oxaspiro[2.4]heptane-1-carboxamide 69 (128 mg, 0.180 mmol) in THF (901 μl). The resulting mixture was stirred for 3 hours and then concentrated under reduced pressure. The residue was filtered and purified by HPLC, eluting acetonitrile/water gradient with 0.1% NH₄OH modifier, linear gradient) and lyophilized to afford the title compound. MS (ESI): m/z calc'd for C₂₅H₃₀ClN₃O₄ [M+H]+: 472, found 472. ¹H NMR (499 MHz, DMSO-d6) δ 10.90 (s, 1H), 9.08 (s, 1H), 8.47 (s, 1H), 8.19 (s, 1H), 7.88 (s, 1H), 4.31 (s, 1H), 4.18 (m, 1H), 3.90-3.85 (m, 2H), 3.84-3.79 (m, 2H), 3.73-3.66 (m, 2H), 3.61 (m, 1H), 3.22 (m, 1H), 3.19-3.13 (m, 1H), 3.08-2.99 (m, 1H), 2.78 (m, 1H), 2.75-2.67 (m, 1H), 2.37-2.27 (m, 1H), 2.25-2.16 (m, 1H), 2.02-1.93 (m, 1H), 1.93-1.77 (m, 4H), 1.36-1.28 (m, 1H), 1.24 (m, 2H), 0.94 (m, 1H).

Compounds in Table 8 below were prepared in accordance with the synthetic sequence illustrated in General Scheme 8 and Scheme 38 using the corresponding starting materials.

TABLE 8

| | | |
|---|---|---|
| 8.2 | 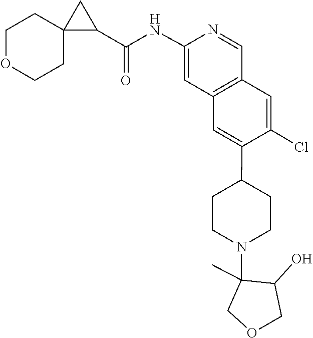 [SMILES: CC1(N2CCC(c3cc4cc(NC(=O)C5CC56CCOCC6)ncc4cc3Cl)CC2)COCC1O] | Cal′c: 500 Found: 500 |
| | N-(7-chloro-6-(1-(4-hydroxy-3-methyltetrahydrofuran-3- | |
| | yl)piperidin-4-yl)isoquinolin-3-yl)-6-oxaspiro[2.5]octane-1- | |
| | carboxamide, (1R or 1S)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-6-oxaspiro[2.5]octane-1-carboxamide | |
| 8.3 | 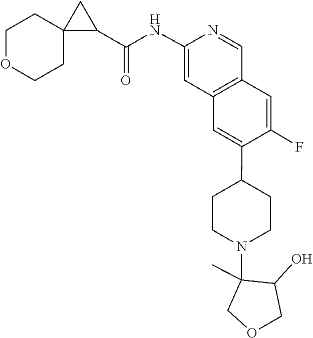 [SMILES: CC1(N2CCC(c3cc4cc(NC(=O)C5CC56CCOCC6)ncc4cc3F)CC2)COCC1O] | Cal′c: 484 Found: 484 |
| | N-(7-fluoro-6-(1-(4-hydroxy-3-methyltetrahydrofuran-3- | |
| | yl)piperidin-4-yl)isoquinolin-3-yl)-6-oxaspiro[2.5]octane-1- | |
| | carboxamide, (1R or 1S)-N-(7-fluoro-6-(1-((3R,4R or 3S,4S)-4- | |
| | hydroxy-3-methyltetrahydrofuran-3-yl)piperidin-4-yl)isoquinolin-3- | |
| | yl)-6-oxaspiro[2.5]octane-1-carboxamide | |
| 8.4 | 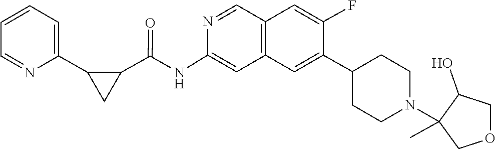 [SMILES: CC1(N2CCC(c3cc4cc(NC(=O)C5CC5c5ccccn5)ncc4cc3F)CC2)COCC1O] | Cal′c: 491 Found: 491 |
| | N-(7-fluoro-6-(1-(4-hydroxy-3-methyltetrahydrofuran-3- | |
| | yl)piperidin-4-yl)isoquinolin-3-yl)-2-(pyridin-2-yl)cyclopropane-1- | |
| | carboxamide, (1R,2R or 1S,2S)-N-(7-fluoro-6-(1-((3R,4R or 3S,4S)- | |
| | 4-hydroxy-3-methyltetrahydrofuran-3-yl)piperidin-4-yl)isoquinolin- | |
| | 3-yl)-2-(pyridin-2-yl)cyclopropane-1-carboxamide | |
| 8.5 | 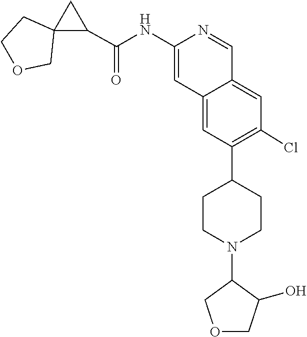 [SMILES: O=C(Nc1cc2cc(C3CCN(C4COCC4O)CC3)c(Cl)cc2cn1)C1CC12CCOC2] | Cal′c: 472 Found: 472 |
| | N-(7-chloro-6-(1-(4-hydroxytetrahydrofuran-3-yl)piperidin-4- | |
| | yl)isoquinolin-3-yl)-5-oxaspiro[2.4]heptane-1-carboxamide, (1R,2R | |
| | or 1S,2S)- N-(7-chloro-6-(1-((3R,4R or 3S,4S)-4- | |
| | hydroxytetrahydrofuran-3-yl)piperidin-4-yl)isoquinolin-3-yl)-5- | |
| | oxaspiro[2.4]heptane-1-carboxamide | |

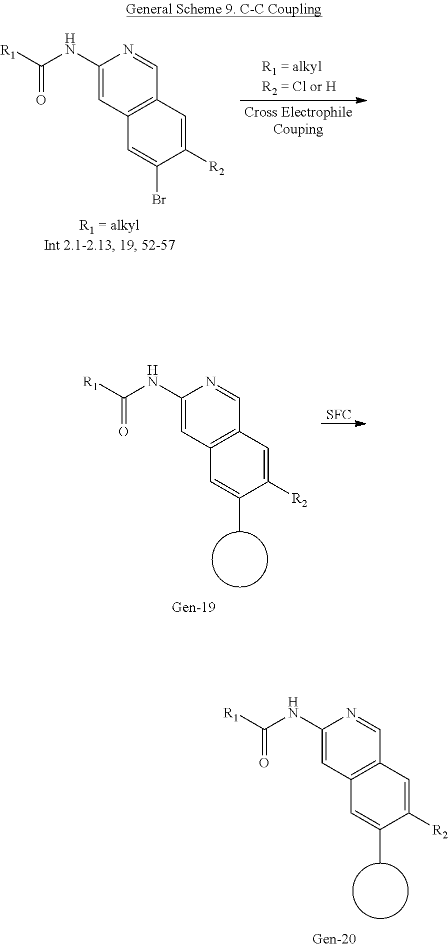

In General Scheme 9, reductive nickel C—C coupling conditions with commercially available or synthetically prepared intermediates Int 2.1-2.13, 19 or 52-57 afforded elaborated compounds of the form Gen-19 and the stereoisomers could then be separated by chiral SFC to provide fully elaborated products in the form of Gen-20. The representative compounds are described in more detail below.

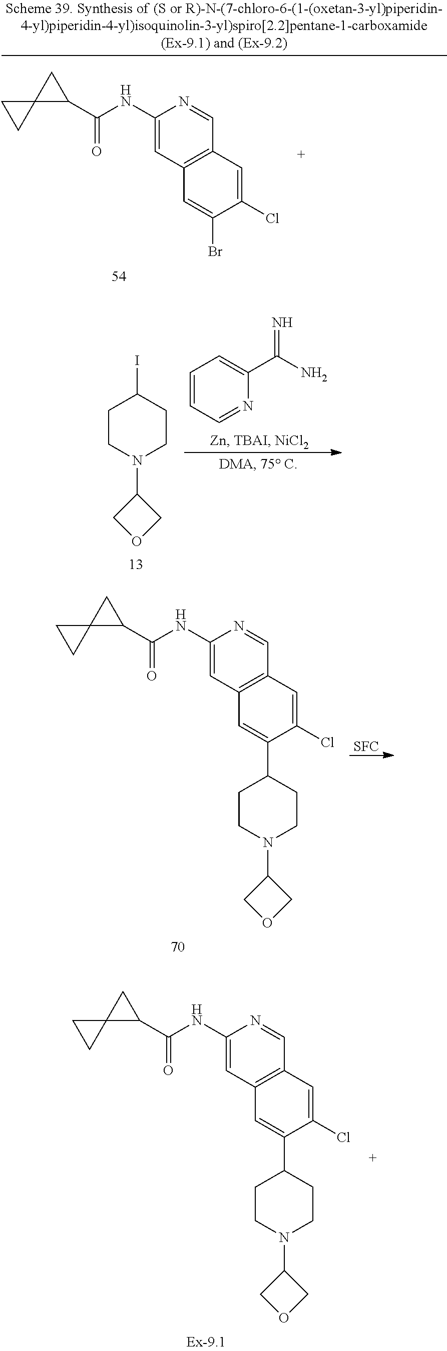

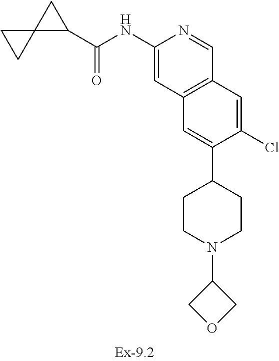 [SMILES: O=C(Nc1cc2cc(C3CCN(C4COC4)CC3)c(Cl)cc2cn1)C1CC12CC2]

Picolinimidamide hydrochloride (10.76 mg, 0.068 mmol) and nickel (II) chloride ethylene glycol dimethyl ether complex (15.00 mg, 0.068 mmol) were added to a vial. The vial was sealed, and its contents were placed under an inert atmosphere by performing 3 vacuum/nitrogen cycles. DMA (853 μl) was added through septum, and the resulting mixture was allowed to stir for 5 minutes at room temperature. In a separate vial, N-(6-bromo-7-chloroisoquinolin-3-yl)spiro[2.2]pentane-1-carboxamide 64 (120 mg, 0.341 mmol), 4-iodo-1-(oxetan-3-yl)piperidine 13 (228 mg, 0.853 mmol), TBAI (25.2 mg, 0.068 mmol), and zinc (66.9 mg, 1.024 mmol) were added. The vial was sealed, and its contents were placed under an inert atmosphere by performing 3 vacuum/nitrogen cycles. DMA (853 μl) was added through the septum followed by the previously prepared nickel complex. The resulting mixture was stirred for 1 hour at 75° C. The reaction mixture was filtered (purified by HPLC, eluting acetonitrile/water gradient with 0.1% TFA modifier, linear gradient) and lyophilized to afford the title compound as a mixture of stereoisomers The mixture of two stereoisomers was purified by chiral SFC (OD-H, 21×250 (mm), 25%/75% Methanol/CO2+0.1% NH₄OH) and lyophilized to afford resolved stereoisomers of the title compound Ex-9.1 (tR=5.7 min) and Ex-9.2 (tR=6.6 min). Ex-9.1: MS (ESI): m/z calc'd for C₂₃H₂₆ClN₃O₂ TFA [M+H]+: 412, found 412. 1H NMR (499 MHz, DMSO-d6) δ 10.74 (s, 1H), 9.07 (s, 1H), 8.50 (s, 1H), 8.19 (s, 1H), 7.85 (s, 1H), 4.53 (d, J=43.4 Hz, 3H), 3.47 (s, 1H), 3.02 (s, 1H), 2.89 (s, 1H), 2.42 (dd, J=7.3, 4.3 Hz, 1H), 2.01-1.75 (m, 6H), 1.44 (t, J=3.7 Hz, 1H), 1.37-1.34 (m, 1H), 1.24 (s, 1H), 1.17 (s, 1H), 0.94-0.90 (m, 2H), 0.89-0.85 (m, 1H), 0.80-0.76 (m, 1H). Ex-9.2: MS (ESI): m/z calc'd for C₂₃H₂₆ClN₃O₂ TFA [M+H]+: 412, found 412. 1H NMR (499 MHz, DMSO-d6) δ 10.74 (s, 1H), 9.07 (s, 1H), 8.50 (s, 1H), 8.19 (s, 1H), 7.85 (s, 1H), 4.53 (d, J=43.4 Hz, 3H), 3.47 (s, 1H), 3.02 (s, 1H), 2.89 (s, 1H), 2.42 (dd, J=7.3, 4.3 Hz, 1H), 2.01-1.75 (m, 6H), 1.44 (t, J=3.7 Hz, 1H), 1.37-1.34 (m, 1H), 1.24 (s, 1H), 1.17 (s, 1H), 0.94-0.90 (m, 2H), 0.89-0.85 (m, 1H), 0.80-0.76 (m, 1H).

Absolute stereochemistry was not assigned.

Compounds in Table 9 below were prepared in accordance with the synthetic sequence illustrated in General Scheme 9 and Scheme 39 using the corresponding starting materials.

TABLE 9

| | | |
|---|---|---|
| 9.3-9.6 | 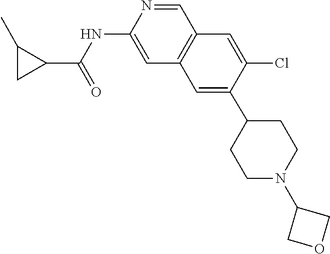 [SMILES: CC1CC1C(=O)Nc1cc2cc(C3CCN(C4COC4)CC3)c(Cl)cc2cn1] | Cal′c: 400 Found: 400 |
| | N-(7-chloro-6-(1-(oxetan-3-yl)piperidin-4-yl)isoquinolin-3-yl)-2- | |
| | methylcyclopropane-1-carboxamide, (1R,2R or 1S,2S)-N-(7-chloro- | |
| | 6-(1-(oxetan-3-yl)piperidin-4-yl)isoquinolin-3-yl)-2- | |
| | methylcyclopropane-1-carboxamide, | |
| 9.7-9.8 | 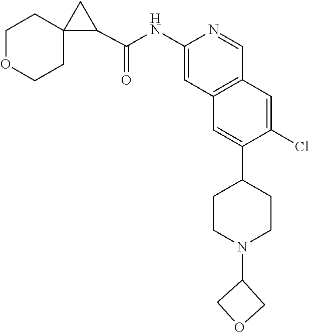 [SMILES: O=C(Nc1cc2cc(C3CCN(C4COC4)CC3)c(Cl)cc2cn1)C1CC12CCOCC2] | Cal′c: 456 Found: 456 |
| | N-(7-chloro-6-(1-(oxetan-3-yl)piperidin-4-yl)isoquinolin-3-yl)-6- | |
| | oxaspiro[2.5]octane-1-carboxamide, (1R or 1S)-N-(7-chloro-6-(1- | |
| | (oxetan-3-yl)piperidin-4-yl)isoquinolin-3-yl)-6-oxaspiro[2.5]octane- | |
| | 1-carboxamide | |
| 9.9-9.10 | 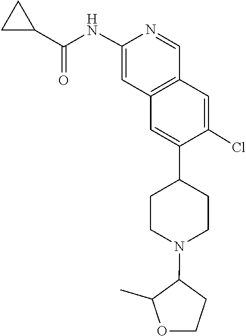 [SMILES: CC1OCCC1N1CCC(c2cc3cc(NC(=O)C4CC4)ncc3cc2Cl)CC1] | Cal′c: 414 Found: 414 |
| | N-(7-chloro-6-(1-(2-methyltetrahydrofuran-3-yl)piperidin-4- | |
| | yl)isoquinolin-3-yl)cyclopropanecarboxamide, N-(7-chloro-6-(1- | |
| | ((2R,3R or 2R,3S or 2S,3R, or 2S,3S)-2-methyltetrahydrofuran-3- | |
| | yl)piperidin-4-yl)isoquinolin-3-yl)cyclopropanecarboxamide | |
| 9.11-9.12 | 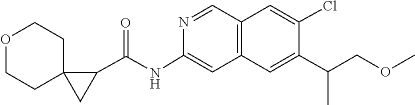 [SMILES: COCC(C)c1cc2cc(NC(=O)C3CC34CCOCC4)ncc2cc1Cl] | Cal′c: 389 Found: 389 |
| | N-(7-chloro-6-(1-methoxypropan-2-yl)isoquinolin-3-yl)-6- | |
| | oxaspiro[2.5]octane-1-carboxamide, (1R or 1S)-N-(7-chloro-6-((R | |
| | or S)-1-methoxypropan-2-yl)isoquinolin-3-yl)-6- | |
| | oxaspiro[2.5]octane-1-carboxamide | |
| 9.13-9.14 | 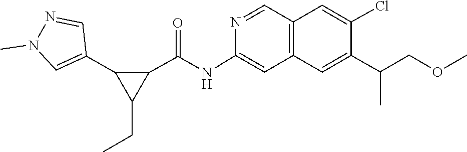 | Cal′c: 427 Found: 427 |
| | N-(7-chloro-6-(1-methoxypropan-2-yl)isoquinolin-3-yl)-2-ethyl-3- | |
| | (1-methyl-1H-pyrazol-4-yl)cyclopropane-1-carboxamide, (1R,2R or | |
| | 1S,2S)-N-(7-chloro-6-((R or S)-1-methoxypropan-2-yl)isoquinolin- | |
| | 3-yl)-2-ethyl-3-(1-methyl-1H-pyrazol-4-yl)cyclopropane-1- | |
| | carboxamide | |
| 9.15-9.16 | 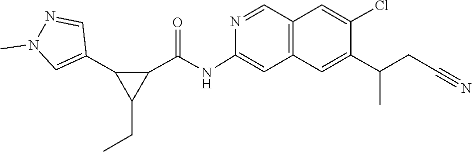 [SMILES: CCC1C(C(=O)Nc2cc3cc(C(C)CC#N)c(Cl)cc3cn2)C1c1cnn(C)c1] | Cal′c: 422 Found: 422 |
| | N-(7-chloro-6-(1-cyanopropan-2-yl)isoquinolin-3-yl)-2-ethyl-3-(1- | |
| | methyl-1H-pyrazol-4-yl)cyclopropane-1-carboxamide, (1R,2R,3R or | |
| | 1R,2S,3R or 1S,2R,3S or 1S,2S,3S)-N-(7-chloro-6-((R or S)-1- | |
| | cyanopropan-2-yl)isoquinolin-3-yl)-2-ethyl-3-(1-methyl-1H- | |
| | pyrazol-4-yl)cyclopropane-1-carboxamide | |
| 9.17-9.18 | 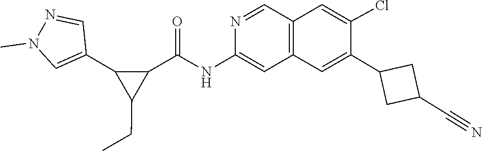 [SMILES: CCC1C(C(=O)Nc2cc3cc(C4CC(C#N)C4)c(Cl)cc3cn2)C1c1cnn(C)c1] | Cal′c: 434 Found: 434 |
| | N-(7-chloro-6-(3-cyanocyclobutyl)isoquinolin-3-yl)-2-ethyl-3-(1- | |
| | methyl-1H-pyrazol-4-yl)cyclopropane-1-carboxamide, (1R,2R,3R or | |
| | 1R,2S,3R or 1S,2R,3S or 1S,2S,3S)-N-(7-chloro-6-(cis or trans-3- | |
| | cyanocyclobutyl)isoquinolin-3-yl)-2-ethyl-3-(1-methyl-1H-pyrazol- | |
| | 4-yl)cyclopropane-1-carboxamide | |
| 9.19-9.20 | 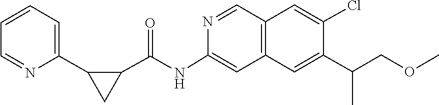 [SMILES: COCC(C)c1cc2cc(NC(=O)C3CC3c3ccccn3)ncc2cc1Cl] | Cal′c: 396 Found: 396 |
| | N-(7-chloro-6-(1-methoxypropan-2-yl)isoquinolin-3-yl)-2-(pyridin- | |
| | 2-yl)cyclopropane-1-carboxamide, (1R,2R or 1S,2S)-N-(7-chloro-6- | |
| | ((R or S)-1-methoxypropan-2-yl)isoquinolin-3-yl)-2-(pyridin-2- | |
| | yl)cyclopropane-1-carboxamide | |
| 9.21-9.22 | 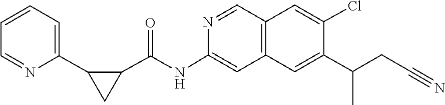 [SMILES: CC(CC#N)c1cc2cc(NC(=O)C3CC3c3ccccn3)ncc2cc1Cl] | Cal′c: 391 Found: 391 |
| | N-(7-chloro-6-(1-cyanopropan-2-yl)isoquinolin-3-yl)-2-(pyridin-2- | |
| | yl)cyclopropane-1-carboxamide, (1R,2R or 1S,2S)-N-(7-chloro-6- | |
| | ((R or S)-1-cyanopropan-2-yl)isoquinolin-3-yl)-2-(pyridin-2- | |
| | yl)cyclopropane-1-carboxamide | |
| 9.23-9.24 | 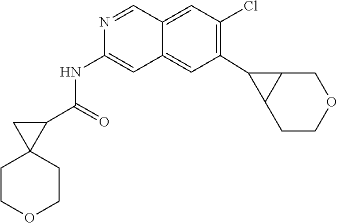 [SMILES: O=C(Nc1cc2cc(C3C4CCOCC43)c(Cl)cc2cn1)C1CC12CCOCC2] | Cal′c: 413 Found: 413 |
| | N-(6-(3-oxabicyclo[4.1.0]heptan-7-yl)-7-chloroisoquinolin-3-yl)-6- | |
| | oxaspiro[2.5]octane-1-carboxamide, (R or S)-(1R,6R or 1S,6S)-N- | |
| | (6-(3-oxabicyclo[4.1.0]heptan-7-yl)-7-chloroisoquinolin-3-yl)-6- | |
| | oxaspiro[2.5]octane-1-carboxamide | |

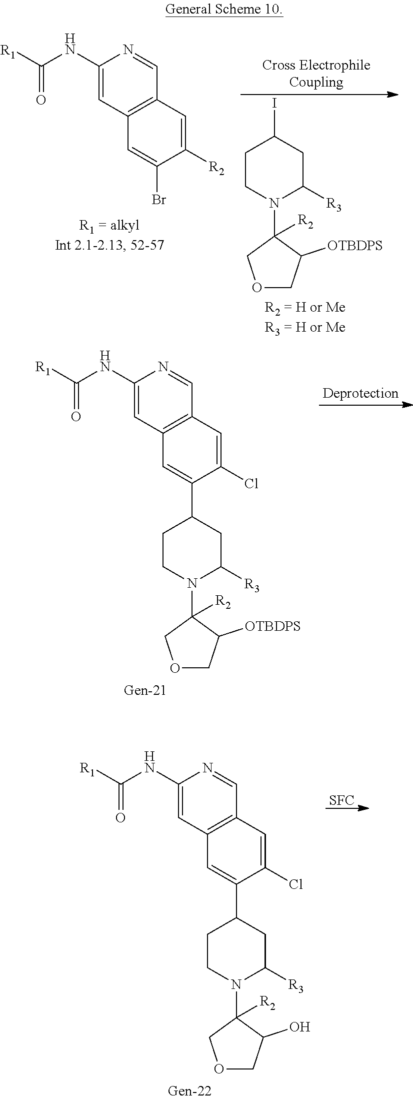

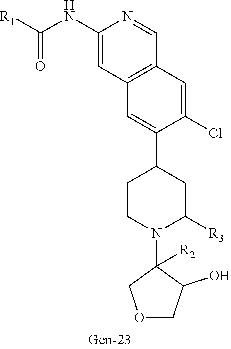

In General Scheme 10, reductive nickel C—C coupling conditions with intermediates Int 2.1-2.13, 52-57 and iodopiperidine intermediate 16 or 17 were performed to afford Gen-21. The corresponding TBDPS protected alcohol was then deprotected to access fully elaborated products in the form of Gen-22 and the stereoisomers could then be separated by chiral SFC to provide fully elaborated products in the form of Gen-23. The representative compounds are shown below.

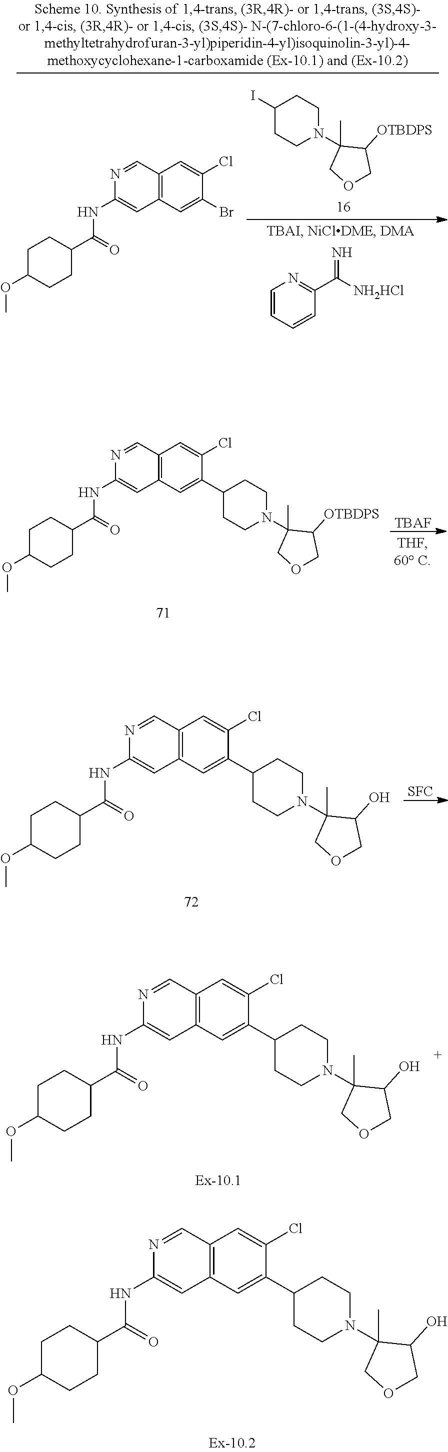

### 1,4-trans, (3R,4R)- or 1,4-trans, (3S,4S)- or 1,4-cis, (3R,4R)- or 1,4-cis, (3S,4S)-N-(6-(1-(4-((tert-butyldiphenylsilyl)oxy)-3-methyltetrahydrofuran-3-yl)piperidin-4-yl)-7-chloroisoquinolin-3-yl)-4-methoxycyclohexane-1-carboxamide (71)

To a solution containing a mixture of cis and trans isomers of N-(6-bromo-7-chloroisoquinolin-3-yl)-4-methoxycyclohexane-1-carboxamide (230 mg, 0.578 mmol) and (3S,4S)- or (3R,4R)-1-(4-((tert-butyldiphenylsilyl)oxy)-3-methyltetrahydrofuran-3-yl)-4-iodopiperidine 16 (318 mg, 0.578 mmol) in DMA (1 mL) was added tetrabutylammonium iodide (214 mg, 0.578 mmol), nickel(II) chloride ethylene glycol dimethyl ether complex (25.4 mg, 0.116 mmol), picolinimidamide hydrochloride (36.5 mg, 0.231 mmol) and manganese (159 mg, 2.89 mmol). The mixture was stirred for 2 h at 55° C. under an inert atmosphere. The mixture was filtered over Celite, and the filtrate was transferred to a separatory funnel containing DI H₂O. The aqueous phase was extracted with EtOAc (3×30 mL), dried over anhydrous Na₂SO₄, filtered, and the solvent removed from the collected filtrate under reduced pressure. The resultant crude residue was subjected to purification by flash chromatography over silica gel (PE/EtOAc, 1:1) to afford the title compound 71.

### 1,4-trans, (3R,4R)- or 1,4-trans, (3S,4S)- or 1,4-cis, (3R,4R)- or 1,4-cis, (3S,4S)-N-(7-chloro-6-(1-(4-hydroxy-3-methyltetrahydrofuran-3-yl)piperidin-4-yl)isoquinolin-3-yl)-4-methoxycyclohexane-1-carboxamide (Ex-10.1) and (Ex-10.2)

To a solution of intermediate 71 (180 mg, 0.243 mmol) in THF (10 mL) was added TBAF (486 μL, 0.486 mmol), and the resultant mixture was stirred at 50° C. for 1 h. The mixture was filtered over Celite, and solvent was removed from the collected filtrate under reduced pressure. The crude residue was purified by reverse phase HPLC (MeCN/H₂O/TFA) to give N-(7-chloro-6-(1-(4-hydroxy-3-methyltetrahydrofuran-3-yl)piperidin-4-yl)isoquinolin-3-yl)-4-methoxycyclohexane-1-carboxamide 72 as a mixture of diastereomers.

The mixture of two stereoisomers was purified by chiral SFC (Chiralpak AS-3 100*4.6 mm I.D., 3 um; Mobile phase: A: CO₂ B: ethanol with 0.05% DEA) and lyophilized to afford the chiral resolved stereoisomers of the title compound Ex-10.1 (tR=3.56 min) and Ex-10.2 (tR=3.84 min). Ex-10.1: MS (ESI): m/z calc'd for C₂₇H₃₇ClN₃O₄ [M+H]⁺: 502.3, found 502.3. ¹H NMR (500 MHz, CDCl₃) δ: 8.84 (s, 1H), 8.51 (s, 1H), 8.13 (br s, 1H), 7.89 (s, 1H), 7.68 (s, 1H), 4.12 (br s, 1H), 3.98 (m, 1H), 3.92-3.78 (m, 2H), 3.63 (m, 1H), 3.37 (s, 3H), 3.24-3.12 (m, 2H), 2.82-2.54 (m, 4H), 2.29 (m, 1H), 2.19 (m, 2H), 2.13-2.04 (m, 4H), 1.78-1.58 (m, 2H), 1.31-1.14 (m, 7H). Ex-10.2: MS (ESI): m/z calc'd for C₂₇H₃₇ClN₃O₄ [M+H]⁺: 502.3, found 502.3. ¹H NMR (500 MHz, CDCl₃) δ: 8.82 (br s, 1H), 8.53 (br s, 1H), 8.14 (br s, 1H), 7.87 (s, 1H), 7.66 (br s, 1H), 4.08 (m, 1H), 4.02-3.92 (m, 1H), 3.83 (br s, 2H), 3.61 (m, 1H), 3.46 (br s, 1H), 3.31 (s, 3H), 3.24-3.01 (m, 1H), 3.01-2.83 (m, 1H), 2.75-2.62 (m, 1H), 2.62-2.48 (m, 2H), 2.48-2.29 (m, 1H), 2.05-1.75 (m, 10H), 1.48 (m, 2H), 1.15 (br s, 3H).

Compounds in Table 10 below were prepared in accordance with the synthetic sequence illustrated in General Scheme 10 and Scheme 40 using the corresponding starting materials.

TABLE 10

| | | |
|---|---|---|
| 10.3-10.4 | 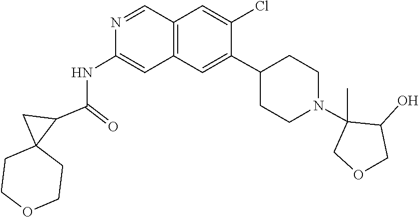 [SMILES: CC1(N2CCC(c3cc4cc(NC(=O)C5CC56CCOCC6)ncc4cc3Cl)CC2)COCC1O] | Cal′c: 500 Found: 500 |
| | N-(7-chloro-6-(1-(4-hydroxy-3-methyltetrahydrofuran-3- | |
| | yl)piperidin-4-yl)isoquinolin-3-yl)-6-oxaspiro[2.5]octane-1- | |
| | carboxamide, (1R or 1S)-N-(7-chloro-6-(1-((3R,4R or 3S,4S)-4- | |
| | hydroxy-3-methyltetrahydrofuran-3-yl)piperidin-4-yl)isoquinolin-3- | |
| | yl)-6-oxaspiro[2.5]octane-1-carboxamide | |
| 10.5-10.8 | 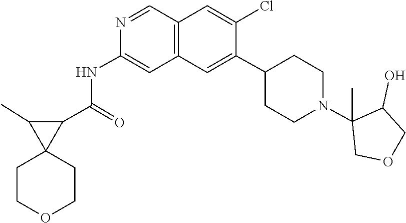 [SMILES: CC1C(C(=O)Nc2cc3cc(C4CCN(C5(C)COCC5O)CC4)c(Cl)cc3cn2)C12CCOCC2] | Cal′c: 514 Found: 514 |
| | N-(7-chloro-6-(1-(4-hydroxy-3-methyltetrahydrofuran-3- | |
| | yl)piperidin-4-yl)isoquinolin-3-yl)-2-methyl-6-oxaspiro[2.5]octane- | |
| | 1-carboxamide, (1R,2R,3R or 1R,2S,3R or 1S,2R,3S or 1S,2S,3S)- | |
| | N-(7-chloro-6-(1-((3R,4R or 3S,4S)-4-hydroxy-3- | |
| | methyltetrahydrofuran-3-yl)piperidin-4-yl)isoquinolin-3-yl)-2- | |
| | methyl-6-oxaspiro[2.5]octane-1-carboxamide | |
| 10.9-10.10 | 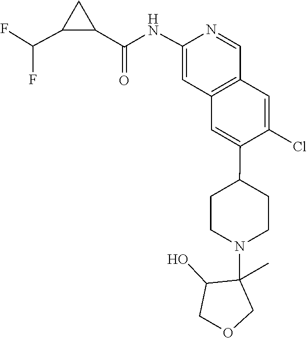 [SMILES: CC1(N2CCC(c3cc4cc(NC(=O)C5CC5C(F)F)ncc4cc3Cl)CC2)COCC1O] | Cal′c: 480 Found: 480 |
| | N-(7-chloro-6-(1-(4-hydroxy-3-methyltetrahydrofuran-3- | |
| | yl)piperidin-4-yl)isoquinolin-3-yl)-2-(difluoromethyl)cyclopropane- | |
| | 1-carboxamide, (1R,2R or 1S,2S)-N-(7-chloro-6-(1-((3R,4R or | |
| | 3S,4S)-4-hydroxy-3-methyltetrahydrofuran-3-yl)piperidin-4- | |
| | yl)isoquinolin-3-yl)-2-(difluoromethyl)cyclopropane-1-carboxamide | |

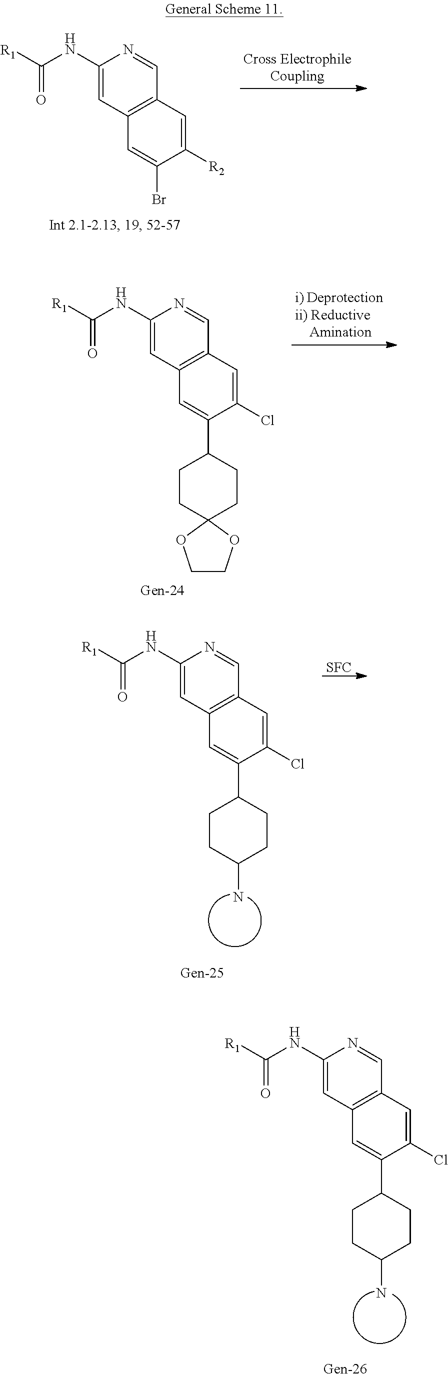

In General Scheme 11, intermediates Int 2.1-2.13, 52-57 and iodocyclohexyl intermediate underwent a cross electrophile coupling to afford products in the form of Gen-24. The intermediate was deprotected followed by a reductive amination to afford Gen-25. The stereoisomers could then be separated by chiral SFC to provide fully elaborated products in the form of Gen-26. The representative compounds are shown below.

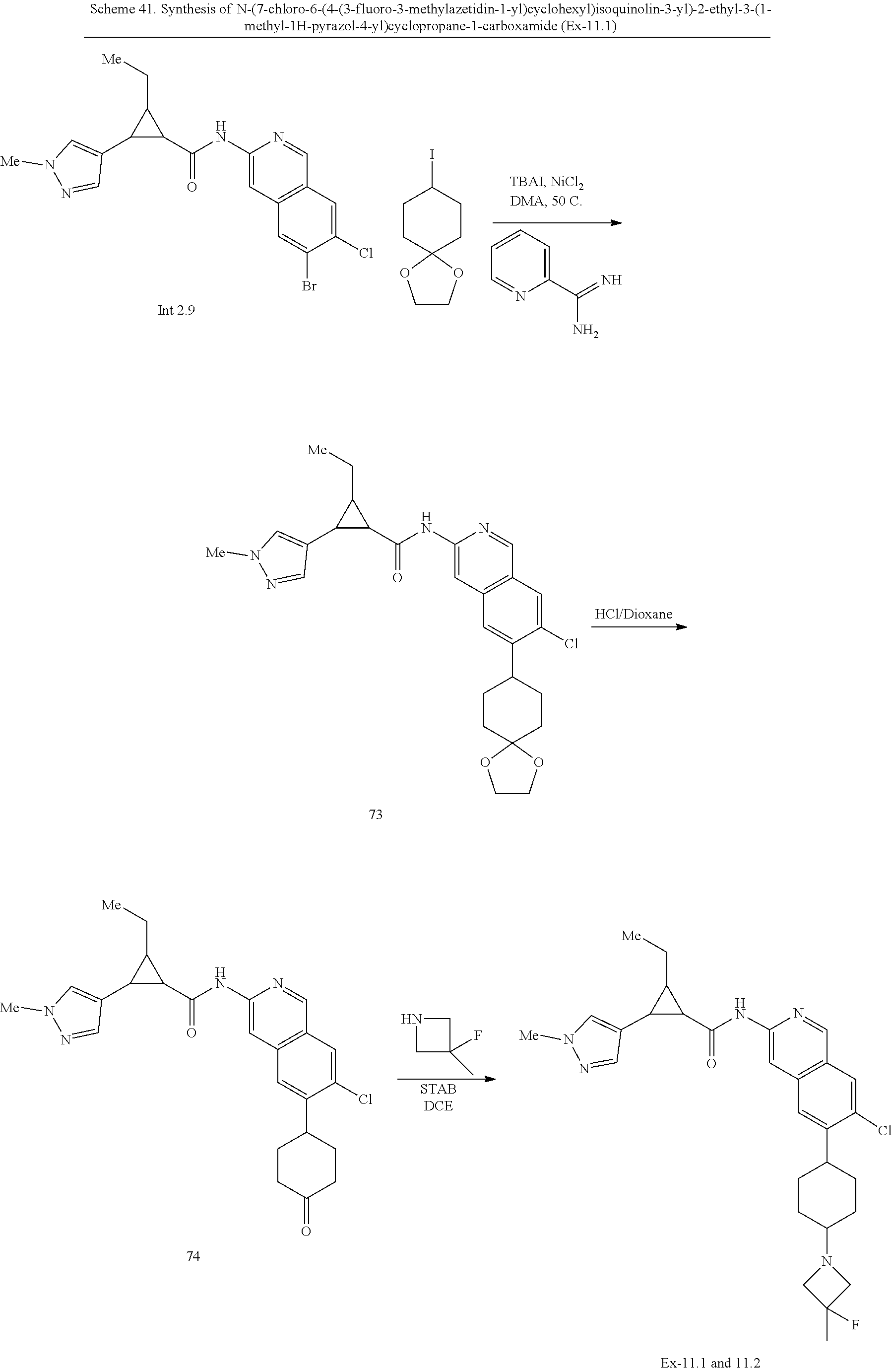

### N-(7-chloro-6-(1,4-dioxaspiro[4.5]decan-8-yl)isoquinolin-3-yl)-2-ethyl-3-(1-methyl-1H-pyrazol-4-yl)cyclopropane-1-carboxamide (73)

To a vial was added N-(6-bromo-7-chloroisoquinolin-3-yl)-2-ethyl-3-(1-methyl-1H-pyrazol-4-yl)cyclopropane-1-carboxamide Int-2.9 (300 mg, 0.692 mmol), 8-iodo-1,4-dioxaspiro[4.5]decane (371 mg, 1.383 mmol), TBAI (51.1 mg, 0.138 mmol) and zinc (136 mg, 2.075 mmol). The flask was evacuated and back filled with nitrogen 3 times. In a separate vial was added nickel(II) chloride ethylene glycol dimethyl ether complex (30.4 mg, 0.138 mmol) and pyridine-2-carboximidamide hydrochloride (21.80 mg, 0.138 mmol). The flask was evacuated and back filled with nitrogen 3 times. The solids were dissolved in DMA (6917 μl) and stirred for 10 minutes to complex. The catalyst solution was added to the main reaction vial and heated to 50° C. overnight. The reaction was crashed out in water and the solid was collected. The residue was purified by column chromatography on silica gel, eluting with hexanes/ethyl acetate to afford title compound 73.

### N-(7-chloro-6-(4-oxocyclohexyl)isoquinolin-3-yl)-2-ethyl-3-(1-methyl-1H-pyrazol-4-yl)cyclopropane-1-carboxamide (74)

In a vial N-(7-chloro-6-(1,4-dioxaspiro[4.5]decan-8-yl)isoquinolin-3-yl)-2-ethyl-3-(1-methyl-1H-pyrazol-4-yl)cyclopropane-1-carboxamide 73 (152 mg, 0.307 mmol) was dissolved in hydrochloric acid (2456 μl, 2.456 mmol). The reaction was stirred overnight at room temperature. The reaction was washed with chloroform, washed with aqueous sodium bicarbonate and extracted with ethyl acetate. The reaction was concentrated and used forward without further purification.

### (1R,2R,3R) or (1R,2S,3R) or (1S,2R,3S) or (1S,2S,3S)-N-(7-chloro-6-((cis or trans)-4-(3-fluoro-3-methylazetidin-1-yl)cyclohexyl)isoquinolin-3-yl)-2-ethyl-3-(1-methyl-1H-pyrazol-4-yl)cyclopropane-1-carboxamide (Ex-11.1) and (Ex-11.2)

To a vial was added N-(7-chloro-6-(4-oxocyclohexyl)isoquinolin-3-yl)-2-ethyl-3-(1-methyl-1H-pyrazol-4-yl)cyclopropane-1-carboxamide (60 mg, 0.133 mmol) and 3-fluoro-3-methylazetidine, HCl (33.4 mg, 0.266 mmol). The reagents were dissolved in DCE (665 μl), and DIPEA (93 μl, 0.532 mmol) was added. The reaction was stirred for 1 hour at 50° C. then sodium triacetoxyborohydride (85 mg, 0.399 mmol) was added and stirred for another hour. The reaction was quenched with ammonium chloride, extracted with DCM and concentrated in vacuo. The reaction mixture was filtered and purified by HPLC eluting acetonitrile/water gradient with 0.1% TFA modifier, linear gradient and lyophilized to afford the title compound.

Ex-11.1: MS (ESI): m/z calc'd for C₂₉H₃₆ClFN₅O [M+H]+: 524, found 524.

¹H NMR (500 MHz, DMSO-d6) δ 10.89 (s, 1H), 9.09 (s, 1H), 8.48 (s, 1H), 8.20 (s, 1H), 7.85 (s, 1H), 7.53 (s, 1H), 7.30 (s, 1H), 4.59-4.44 (m, 2H), 4.44-4.28 (m, 3H), 3.35 (s, 1H), 3.02 (s, 1H), 2.35 (m, 1H), 2.24-2.16 (m, 2H), 2.12-2.02 (m, 3H), 1.69 (s, 1H), 1.65-1.57 (m, 4H), 1.53 (m, 3H), 1.45-1.36 (m, 2H), 1.24 (m, 2H), 0.89 (t, J=7.3 Hz, 3H).

Ex-11.2: MS (ESI): m/z calc'd for C₂₉H₃₆ClFN₅O [M+H]+: 524, found 524.

¹H NMR (500 MHz, DMSO-d₆) δ 10.92 (s, 1H), 9.11 (s, 1H), 8.48 (m, 1H), 8.21 (s, 1H), 7.81 (m, 1H), 7.53 (s, 1H), 4.64-4.34 (m, 4H), 3.80 (m, 6H), 2.35 (m, 1H), 3.16 (s, 1H), 2.21 (s, 2H), 1.96-1.79 (m, 4H), 1.67 (m, 3H), 1.52 (m, 2H), 1.23 (m, 2H), 0.89 (m, 3H).

Compounds in Table 11 below were prepared in accordance with the synthetic sequence illustrated in General Scheme 11 and Scheme 41 using the corresponding starting materials.

TABLE 11

| | | |
|---|---|---|
| 11.3-11.4 | 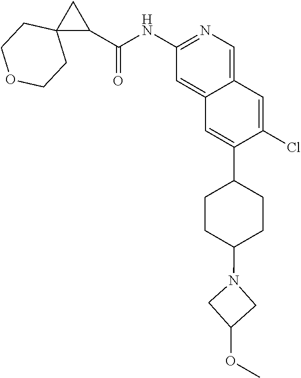 [SMILES: COC1CN(C2CCC(c3cc4cc(NC(=O)C5CC56CCOCC6)ncc4cc3Cl)CC2)C1] | Cal′c: 484 Found: 484 |
| | N-(7-chloro-6-(4-(3-methoxyazetidin-1-yl)cyclohexyl)isoquinolin- | |
| | 3-yl)-6-oxaspiro[2.5]octane-1-carboxamide, (1R or 1S)-N-(7-chloro- | |
| | 6-(cis or trans-4-(3-methoxyazetidin-1-yl)cyclohexyl)isoquinolin-3- | |
| | yl)-6-oxaspiro[2.5]octane-1-carboxamide | |
| 11.5-11.6 | 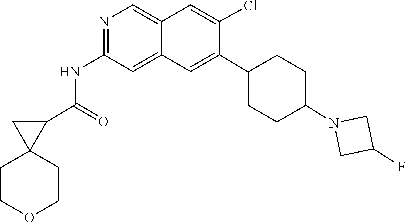 [SMILES: O=C(Nc1cc2cc(C3CCC(N4CC(F)C4)CC3)c(Cl)cc2cn1)C1CC12CCOCC2] | Cal′c: 472 Found: 472 |
| | N-(7-chloro-6-(4-(3-fluoroazetidin-1-yl)cyclohexyl)isoquinolin-3- | |
| | yl)-6-oxaspiro[2.5]octane-1-carboxamide, (1R or 1S)-N-(7-chloro-6- | |
| | (cis or trans-4-(3-fluoroazetidin-1-yl)cyclohexyl)isoquinolin-3-yl)- | |
| | 6-oxaspiro[2.5]octane-1-carboxamide | |
| 11.7-11.8 | 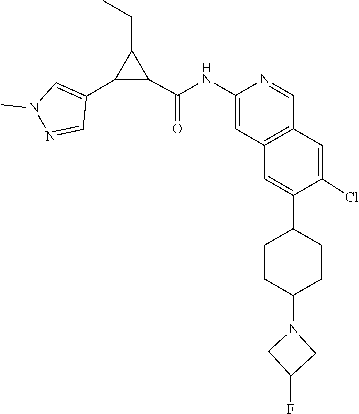 [SMILES: CCC1C(C(=O)Nc2cc3cc(C4CCC(N5CC(F)C5)CC4)c(Cl)cc3cn2)C1c1cnn(C)c1] | Cal′c: 510 Found: 510 |
| | N-(7-chloro-6-(4-(3-fluoroazetidin-1-yl)cyclohexyl)isoquinolin-3- | |
| | yl)-2-ethyl-3-(1-methyl-1H-pyrazol-4-yl)cyclopropane-1- | |
| | carboxamide, (1R,2R,3R or 1R,2S,3R or 1S,2R,3S or 1S,2S,3S)-N- | |
| | (7-chloro-6-(cis or tras-4-(3-fluoroazetidin-1- | |
| | yl)cyclohexyl)isoquinolin-3-yl)-2-ethyl-3-(1-methyl-1H-pyrazol-4- | |
| | yl)cyclopropane-1-carboxamide | |

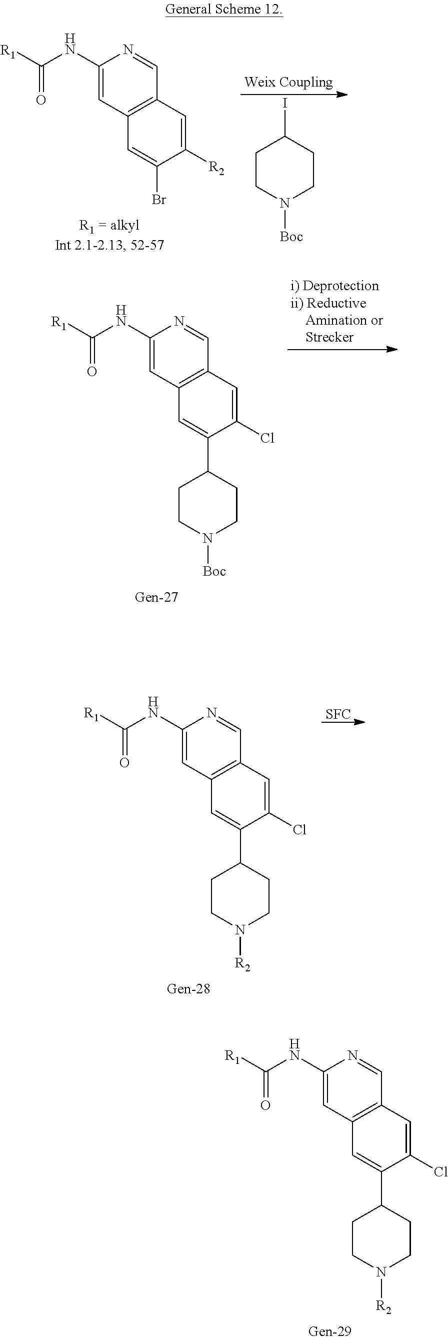

In General Scheme 12, reductive nickel C—C coupling conditions with intermediates Int 2.1-2.13, 52-57 and boc (tert-butyloxycarbonyl)-iodopiperidine were performed to afford Gen-27. The corresponding Boc-protected piperidine was then deprotected followed by a reductive amination or Strecker reaction to afford Gen-28 and the stereoisomers could then be separated by chiral SFC to provide fully elaborated products in the form of Gen-29. The representative compounds are shown below.

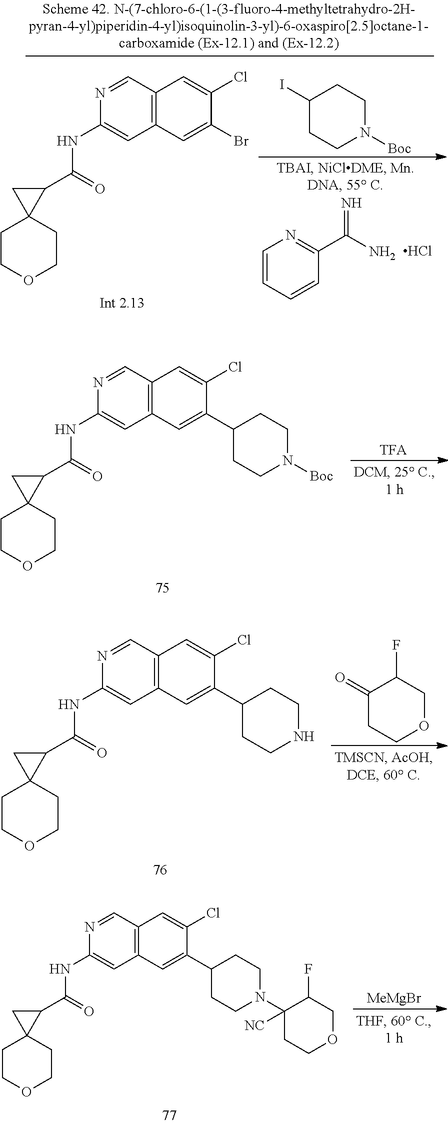

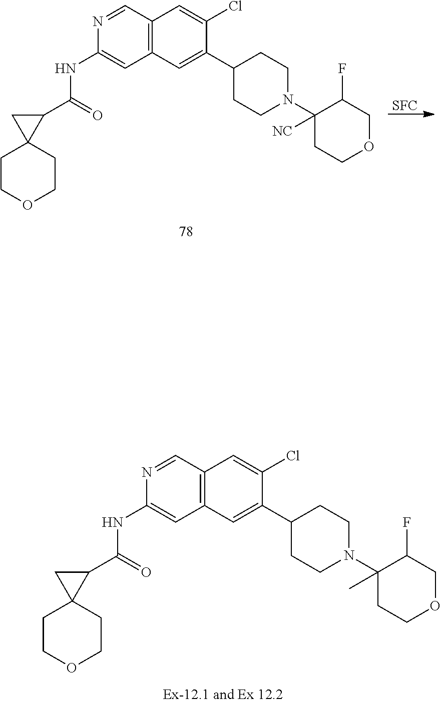

### tert-butyl4-(7-chloro-3-(6-oxaspiro[2.5]octane-1-carboxamido)isoquinolin-6-yl)piperidine-1-carboxylate (75)

To a solution of N-(6-bromo-7-chloroisoquinolin-3-yl)-6-oxaspiro[2.5]octane-1-carboxamide Int-2.13 (617 mg, 0.650 mmol) and tert-butyl 4-iodopiperidine-1-carboxylate (582.6 mg, 0.779 mmol) in DMA (12.0 mL) were added manganese (427.7 mg, 3.25 mmol), nickel (II) chloride ethylene glycol dimethyl ether complex (68.46 mg, 0.130 mmol), tetrabutylammonium iodide (576 mg, 0.650 mmol) and picolinimidamide hydrochloride (98.26 mg, 0.260 mmol). The mixture was stirred for 1 h at 55° C. Water (10 mL) was added to the mixture and the mixture was extracted with EtOAc (20 mL×3). The combined organic layers were washed with brine (20 mL). The mixture was dried over Na₂SO₄ and filtered. The filtrate was concentrated in vacuo to give a crude product which was purified by prep-TLC to afford title compound 75. MS (ESI): m/z calc'd for C₂₇H₃₅ClN₃O₄ [M+H]+: 500, found 500.

### N-(7-chloro-6-(piperidin-4-yl)isoquinolin-3-yl)-6-oxaspiro[2.5]octane-1-carboxamide (76)

To a solution of tert-butyl-4-(7-chloro-3-(6-oxaspiro[2.5]octane-1-carboxamido)isoquinolin-6-yl)piperidine-1-carboxylate 75 (550 mg, 0.312 mmol) in DCM (6.5 mL) was added TFA (1.3 mL, 5.19 mmol). The mixture was stirred at 25° C. for 1 hour. The mixture was concentrated in vacuo to give the crude mixture. The crude product was dissolved with MeOH (10 mL) and then K₂CO₃ was added to the mixture to adjust to about pH ˜9. The mixture was concentrated in vacuo followed by the addition DCM. The mixture was filtered, and the filtrate was concentrated in vacuo to afford title compound 76. MS (ESI): m/z calc'd for C₂₂H₂₇ClN₃O₂ [M+H]+: 400, found 400.

### N-(7-chloro-6-(1-(4-cyano-3-fluorotetrahydro-2H-pyran-4-yl)piperidin-4-yl)isoquinolin-3-yl)-6-oxaspiro[2.5]octane-1-carboxamide (77)

To a solution of N-(7-chloro-6-(piperidin-4-yl)isoquinolin-3-yl)-6-oxaspiro[2.5]octane-1-carboxamide 76 (340 mg, 0.275 mmol) in DCE (3.0 mL) was added 3-fluorotetrahydro-4H-pyran-4-one (223.1 mg, 0.688 mmol) and AcOH (0.251 mL, 1.375 mmol). The mixture was stirred at 60° C. and then trimethylsilanecarbonitrile (571 mg, 2.75 mmol) was added to the mixture. The resulting mixture was stirred at 60° C. for 24 hours. Water (10 mL) was added to the mixture and the mixture was extracted with EtOAc (10 mL×3). The combined organic layers were dried over Na₂SO₄ and filtered. The filtrate was concentrated in vacuo to give a crude product. The crude product was purified by pre-TLC to afford title compound 77. MS (ESI): m/z calc'd for C₂₈H₃₃ClFN₄O₃ [M+H]⁺: 527, found 527.

### N-(7-chloro-6-(1-(3-fluoro-4-methyltetrahydro-2H-pyran-4-yl)piperidin-4-yl)isoquinolin-3-yl)-6-oxaspiro[2.5]octane-1-carboxamide (78)

To a solution of N-(7-chloro-6-(1-(4-cyano-3-fluorotetrahydro-2H-pyran-4-yl)piperidin-4-yl)isoquinolin-3-yl)-6-oxaspiro[2.5]octane-1-carboxamide 77 (175 mg, 0.237 mmol) in THF (1 mL) was added methylmagnesium bromide (1.107 mL, 2.372 mmol) at 0° C. The resulting solution was heated to 60° C. The reaction was quenched with saturated aqueous NH₄Cl, and extracted with EtOAc (5 mL×3), the mixture was dried over Na₂SO₄ and filtered. The filtrate was concentrated in vacuo to give a crude product. The crude product was purified by prep-HPLC (TFA) afford title compound 78. MS (ESI): m/z calc'd for C₂₈H₃₆ClFN₃O₃ [M+H]+: 516, found 516.

### N-(7-chloro-6-(1-((3R,4R or 3S,4S)-3-fluoro-4-methyltetrahydro-2H-pyran-4-yl)piperidin-4-yl)isoquinolin-3-yl)-6-oxaspiro[2.5]octane-1-carboxamide (Ex-12.1) and (Ex-12.2)

N-(7-chloro-6-(1-(3-fluoro-4-methyltetrahydro-2H-pyran-4-yl)piperidin-4-yl)isoquinolin-3-yl)-6-oxaspiro[2.5]octane-1-carboxamide was separated by SFC Column: Chiralpak IG-3 50×4.6 mm I.D., 3 um, Mobile phase: A: CO₂, B: ethanol (0.05% DEA), Isocratic: 40% B, Flow rate: 4 mL/min, Column temp.: 35° C., ABPR: 1500 psi to afford title compounds (Ex-12.1) and (Ex-12.2).

Ex-12.1: MS (ESI): m/z calc'd for C₂₈H₃₆ClFN₃O₃ [M+H]+: 516, found 516.

¹H NMR (400 MHz, CDCl₃) δ: 8.85 (s, 1H), 8.49-8.33 (m, 2H), 7.89 (s, 1H), 7.70 (s, 1H), 7.27 (s, 1H), 4.55-4.38 (m, 1H), 4.14-4.04 (m, 1H), 3.98-3.92 (m, 1H), 3.75-3.70 (m, 6H), 3.61-3.52 (m, 1H), 3.29 (d, J=10.8 Hz, 1H), 3.14-3.05 (m, 2H), 2.49-2.31 (m, 2H), 2.19-2.10 (m, 1H), 2.01 (d, J=12.0 Hz, 2H), 1.85 (s, 1H), 1.57 (d, J=11.5 Hz, 3H), 1.41-1.37 (m, 1H), 1.25 (t, J=7.1 Hz, 6H), 1.13 (s, 3H), 1.00 (dd, J=4.5, 7.7 Hz, 1H)

Ex-12.2: MS (ESI): m/z calc'd for C₂₈H₃₆ClFN₃O₃ [M+H]+: 516, found 516.

¹H NMR (400 MHz, CDCl₃) δ: 8.85 (s, 1H), 8.46-8.33 (m, 2H), 7.89 (s, 1H), 7.70 (s, 1H), 4.56-4.39 (m, 1H), 4.09 (m, 1H), 3.97-3.92 (m, 1H), 3.74-3.69 (m, 4H), 3.58-3.53 (m, 1H), 3.29 (d, J=10.5 Hz, 1H), 3.12-3.05 (m, 2H), 2.47-2.34 (m, 2H), 2.14 (t, J=11.4 Hz, 1H), 2.04-1.97 (m, 2H), 1.85 (d, J=2.2 Hz, 1H), 1.57 (dd, J=5.1, 13.2 Hz, 3H), 1.39 (t, J=4.9 Hz, 1H), 1.26-1.23 (m, 3H), 1.13 (s, 3H), 1.00 (dd, J=4.6, 7.8 Hz, 1H)

Compounds in Table 12 below were prepared in accordance with the synthetic sequence illustrated in General Scheme 12 and Scheme 42 using the corresponding starting materials.

TABLE 12

| | | |
|---|---|---|
| 12.3-12.5 | 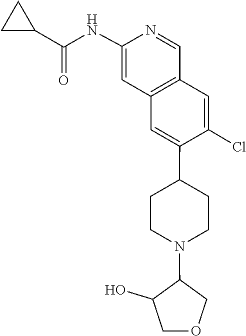 [SMILES: O=C(Nc1cc2cc(C3CCN(C4COCC4O)CC3)c(Cl)cc2cn1)C1CC1] | Cal′c: 416 Found:416 |
| | N-(7-chloro-6-(1-(4-hydroxytetrahydrofuran-3-yl)piperidin-4- | |
| | yl)isoquinolin-3-yl)cyclopropanecarboxamide, N-(7-chloro-6-(1- | |
| | ((3R,4R or 3S,4S)-4-hydroxytetrahydrofuran-3-yl)piperidin-4- | |
| | yl)isoquinolin-3-yl)cyclopropanecarboxamide | |
| 12.6-12.9 | 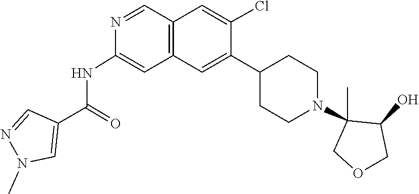 [SMILES: Cn1cc(C(=O)Nc2cc3cc(C4CCN([C@]5(C)COC[C@@H]5O)CC4)c(Cl)cc3cn2)cn1] | Cal′c: 470 Found: 470 |
| | N-(7-chloro-6-(1-(4-hydroxy-3-methyltetrahydrofuran-3- | |
| | yl)piperidin-4-yl)isoquinolin-3-yl)-1-methyl-1H-pyrazole-4- | |
| | carboxamide, N-(7-chloro-6-(1-((3R,4R or 3S,4S)-4-hydroxy-3- | |
| | methyltetrahydrofuran-3-yl)piperidin-4-yl)isoquinolin-3-yl)-1- | |
| | methyl-1H-pyrazole-4-carboxamide | |

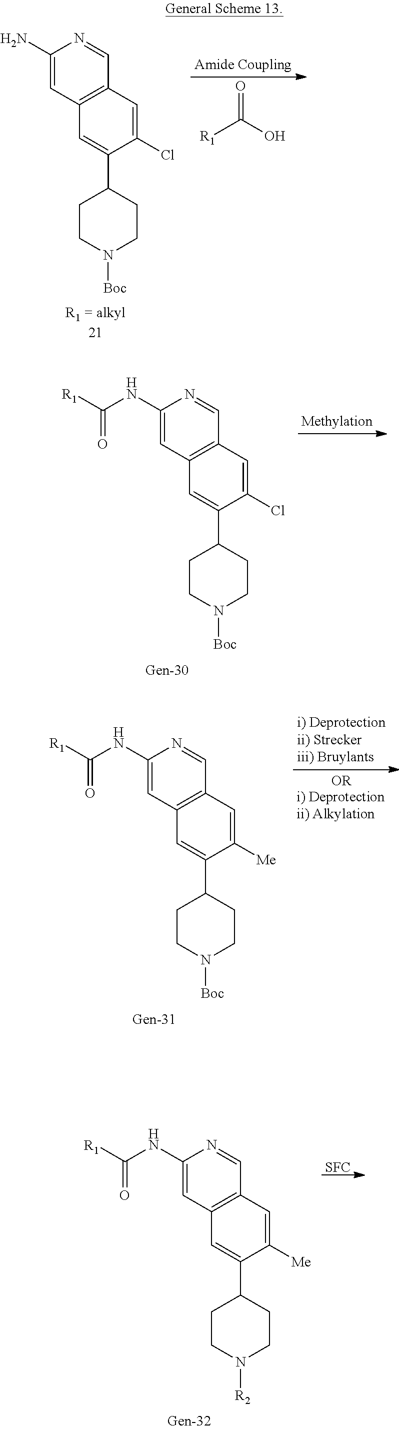

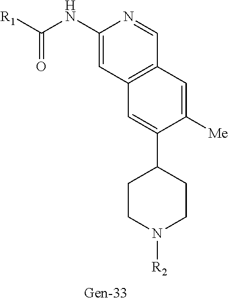

In General Scheme 13, amide coupling conditions with intermediate 21 and commercially available carboxylic acids were performed to afford Gen-30. The corresponding Boc-protected piperidine was then methylated to afford Gen-31 which was subsequently deprotected, followed by a reductive amination or Strecker reaction to afford Gen-32. The stereoisomers could then be separated by chiral SFC to provide fully elaborated products in the form of Gen-33. The representative compounds are shown below.

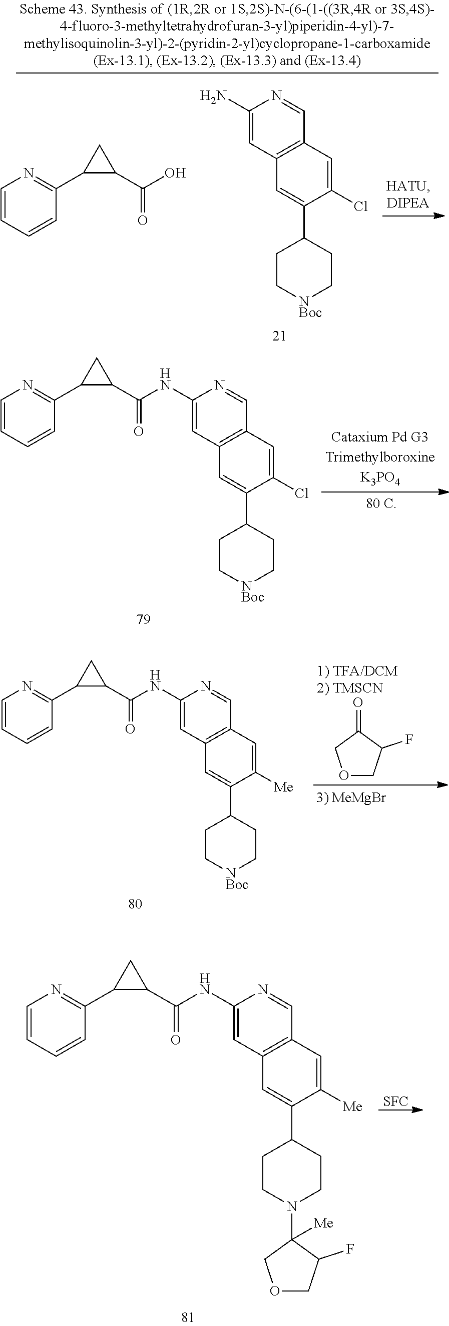

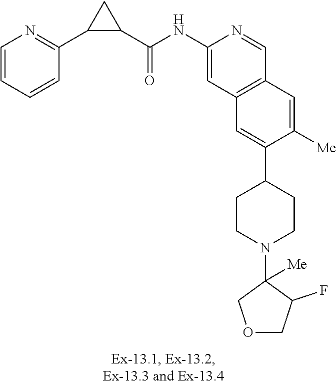 [SMILES: Cc1cc2cnc(NC(=O)C3CC3c3ccccn3)cc2cc1C1CCN(C2(C)COCC2F)CC1]

### tert-butyl 4-(7-chloro-3-(2-(pyridin-2-yl)cyclopropane-1-carboxamido)isoquinolin-6-yl)piperidine-1-carboxylate (79)

To a vial was added 2-(pyridin-2-yl)cyclopropane-1-carboxylic acid (0.902 g, 5.53 mmol), tert-butyl 4-(3-amino-7-chloroisoquinolin-6-yl)piperidine-1-carboxylate 21 (1 g, 2.76 mmol), HATU (3.15 g, 8.29 mmol), DMF (13.82 ml) and Hunig's Base (2.413 ml, 13.82 mmol). The reaction mixture was stirred at rt overnight. The reaction mixture was added dropwise to an Erlenmeyer flask of deionized water. The desired product was sonicated to precipitate out the desired product. The solid was filtered to afford title compound 79.

### tert-butyl 4-(7-methyl-3-(2-(pyridin-2-yl)cyclopropane-1-carboxamido)isoquinolin-6-yl)piperidine-1-carboxylate (80)

To a vial was added tert-butyl 4-(7-chloro-3-(2-(pyridin-2-yl)cyclopropane-1-carboxamido)isoquinolin-6-yl)piperidine-1-carboxylate 79 (800 mg, 1.578 mmol), Cataxium Pd G3 (230 mg, 0.316 mmol), and potassium phosphate, tribasic (1005 mg, 4.73 mmol). Dioxane (7100 μl) and Water (789 μl) were added, and the vial was purged with nitrogen. Trimethylboroxine (2211 μl, 15.78 mmol) was added, and the mixture was stirred at 80° C. overnight. The reaction mixture was filtered and concentrated. The crude reaction mixture was diluted in DCM and purified by column chromatography to afford title compound 80.

### N-(7-methyl-6-(piperidin-4-yl)isoquinolin-3-yl)-2-(pyridin-2-yl)cyclopropane-1-carboxamide

tert-butyl 4-(7-methyl-3-(2-(pyridin-2-yl)cyclopropane-1-carboxamido)isoquinolin-6-yl)piperidine-1-carboxylate (600 mg, 1.233 mmol) was dissolved in DCM (6165 μl), and TFA (950 μl, 12.33 mmol) was added. The reaction mixture was stirred at rt overnight. Saturated aqueous sodium bicarbonate was slowly added to achieve a neutral pH. The desired product was extracted with DCM. The organic layers were combined, dried, and concentrated to afford N-(7-methyl-6-(piperidin-4-yl)isoquinolin-3-yl)-2-(pyridin-2-yl)cyclopropane-1-carboxamide.

### N-(6-(1-(3-cyano-4-fluorotetrahydrofuran-3-yl)piperidin-4-yl)-7-methylisoquinolin-3-yl)-2-(pyridin-2-yl)cyclopropane-1-carboxamide

N-(7-methyl-6-(piperidin-4-yl)isoquinolin-3-yl)-2-(pyridin-2-yl)cyclopropane-1-carboxamide (300 mg, 0.776 mmol) was added to a 30 mL vial. DCE (3881 μl) was added under positive flow of nitrogen, and 4-fluorodihydrofuran-3(2H)-one (404 mg, 3.88 mmol) was added followed by addition of acetic acid (133 μl, 2.329 mmol). The vial was stirred at 65° C. for 30 min after which TMS-CN (520 μl, 3.88 mmol) was added. The reaction vessel was stirred at 65° C. for 16 h. The reaction mixture was extracted with DCM and 1M NaOH. The organic phase was combined, dried using magnesium sulfate, and concentrated in vacuo. The crude product was diluted in DCM and purified by column chromatography to afford title compound N-(6-(1-(3-cyano-4-fluorotetrahydrofuran-3-yl)piperidin-4-yl)-7-methylisoquinolin-3-yl)-2-(pyridin-2-yl)cyclopropane-1-carboxamide.

### N-(6-(1-(4-fluoro-3-methyltetrahydrofuran-3-yl)piperidin-4-yl)-7-methylisoquinolin-3-yl)-2-(pyridin-2-yl)cyclopropane-1-carboxamide (81)

N-(6-(1-(3-cyano-4-fluorotetrahydrofuran-3-yl)piperidin-4-yl)-7-methylisoquinolin-3-yl)-2-(pyridin-2-yl)cyclopropane-1-carboxamide (100 mg, 0.200 mmol) was taken up in THF (1001 μl), and the reaction vial was purged with nitrogen 3 times. Under positive flow of nitrogen methylmagnesium bromide (334 μl, 1.001 mmol) was added. The reaction mixture was stirred overnight at 50° C. The reaction mixture was diluted in DCM and extracted with saturated ammonium chloride. Organic layers were combined, dried, and concentrated. The crude reaction mixture was diluted in DCM and purified by column chromatography to afford title compound 81.

### (1R,2R or 1S,2S)-N-(6-(1-((3R,4R or 3S,4S)-4-fluoro-3-methyltetrahydrofuran-3-yl)piperidin-4-yl)-7-methylisoquinolin-3-yl)-2-(pyridin-2-yl)cyclopropane-1-carboxamide (Ex-13.1), (Ex-13.2), (Ex-13.3) and (Ex-13.4)

N-(6-(1-(4-fluoro-3-methyltetrahydrofuran-3-yl)piperidin-4-yl)-7-methylisoquinolin-3-yl)-2-(pyridin-2-yl)cyclopropane-1-carboxamide was separated by chiral SFC column: CCO F4, 21×250 mm, 5 um, Mobile phase: A CO₂, B: methanol (0.1% NH₄OH), Flow rate: 70 mL/min, to afford title compounds Ex-13.1 (rT=4.55 min), Ex-13.2 (rT=5.20 min), Ex-13.3 (rT=5.75 min), and Ex-13.4 (rT=6.80 min).

Compounds in Table 13 below were prepared in accordance with the synthetic sequence illustrated in General Scheme 13 and Scheme 43 using the corresponding starting materials.

TABLE 13

| | | |
|---|---|---|
| 13.5 | 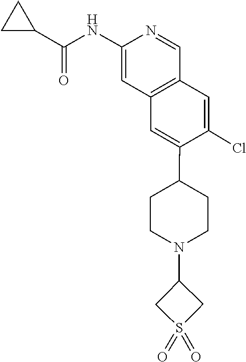 [SMILES: O=C(Nc1cc2cc(C3CCN(C4CS(=O)(=O)C4)CC3)c(Cl)cc2cn1)C1CC1] | Cal′c: 434 Found: 434 |
| | N-(7-chloro-6-(1-(1,1-dioxidothietan-3-yl)piperidin-4- | |
| | yl)isoquinolin-3-yl)cyclopropanecarboxamide | |

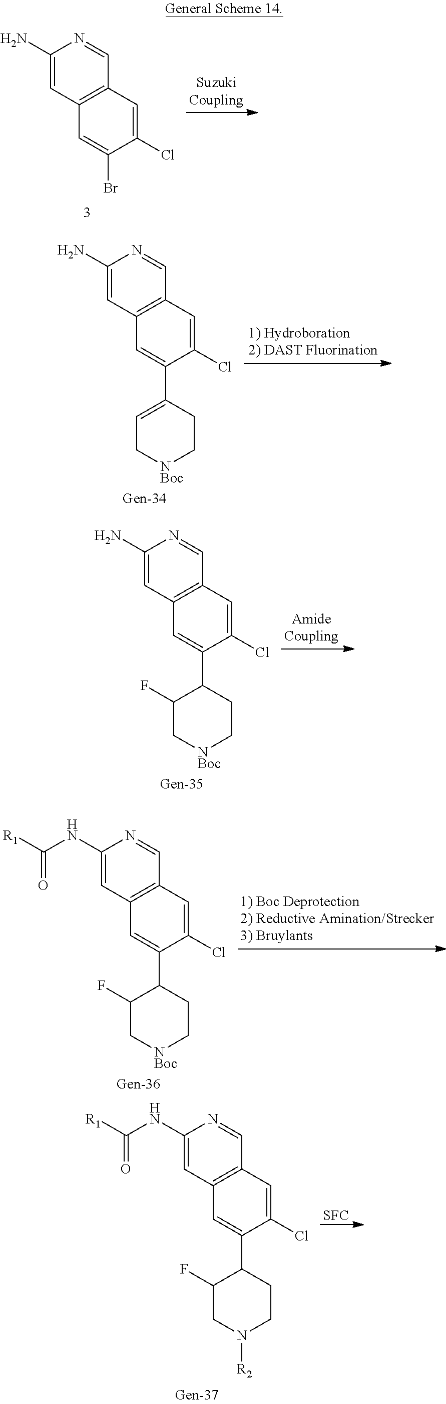

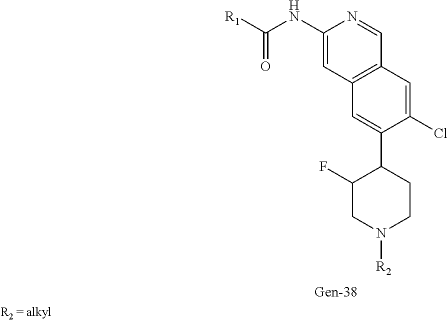

In General Scheme 14, intermediate 3 underwent a Suzuki coupling with commercially available boronic acids to afford Gen-34. The subsequent alkene then subjected to hydroboration conditions followed by DAST fluorination to provide a fluorinated heterocycle in the form of Gen-35. Gen-35 was then acylated with amide coupling conditions to afford Gen-36. Finally, a Boc deprotection followed by a reductive amination with commercially available ketones or intermediates was performed to provide fully elaborated compounds in the form of Gen-37. The stereoisomers could then be separated by chiral SFC to provide fully elaborated products in the form of Gen-38. The representative compounds are described in more detail below.

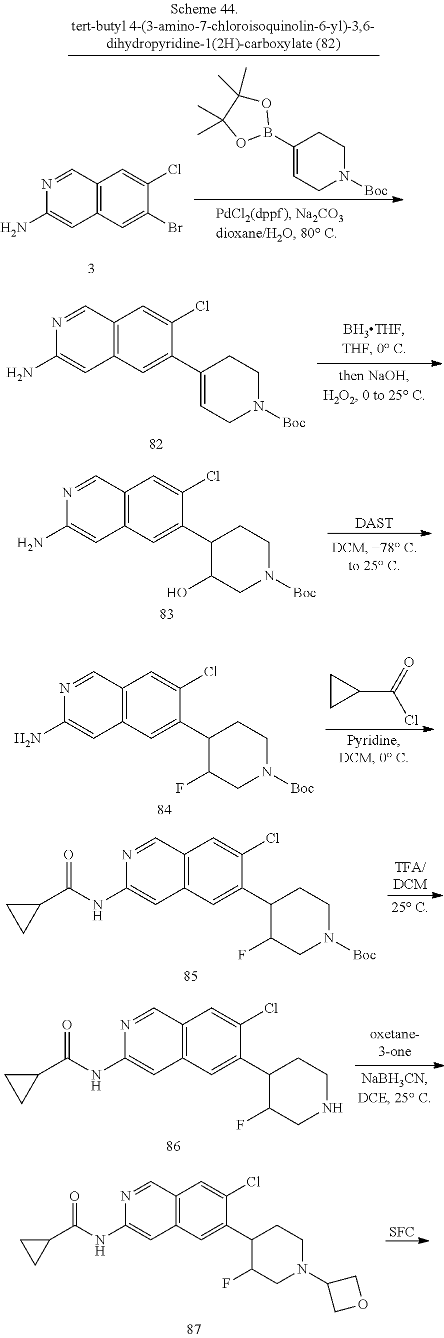

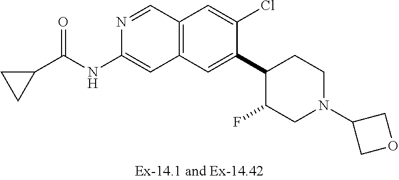

A vial was charged with 6-bromo-7-chloroisoquinolin-3-amine (0.5 g, 1.942 mmol), tert-butyl 4-(4,4,5,5-tetramethyl-1,3,2-dioxaborolan-2-yl)-5,6-dihydropyridine-1(2H)-carboxylate (0.720 g, 2.330 mmol), PdCl₂(dppf) (0.142 g, 0.194 mmol) and Na₂CO₃ (0.617 g, 5.82 mmol) in dioxane (12 ml) and Water (3.00 ml) was stirred at 80° C. for 5 h. The reaction was diluted with EtOAc (50 mL), filtered and concentrated under reduced pressure. The residue was purified by flash silica gel chromatography (ISCO®; 12 g SepaFlash® Silica Flash Column, eluent of 0-30% EtOAc/PE gradient at a flow rate of 30 mL/min) to afford the title compound. MS (ESI): m/z calc'd for C₁₉H₂₃ClN₃O₂ [M+H]⁺: 360, found 360.

### tert-butyl 4-(3-amino-7-chloroisoquinolin-6-yl)-3-hydroxypiperidine-1-carboxylate (83)

A vial was charged with tert-butyl 4-(3-amino-7-chloroisoquinolin-6-yl)-5,6-dihydropyridine-1(2H)-carboxylate (480 mg, 1.334 mmol) in THF (5 ml) at 0° C. BH₃·THF (1M in THF) (6.67 ml, 6.67 mmol) was added to a solution at 0° C. and stirred for 1 h followed by at 25° C. for 14 h, the reaction was cooled to 0° C. Then NaOH (3.33 ml, 6.67 mmol) was added to the above mixture followed by addition of H₂O₂ (1.168 ml, 13.34 mmol) at 0° C. The resulting mixture was stirred at 0° C. for another 5 h. The reaction mixture was quenched with sat. Na₂SO₃ (20 mL) and extracted with EtOAc (30 mL×3). The combined organic phases were washed with brine (50 mL), dried over anhydrous Na₂SO₄, filtered and concentrated under reduced pressure. The residue was purified by Pre-TLC (silica gel, EtOAc/PE=100 mL/100 mL, v/v) to afford the title compound. MS (ESI): m/z calc'd for C₁₉H₂₅ClFN₃O₃ [M+H]⁺: 378, found 378.

### tert-butyl 4-(3-amino-7-chloroisoquinolin-6-yl)-3-fluoropiperidine-1-carboxylate (84)

A vial was charged with tert-butyl 4-(3-amino-7-chloroisoquinolin-6-yl)-3-hydroxypiperidine-1-carboxylate (125 mg, 0.331 mmol) in DCM (5 ml) at −78° C. DAST (0.219 ml, 1.654 mmol) was added to a solution. The resulting mixture was stirred for 16 h, then quenched with MeOH (20 mL) and concentrated under reduced pressure. The residue was dissolved in water (10 mL) and EtOAc (10 mL). The organic layer was separated, and the aqueous was re-extracted with EtOAc (5 mL×3). The combined organic layers were washed with brine (20 mL), dried over anhydrous Na₂SO₄, filtered and concentrated under reduced pressure to afford the title compound. MS (ESI): m/z calc'd for C₁₉H₂₄ClFN₃O₂ [M+H]⁺: 380, found 380.

### tert-butyl 4-(7-chloro-3-(cyclopropanecarboxamido)isoquinolin-6-yl)-3-fluoropiperidine-1-carboxylate (85)

A mixture of tert-butyl 4-(3-amino-7-chloroisoquinolin-6-yl)-3-fluoropiperidine-1-carboxylate (120 mg, 0.316 mmol), cyclopropanecarbonyl chloride (0.034 ml, 0.379 mmol) and pyridine (0.102 ml, 1.264 mmol) in DCM (5 ml) was stirred at 25° C. for 1 h. The solvent was removed under reduced pressure, and the residue was dissolved in water (10 mL) and EtOAc (10 mL). The organic layer was separated, and the aqueous was re-extracted with EtOAc (5 mL×3). The combined organic layers were washed with brine (50 mL), dried over anhydrous Na₂SO₄, filtered and concentrated under reduced pressure to afford the title compound. MS (ESI): m/z calc'd for C₂₃H₂₈ClFN₃O₃ [M+H]⁺: 448, found 448.

### N-(7-chloro-6-(3-fluoropiperidin-4-yl)isoquinolin-3-yl)cyclopropanecarboxamide (86)

A mixture of tert-butyl 4-(7-chloro-3-(cyclopropanecarboxamido)isoquinolin-6-yl)-3-fluoropiperidine-1-carboxylate (40 mg, 0.089 mmol) and TFA (1 ml) in DCM (5 ml) was stirred at 25° C. for 16 h. The reaction mixture was quenched with NaHCO₃ (20 mL) and extracted with EtOAc (15 mL×3). The combined organic phases were washed with brine (50 mL), dried over anhydrous Na₂SO₄, filtered and concentrated under reduced pressure to afford the title compound. MS (ESI): m/z calc'd for C₁₈H₂₀ClFN₃O [M+H]+: 348, found 348.

### N-(7-chloro-6-(3-fluoro-1-(oxetan-3-yl)piperidin-4-yl)isoquinolin-3-yl)cyclopropanecarboxamide (87)

A mixture of N-(7-chloro-6-(3-fluoropiperidin-4-yl)isoquinolin-3-yl)cyclopropanecarboxamide (10 mg, 0.029 mmol), oxetan-3-one (4 μl, 0.058 mmol) and NaBH₃(CN) (9.0 mg, 0.144 mmol) in EtOH (1 mL) was stirred at 25° C. for 16 h. The reaction mixture was quenched with sat. NH₄Cl (10 mL) and extracted with EtOAc (10 mL×3). The combined organic phases were washed with brine (30 mL), dried over anhydrous Na₂SO₄, filtered and concentrated under reduced pressure to afford the title compound.

### N-(7-chloro-6-((3R,4R,3S,4R,3R,4S or 3S,4S)-3-fluoro-1-(oxetan-3-yl)piperidin-4-yl)isoquinolin-3-yl)cyclopropanecarboxamide (Ex-14.1 and 14.2)

The mixture of two stereoisomers was purified by chiral SFC (Diacel Chiralcel OD, 40%/60% Ethanol/CO2+0.10% NH₄OH) and lyophilized to afford resolved stereoisomers of the title compound Ex-14.1 (tR=4.02 min) and Ex-14.2 (tR=4.28 min).

Ex-14.1: MS (ESI): m/z calc'd for C₂₁H₂₄ClFN₃O₂ [M+H]+: 404, found 404.

¹H NMR (400 MHz, CDCl₃) (10.11 (br s, 1H), 8.87 (s, 1H), 8.74 (s, 1H), 8.01 (s, 1H), 7.84 (s, 1H), 5.15-4.98 (m, 1H), 4.78-4.70 (m, 4H), 3.80-3.74 (m, 1H), 3.54-3.43 (m, 1H), 3.40-3.34 (m, 1H), 2.98-2.92 (m, 1H), 2.27-2.20 (m, 2H), 2.18-2.09 (m, 1H), 1.84-1.76 (m, 2H), 1.21-1.14 (m, 2H), 1.02-0.94 (m, 2H)

Ex-14.2: MS (ESI): m/z calc'd for C₂₁H₂₄ClFN₃O₂ [M+H]⁺: 404, found 404.

¹H NMR (500 MHz, CDCl₃) (11.37 (br s, 1H), 8.91 (s, 1H), 8.87 (s, 1H), 8.05 (s, 1H), 7.89 (s, 1H), 5.32-5.10 (m, 1H), 4.84 (q, J=6.7 Hz, 2H), 4.79 (q, J=7.2 Hz, 2H), 3.94 (m, 1H), 3.59-3.50 (m, 2H), 3.20-3.14 (m, 1H), 2.45-2.34 (m, 2H), 2.25-2.17 (m, 1H), 2.02-1.88 (m, 2H), 1.20-1.14 (m, 2H), 1.03-0.97 (m, 2H).

Compounds in Table 14 below were prepared in accordance with the synthetic sequence illustrated in General Scheme 14 and Scheme 44 using the corresponding starting materials.

TABLE 14

| | | |
|---|---|---|
| 14.3 | 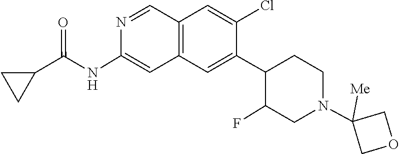 [SMILES: CC1(N2CCC(c3cc4cc(NC(=O)C5CC5)ncc4cc3Cl)C(F)C2)COC1] | Cal′c: 418 Found: 418 |
| | N-(7-chloro-6-(3-fluoro-1-(3-methyloxetan-3-yl)piperidin-4- | |
| | yl)isoquinolin-3-yl)cyclopropanecarboxamide, N-(7-chloro-6- | |
| | ((3R,4R or 3R,4S or 3S,4R or 3S,4S)-3-fluoro-1-(3- | |
| | methyloxetan-3-yl)piperidin-4-yl)isoquinolin-3- | |
| | yl)cyclopropanecarboxamide | |
| 14.4-14.6 | 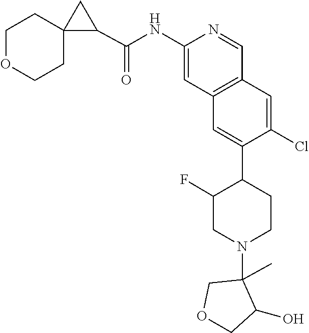 [SMILES: CC1(N2CCC(c3cc4cc(NC(=O)C5CC56CCOCC6)ncc4cc3Cl)C(F)C2)COCC1O] | Cal′c: 518 Found: 518 |
| | N-(7-chloro-6-(3-fluoro-1-(4-hydroxy-3-methyltetrahydrofuran- | |
| | 3-yl)piperidin-4-yl)isoquinolin-3-yl)-6-oxaspiro[2.5]octane-1- | |
| | carboxamide, (1R or 1S)-N-(7-chloro-6-((3R,4R or 3R,4S or | |
| | 3S,4R or 3S,4S)-3-fluoro-1-(4-hydroxy-3-methyltetrahydrofuran- | |
| | 3-yl)piperidin-4-yl)isoquinolin-3-yl)-6-oxaspiro[2.5]octane-1- | |
| | carboxamide | |

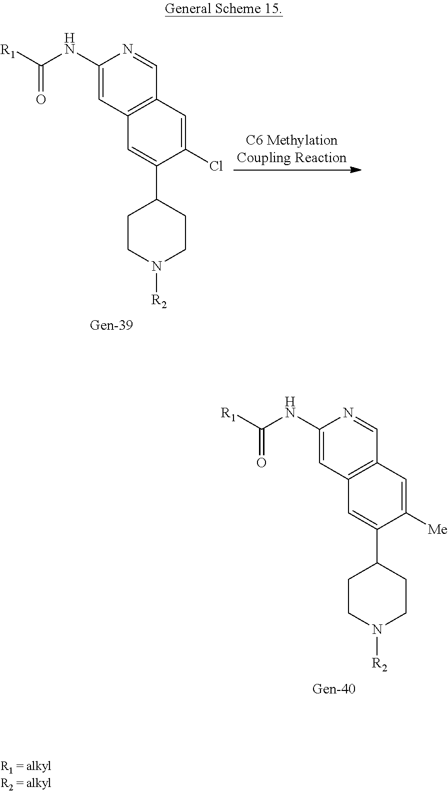

In General Scheme 15, Suzuki coupling conditions were used to provide the methylated derivatives of the fully elaborated compounds in the form of Gen-40. The representative compounds are described in more detail below.

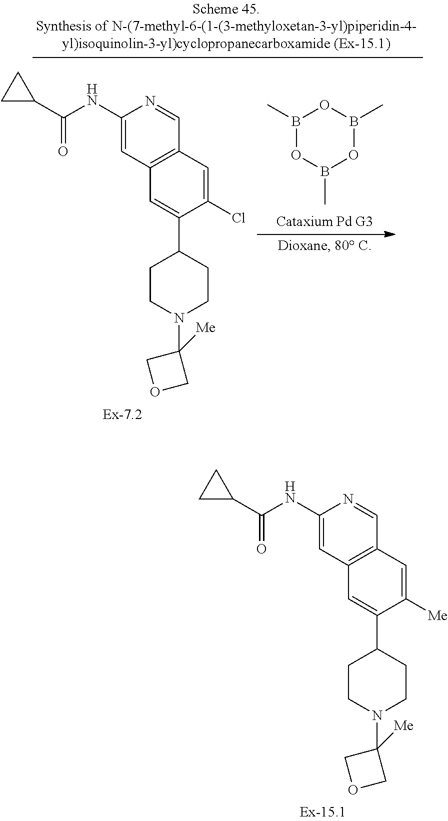

N-(7-chloro-6-(1-(3-methyloxetan-3-yl)piperidin-4-yl)isoquinolin-3-yl)cyclopropanecarboxamide Ex-7.2 (50 mg, 0.125 mmol), trimethylboroxine (62.8 mg, 0.500 mmol), and Cataxium A Pd G3 (18.21 mg, 0.025 mmol) were added to a vial. The vial was sealed, and its contents were placed under an inert atmosphere by performing 3 vacuum/nitrogen cycles. Dioxane (625 μl) and aqueous potassium phosphate, tribasic (188 μl, 0.375 mmol) were added through the septum. The resulting mixture was allowed to stir for 2 hours at 80° C., then filtered, purified by HPLC (eluting acetonitrile/water gradient with 0.10% TFA modifier, linear gradient) and lyophilized to afford the title compound. MS (ESI): m/z calc'd for C₂₃H₂₉N₃O₂ [M+H]⁺: 380, found 380. ¹H NMR (499 MHz, DMSO-d6) δ 10.83 (s, 1H), 9.01 (s, 1H), 8.39 (s, 1H), 7.84 (s, 1H), 7.58 (s, 1H), 4.88 (m, 2H), 4.42 (m, 2H), 3.35 (m, 2H), 3.27 (m, 3H), 2.51 (s, 3H), 2.16-2.02 (m, 5H), 1.69 (s, 3H), 1.00-0.76 (m, 4H).

Compounds in Table 15 below were prepared in accordance with the synthetic sequence illustrated in General Scheme 15 and Scheme 55 using the corresponding starting materials.

TABLE 15

| | | |
|---|---|---|
| 15.2 | 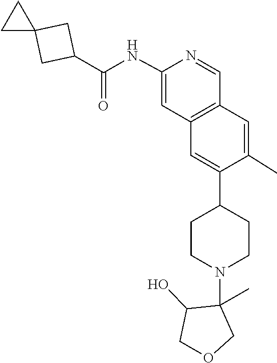 [SMILES: Cc1cc2cnc(NC(=O)C3CC4(CC4)C3)cc2cc1C1CCN(C2(C)COCC2O)CC1] | Cal′c: 450 Found: 450 |
| | N-(6-(1-(4-hydroxy-3-methyltetrahydrofuran-3-yl)piperidin-4- | |
| | yl)-7-methylisoquinolin-3-yl)spiro[2.3]hexane-5-carboxamide, | |
| | N-(6-(1-((3R,4R or 3S,4S)-4-hydroxy-3-methyltetrahydrofuran- | |
| | 3-yl)piperidin-4-yl)-7-methylisoquinolin-3-yl)spiro[2.3]hexane- | |
| | 5-carboxamide | |
| 15.3-15.4 | 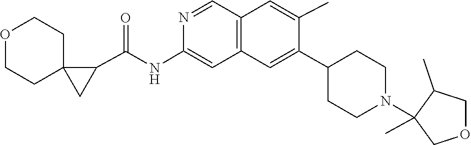 [SMILES: Cc1cc2cnc(NC(=O)C3CC34CCOCC4)cc2cc1C1CCN(C2(C)COCC2C)CC1] | Cal′c: 478 Found: 478 |
| | N-(6-(1-(3,4-dimethyltetrahydrofuran-3-yl)piperidin-4-yl)-7- | |
| | methylisoquinolin-3-yl)-6-oxaspiro[2.5]octane-1-carboxamide, | |
| | (S or R)-N-(6-(1-((3S,4R or 3R,4S)-3,4-dimethyltetrahydrofuran- | |
| | 3-yl)piperidin-4-yl)-7-methylisoquinolin-3-yl)-6- | |
| | oxaspiro[2.5]octane-1-carboxamide | |
| 15.5-15.6 | 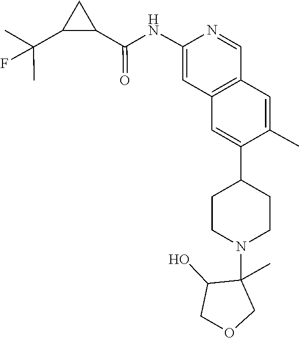 [SMILES: Cc1cc2cnc(NC(=O)C3CC3C(C)(C)F)cc2cc1C1CCN(C2(C)COCC2O)CC1] | Cal′c: 470 Found: 470 |
| | 2-(2-fluoropropan-2-yl)-N-(6-(1-(4-hydroxy-3- | |
| | methyltetrahydrofuran-3-yl)piperidin-4-yl)-7-methylisoquinolin- | |
| | 3-yl)cyclopropane-1-carboxamide, (1R,2R or 1S,2S)-2-(2- | |
| | fluoropropan-2-yl)-N-(6-(1-((3R,4R or 3S,4S)-4-hydroxy-3- | |
| | methyltetrahydrofuran-3-yl)piperidin-4-yl)-7-methylisoquinolin- | |
| | 3-yl)cyclopropane-1-carboxamide | |
| 15.7-15.14 | 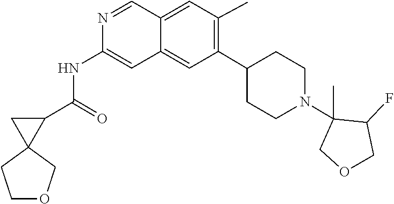 [SMILES: Cc1cc2cnc(NC(=O)C3CC34CCOC4)cc2cc1C1CCN(C2(C)COCC2F)CC1] | Cal′c: 468 Found: 468 |
| | N-(6-(1-(4-fluoro-3-methyltetrahydrofuran-3-yl)piperidin-4-yl)- | |
| | 7-methylisoquinolin-3-yl)-5-oxaspiro[2.4]heptane-1- | |
| | carboxamide, (1R,2R or 1R,2S or 1S,2R or 1S,2S)-N-(6-(1- | |
| | ((3R,4R or 3S,4S)-4-fluoro-3-methyltetrahydrofuran-3- | |
| | yl)piperidin-4-yl)-7-methylisoquinolin-3-yl)-5- | |
| | oxaspiro[2.4]heptane-1-carboxamide | |
| 15.15 | 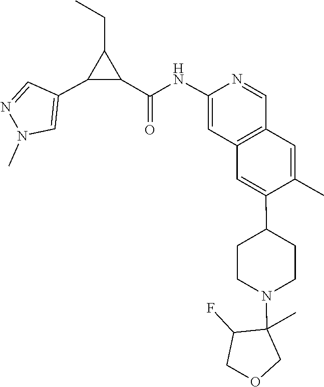 [SMILES: CCC1C(C(=O)Nc2cc3cc(C4CCN(C5(C)COCC5F)CC4)c(C)cc3cn2)C1c1cnn(C)c1] | Cal′c: 520 Found: 520 |
| | 2-ethyl-N-(6-(1-(4-fluoro-3-methyltetrahydrofuran-3- | |
| | yl)piperidin-4-yl)-7-methylisoquinolin-3-yl)-3-(1-methyl-1H- | |
| | pyrazol-4-yl)cyclopropane-1-carboxamide, (1R,2R,3R or | |
| | 1R,2S,3R or 1S,2R,3S, or 1S,2S,3S)-2-ethyl-N-(6-(1-((3R,4R or | |
| | 3S,4S)-4-fluoro-3-methyltetrahydrofuran-3-yl)piperidin-4-yl)-7- | |
| | methylisoquinolin-3-yl)-3-(1-methyl-1H-pyrazol-4- | |
| | yl)cyclopropane-1-carboxamide | |
| 15.16 | 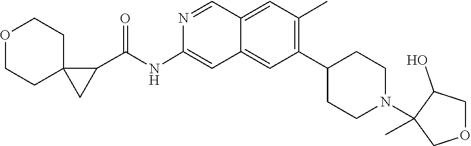 [SMILES: Cc1cc2cnc(NC(=O)C3CC34CCOCC4)cc2cc1C1CCN(C2(C)COCC2O)CC1] | Cal′c: 480 Found: 480 |
| | N-(6-(1-(4-hydroxy-3-methyltetrahydrofuran-3-yl)piperidin-4- | |
| | yl)-7-methylisoquinolin-3-yl)-6-oxaspiro[2.5]octane-1- | |
| | carboxamide, (R or S)-N-(6-(1-((3R,4R or 3S,4S)-4-hydroxy-3- | |
| | methyltetrahydrofuran-3-yl)piperidin-4-yl)-7-methylisoquinolin- | |
| | 3-yl)-6-oxaspiro[2.5]octane-1-carboxamide | |

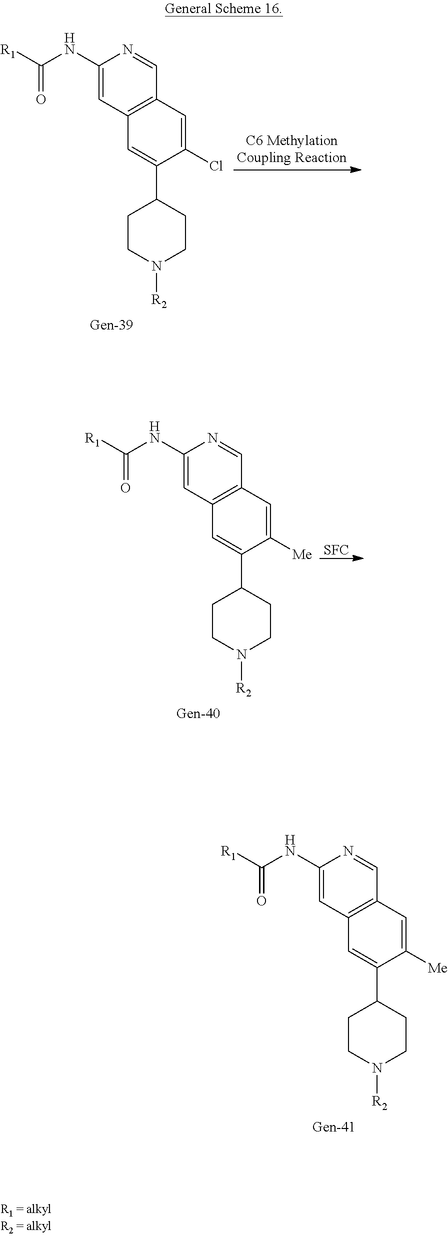

In General Scheme 16, Suzuki coupling conditions were used to provide the methylated derivatives of the fully elaborated compounds in the form of Gen-40 and the stereoisomers could then be separated by chiral SFC to provide fully elaborated products in the form of Gen-41. The representative compounds are described in more detail below.

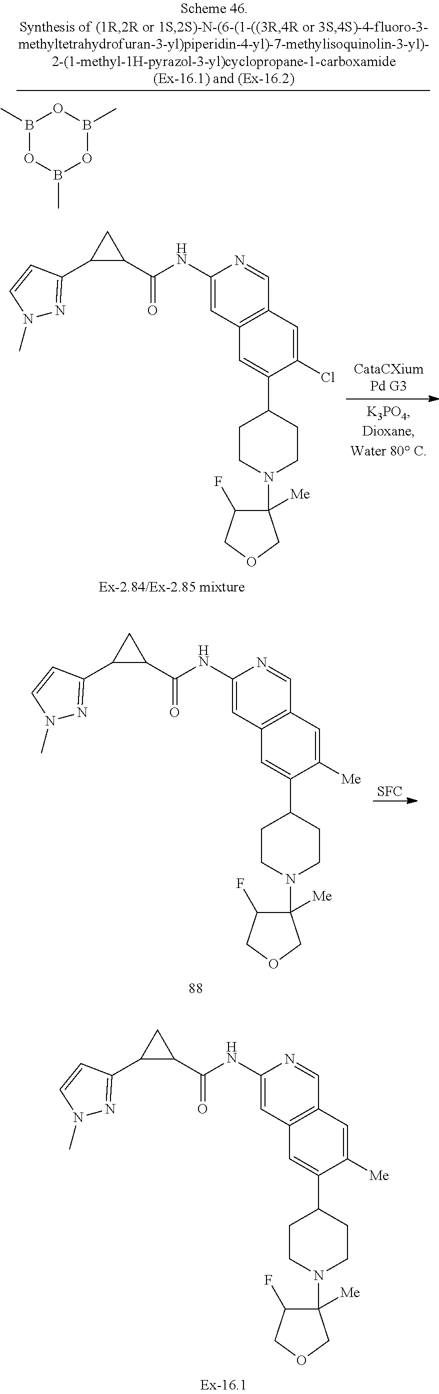

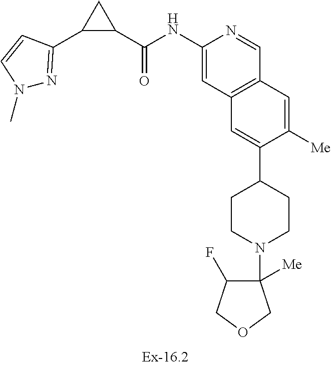 [SMILES: Cc1cc2cnc(NC(=O)C3CC3c3ccn(C)n3)cc2cc1C1CCN(C2(C)COCC2F)CC1]

A vial was charged with N-(7-chloro-6-(1-(4-fluoro-3-methyltetrahydrofuran-3-yl)piperidin-4-yl)isoquinolin-3-yl)-2-(1-methyl-1H-pyrazol-3-yl)cyclopropane-1-carboxamide (100 mg, 0.195 mmol), methanesulfonato(diadamantyl-n-butylphosphino)-2′-amino-1,1′-biphenyl-2-yl)palladium(II) dichloromethane adduct (31.8 mg, 0.039 mmol), trimethylboroxine (273 μl, 1.953 mmol), potassium phosphate, tribasic (166 mg, 0.781 mmol). The vial was sealed, and its contents were placed under an inert atmosphere by performing 3 vacuum/nitrogen cycles. Dioxane (1758 μl) and water (195 μl) were added through the septum, and the resulting mixture was stirred overnight at 80° C. The crude mixture was filtered and concentrated under reduced pressure. The crude residue was subject to purification by reversed phase HPLC, eluting with water (0.1% NH₄OH)-ACN to afford the racemate. The racemic material could be resolved to its component enantiomers by chiral preparative SFC (Column & dimensions: CCA F4, 21 mm×250 mm; Mobile phase A: C₀₂; Mobile phase B: MeOH with 0.1% NH₄OH) to afford Ex-16.1 (tR=5.3 min) and Ex-16.1 (tR=7.5 min).

Ex-16.1: MS (ESI) m/z calc'd for C₂₈H₃₄FN₅O₂ [M+H]+: 492, found 492. ¹H NMR (400 MHz, d-DMSO, 25° C.) δ 10.79 (s, J=32.9 Hz, 1H), 9.81 (s, 1H), 8.98 (s, J=21.6 Hz, 1H), 8.39 (s, J=11.4 Hz, 1H), 7.81 (s, J=29.3 Hz, 1H), 7.66 (s, J=27.5 Hz, 1H), 7.56 (s, 1H), 7.30 (s, 1H), 7.20 (s, 1H), 7.10 (s, 1H), 6.07 (s, 1H), 5.45 (d, J=54.6 Hz, 1H), 4.39-3.99 (m, 3H), 3.90 (d, J=31.2 Hz, 1H), 3.54 (s, 2H), 3.24-2.72 (m, 2H), 2.35 (d, J=6.6 Hz, 3H), 2.24-1.92 (m, 2H), 1.79 (s, 2H), 1.43 (d, J=23.0 Hz, 3H), 1.27 (d, J=30.3 Hz, 2H), 1.03 (s, 2H).

Ex-16.2: MS (ESI) m/z calc'd for C₂₈H₃₄FN₅O₂ [M+H]+: 492, found 492. ¹H NMR (400 MHz, d-DMSO, 25° C.) δ: 10.79 (s, J=35.1 Hz, 1H), 9.78 (s, 0H), 8.98 (s, J=22.7 Hz, 1H), 8.39 (s, J=14.2 Hz, 1H), 7.84 (s, 1H), 7.66 (s, J=30.2 Hz, 1H), 7.56 (s, 1H), 7.28 (s, 1H), 7.18 (s, 1H), 7.08 (s, 1H), 6.07 (s, 1H), 5.59-4.73 (m, 1H), 4.39-4.00 (m, 2H), 3.96-3.79 (m, 1H), 3.62 (d, J=70.7 Hz, 2H), 3.22-2.75 (m, 2H), 2.48 (s, 2H), 2.39-2.32 (m, 2H), 2.05 (dd, J=88.2, 42.3 Hz, 2H), 1.79 (s, 2H), 1.49-1.37 (m, 2H), 1.33-1.13 (m, 2H), 1.03 (s, 3H).

Compounds in Table 16 below were prepared in accordance with the synthetic sequence illustrated in General Scheme 16 and Scheme 46 using the corresponding starting materials.

TABLE 16

| | | |
|---|---|---|
| 16.3-16.4 | 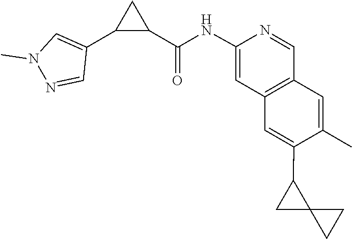 | Cal′c: 373 Found: 373 |
| | 2-(1-methyl-1H-pyrazol-4-yl)-N-(7-methyl-6-(spiro[2.2]pentan- | |
| | 1-yl)isoquinolin-3-yl)cyclopropane-1-carboxamide, (1R,2R or | |
| | 1S,2S)- 2-(1-methyl-1H-pyrazol-4-yl)-N-(7-methyl-6-((R or S)- | |
| | spiro[2.2]pentan-1-yl)isoquinolin-3-yl)cyclopropane-1- | |
| | carboxamide | |
| 16.5-16.6 | 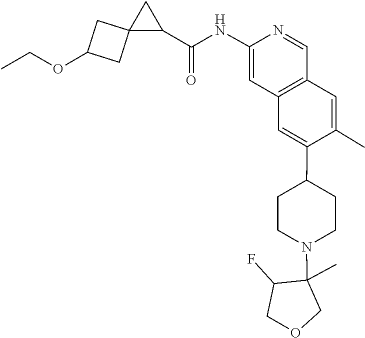 [SMILES: CCOC1CC2(C1)CC2C(=O)Nc1cc2cc(C3CCN(C4(C)COCC4F)CC3)c(C)cc2cn1] | Cal′c: 496 Found: 496 |
| | 5-ethoxy-N-(6-(1-(4-fluoro-3-methyltetrahydrofuran-3- | |
| | yl)piperidin-4-yl)-7-methylisoquinolin-3-yl)spiro[2.3]hexane-1- | |
| | carboxamide, (1R or 1S)-5-ethoxy-N-(6-(1-((3R,4R or 3S,4S)-4- | |
| | fluoro-3-methyltetrahydrofuran-3-yl)piperidin-4-yl)-7- | |
| | methylisoquinolin-3-yl)spiro[2.3]hexane-1-carboxamide | |
| 16.7-16.8 | 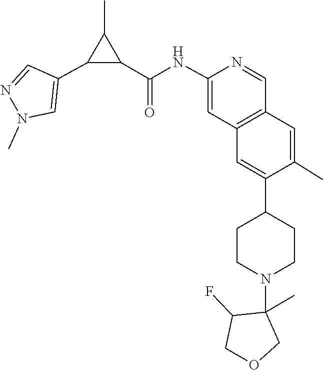 [SMILES: Cc1cc2cnc(NC(=O)C3C(C)C3c3cnn(C)c3)cc2cc1C1CCN(C2(C)COCC2F)CC1] | Cal′c: 506 Found: 506 |
| | N-(6-(1-(4-fluoro-3-methyltetrahydrofuran-3-yl)piperidin-4-yl)- | |
| | 7-methylisoquinolin-3-yl)-2-methyl-3-(1-methyl-1H-pyrazol-4- | |
| | yl)cyclopropane-1-carboxamide, (1R,2R,3R or 1R,2S,3R or | |
| | 1S,2R,3S or 1S,2S,3S)-N-(6-(1-((3R,4R or 3S,4S)-4-fluoro-3- | |
| | methyltetrahydrofuran-3-yl)piperidin-4-yl)-7-methylisoquinolin- | |
| | 3-yl)-2-methyl-3-(1-methyl-1H-pyrazol-4-yl)cyclopropane-1- | |
| | carboxamide | |
| 16.9-16.12 | 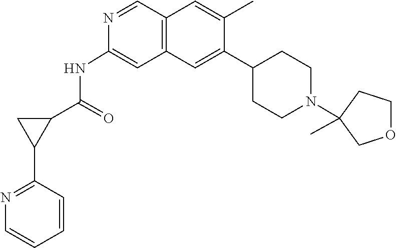 [SMILES: Cc1cc2cnc(NC(=O)C3CC3c3ccccn3)cc2cc1C1CCN(C2(C)CCOC2)CC1] | Cal′c: 471 Found:471 |
| | N-(7-methyl-6-(1-(3-methyltetrahydrofuran-3-yl)piperidin-4- | |
| | yl)isoquinolin-3-yl)-2-(pyridin-2-yl)cyclopropane-1- | |
| | carboxamide, (1R,2R or 1S,2S)- N-(7-methyl-6-(1-((R or S)-3- | |
| | methyltetrahydrofuran-3-yl)piperidin-4-yl)isoquinolin-3-yl)-2- | |
| | (pyridin-2-yl)cyclopropane-1-carboxamide | |
| 16.13-16.14 | 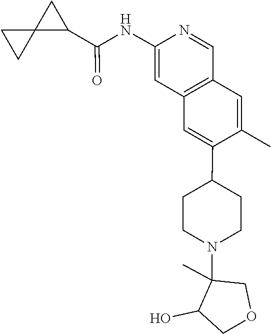 [SMILES: Cc1cc2cnc(NC(=O)C3CC34CC4)cc2cc1C1CCN(C2(C)COCC2O)CC1] | Cal′c: 436 Found: 436 |
| | N-(6-(1-(4-hydroxy-3-methyltetrahydrofuran-3-yl)piperidin-4- | |
| | yl)-7-methylisoquinolin-3-yl)spiro[2.2]pentane-1-carboxamide, | |
| | (1R or 1S)-N-(6-(1-((3R,4R or 3S,4S)-4-hydroxy-3- | |
| | methyltetrahydrofuran-3-yl)piperidin-4-yl)-7-methylisoquinolin- | |
| | 3-yl)spiro[2.2]pentane-1-carboxamide | |

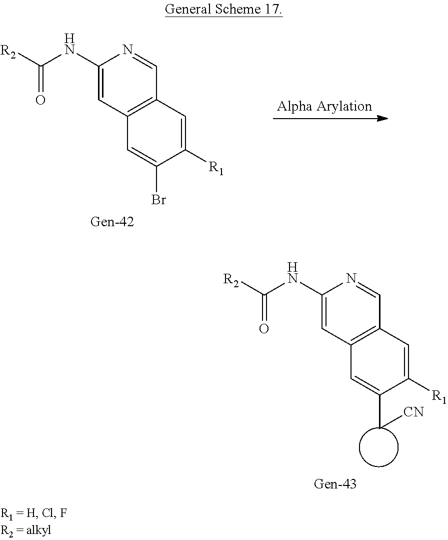

In General Scheme 17, alpha arylation couplings to Gen-42 provided the fully elaborated compounds in the form of Gen-43. The representative compounds are described in more detail below.

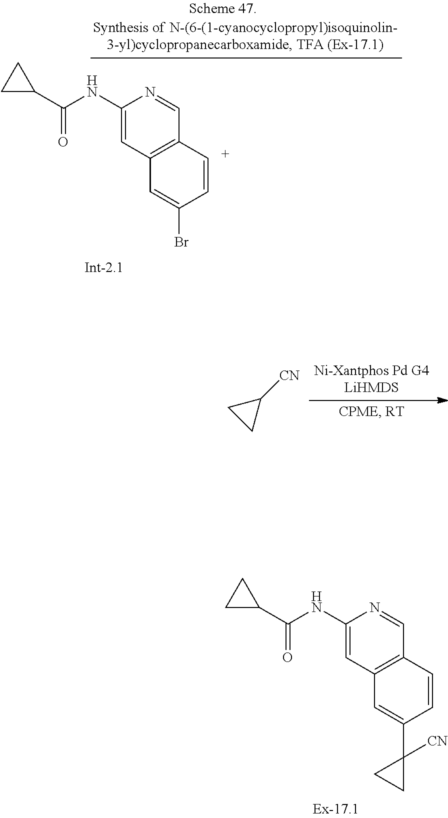 [SMILES: O=C(Nc1cc2cc(Br)ccc2cn1)C1CC1 ; N#CC1CC1 ; N#CC1(c2ccc3cnc(NC(=O)C4CC4)cc3c2)CC1]

N-(6-bromoisoquinolin-3-yl)cyclopropanecarboxamide Int-2.1 (60 mg, 0.206 mmol), cyclopropanecarbonitrile (15.21 mg, 0.227 mmol), and Ni-Xantphos Pd G4 (38.0 mg, 0.041 mmol) were added to a vial. The vial was sealed, and its contents were placed under an inert atmosphere by performing 3 vacuum/nitrogen cycles. CPME (1030 μl) and LiHMDS (1M in THF) (824 μl, 0.824 mmol) were added through the septum and the resulting mixture was allowed to stir overnight at room temperature. The reaction mixture was filtered and purified by HPLC, eluting acetonitrile/water gradient with 0.1% TFA modifier, linear gradient and lyophilized to afford the title product. MS (ESI): m/z calc'd for C₁₇H₁₅N₃O TFA [M+H]+: 278, found 278. ¹H NMR (499 MHz, DMSO-d6) δ 10.93 (s, 1H), 9.13 (s, 1H), 8.45 (s, 1H), 8.07 (d, J=8.6 Hz, 1H), 7.80 (s, 1H), 7.44 (dd, J=8.6, 1.8 Hz, 1H), 2.08 (s, 1H), 1.89-1.85 (m, 2H), 1.73-1.69 (m, 2H), 0.89-0.81 (m, 4H).

Compounds in Table 17 below were prepared in accordance with the synthetic sequence illustrated in General Scheme 17 and Scheme 47 using the corresponding starting materials.

TABLE 18

| | | |
|---|---|---|
| 17.2 | 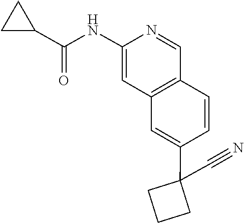 [SMILES: N#CC1(c2ccc3cnc(NC(=O)C4CC4)cc3c2)CCC1] | Cal′c: 292 Found: 292 |
| | N-(6-(1-cyanocyclobutyl)isoquinolin-3- | |
| | yl)cyclopropanecarboxamide | |

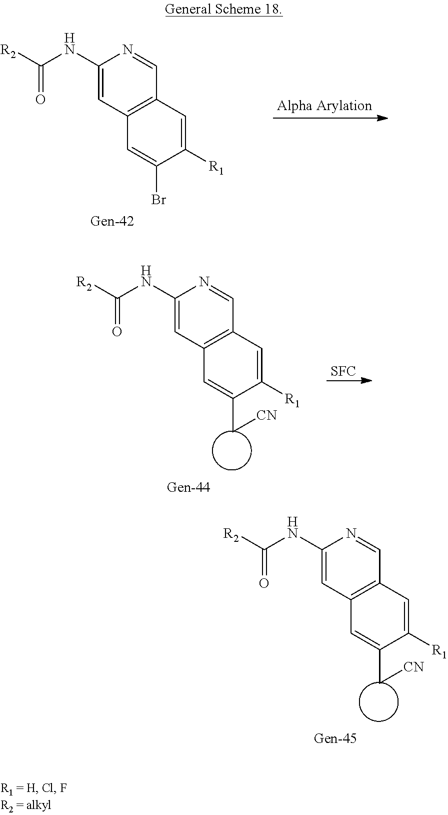

In General Scheme 18, alpha arylation of alkyl nitriles using intermediates of the form Gen-42 could provide the fully elaborated compounds in the form of Gen-44 and the stereoisomers could then be separated by chiral SFC to provide fully elaborated products in the form of Gen-45. The representative compounds are described in more detail below.

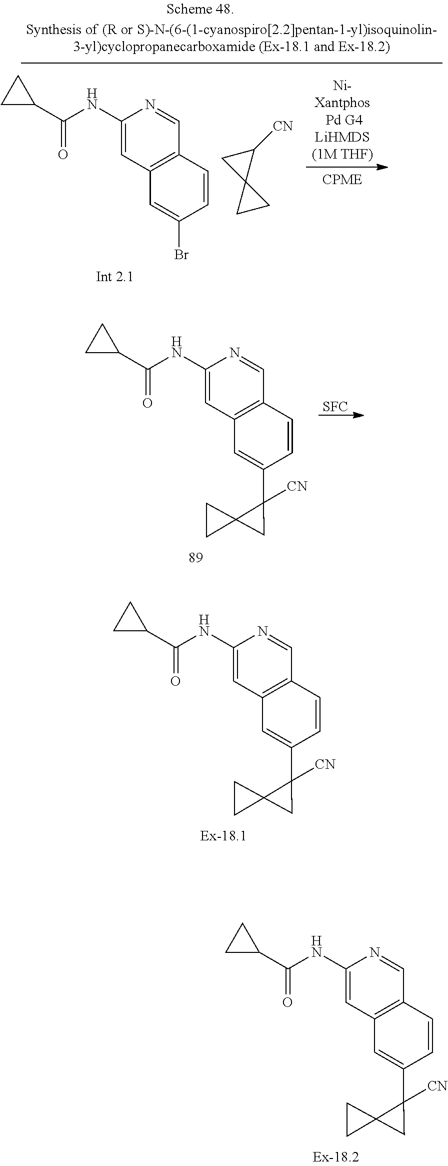

N-(6-(1-cyanospiro[2.2]pentan-1-yl)isoquinolin-3-yl)cyclopropanecarboxamide (89) N-(6-bromoisoquinolin-3-yl)cyclopropanecarboxamide Int 2.1 (75 mg, 0.258 mmol), spiro[2.2]pentane-1-carbonitrile (26.4 mg, 0.283 mmol), and Ni-Xantphos Pd G4 (47.5 mg, 0.052 mmol) were added to a vial. The vial was sealed and its contents were placed under an inert atmosphere. CPME (1288 μl) and LiHMDS (1M in THF) (1030 μl, 1.030 mmol) were added through the septum, and the resulting mixture was allowed to stir for 2 hours at room temperature. The reaction mixture was diluted with ethyl acetate and washed twice with saturated sodium bicarbonate and once with brine. The combined organic fractions were dried over anhydrous sodium sulfate, filtered and concentrated under reduced pressure. The residue was purified by column chromatography on silica (0-100% EtOAc/hexane). The desired fractions were concentrated under reduced pressure to afford the title compound as a mixture of stereoisomers. The mixture of two stereoisomers was purified by chiral SFC (Lux-2, 21×250 (mm), Mobile phase A: 30% CO₂ Mobile phase B: 70% MeOH 0.1% NH₄OH) and concentrated to afford the title compounds Ex-18.1 (tR=4.8 m) and Ex-18.2 (tR=6.0 mi). MS (ESI): M/z calc'd for C₁₉H₁₇N₃O [M+H]⁺: 304, found 304. ¹H NMR (499 MHz, DMSO-d6) δ 10.92 (s, 1H), 9.13 (s, 1H), 8.45 (s, 1H), 8.07 (m, 1H), 7.76 (s, 1H), 7.38 (m, 1H), 2.38 (m, 1H), 2.18 (m, 1H), 2.12-2.03 (m, 1H), 1.31-1.20 (m, 3H), 1.01-0.94 (m, 1H), 0.88-0.80 (m, 4H).

Compounds in Table 18 below were prepared in accordance with the synthetic sequence illustrated in General Scheme 18 and Scheme 48 using the corresponding starting materials.

TABLE 18

| | | |
|---|---|---|
| 18.3-18.4 | 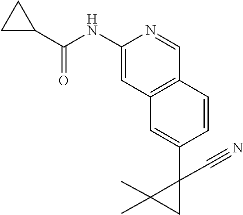 [SMILES: CC1(C)CC1(C#N)c1ccc2cnc(NC(=O)C3CC3)cc2c1] | Cal′c: 306 Found: 306 |
| | N-(6-(1-cyano-2,2-dimethylcyclopropyl)isoquinolin-3- | |
| | yl)cyclopropanecarboxamide, (R or S)-N-(6-(1-cyano-2,2- | |
| | dimethylcyclopropyl)isoquinolin-3-yl)cyclopropanecarboxamide | |
| 18.5-18.8 | 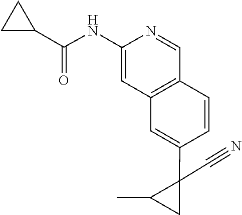 [SMILES: CC1CC1(C#N)c1ccc2cnc(NC(=O)C3CC3)cc2c1] | Cal′c: 292 Found: 292 |
| | N-(6-(1-cyano-2-methylcyclopropyl)isoquinolin-3- | |
| | yl)cyclopropanecarboxamide, (R or S)- N-(6-(1-cyano-2- | |
| | methylcyclopropyl)isoquinolin-3-yl)cyclopropanecarboxamide | |
| 18.9-18.10 | 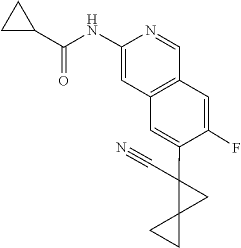 [SMILES: N#CC1(c2cc3cc(NC(=O)C4CC4)ncc3cc2F)CC12CC2] | Cal′c: 322 Found: 322 |
| | N-(6-(1-cyanospiro[2.2]pentan-1-yl)-7-fluoroisoquinolin-3- | |
| | yl)cyclopropanecarboxamide, (R or S)-N-(6-(1- | |
| | cyanospiro[2.2]pentan-1-yl)-7-fluoroisoquinolin-3- | |
| | yl)cyclopropanecarboxamide | |
| 18.11-18.12 | 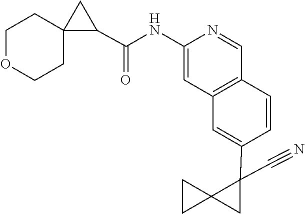 [SMILES: N#CC1(c2ccc3cnc(NC(=O)C4CC45CCOCC5)cc3c2)CC12CC2] | Cal′c: 374 Found: 374 |
| | N-(6-(1-cyanospiro[2.2]pentan-1-yl)isoquinolin-3-yl)-6- | |
| | oxaspiro[2.5]octane-1-carboxamide, (R or S)-N-(6-((R or S)-1- | |
| | cyanospiro[2.2]pentan-1-yl)isoquinolin-3-yl)-6- | |
| | oxaspiro[2.5]octane-1-carboxamide | |
| 18.13-18.14 | 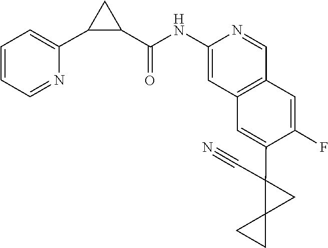 [SMILES: N#CC1(c2cc3cc(NC(=O)C4CC4c4ccccn4)ncc3cc2F)CC12CC2] | Cal′c: 399 Found: 399 |
| | N-(6-(1-cyanospiro[2.2]pentan-1-yl)-7-fluoroisoquinolin-3-yl)-2- | |
| | (pyridin-2-yl)cyclopropane-1-carboxamide, (1R,2R or 1S,2S)-N- | |
| | (6-((R or S)-1-cyanospiro[2.2]pentan-1-yl)-7-fluoroisoquinolin-3- | |
| | yl)-2-(pyridin-2-yl)cyclopropane-1-carboxamide | |
| 18.15-18.16 | 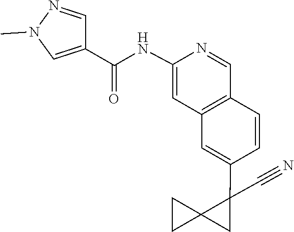 [SMILES: Cn1cc(C(=O)Nc2cc3cc(C4(C#N)CC45CC5)ccc3cn2)cn1] | Cal′c: 344 Found: 344 |
| | N-(6-(1-cyanospiro[2.2]pentan-1-yl)isoquinolin-3-yl)-1-methyl- | |
| | 1H-pyrazole-4-carboxamide, (R or S)-N-(6-(1- | |
| | cyanospiro[2.2]pentan-1-yl)isoquinolin-3-yl)-1-methyl-1H- | |
| | pyrazole-4-carboxamide | |

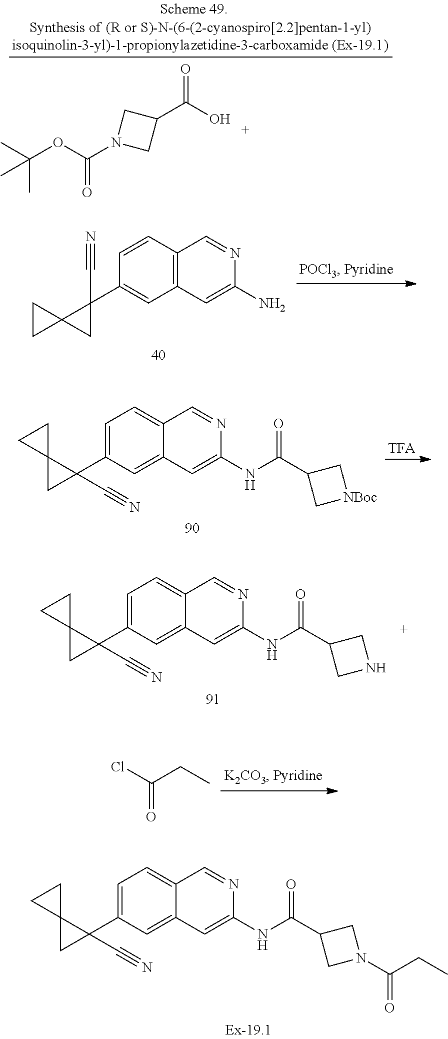

### tert-butyl 3-((6-(1-cyanospiro[2.2]pentan-1-yl)isoquinolin-3-yl)carbamoyl)azetidine-1-carboxylate (90)

Pyridine (83 μL, 1.020 mmol) and POCl₃ (24 μL, 0.257 mmol) were added to a solution of 1-(3-aminoisoquinolin-6-yl)spiro[2.2]pentane-1-carbonitrile 40 (30 mg, 0.128 mmol) and 1-(tert-butoxycarbonyl)azetidine-3-carboxylic acid (30.8 mg, 0.153 mmol) in anhydrous DCM (2 ml) at 0° C. The resulting mixture was stirred at 0° C. for 30 minutes. The reaction mixture was poured into H₂O (5 mL), extracted with EtOAc (20 mL×3), and dried over Na₂SO₄. The mixture was filtered and concentrated under reduced pressure to afford the title compound, which was used in the next step without further purification. MS (ESI): m/z calc'd for C₂₄H₂₆N₄O₃ [M+H]⁺: 419, found 419.

### N-(6-(1-cyanospiro[2.2]pentan-1-yl)isoquinolin-3-yl)azetidine-3-carboxamide (91)

TFA (0.4 ml, 5.19 mmol) was added to a solution of tert-butyl 3-((6-(1-cyanospiro[2.2]pentan-1-yl)isoquinolin-3-yl)carbamoyl)azetidine-1-carboxylate 90 (45 mg, 0.108 mmol) in anhydrous DCM (2 ml). The mixture was stirred at room temperature for 1 h. The mixture was adjusted pH to 7-8 using aq NaHCO₃ (10 ml), extracted with EtOAc (30 mL×3), and dried over Na₂SO₄. The mixture was filtered and concentrated under reduced pressure to afford the title compound which was used in the next step without further purification. MS (ESI): m/z calc'd for C₁₉H₁₈N₄O [M+H]⁺ 319, found 319.

### (R or S)-N-(6-(1-cyanospiro[2.2]pentan-1-yl)isoquinolin-3-yl)-1-propionylazetidine-3-carboxamide (Ex-19.1)

A solution of N-(6-(1-cyanospiro[2.2]pentan-1-yl)isoquinolin-3-yl)azetidine-3-carboxamide 91 (30 mg, 0.094 mmol) in anhydrous DCM (4 ml) was added to K₂CO₃ (39.1 mg, 0.283 mmol), and the resulting mixture was stirred at 0° C. A solution of propionyl chloride (10.46 mg, 0.113 mmol) was added, and the resulting mixture was stirred at room temperature for 12 hours. The mixture was poured in water (10 ml), extracted with EtOAc (10 mL×3), and dried over Na₂SO₄. The mixture was filtered and concentrated under reduced pressure. The reaction mixture was purified by HPLC, eluting acetonitrile/water gradient with 0.1% TFA modifier, linear gradient and lyophilized to afford the title compound as a TFA salt. MS (ESI): m/z calc'd for C₂₂H₂₂N₄O₂ [M+H]⁺ 375, found 375. ¹H NMR (400 MHz, CHLOROFORM-d) δ 8.92 (br d, J=12.91 Hz, 2H), 8.00 (br d, J=8.61 Hz, 1H), 7.87 (s, 1H), 7.53 (br d, J=8.61 Hz, 1H), 4.42-4.49 (m, 1H), 4.34-4.41 (m, 1H), 4.29 (br d, J=5.09 Hz, 2H), 3.71 (br t, J=6.06 Hz, 1H), 2.48 (d, J=5.09 Hz, 1H), 2.15 (q, J=7.43 Hz, 2H), 2.00 (br d, J=5.09 Hz, 1H), 1.39-1.46 (m, 1H), 1.29-1.38 (m, 2H), 1.15 (t, J=7.43 Hz, 3H), 1.10 (br d, J=5.48 Hz, 1H)

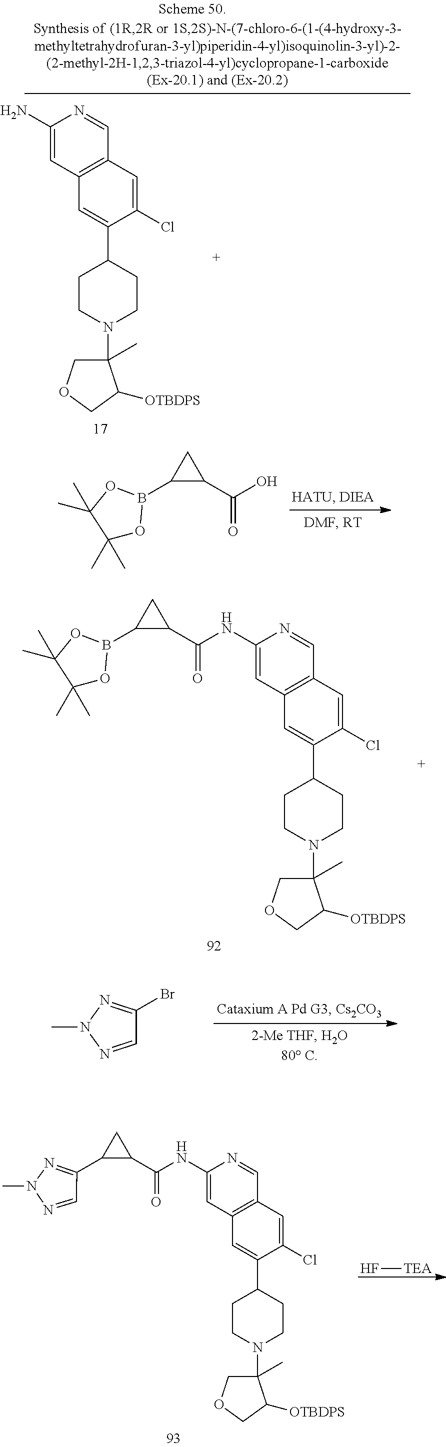

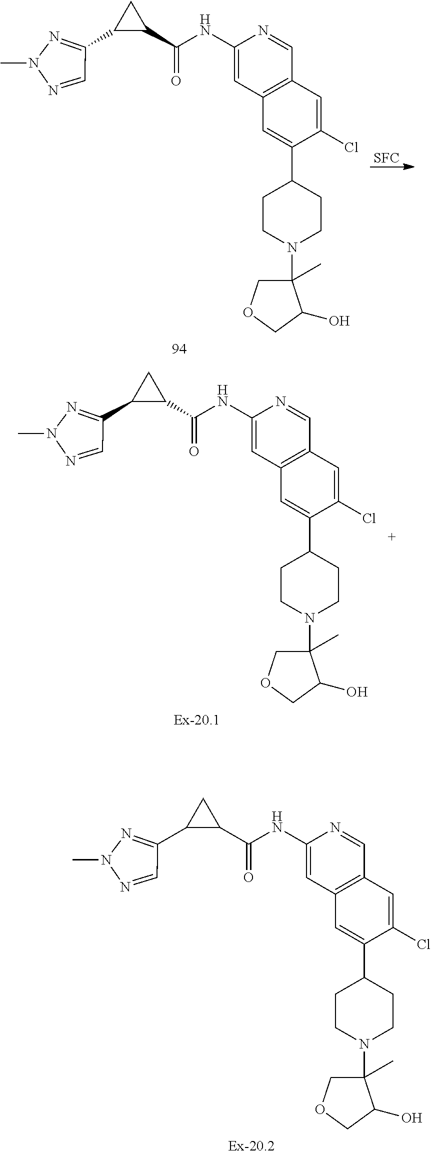

### N-(6-(1-(4-((tert-butyldiphenylsilyl)oxy)-3-methyltetrahydrofuran-3-yl)piperidin-4-yl)-7-chloroisoquinolin-3-yl)-2-(4,4,5,5-tetramethyl-1,3,2-dioxaborolan-2-yl)cyclopropane-1-carboxamide (92)

6-(1-(4-((tert-butyldiphenylsilyl)oxy)-3-methyltetrahydrofuran-3-yl)piperidin-4-yl)-7-chloroisoquinolin-3-amine, 2HCl 17 (202 mg, 0.3 mmol), 2-(4,4,5,5-tetramethyl-1,3,2-dioxaborolan-2-yl)cyclopropane-1-carboxylic acid (105 mg, 0.495 mmol), HATU (188 mg, 0.495 mmol), DMF (1500 μl), and DIEA (262 μl, 1.500 mmol) were added to a vial. The resulting mixture was stirred overnight at room temperature. The reaction mixture was added to water to form a precipitate. The solids were collected by vacuum filtration and dried to afford the title compound, which was used without further purification in the next step. MS (ESI): m/z calc'd for C₄₅H₅₇BClN₃O₅Si [M+H]+: 794, found 794.

### N-(6-(1-(4-((tert-butyldiphenylsilyl)oxy)-3-methyltetrahydrofuran-3-yl)piperidin-4-yl)-7-chloroisoquinolin-3-yl)-2-(2-methyl-2H-1,2,3-triazol-4-yl)cyclopropane-1-carboxamide (93)

N-(6-(1-(4-((tert-butyldiphenylsilyl)oxy)-3-methyltetrahydrofuran-3-yl)piperidin-4-yl)-7-chloroisoquinolin-3-yl)-2-(4,4,5,5-tetramethyl-1,3,2-dioxaborolan-2-yl)cyclopropane-1-carboxamide (200 mg, 0.252 mmol), 4-bromo-2-methyl-2H-1,2,3-triazole (82 mg, 0.504 mmol), Cataxium-A Pd G3 (36.7 mg, 0.050 mmol), and cesium carbonate (246 mg, 0.755 mmol) were added to a vial. The vial was sealed, and its contents were placed under an inert atmosphere by performing 3 vacuum/nitrogen cycles. 2-Methyltetrahydrofuran (1000 μl) and Water (100 μl) were added through the septum and the resulting mixture was allowed to stir overnight at 80° C. The crude reaction mixture was scavenged for 72 hours at room temperature with Si-DMT. The reaction mixture was filtered, and the residue was washed with 3:1 Chloroform: iPrOH. The reaction mixture was diluted with 3:1 Chloroform:iPrOH and washed with saturated ammonium chloride, the biphasic mixture was passed through a phase separator cartridge and concentrated under reduced pressure to afford the title compound, which was used without further purification in the next step. MS (ESI): m/z calc'd for C₄₂H₄₉ClN₆O₃Si [M+H]+: 749, found 749.

### (1R,2R and 1S,2S)-N-(7-chloro-6-(1-(4-hydroxy-3-methyltetrahydrofuran-3-yl)piperidin-4-yl)isoquinolin-3-yl)-2-(2-methyl-2H-1,2,3-triazol-4-yl)cyclopropane-1-carboxamide (Ex-20.1) and (Ex-20.2)

N-(6-(1-(4-((tert-butyldiphenylsilyl)oxy)-3-methyltetrahydrofuran-3-yl)piperidin-4-yl)-7-chloroisoquinolin-3-yl)-2-(2-methyl-2H-1,2,3-triazol-4-yl)cyclopropane-1-carboxamide (189 mg, 0.252 mmol) was dissolved in DCM (2522 μl) and transferred to a plastic 50 mL centrifuge tube. HF-TEA (100 μl, 0.620 mmol) was added carefully, and the resulting mixture was allowed to stir for 2 hours at room temperature. The reaction mixture was quenched with saturated sodium bicarbonate (stirring for 20 minutes until the pH was ˜8). The reaction mixture was diluted with DCM and washed with saturated sodium bicarbonate. The biphasic mixture was passed through a phase separator cartridge and concentrated under reduced pressure. The reaction mixture was filtered, purified by HPLC, eluting acetonitrile/water gradient with 0.1% Ammonium hydroxide modifier, linear gradient, and lyophilized to afford a mixture of product and impurity. 3 mL of diethyl ether and 1 drop of MeOH was added to the vial, and the contents were sonicated. The solid was collected by vacuum filtration and dried in vacuo to afford the title compound as a mixture of stereoisomers. The mixture of two stereoisomers was purified by chiral SFC (OJ-H, 21×250 (mm), 25%/75% Methanol/CO₂+0.1% NH₄OH) and lyophilized to afford resolved stereoisomers of the title compound Ex-20.1 (tR=4.75 min) and Ex-20.2 (tR=6.3 min). Ex-20.1: MS (ESI): m/z calc'd for C₂₆H₃₁ClN₆O₃ [M+H]⁺: 511, found 511. ¹H NMR (499 MHz, DMSO-d₆) δ 10.97 (s, 1H), 9.08 (s, 1H), 8.47 (s, 1H), 8.19 (s, 1H), 7.95 (s, 1H), 7.65 (s, 1H), 4.37 (s, 1H), 4.08 (s, 3H), 3.96 (d, J=7.0 Hz, 1H), 3.80 (s, 1H), 3.71 (d, J=9.7 Hz, 1H), 3.62 (d, J=7.2 Hz, 1H), 3.55 (d, J=7.2 Hz, 1H), 3.05 (s, 1H), 2.86 (d, J=10.9 Hz, 1H), 2.61-2.55 (m, 1H), 2.47-2.42 (m, 3H), 1.92-1.84 (m, 4H), 1.53-1.48 (m, 1H), 1.42-1.37 (m, 1H), 1.25-1.16 (m, 1H), 1.05 (s, 3H). Ex-20.2: MS (ESI): m/z calc'd for C₂₆H₃₁ClN₆O₃ [M+H]⁺: 511, found 511. ¹H NMR (499 MHz, DMSO-d₆) δ 10.97 (s, 1H), 9.08 (s, 1H), 8.47 (s, 1H), 8.19 (s, 1H), 7.95 (s, 1H), 7.65 (s, 1H), 4.37 (s, 1H), 4.08 (s, 3H), 3.96 (d, J=7.0 Hz, 1H), 3.80 (s, 1H), 3.71 (d, J=9.7 Hz, 1H), 3.62 (d, J=7.2 Hz, 1H), 3.55 (d, J=7.2 Hz, 1H), 3.05 (s, 1H), 2.86 (d, J=10.9 Hz, 1H), 2.61-2.55 (m, 1H), 2.47-2.42 (m, 3H), 1.92-1.84 (m, 4H), 1.53-1.48 (m, 1H), 1.42-1.37 (m, 1H), 1.25-1.16 (m, 1H), 1.05 (s, 3H).

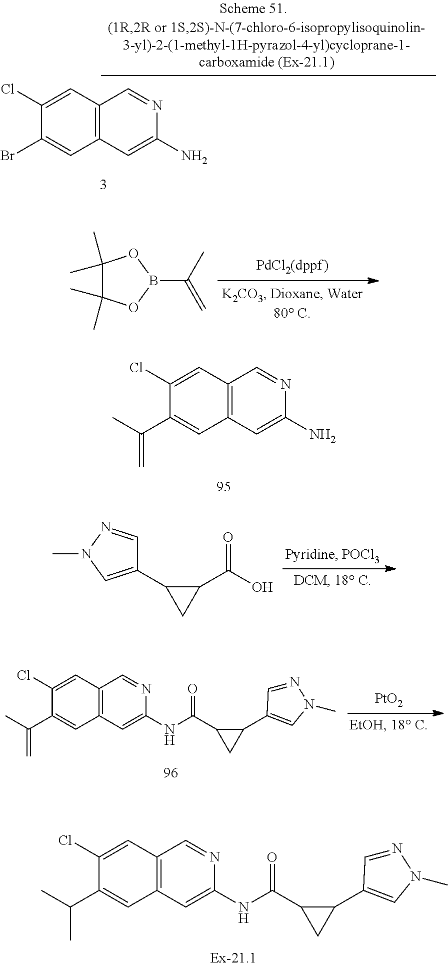

### 7-chloro-6-(prop-1-en-2-yl)isoquinolin-3-amine (95)

To a solution of 6-bromo-7-chloroisoquinolin-3-amine (100 mg, 0.388 mmol), 4,4,5,5-tetramethyl-2-(prop-1-en-2-yl)-1,3,2-dioxaborolane (196 mg, 1.165 mmol) and K₂CO₃ (107 mg, 0.777 mmol) in 1,4-Dioxane (3 mL) and Water (0.6 mL) was added PdCl₂(dppf) (28.4 mg, 0.039 mmol) in a glovebox. The resulting mixture was stirred at 80° C. for 16 hours, then slowly added into water (20 mL). EtOAc (20 mL), was added into the mixture, and the organic layer was separated. The aqueous was extracted with EtOAc. The combined organic layers were dried by anhydrous Na₂SO₄, filtered and concentrated in vacuo to give the crude product which was purified by flash silica gel chromatography to afford title compound 96. MS (ESI): m/z calc'd for C₁₂H₁₂ClN₂ [M+H]⁺: 219, found 219.

### N-(7-chloro-6-(prop-1-en-2-yl)isoquinolin-3-yl)-2-(1-methyl-1H-pyrazol-4-yl)cyclopropane-1-carboxamide (96)

To a solution of 7-chloro-6-(prop-1-en-2-yl)isoquinolin-3-amine (50 mg, 0.229 mmol) and 2-(1-methyl-1H-pyrazol-4-yl)cyclopropane-1-carboxylic acid (57.0 mg, 0.343 mmol) in DCM (2 mL) was added pyridine (0.148 mL, 1.829 mmol) and phosphoryl trichloride (0.043 mL, 0.457 mmol) at 0° C. The mixture was stirred at 18° C. for 0.5 hour, then slowly added into ice water. The resulting mixture was extracted with EtOAc. The combined organic layers were washed with water, dried over Na₂SO₄. After filtration and concentration, the residue was purified by pre-TLC to afford title compound. MS (ESI): m/z calc'd for C₂₀H₂₀ClN₂O₂ [M+H]⁺: 367, found 367.

### N-(7-chloro-6-isopropylisoquinolin-3-yl)-2-(1-methyl-1H-pyrazol-4-yl)cyclopropane-1-carboxamide (Ex-21.1)

To a solution of N-(7-chloro-6-(prop-1-en-2-yl)isoquinolin-3-yl)-2-(1-methyl-1H-pyrazol-4-yl)cyclopropane-1-carboxamide (50 mg, 0.136 mmol) in anhydrous EtOH (5 mL) was added platinum(IV) oxide (3.10 mg, 0.014 mmol), and the resulting mixture was stirred at 18° C. under H₂ (15 psi) for 0.5 hour. After filtration and concentration, the residue was purified by Prep-HPLC (TFA) to afford title compound Ex-21.1.

¹H NMR (400 MHz, MeOD-d₄) δ 9.00 (s, 1H), 8.24 (s, 1H), 8.10 (s, 1H), 7.82 (s, 1H), 7.48 (s, 1H), 7.36 (s, 1H), 3.82 (s, 3H), 3.56-3.46 (m, 1H), 2.43-2.35 (m, 1H), 2.08-2.00 (m, 1H), 1.58 (m, 1H), 1.35 (s, 3H), 1.34 (s, 3H), 1.27 (m, 1H)

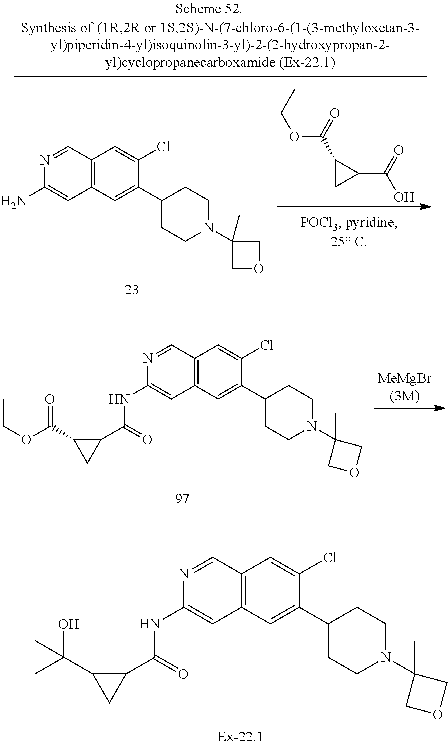 [SMILES: CC1(N2CCC(c3cc4cc(N)ncc4cc3Cl)CC2)COC1 ; CCOC(=O)[C@H]1CC1C(=O)O ; CCOC(=O)[C@H]1CC1C(=O)Nc1cc2cc(C3CCN(C4(C)COC4)CC3)c(Cl)cc2cn1 ; CC(C)(O)C1CC1C(=O)Nc1cc2cc(C3CCN(C4(C)COC4)CC3)c(Cl)cc2cn1]

### (1R,2R)-ethyl 2-((7-chloro-6-(1-(3-methyloxetan-3-yl)piperidin-4-yl)isoquinolin-3-yl) carbamoyl) cyclopropane carboxylate (97)

To a solution of (1R,2R)-2-(ethoxycarbonyl)cyclopropanecarboxylic acid (28.6 mg, 0.181 mmol) and 7-chloro-6-(1-(3-methyloxetan-3-yl)piperidin-4-yl)isoquinolin-3-amine (50.0 mg, 0.151 mmol) in pyridine (1.0 mL) was added POCl₃ (0.028 mL, 0.301 mmol) at 25° C. The reaction mixture was stirred for 10 min at 25° C. The reaction mixture was concentrated and purified by pre-TLC (silica gel, Pet·ether/EtOAc=1:1) to afford title compound 97. MS (ESI): m/z calc'd for C₂₅H₃₁ClN₃O₄ [M+H]⁺: 472, found 472.

### (1R,2R or 1S,2S)-N-(7-chloro-6-(1-(3-methyloxetan-3-yl)piperidin-4-yl)isoquinolin-3-yl)-2-(2-hydroxypropan-2-yl)cyclopropanecarboxamide (Ex-22.1)

To a solution of ethyl 2-((7-chloro-6-(1-(3-methyloxetan-3-yl)piperidin-4-yl)isoquinolin-3-yl)carbamoyl)cyclopropanecarboxylate (15.0 mg, 0.032 mmol) in THF (1 mL) was added methylmagnesium bromide (0.424 mL, 1.27 mmol) at 25° C. The mixture was stirred for 1 hour at 60° C. under microwave condition. The mixture was poured into the water and extracted with EtOAc. The organic layer was separated. The aqueous was extracted with EtOAc and treated with brine. The combined organic layers were dried by anhydrous Na₂SO₄, filtered and the filtrate was concentrated under reduced pressure and was purified by prep-HPLC (TFA) to afford title compound Ex-22.1. ¹H NMR (400 MHz, CDCl₃) δ 8.85 (s, 1H), 8.61 (b, 1H), 8.49 (s, 1H), 7.91 (s, 1H), 7.66 (s, 1H), 5.25 (d, J=6.7 Hz, 2H), 4.33 (d, J=7.0 Hz, 2H), 3.38 (d, J=11.0 Hz, 2H), 3.30 (d, J=11.3 Hz, 1H), 2.86 (t, J=11.3 Hz, 2H), 2.37 (s, 2H), 2.25-2.18 (m, 2H), 2.01 (d, J=5.9 Hz, 1H), 1.80-1.75 (m, 3H), 1.73-1.68 (m, 1H), 1.33 (d, J=12.1 Hz, 6H), 1.11-1.02 (m, 2H), 0.87-0.78 (m, 2H). MS (ESI): m/z calc'd for C₂₅H₃₃ClN₃O₃[M+H]⁺: 458, found 458.

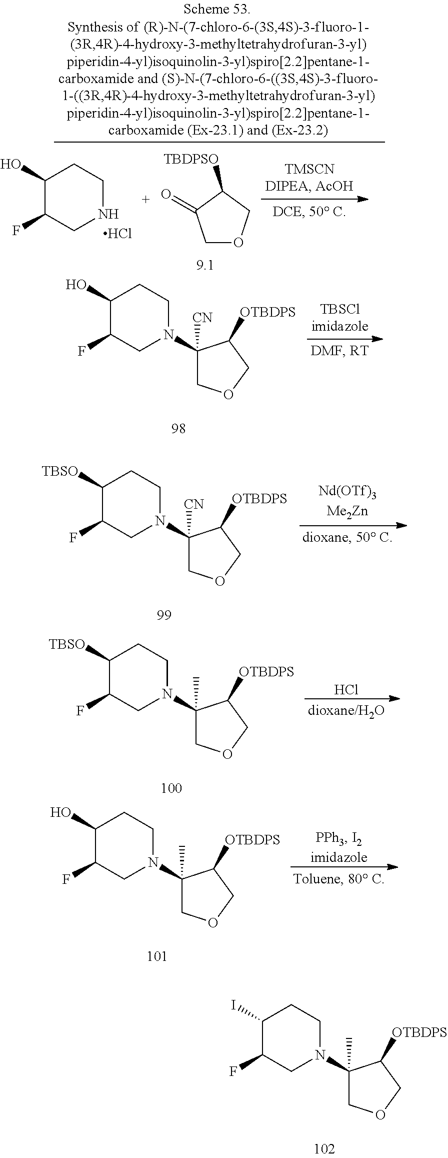

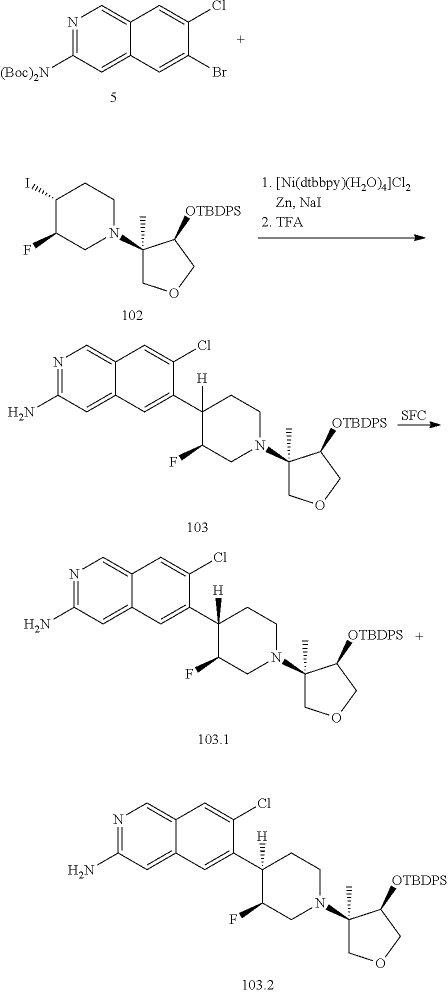

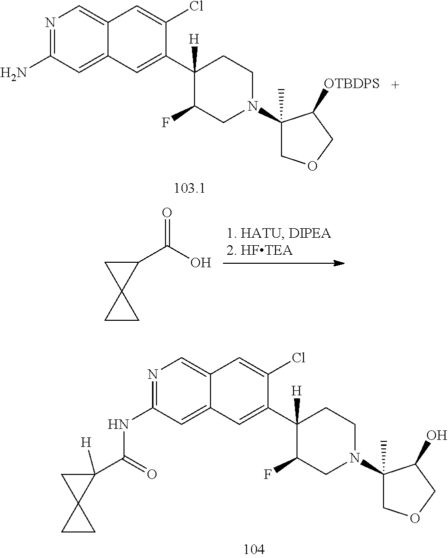

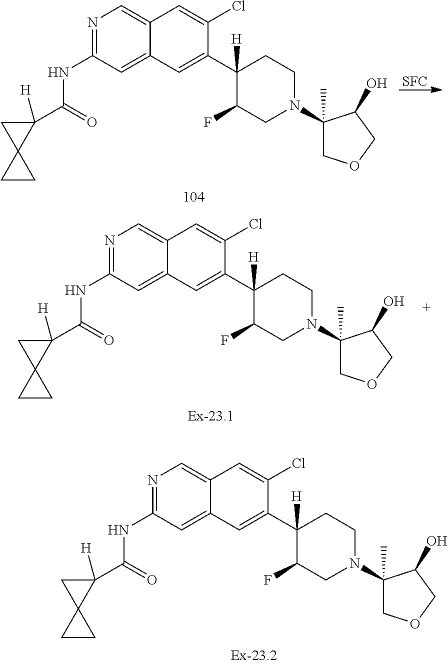

### (3R,4R)-4-((tert-butyldiphenylsilyl)oxy)-3-((3R,4S)-3-fluoro-4-hydroxypiperidin-1-yl)tetrahydrofuran-3-carbonitrile (98)

In a ventilated balance enclosure, a 250 mL round bottom flask equipped with a magnetic stirrer was charged with (3R,4S)-3-fluoropiperidin-4-ol, HCl (1.15 g, 7.39 mmol), (S)-4-((tert-butyldiphenylsilyl)oxy)dihydrofuran-3(2H)-one 9.1 (3.02 g, 8.87 mmol), and approximately ˜1 weight equivalent of oven-dried 4-angstrom molecular sieves. The flask was then equipped with a reflux condenser and sealed with a rubber septum. Then, under a positive flow of argon anhydrous DCE (50 mL) was added and stirring was commenced. To the stirring mixture at RT was first added Hunig's Base (2.58 ml, 14.8 mmol). After ˜10 minutes of vigorous stirring at RT, trimethylsilyl cyanide (1.73 ml, 12.9 mmol), then acetic acid (1.69 ml, 29.6 mmol) were added, and the reaction was heated to 50° C. overnight. The mixture was diluted with DCM and filtered through a medium porosity frit to remove debris from the molecular sieves. The filtrate was then carefully transferred to an oversized Erlenmeyer flask containing vigorously stirring sat. aq. NaHCO₃ (foaming occurs). This mixture was stirred for ˜5 minutes, then transferred to a separatory funnel where the phases were separated and the aqeuous phase extracted with DCM (2×). The combined organic layers were dried over Na₂SO₄, filtered, and the collected filtrate concentrated to dryness in vacuo. The crude residue was purified by column chromatography on silica gel (0-8% MeOH/DCM) to afford the title compound 98. An inseparable mixture of two diastereomers was carried onward to the next step where the two isomers could be resolved. MS (ESI): m/z calc'd for C₂₆H₃₄FN₂O₃Si [M+H]⁺: 469, found 469.

### (3R,4R)-3-((3R,4S)-4-((tert-butyldimethylsilyl)oxy)-3-fluoropiperidin-1-yl)-4-((tert-butyldiphenylsilyl)oxy)tetrahydrofuran-3-carbonitrile (99)

In a ventilated balance enclosure, a 250 mL round bottom flask equipped with a magnetic stirrer was charged with intermediate 98 (3.05 g, 6.51 mmol), TBSCl (2.94 g, 19.5 mmol), and imidazole (1.44 g, 21.2 mmol). Then, under a positive flow of argon anhydrous DMF (43.4 ml) was added. The mixture was stirred at RT over 2 days. The reaction was quenched by careful addition of sat. aq. sodium bicarbonate. The mixture was diluted with EtOAc and poured into a separatory funnel. The phases were separated and the aqueous phase was extracted once more with EtOAc. The combined organic layers were washed with brine, dried over anhydrous sodium sulfate, filtered, and the collected filtrate was concentrated to dryness in vacuo. The crude residue was purified by column chromatography on silica gel (0-15% EtOAc/Hexanes) to afford the title compound 99 as well as a minor diastereomer (>4:1 dr). MS (ESI): m/z calc'd for C₃₂H₄₈FN₂O₃Si₂ [M+H]⁺: 583, found 583.

### (3R,4S)-4-((tert-butyldimethylsilyl)oxy)-1-((3R,4R)-4-((tert-butyldiphenylsilyl)oxy)-3-methyltetrahydrofuran-3-yl)-3-fluoropiperidine (100)

In a ventilated balance enclosure, a 250 mL round bottom flask equipped with a magnetic stirrer was charged with intermediate 99 (1.85 g, 3.17 mmol) and Nd(OTf)₃ (0.469 g, 0.793 mmol). Then, under a positive flow of argon the mixture was dissolved in dioxane (31 mL) and stirred. To the stirring mixture at RT was very slowly added dimethylzinc (7.93 ml, 15.9 mmol) (2 M solution in toluene). Upon complete addition of the volume of Me₂Zn, the reaction was warmed to 50° C. and stirred at this temperature for 2.5 hrs. The reaction was cooled, and carefully poured into an Erlenmeyer flask containing sat. aq. NaHCO₃ (foaming occurs). The mixture was transferred to a separatory funnel and extracted with DCM. The combined organic layers were dried over Na₂SO₄, filtered, and the collected filtrate concentrated to dryness in vacuo. The crude residue was purified by column chromatography on silica gel (0-50% EtOAc/Hexanes) to afford the title compound 100. MS (ESI): m/z calc'd for C₃₂H₅₁FNO₃Si₂ [M+H]⁺: 572, found 572.

### (3R,4S)-1-((3R,4R)-4-((tert-butyldiphenylsilyl)oxy)-3-methyltetrahydrofuran-3-yl)-3-fluoropiperidin-4-ol (101)

In a ventilated balance enclosure, a 250 mL round bottom equipped with a magnetic stirrer was charged with intermediate 100 (1.45 g, 2.54 mmol). Then, under a positive flow of argon anhydrous dioxane (17 mL) and water (2.3 mL) were added. Finally, hydrochloric acid (4 M in dioxane, 3.17 mL, 12.7 mmol) was added, and the reaction mixture was stirred overnight at RT. The mixture was carefully quenched by pouring into an Erlenmeyer flask containing sat. aq. NaHCO₃. The mixture was then extracted with 3:1 CHCl₃/IPA (3×), and the combined organic layers were dried over anhydrous Na₂SO₄, filtered, and the collected filtrate was concentrated to dryness in vacuo. The crude residue containing the title compound 101 in reasonably pure form was carried on directly without additional purification (note—mass balance exceeds 100%, likely due to presence of silanol byproduct). MS (ESI): m/z calc'd for C₂₆H₃₇FNO₃Si [M+H]⁺: 458, found 458.

### (3R,4R)-1-((3R,4R)-4-((tert-butyldiphenylsilyl)oxy)-3-methyltetrahydrofuran-3-yl)-3-fluoro-4-iodopiperidine (102)

In a ventilated balance enclosure, a 250 mL round bottom flask equipped with a magnetic stirrer and reflux condenser was charged with triphenylphosphine (1.33 g, 5.08 mmol), imidazole (0.346 g, 5.08 mmol), and the crude intermediate 101 (1.16 g, 2.54 mmol). Then, under a positive flow of argon anhydrous toluene (15 mL) was added. Finally, a solution of iodine (1.29 g, 5.08 mmol) in toluene (10 mL—heating and sonication required) was added slowly via syringe. On complete addition, the mixture was heated to 80° C. for 1 hr. The mixture was diluted with EtOAc and transferred to a separatory funnel where it was washed with saturated aqueous Na₂S₂O₃, then brine. The organic layer was then dried over Na₂SO₄, filtered, and the collected filtrate concentrated to dryness in vacuo. The crude residue was purified by column chromatography on silica gel (0-40% EtOAc/hexanes) to afford the desired product as a mixture of diastereomers. However, the major isomer was present in 7× the abundance of the next closest isomer by mass balance. The mixture from was submitted to preparative SFC (Lux-4, 21×250 mm, 5 μm; 70 ml/min; 20% MeOH w/0.1% NH₄OH) to obtain 5 elutions of the desired product. The first elution whose mass corresponds to the desired product (t_{R}=2.44 min) represents the major diastereomer, and thusly the title compound 102 was obtained as a single isomer. MS (ESI): m/z calc'd for C₂₆H₃₆FINO₂Si [M+H]⁺: 568, found 568.

### 6-((3S,4S)-1-((3R,4R)-4-((tert-butyldiphenylsilyl)oxy)-3-methyltetrahydrofuran-3-yl)-3-fluoropiperidin-4-yl)-7-chloroisoquinolin-3-amine and 6-((3S,4R)-1-((3R,4R)-4-((tert-butyldiphenylsilyl)oxy)-3-methyltetrahydrofuran-3-yl)-3-fluoropiperidin-4-yl)-7-chloroisoquinolin-3-amine (103.1 and 103.2)

In a ventilated balance enclosure, a 30 mL scintillation vial equipped with a magnetic stirrer was charged with intermediate 5 (225 mg, 0.492 mmol), intermediate 102 (307 mg, 0.541 mmol), sodium iodide (74 mg, 0.49 mmol), zinc (96 mg, 1.475 mmol), and [Ni(dtbbpy)(H₂O)₄]Cl₂ (46 mg, 0.098 mmol). The vial was sealed with a septum cap and then with an inverted 24/40 rubber septum which was sealed to the sides of the vial with parafilm. The vial was evacuated then back-filled with N2 (3 cycles). Then, under a positive flow of N2, anhydrous MeCN (4.9 mL) was added. The mixture was immediately sonicated for ˜5 min with manual swirling, over which time the green-blue suspension becomes tan-brown. The reaction was then stirred at RT overnight under inert atmosphere. The mixture was diluted with EtOAc and filtered through a pad of Celite, eluting with additional EtOAc. The collected filtrate was concentrated to dryness in vacuo. The resultant crude residue was transferred to a 30 mL scintillation vial and again concentrated to dryness in vacuo. The residue was then dissolved in DCM (5 mL), and to the stirring mixture at RT was added trifluoroacetic acid (0.947 mL, 12.3 mmol). The reaction was stirred at RT for 4 hrs, at which point volatiles were removed on the Biotage® V-10 evaporator. The residue was taken up in MeCN (10 mL) and again concentrated to dryness using the Biotage® V-10 in order to drive off excess TFA. The crude residue was submitted for purification by preparative HPLC (MeCN/H₂O/TFA). The isolated material was then free-based by liquid-liquid extraction (sat. aq. NaHCO₃/3:1 CHCl₃:IPA) to afford the product 103 as a mixture of stereoisomers. This material was then submitted for chiral resolution by preparative SFC ((R,R)-Whelk-O, 21×250 mm, 5 μm; 70 ml/min; 25% MeOH w/0.1% NH₄OH) to provide the enantiomerically pure title compounds 103.1 (major, t_{R}=8.25 min) and 103.2 (minor, t_{R}=9.65 min). For 103.1—MS (ESI): m/z calc'd for C₃₅H₄₁ClFN₃O₂Si [M+H]⁺: 618, found 618. For 103.2—MS (ESI): m/z calc'd for C₃₅H₄₁ClFN₃O₂Si [M+H]⁺: 618, found 618.

### (S)-N-(7-chloro-6-((3S,4S)-3-fluoro-1-((3R,4R)-4-hydroxy-3-methyltetrahydrofuran-3-yl)piperidin-4-yl)isoquinolin-3-yl)spiro[2.2]pentane-1-carboxamide and (R)-N-(7-chloro-6-((3S,4S)-3-fluoro-1-((3R,4R)-4-hydroxy-3-methyltetrahydrofuran-3-yl)piperidin-4-yl)isoquinolin-3-yl)spiro[2.2]pentane-1-carboxamide (Ex-23.1 and Ex-23.2)

In a ventilated balance enclosure, a 30 mL scintillation vial equipped with a magnetic stirrer was charged with intermediate 103.1 (64 mg, 0.104 mmol), spiro[2.2]pentane-1-carboxylic acid (20 mg, 0.181 mmol), and HATU (79 mg, 0.207 mmol). Then, under a positive flow of nitrogen anhydrous DMF (1.26 mL) was added. Finally, Hunig's base (90 μL, 0.518 mmol) was added, the nitrogen inlet was removed, and the reaction was stirred in the sealed vial overnight at RT. After overnight stirring at RT, LC/MS analysis revealed an ˜1:1 mixture of starting material to desired pdt. At this point, all reaction components (acid, HATU, Hunig's base) were doubled, and the reaction was placed back on the stir plate where it was warmed to 40° C. and stirred overnight. After these steps, LC/MS revealed a significantly higher ratio of product:starting material, with most of the starting material consumed. Triethylamine trihydrofluoride (0.308 mL, 1.89 mmol) was added at 40° C. Full desilylation was observed after just 30 min. The reaction was quenched by diluting with DCM and pouring into sat. aq. NaHCO₃. After thorough mixing, the biphasic mixture was transferred to a separatory funnel where the phases were separated. The aqueous phase was extracted with 3:1 CHCl₃/IPA, and the combined organic layers were dried over anhydrous Na₂SO₄, filtered, and the collected filtrate was concentrated to dryness in vacuo. The crude, DMF-containing residue was submitted for purification by preparative HPLC (MeCN/H₂O/TFA). The mixture of isomers 104 was then free-based by liquid-liquid extraction (sat. aq. NaHCO₃/3:1 CHCl₃:IPA), and the combined organic layers were dried over anhydrous Na₂SO₄, filtered, and the collected filtrate was concentrated to dryness in vacuo. This mixture of was then submitted to preparative SFC (AS-H, 21×250 mm, 5 μm; 70 ml/min; 20% MeOH w/0.1% NH₄OH) to afford the enantiomerically pure title compounds Ex-23.1 (t_{R}=4.40 min) and Ex-23.2 (t_{R}=5.65 min). For Ex-23.1—MS (ESI): m/z calc'd for C₂₅H₃₀ClFN₃O₃ [M+H]⁺: 474, found 474; ¹H NMR (400 MHz, d6-DMSO) δ: 10.73 (s, 1H), 9.08 (s, 1H), 8.49 (s, 1H), 8.20 (s, 1H), 8.17 (s, 1H), 5.25 (dtd, J=49.1, 9.8, 4.7 Hz, 1H), 4.32 (s, 1H), 3.97 (dd, J=9.6, 3.1 Hz, 1H), 3.86 (s, 1H), 3.70 (d, J=9.7 Hz, 1H), 3.61 (d, J=7.4 Hz, 1H), 3.54 (d, J=7.3 Hz, 1H), 3.27-3.17 (m, 1H), 2.46-2.35 (m, 3H), 1.92 (d, J=5.0 Hz, 1H), 1.81-1.65 (m, 1H), 1.44 (s, 1H), 1.38-1.30 (m, 1H), 1.20 (d, J=35.5 Hz, 2H), 1.05 (s, 3H), 0.91 (d, J=5.0 Hz, 2H), 0.89-0.82 (m, 1H), 0.76 (t, J=7.8 Hz, 2H). For Ex-23.2—MS (ESI): m/z calc'd for C₂₅H₃₀ClFN₃O₃ [M+H]⁺: 474, found 474; ¹H NMR (400 MHz, d₆-DMSO) δ: 10.73 (s, 1H), 9.08 (s, 1H), 8.48 (s, 1H), 8.20 (s, 1H), 8.17 (s, 1H), 5.37-5.12 (m, 1H), 4.32 (s, 1H), 3.97 (dd, J=9.6, 3.0 Hz, 1H), 3.86 (s, 1H), 3.70 (d, J=9.7 Hz, 1H), 3.61 (d, J=7.3 Hz, 1H), 3.54 (d, J=7.3 Hz, 1H), 3.28-3.14 (m, 1H), 2.46-2.35 (m, 3H), 1.91 (s, 1H), 1.81-1.63 (m, 1H), 1.44 (s, 1H), 1.39-1.29 (m, 1H), 1.20 (d, J=36.6 Hz, 2H), 1.05 (s, 3H), 0.98-0.88 (m, 2H), 0.88-0.81 (m, 1H), 0.76 (t, J=7.8 Hz, 2H).

| | LRRK2 |
|---|---|
| Ex | pIC₅₀ |
| Ex-1.1 | 8.96 |
| Ex-1.2 | 9.18 |
| Ex-1.3 | 9.20 |
| Ex-1.4 | 8.44 |
| Ex-1.5 | 8.23 |
| Ex-1.6 | 8.73 |
| Ex-1.7 | 8.95 |
| Ex-1.8 | 7.67 |
| Ex-1.9 | 8.54 |
| Ex-1.10 | 8.40 |
| Ex-1.11 | 7.40 |
| Ex-1.12 | 8.03 |
| Ex-1.13 | 8.61 |
| Ex-1.14 | 8.87 |
| Ex-1.15 | 8.49 |
| Ex-1.16 | 8.26 |
| Ex-1.17 | 9.75 |
| Ex-1.18 | 8.87 |
| Ex-1.19 | 9.42 |
| Ex-1.20 | 9.74 |
| Ex-1.21 | 10.04 |
| Ex-1.22 | 9.01 |
| Ex-1.23 | 8.80 |
| Ex-1.24 | 10.09 |
| Ex-1.25 | 9.18 |
| Ex-1.26 | 8.47 |
| Ex-1.27 | 8.01 |
| Ex-1.28 | 7.62 |
| Ex-1.29 | 7.69 |
| Ex-1.30 | 7.85 |
| Ex-1.31 | 7.93 |
| Ex-1.32 | 8.63 |
| Ex-1.33 | 7.55 |
| Ex-1.34 | 10.09 |
| Ex-1.35 | 7.58 |
| Ex-1.36 | 8.23 |
| Ex-1.37 | 7.90 |
| Ex-1.38 | 7.94 |
| Ex-1.39 | 9.40 |
| Ex-1.40 | 9.95 |
| Ex-1.41 | 9.15 |
| Ex-1.42 | 8.33 |
| Ex-1.43 | 8.15 |
| Ex-1.44 | 7.64 |
| Ex-1.45 | 7.89 |
| Ex-1.46 | 7.86 |
| Ex-1.47 | 7.70 |
| Ex-1.48 | 7.26 |
| Ex-1.49 | 7.41 |
| Ex-1.50 | 10.09 |
| Ex-1.51 | 9.95 |
| Ex-1.52 | 8.79 |
| Ex-1.53 | 9.17 |
| Ex-2.1 | 9.06 |
| Ex-2.2 | 9.40 |
| Ex-2.3 | 8.25 |
| Ex-2.4 | 7.85 |
| Ex-2.5 | 8.01 |
| Ex-2.6 | 8.04 |
| Ex-2.7 | 7.98 |
| Ex-2.8 | 8.08 |
| Ex-2.9 | 8.08 |
| Ex-2.10 | 8.94 |
| Ex-2.11 | 8.70 |
| Ex-2.12 | 9.45 |
| Ex-2.13 | 9.76 |
| Ex-2.14 | 9.73 |
| Ex-2.15 | 9.67 |
| Ex-2.16 | 9.39 |
| Ex-2.17 | 8.37 |
| Ex-2.18 | 8.94 |
| Ex-2.19 | 9.95 |
| Ex-2.20 | 7.94 |
| Ex-2.21 | 10.09 |
| Ex-2.22 | 8.34 |
| Ex-2.23 | 9.78 |
| Ex-2.24 | 9.59 |
| Ex-2.25 | 10.09 |
| Ex-2.26 | 10.09 |
| Ex-2.27 | 9.49 |
| Ex-2.28 | 9.29 |
| Ex-2.29 | 9.63 |
| Ex-2.30 | 9.90 |
| Ex-2.31 | 9.65 |
| Ex-2.32 | 10.09 |
| Ex-2.33 | 10.09 |
| Ex-2.34 | 8.77 |
| Ex-2.35 | 8.58 |
| Ex-2.36 | 9.26 |
| Ex-2.37 | 10.09 |
| Ex-2.38 | 8.69 |
| Ex-2.39 | 8.64 |
| Ex-2.40 | 9.24 |
| Ex-2.41 | 8.73 |
| Ex-2.42 | 8.63 |
| Ex-2.43 | 7.50 |
| Ex-2.44 | 7.40 |
| Ex-2.45 | 7.95 |
| Ex-2.46 | 9.07 |
| Ex-2.47 | 6.34 |
| Ex-2.48 | 6.49 |
| Ex-2.49 | 9.29 |
| Ex-2.50 | 8.13 |
| Ex-2.51 | 9.12 |
| Ex-2.52 | 9.17 |
| Ex-2.53 | 9.09 |
| Ex-2.54 | 9.31 |
| Ex-2.55 | 8.40 |
| Ex-2.56 | 8.80 |
| Ex-2.57 | 8.67 |
| Ex-2.58 | 7.95 |
| Ex-2.59 | 8.29 |
| Ex-2.60 | 7.32 |
| Ex-2.61 | 7.36 |
| Ex-2.62 | 8.29 |
| Ex-2.63 | 7.91 |
| Ex-2.64 | 7.98 |
| Ex-2.65 | 7.59 |
| Ex-2.66 | 7.84 |
| Ex-2.67 | 8.25 |
| Ex-2.68 | 8.73 |
| Ex-2.69 | 7.04 |
| Ex-2.70 | 6.85 |
| Ex-2.71 | 8.24 |
| Ex-2.72 | 8.48 |
| Ex-2.73 | 8.27 |
| Ex-2.74 | 8.95 |
| Ex-2.75 | 7.36 |
| Ex-2.76 | 7.58 |
| Ex-2.77 | 7.99 |
| Ex-2.78 | 7.79 |
| Ex-2.79 | 7.46 |
| Ex-2.80 | 10.09 |
| Ex-2.81 | 10.09 |
| Ex-2.82 | 10.09 |
| Ex-2.83 | 10.09 |
| Ex-2.84 | 10.09 |
| Ex-2.85 | 10.09 |
| Ex-2.86 | 10.09 |
| Ex-2.87 | 10.09 |
| Ex-2.88 | 10.09 |
| Ex-2.89 | 10.09 |
| Ex-2.90 | 7.28 |
| Ex-2.91 | 6.62 |
| Ex-2.92 | 10.09 |
| Ex-2.93 | 10.09 |
| Ex-2.94 | 10.09 |
| Ex-2.95 | 10.09 |
| Ex-2.96 | 10.09 |
| Ex-2.97 | 10.09 |
| Ex-2.98 | 7.81 |
| Ex-2.99 | 6.93 |
| Ex-2.100 | 10.09 |
| Ex-2.101 | 8.22 |
| Ex-2.102 | 9.14 |
| Ex-2.103 | 9.00 |
| Ex-2.104 | 9.18 |
| Ex-2.105 | 8.14 |
| Ex-2.106 | 9.54 |
| Ex-2.107 | 9.18 |
| Ex-2.108 | 8.53 |
| Ex-2.109 | 8.72 |
| Ex-2.110 | 9.24 |
| Ex-2.111 | 8.65 |
| Ex-2.112 | 6.87 |
| Ex-2.113 | 7.55 |
| Ex-3.1 | 7.87 |
| Ex-3.2 | 8.94 |
| Ex-3.3 | 8.48 |
| Ex-3.4 | 8.81 |
| Ex-3.5 | 9.20 |
| Ex-3.6 | 9.62 |
| Ex-3.7 | 10.09 |
| Ex-3.8 | 10.09 |
| Ex-3.9 | 9.01 |
| Ex-3.10 | 9.00 |
| Ex-3.11 | 9.20 |
| Ex-3.12 | 9.60 |
| Ex-4.1 | 9.20 |
| Ex-4.2 | 8.55 |
| Ex-4.3 | 7.74 |
| Ex-4.4 | 6.93 |
| Ex-4.5 | 10.09 |
| Ex-4.6 | 9.83 |
| Ex-4.7 | 10.09 |
| Ex-4.8 | 10.09 |
| Ex-4.9 | 9.82 |
| Ex-4.10 | 10.09 |
| Ex-4.11 | 9.44 |
| Ex-4.12 | 9.40 |
| Ex-4.13 | 9.74 |
| Ex-4.14 | 10.09 |
| Ex-4.15 | 10.09 |
| Ex-4.16 | 10.09 |
| Ex-4.17 | 9.99 |
| Ex-4.18 | 9.78 |
| Ex-4.19 | 9.11 |
| Ex-4.20 | 9.23 |
| Ex-4.21 | 10.09 |
| Ex-4.22 | 8.76 |
| Ex-4.23 | 8.63 |
| Ex-4.24 | 9.16 |
| Ex-4.25 | 9.11 |
| Ex-4.26 | 9.37 |
| Ex-4.27 | 8.59 |
| Ex-4.28 | 10.09 |
| Ex-4.29 | 8.10 |
| Ex-4.30 | 8.52 |
| Ex-4.31 | 8.25 |
| Ex-4.32 | 8.59 |
| Ex-4.33 | 8.93 |
| Ex-4.34 | 8.86 |
| Ex-4.35 | 9.16 |
| Ex-4.36 | 9.25 |
| Ex-4.37 | 10.09 |
| Ex-4.38 | 10.09 |
| Ex-4.39 | 6.87 |
| Ex-4.40 | 6.59 |
| Ex-4.41 | 7.34 |
| Ex-4.42 | 6.70 |
| Ex-4.43 | 8.83 |
| Ex-4.44 | 9.25 |
| Ex-4.45 | 8.15 |
| Ex-4.46 | 8.45 |
| Ex-4.47 | 8.70 |
| Ex-4.48 | 8.90 |
| Ex-4.49 | 9.05 |
| Ex-4.50 | 9.22 |
| Ex-4.51 | 7.08 |
| Ex-4.52 | 7.82 |
| Ex-4.53 | 8.40 |
| Ex-4.54 | 8.04 |
| Ex-4.55 | 7.51 |
| Ex-4.56 | 7.74 |
| Ex-4.57 | 7.38 |
| Ex-4.58 | 8.82 |
| Ex-4.59 | 6.89 |
| Ex-4.60 | 10.09 |
| Ex-4.61 | 7.14 |
| Ex-4.62 | 1.09 |
| Ex-4.63 | 7.65 |
| Ex-4.64 | 8.61 |
| Ex-4.65 | 9.15 |
| Ex-4.66 | 10.09 |
| Ex-4.67 | 10.09 |
| Ex-4.68 | 10.09 |
| Ex-4.69 | 10.09 |
| Ex-4.70 | 10.09 |
| Ex-4.71 | 10.09 |
| Ex-4.72 | 10.09 |
| Ex-4.73 | 6.18 |
| Ex-4.74 | 5.75 |
| Ex-4.75 | 7.00 |
| Ex-4.76 | 7.38 |
| Ex-4.77 | 8.25 |
| Ex-4.78 | 10.09 |
| Ex-5.1 | 7.28 |
| Ex-5.2 | 6.18 |
| Ex-5.3 | 6.96 |
| Ex-5.4 | 7.32 |
| Ex-5.5 | 9.56 |
| Ex-5.6 | 6.04 |
| Ex-5.7 | 8.72 |
| Ex-5.8 | 8.06 |
| Ex-5.9 | 8.75 |
| Ex-5.10 | 8.52 |
| Ex-5.11 | 7.15 |
| Ex-5.12 | 8.65 |
| Ex-5.13 | 6.32 |
| Ex-5.14 | 7.41 |
| Ex-5.15 | 8.89 |
| Ex-5.16 | 6.41 |
| Ex-5.17 | 8.25 |
| Ex-5.18 | 5.99 |
| Ex-5.19 | 8.63 |
| Ex-5.20 | 7.98 |
| Ex-5.21 | 5.44 |
| Ex-5.22 | 8.21 |
| Ex-5.23 | 5.52 |
| Ex-5.24 | 5.37 |
| Ex-5.25 | 9.24 |
| Ex-5.26 | 8.63 |
| Ex-6.1 | 7.37 |
| Ex-6.2 | 6.95 |
| Ex-6.3 | 10.09 |
| Ex-6.4 | 6.85 |
| Ex-6.5 | 9.27 |
| Ex-6.6 | 8.93 |
| Ex-6.7 | 8.74 |
| Ex-6.8 | 7.46 |
| Ex-6.9 | 9.09 |
| Ex-6.10 | 6.96 |
| Ex-6.11 | 8.71 |
| Ex-6.12 | 8.09 |
| Ex-6.13 | 8.23 |
| Ex-6.14 | 8.57 |
| Ex-6.15 | 9.94 |
| Ex-6.16 | 9.67 |
| Ex-6.17 | 8.45 |
| Ex-6.18 | 9.15 |
| Ex-6.19 | 7.40 |
| Ex-6.20 | 7.42 |
| Ex-6.21 | 8.02 |
| Ex-6.22 | 10.09 |
| Ex-6.23 | 8.74 |
| Ex-6.24 | 10.09 |
| Ex-6.25 | 8.78 |
| Ex-6.26 | 9.63 |
| Ex-6.27 | 6.95 |
| Ex-6.28 | 9.44 |
| Ex-6.29 | 7.61 |
| Ex-6.30 | 8.84 |
| Ex-6.31 | 9.75 |
| Ex-6.32 | 8.25 |
| Ex-6.33 | 7.06 |
| Ex-6.34 | 9.01 |
| Ex-6.35 | 8.18 |
| Ex-6.36 | 10.09 |
| Ex-6.37 | 10.09 |
| Ex-6.38 | 9.06 |
| Ex-6.39 | 7.05 |
| Ex-6.40 | 10.09 |
| Ex-6.41 | 10.09 |
| Ex-6.42 | 9.42 |
| Ex-6.43 | 8.55 |
| Ex-6.44 | 8.27 |
| Ex-6.45 | 8.67 |
| Ex-6.46 | 8.92 |
| Ex-6.47 | 9.14 |
| Ex-6.48 | 10.09 |
| Ex-6.49 | 9.50 |
| Ex-6.50 | 10.09 |
| Ex-6.51 | 9.05 |
| Ex-6.52 | 9.70 |
| Ex-6.53 | 8.47 |
| Ex-6.54 | 7.56 |
| Ex-6.55 | 6.35 |
| Ex-7.1 | 7.94 |
| Ex-7.2 | 8.52 |
| Ex-7.3 | 7.78 |
| Ex-7.4 | 8.23 |
| Ex-7.5 | 8.26 |
| Ex-7.6 | 8.67 |
| Ex-7.7 | 8.04 |
| Ex-7.8 | 8.04 |
| Ex-7.9 | 8.58 |
| Ex-7.10 | 8.56 |
| Ex-8.1 | 10.0 |
| Ex-8.2 | 9.63 |
| Ex-8.3 | 9.71 |
| Ex-8.4 | 10.09 |
| Ex-8.5 | 10.00 |
| Ex-9.1 | 8.69 |
| Ex-9.2 | 8.84 |
| Ex-9.3 | 9.20 |
| Ex-9.4 | 8.24 |
| Ex-9.5 | 8.96 |
| Ex-9.6 | 8.05 |
| Ex-9.7 | 8.71 |
| Ex-9.8 | 7.72 |
| Ex-9.9 | 8.25 |
| Ex-9.10 | 7.41 |
| Ex-9.11 | 7.35 |
| Ex-9.12 | 7.45 |
| Ex-9.13 | 8.16 |
| Ex-9.14 | 8.93 |
| Ex-9.15 | 9.37 |
| Ex-9.16 | 8.74 |
| Ex-9.17 | 7.48 |
| Ex-9.18 | 7.65 |
| Ex-9.19 | 7.96 |
| Ex-9.20 | 7.53 |
| Ex-9.21 | 7.58 |
| Ex-9.22 | 6.89 |
| Ex-9.23 | 7.72 |
| Ex-9.24 | 8.18 |
| Ex-10.1 | 7.98 |
| Ex-10.2 | 7.33 |
| Ex-10.3 | 6.41 |
| Ex-10.4 | 7.41 |
| Ex-10.5 | 7.21 |
| Ex-10.6 | 7.96 |
| Ex-10.7 | 7.35 |
| Ex-10.8 | 6.84 |
| Ex-10.9 | 8.14 |
| Ex-10.10 | 7.81 |
| Ex-11.1 | 10.09 |
| Ex-11.2 | 8.04 |
| Ex-11.3 | 10.09 |
| Ex-11.4 | 8.77 |
| Ex-11.5 | 6.73 |
| Ex-11.6 | 8.42 |
| Ex-11.7 | 9.55 |
| Ex-11.8 | 10.09 |
| Ex-12.1 | 7.38 |
| Ex-12.2 | 8.19 |
| Ex-12.3 | 8.93 |
| Ex-12.4 | 7.18 |
| Ex-12.5 | 8.54 |
| Ex-12.6 | 9.11 |
| Ex-12.7 | 7.66 |
| Ex-12.8 | 6.50 |
| Ex-12.9 | 6.85 |
| Ex-13.1 | 8.60 |
| Ex-13.2 | 7.41 |
| Ex-13.3 | 9.30 |
| Ex-13.4 | 7.66 |
| Ex-13.5 | 8.38 |
| Ex-14.1 | 8.23 |
| Ex-14.2 | 8.39 |
| Ex-14.3 | 9.23 |
| Ex-14.4 | 9.73 |
| Ex-14.5 | 8.80 |
| Ex-14.6 | 8.59 |
| Ex-15.1 | 8.49 |
| Ex-15.2 | 9.52 |
| Ex-15.3 | 7.32 |
| Ex-15.4 | 10.09 |
| Ex-15.5 | 7.67 |
| Ex-15.6 | 9.30 |
| Ex-15.7 | 8.91 |
| Ex-15.8 | 6.66 |
| Ex-15.9 | 8.62 |
| Ex-15.10 | 8.70 |
| Ex-15.11 | 7.15 |
| Ex-15.12 | 7.07 |
| Ex-15.13 | 8.57 |
| Ex-15.14 | 8.43 |
| Ex-15.15 | 7.23 |
| Ex-15.16 | 10.09 |
| Ex-16.1 | 10.09 |
| Ex-16.2 | 10.09 |
| Ex-16.3 | 7.98 |
| Ex-16.4 | 7.54 |
| Ex-16.5 | 10.09 |
| Ex-16.6 | 10.09 |
| Ex-16.7 | 10.09 |
| Ex-16.8 | 10.09 |
| Ex-16.9 | 9.88 |
| Ex-16.10 | 10.09 |
| Ex-16.11 | 9.02 |
| Ex-16.12 | 10.09 |
| Ex-16.13 | 10.09 |
| Ex-16.14 | 10.09 |
| Ex-17.1 | 7.72 |
| Ex-17.2 | 7.84 |
| Ex-18.1 | 8.64 |
| Ex-18.2 | 8.44 |
| Ex-18.3 | 7.62 |
| Ex-18.4 | 7.21 |
| Ex-18.5 | 8.24 |
| Ex-18.6 | 7.22 |
| Ex-18.7 | 8.34 |
| Ex-18.8 | 8.27 |
| Ex-18.9 | 7.45 |
| Ex-18.10 | 7.90 |
| Ex-18.11 | 8.02 |
| Ex-18.12 | 8.79 |
| Ex-18.13 | 8.13 |
| Ex-18.14 | 7.22 |
| Ex-18.15 | 9.20 |
| Ex-18.16 | 8.51 |
| Ex-19.1 | 7.97 |
| Ex-20.1 | 10.09 |
| Ex-20.2 | 10.09 |
| Ex-21.1 | 10.09 |
| Ex-22.1 | 8.35 |
| Ex-23.1 | 10.09 |
| Ex-23.2 | 10.09 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound having a structural Formula (I): 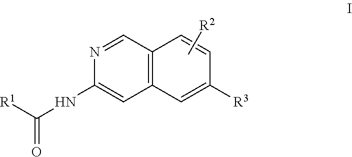
or a pharmaceutically acceptable salt thereof, wherein:
R¹ is selected from C₁₋₆ alkyl, —(R)-phenyl, monocyclic, spirocyclic or bicyclic —(R)—C₃₋₈ cycloalkyl, —(R)—C₃₋₁₀ heterocyclyl, and —(R)₀₋₃C₅₋₁₀ heteroaryl, said alkyl, phenyl, cycloalkyl, heterocyclyl and heteroaryl optionally substituted with 1 to 4 groups selected from R^{a};
R is a bond or straight or branched C₁₋₆ alkylenyl,
R² is selected from hydrogen, C₁₋₆ alkyl, and halogen;
R³ is selected from C₁₋₆ alkyl, spiropentanyl, cyclopropyl, cyclobutyl, cyclohexyl, and C-linked tetrahyropyranyl, C-linked piperidinyl, and C-linked oxabicycloheptanyl, said alkyl, spiropentanyl, cyclopropyl, cyclobutyl, cyclohexyl, tetrahyropyranyl, piperidinyl, and oxabicycloheptanyl are unsubstituted or substituted with 1 to 3 groups independently selected from 1 to 3 groups from R^{c}.
R^{a} is independently selected from the group consisting of H, halogen, —OH, CN, —OC₁₋₆ alkyl, C₁₋₆ alkyl, C₃₋₆ cycloalkyl, C(O)CF₃, C(O)C₁₋₆ alkyl, C₁₋₃ haloalkyl, C₁₋₃ haloalkoxy, NH(C₁₋₆alkyl), N(C₁₋₆alkyl)₂, —OC₁₋₃ haloalkyl, (CH₂)ₙC₅₋₆ heteroaryl, (CH₂)ₙC₃₋₁₀ heterocyclyl, —NH₂, said alkyl, heterocyclyl, and heteroaryl optionally substituted with 1 to 3 groups of R^{b};
R^{b} is independently selected from the group consisting of C₁₋₆ alkyl, —OC₁₋₆ alkyl, halogen, OH, CN, and C₁₋₃ haloalkyl;
R^{c} is independently selected from the group consisting of hydrogen, C₁₋₆ alkyl, OC₁₋₆ alkyl, C₃₋₆ cycloalkyl, CN, OH, halogen, azetidinyl, pyrrolidinyl, oxetanyl, tetrahydrofuranyl, tetrahydropyranyl, and dioxidothietanyl, wherein said alkyl, cycloalkyl, azetidinyl, pyrrolidinyl, oxetanyl, tetrahydrofuranyl, tetrahydropyranyl, and dioxidothietanyl are optionally substituted with 1 to 3 groups independently selected from C₁₋₆ alkyl, halogen, CN, OC₁₋₆ alkyl, C₃₋₆ cycloalkyl and OH;
and
n is selected from 0 to 3.

2. The compound according to claim 1 or a pharmaceutically acceptable salt thereof, wherein R¹ is selected from C₁₋₆ alkyl, —(R)-phenyl, —(R)-pyrazolyl, —(R)-oxazolyl, —(R)-pyridyl, cyclopropafuropyridyl, —(R)-cyclopropyl, —(R)-cyclobutyl, —(R)-cyclopentyl, —(R)-cyclohexyl, —(R)-bicyclopentanyl, —(R)-bicyclohexanyl, —(R)-spirohexanyl, —(R)-spiropentanyl, —(R)-spirooctanyl, —(R)-piperidinyl, —(R)-morpholinyl, —(R)-pyrrolidinyl, —(R)-azetidinyl, —(R)-oxaspirooctanyl, —(R)-oxabicyclohexanyl, —(R)-oxabicycloheptanyl, —(R)-oxaspiroheptanyl, —(R)-oxaspirononanyl, —(R)-tetrahydrofuranyl, and —(R)-tetrahydropyranyl said alkyl, —(R)-phenyl, —(R)-pyrazolyl, —(R)-oxazolyl, —(R)-pyridyl, —(R)-cyclopropafuropyridyl, —(R)-cyclopropyl, —(R)-cyclobutyl, —(R)-cyclopentyl, —(R)-cyclohexyl, —R)-bicyclopentanyl, —(R)-bicyclohexanyl, —(R)-spirohexanyl, —(R)-spiropentanyl, —(R)-spirooctanyl, —(R)-piperidinyl, —(R)-morpholinyl, —(R)-pyrrolidinyl, —(R)-azetidinyl, —(R)-oxaspirooctanyl, —(R)-oxabicyclohexanyl, —(R)-oxabicycloheptanyl, —(R)-oxaspiroheptanyl, —(R)-oxaspirononanyl, —(R)-tetrahydrofuranyl, and —(R)-tetrahydropyranyl unsubstituted or substituted with 1 to 3 groups selected from R^{a} and R is a bond or straight or branched C₁₋₆ alkylenyl.

3. The compound according to claim 1 or a pharmaceutically acceptable salt thereof, wherein R¹ is selected from —(R)pyrazolyl, —(R)oxazolyl, —(R)pyridyl, —(R)cyclopropyl, —(R)cyclobutyl, —(R)bicyclopentanyl, —(R)bicyclohexanyl, —(R)spirohexanyl, —(R)spiropentanyl, —(R)spirooctanyl, —(R)oxaspirooctanyl, —(R)oxabicyclohexanyl, —(R)oxabicycloheptanyl, —(R)oxaspiroheptanyl, —(R)tetrahydrofuranyl, and —(R)tetrahydropyranyl said —(R)-pyrazolyl, —(R)-oxazolyl, —(R)-pyridyl, —(R)-cyclopropyl, —(R)-cyclobutyl, —(R)-bicyclopentanyl, —(R)-bicyclohexanyl, —(R)-spirohexanyl, —(R)-spiropentanyl, —(R)-spirooctanyl, —(R)-oxaspirooctanyl, —(R)-oxabicycloheptanyl, —(R)-oxabicyclohexanyl, —(R)-oxaspiroheptanyl, —(R)-tetrahydrofuranyl, and —(R)-tetrahydropyranyl unsubstituted or substituted with 1 to 3 groups selected from R^{a} and R is a bond or straight or branched C₁₋₆ alkylenyl.

4. The compound according to claim 1 or a pharmaceutically acceptable salt thereof, wherein R¹ is selected from —(R)pyrazolyl, —(R)cyclopropyl, —(R)cyclobutyl, —(R)bicyclopentanyl, —(R)bicyclohexanyl, —(R)spirohexanyl, —(R)oxaspirooctanyl, —(R)oxabicycloheptanyl, —(R)oxaspiroheptanyl, and —(R)tetrahydropyranyl said —(R)-pyrazolyl, —(R)-cyclopropyl, —(R)-cyclobutyl, —(R)-bicyclopentanyl, —(R)-bicyclohexanyl, —(R)-spirohexanyl, —(R)-oxaspirooctanyl, —(R)oxabicycloheptanyl, —(R)oxaspiroheptanyl, and —(R)-tetrahydropyranyl unsubstituted or substituted with 1 to 3 groups of R^{a} independently selected from the group consisting of H, fluorine, chlorine, —OH, CN, CH₃, CH₂CH₃, CH₂CH(CH₃)₂, CH(CH₃)₂, C(CH₃)₂OCH₃, C(CH₃)₂OH, CH₂CH(OH)CF₃, C(O)CH₂CH₃, OCH₂CH₃, OCH₃, OCH(CH₃)₂, CH₂CF₃, CHF₂, C(CH₃)F, CF₃, OCF₂, OCF₃, C(O)CF₃, (CH₂)ₙpyrazolyl, and (CH₂)ₙpyridyl, said pyrazolyl and pyridyl unsubstituted or substituted with 1 to 3 groups of R^{b} and R is a bond or straight or branched C₁₋₆ alkylenyl.

5. The compound according to claim 1 or a pharmaceutically acceptable salt thereof, wherein R² is selected from the group consisting of hydrogen, chlorine, fluorine, and methyl.

6. The compound according to claim 1 or a pharmaceutically acceptable salt thereof, wherein R³ is selected from the group consisting of C₁₋₆ alkyl, —CH(CH₃)CH₂—OC₁₋₆ alkyl, —CH(CH₃)CH₂—CN, spiropentanyl, cyclopropyl, cyclobutyl, cyclohexyl, C-linked tetrahydropyranyl, C-linked piperidinyl, and C-linked oxabicycloheptanyl, said alkyl, spiropentanyl, cyclopropyl, cyclobutyl, cyclohexyl, C-linked tetrahydropyranyl, C-linked piperidinyl, and C-linked oxabicycloheptanyl unsubstituted or substituted with 1 to 3 groups independently selected from 1 to 3 groups from R^{c}.

7. The compound according to claim 1 or a pharmaceutically acceptable salt thereof, wherein R³ is unsubstituted or substituted C₁₋₆ alkyl.

8. The compound according to claim 1 or a pharmaceutically acceptable salt thereof, wherein R³ is unsubstituted or substituted spiropentanyl.

9. The compound according to claim 1 or a pharmaceutically acceptable salt thereof, wherein R³ is selected from the group consisting of unsubstituted or substituted cyclopropyl, cyclobutyl, and cyclohexyl.

10. The compound according to claim 1 or a pharmaceutically acceptable salt thereof, wherein R³ is unsubstituted or substituted cyclohexyl.

11. The compound according to claim 1 or a pharmaceutically acceptable salt thereof, wherein R³ is unsubstituted or substituted piperidinyl.

12. The compound according to Formula I of claim 1 or a pharmaceutically acceptable salt thereof represented by structural Formula II: 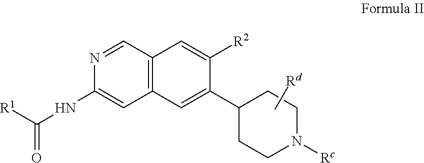
or a pharmaceutically acceptable salt thereof, wherein R¹, R² and R^{c} are as provided in Formula I and R^{d} is hydrogen or halogen.

13. The compound according to claim 12 wherein R^{d} is hydrogen, R² is selected from the group consisting of hydrogen, chlorine, and CH₃, and R^{c} is selected from the group consisting of oxetanyl, tetrahydrofuranyl, tetrahydropyranyl and cyclopentyl, said oxetanyl, tetrahydrofuranyl, tetrahydropyranyl and cyclopentyl unsubstituted or substituted with 1 to 3 groups selected from halogen, C₁₋₆ alkyl, —OC₁₋₆ alky, and OH.

14. The compound according to claim 12 or a pharmaceutically acceptable salt thereof, wherein R¹ is selected from the group consisting of C₁₋₆ alkyl, —(R)phenyl, —(R)pyrazolyl, —(R)oxazolyl, —(R)pyridyl, and cyclopropafuropyridyl —(R)cyclopropyl, —(R)cyclobutyl, —(R)cyclopentyl, —(R)cyclohexyl, —(R)bicyclopentanyl, —(R)bicyclohexanyl, —(R)spirohexanyl, —(R)spiropentanyl, —(R)spirooctanyl, —(R)piperidinyl, —(R)morpholinyl, —(R)pyrrolidinyl, —(R)azetidinyl, —(R)oxaspirooctanyl, —(R)oxabicyclohexanyl, —(R)oxabicycloheptanyl, —(R)oxaspiroheptanyl, —(R)oxaspirononanyl, —(R)tetrahydrofuranyl, and —(R)tetrahydropyranyl said alkyl, —(R)-phenyl, —(R)-pyrazolyl, —(R)-oxazolyl, —(R)-pyridyl, —(R)-cyclopropafuropyridyl, —(R)-cyclopropyl, —(R)-cyclobutyl, —(R)-cyclopentyl, —(R)-cyclohexyl, —(R)-bicyclopentanyl, —(R)-bicyclohexanyl, —(R)-oxabicycloheptanyl, —(R)oxaspiroheptanyl, —(R)-oxaspirononanyl, —(R)-tetrahydrofuranyl, and tetrahydropyranyl unsubstituted or substituted with 1 to 3 groups selected from R^{a} and R is a bond or straight or branched C₁₋₆ alkylenyl.

15. The compound according to Formula I of claim 1 or a pharmaceutically acceptable salt thereof represented by structural Formula III: 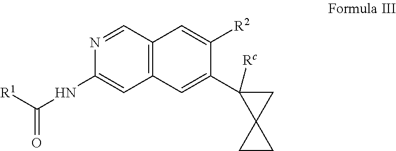
or a pharmaceutically acceptable salt thereof, wherein R¹, R² and R^{c} are as provided in Formula I.

16. The compound according to claim 15 or a pharmaceutically acceptable salt thereof, wherein R² is selected from the group consisting of hydrogen, chlorine, and CH₃, R^{c} is selected from the group consisting of hydrogen, CN, halogen, and C₁₋₆ alkyl.

17. The compound according to claim 15 or a pharmaceutically acceptable salt thereof, wherein R¹ is selected from the group consisting of C₁₋₆ alkyl, —(R)₀₋₁phenyl, —(R)₀₋₁pyrazolyl, —(R)₀₋₁oxazolyl, —(R)₀₋₁pyridyl, and cyclopropafuropyridyl —(R)₀₋₁cyclopropyl, —(R)₀₋₁cyclobutyl, —(R)₀₋₁cyclopentyl, —(R)₀₋₁cyclohexyl, —(R)₀₋₁bicyclopentanyl, —(R)₀₋₁bicyclohexanyl, —(R)₀₋₁spirohexanyl, —(R)₀₋₁spiropentanyl, —(R)₀₋₁spirooctanyl, —(R)₀₋₁piperidinyl, —(R)₀₋₁morpholinyl, —(R)₀₋₁pyrrolidinyl, —(R)₀₋₁azetidinyl, —(R)₀₋₁oxaspirooctanyl, —(R)₀₋₁oxabicyclohexanyl, —(R)₀₋₁oxabicycloheptanyl, —(R)₀₋₁oxaspiroheptanyl, —(R)₀₋₁oxaspirononanyl, —(R)₀₋₁tetrahydrofuranyl, and —(R)₀₋₁tetrahydropyranyl said alkyl, —(R)-phenyl, —(R)-pyrazolyl, —(R)-oxazolyl, —(R)pyridyl, —(R)-cyclopropafuropyridyl, —(R)-cyclopropyl, —(R)-cyclobutyl, —(R)-cyclopentyl, —(R)-cyclohexyl, —(R)-bicyclopentanyl, —(R)-bicyclohexanyl, —(R)-oxabicycloheptanyl, —(R)-oxaspiroheptanyl, —(R)-oxaspirononanyl, —(R)-tetrahydrofuranyl, and tetrahydropyranyl unsubstituted or substituted with 1 to 3 groups selected from R^{a} and R is a bond or straight or branched C₁₋₆ alkylenyl.

18. The compound according to Formula I of claim 1 or a pharmaceutically acceptable salt thereof represented by structural Formula IV: 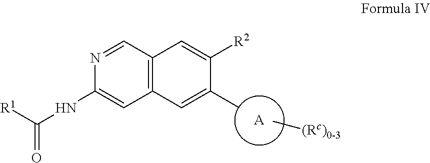
or a pharmaceutically acceptable salt thereof, wherein R¹, R² and R^{c} are as provided in Formula I and A is selected from the group consisting of cyclohexyl, cyclopropyl, cyclobutyl, oxabicycloheptanyl and pyranyl.

19. A compound or a pharmaceutically acceptable salt thereof selected from the group consisting of, (1R,3R)-N-(6-(1-cyanospiro[2.2]pentan-1-yl)isoquinolin-3-yl)-3-methoxycyclobutane-1-carboxamide,
N-(6-(1-cyanospiro[2.2]pentan-1-yl)isoquinolin-3-yl)-3-methoxycyclobutane-1-carboxamide,
(1R,3R)-N-(6-((R)-1-cyanospiro[2.2]pentan-1-yl)isoquinolin-3-yl)-3-methoxycyclobutane-1-carboxamide, (1R,3R)-N-(6-((S)-1-cyanospiro[2.2]pentan-1-yl)isoquinolin-3-yl)-3-methoxycyclobutane-1-carboxamide,
(1S,3S)-N-(6-((R)-1-cyanospiro[2.2]pentan-1-yl)isoquinolin-3-yl)-3-methoxycyclobutane-1-carboxamide,
(1S,3S)-N-(6-((S)-1-cyanospiro[2.2]pentan-1-yl)isoquinolin-3-yl)-3-methoxycyclobutane-1-carboxamide,
N-(7-chloro-6-(1-(oxetan-3-yl)piperidin-4-yl)isoquinolin-3-yl)-2,2-difluorocyclopropane-1-carboxamide,
(R)-N-(7-chloro-6-(1-(oxetan-3-yl)piperidin-4-yl)isoquinolin-3-yl)-2,2-difluorocyclopropane-1-carboxamide,
(S)-N-(7-chloro-6-(1-(oxetan-3-yl)piperidin-4-yl)isoquinolin-3-yl)-2,2-difluorocyclopropane-1-carboxamide,
N-(7-chloro-6-(1-(oxetan-3-yl)piperidin-4-yl)isoquinolin-3-yl)-2,2-difluoro-3-methylcyclopropane-1-carboxamide,
(1R,3R)-N-(7-chloro-6-(1-(oxetan-3-yl)piperidin-4-yl)isoquinolin-3-yl)-2,2-difluoro-3-methylcyclopropane-1-carboxamide,
(1R,3S)-N-(7-chloro-6-(1-(oxetan-3-yl)piperidin-4-yl)isoquinolin-3-yl)-2,2-difluoro-3-methylcyclopropane-1-carboxamide,
(1S,3R)-N-(7-chloro-6-(1-(oxetan-3-yl)piperidin-4-yl)isoquinolin-3-yl)-2,2-difluoro-3-methylcyclopropane-1-carboxamide,
(1S,3S)-N-(7-chloro-6-(1-(oxetan-3-yl)piperidin-4-yl)isoquinolin-3-yl)-2,2-difluoro-3-methylcyclopropane-1-carboxamide,
trans-N-(7-chloro-6-(1-(oxetan-3-yl)piperidin-4-yl)isoquinolin-3-yl)-2-fluorocyclopropane-1-carboxamide,
(1R,3S)-N-(7-chloro-6-(1-(oxetan-3-yl)piperidin-4-yl)isoquinolin-3-yl)-2-fluorocyclopropane-1-carboxamide,
(1S,3R)-N-(7-chloro-6-(1-(oxetan-3-yl)piperidin-4-yl)isoquinolin-3-yl)-2-fluorocyclopropane-1-carboxamide,
N-(7-chloro-6-(1-(oxetan-3-yl)piperidin-4-yl)isoquinolin-3-yl)-6,6-difluorospiro[2.5]octane-1-carboxamide,
(R)-N-(7-chloro-6-(1-(oxetan-3-yl)piperidin-4-yl)isoquinolin-3-yl)-6,6-difluorospiro[2.5]octane-1-carboxamide,
(S)-N-(7-chloro-6-(1-(oxetan-3-yl)piperidin-4-yl)isoquinolin-3-yl)-6,6-difluorospiro[2.5]octane-1-carboxamide,
Rac-N-(7-chloro-6-(1-(oxetan-3-yl)piperidin-4-yl)isoquinolin-3-yl)-6-oxaspiro[2.5]octane-1-carboxamide,
(S)-N-(7-chloro-6-(1-(oxetan-3-yl)piperidin-4-yl)isoquinolin-3-yl)tetrahydro-2H-pyran-2-carboxamide,
(1R,2S)-N-(7-chloro-6-(1-(oxetan-3-yl)piperidin-4-yl)isoquinolin-3-yl)-4,4-difluoro-2-methylcyclohexane-1-carboxamide,
(1R,3R,4R)-N-(7-chloro-6-(1-(oxetan-3-yl)piperidin-4-yl)isoquinolin-3-yl)-3-fluoro-4-hydroxycyclopentane-1-carboxamide,
Cis-N-(7-chloro-6-(1-(oxetan-3-yl)piperidin-4-yl)isoquinolin-3-yl)-2-fluorocyclopropane-1-carboxamide,
(1R,2R)-N-(7-chloro-6-(1-(oxetan-3-yl)piperidin-4-yl)isoquinolin-3-yl)-2-fluorocyclopropane-1-carboxamide,
N-(7-chloro-6-(1-(oxetan-3-yl)piperidin-4-yl)isoquinolin-3-yl)spiro[2.3]hexane-1-carboxamide,
N-(6-(1-cyanospiro[2.2]pentan-1-yl)isoquinolin-3-yl)cyclobutanecarboxamide,
(R)-N-(6-(1-cyanospiro[2.2]pentan-1-yl)isoquinolin-3-yl)cyclobutanecarboxamide,
(S)-N-(6-(1-cyanospiro[2.2]pentan-1-yl)isoquinolin-3-yl)cyclobutanecarboxamide,
Rac-N-(7-chloro-6-(1-(3-methyloxetan-3-yl)piperidin-4-yl)isoquinolin-3-yl)-3-methylcyclobutane-1-carboxamide,
(2S)-N-(7-chloro-6-(1-(4-hydroxy-3-methyltetrahydrofuran-3-yl)piperidin-4-yl)isoquinolin-3-yl)tetrahydro-2H-pyran-2-carboxamide,
(S)-N-(7-chloro-6-(1-((3R,4R)-4-hydroxy-3-methyltetrahydrofuran-3-yl)piperidin-4-yl)isoquinolin-3-yl)tetrahydro-2H-pyran-2-carboxamide,
(S)-N-(7-chloro-6-(1-((3S,4S)-4-hydroxy-3-methyltetrahydrofuran-3-yl)piperidin-4-yl)isoquinolin-3-yl)tetrahydro-2H-pyran-2-carboxamide,
N-(7-chloro-6-(1-(4-hydroxy-3-methyltetrahydrofuran-3-yl)piperidin-4-yl)isoquinolin-3-yl)-3,3-difluorocyclobutane-1-carboxamide,
N-(7-chloro-6-(1-((3R,4R)-4-hydroxy-3-methyltetrahydrofuran-3-yl)piperidin-4-yl)isoquinolin-3-yl)-3,3-difluorocyclobutane-1-carboxamide,
N-(7-chloro-6-(1-((3S,4S)-4-hydroxy-3-methyltetrahydrofuran-3-yl)piperidin-4-yl)isoquinolin-3-yl)-3,3-difluorocyclobutane-1-carboxamide,
N-(7-chloro-6-(1-(4-hydroxy-3-methyltetrahydrofuran-3-yl)piperidin-4-yl)isoquinolin-3-yl)-1-methylpiperidine-4-carboxamide,
N-(7-chloro-6-(1-((3R,4R)-4-hydroxy-3-methyltetrahydrofuran-3-yl)piperidin-4-yl)isoquinolin-3-yl)-1-methylpiperidine-4-carboxamide,
N-(7-chloro-6-(1-((3S,4S)-4-hydroxy-3-methyltetrahydrofuran-3-yl)piperidin-4-yl)isoquinolin-3-yl)-1-methylpiperidine-4-carboxamide,
N-(7-chloro-6-(1-(4-hydroxy-3-methyltetrahydrofuran-3-yl)piperidin-4-yl)isoquinolin-3-yl)bicyclo[3.1.0]hexane-6-carboxamide,
N-(7-chloro-6-(1-((3R,4R)-4-hydroxy-3-methyltetrahydrofuran-3-yl)piperidin-4-yl)isoquinolin-3-yl)bicyclo[3.1.0]hexane-6-carboxamide,
N-(7-chloro-6-(1-((3S,4S)-4-hydroxy-3-methyltetrahydrofuran-3-yl)piperidin-4-yl)isoquinolin-3-yl)bicyclo[3.1.0]hexane-6-carboxamide,
N-(7-chloro-6-(1-(4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(2-hydroxypropan-2-yl)cyclopropane-1-carboxamide,
Rac-N-(7-chloro-6-(1-(4-hydroxy-3-methyltetrahydrofuran-3-yl)piperidin-4-yl)isoquinolin-3-yl)-3-isopropylcyclobutane-1-carboxamide,
N-(6-(1-cyanospiro[2.2]pentan-1-yl)isoquinolin-3-yl)-2-(1-methyl-1H-pyrazol-4-yl)cyclopropane-1-carboxamide, (1R,2R)-N-(6-((R)-1-cyanospiro[2.2]pentan-1-yl)isoquinolin-3-yl)-2-(1-methyl-1H-pyrazol-4-yl)cyclopropane-1-carboxamide,
(1R,2R)-N-(6-((S)-1-cyanospiro[2.2]pentan-1-yl)isoquinolin-3-yl)-2-(1-methyl-1H-pyrazol-4-yl)cyclopropane-1-carboxamide,
(1S,2S)-N-(6-((R)-1-cyanospiro[2.2]pentan-1-yl)isoquinolin-3-yl)-2-(1-methyl-1H-pyrazol-4-yl)cyclopropane-1-carboxamide,
(1S,2S)-N-(6-((S)-1-cyanospiro[2.2]pentan-1-yl)isoquinolin-3-yl)-2-(1-methyl-1H-pyrazol-4-yl)cyclopropane-1-carboxamide,
N-(6-(1-cyanospiro[2.2]pentan-1-yl)isoquinolin-3-yl)-2-ethyl-3-(1-methyl-1H-pyrazol-4-yl)cyclopropane-1-carboxamide,
(1R,3R)-N-(6-((R)-1-cyanospiro[2.2]pentan-1-yl)isoquinolin-3-yl)-2-ethyl-3-(1-methyl-1H-pyrazol-4-yl)cyclopropane-1-carboxamide,
(1R,3R)-N-(6-((S)-1-cyanospiro[2.2]pentan-1-yl)isoquinolin-3-yl)-2-ethyl-3-(1-methyl-1H-pyrazol-4-yl)cyclopropane-1-carboxamide,
(1S,3S)-N-(6-((R)-1-cyanospiro[2.2]pentan-1-yl)isoquinolin-3-yl)-2-ethyl-3-(1-methyl-1H-pyrazol-4-yl)cyclopropane-1-carboxamide,
(1S,3S)-N-(6-((S)-1-cyanospiro[2.2]pentan-1-yl)isoquinolin-3-yl)-2-ethyl-3-(1-methyl-1H-pyrazol-4-yl)cyclopropane-1-carboxamide,
N-(6-(1-cyanospiro[2.2]pentan-1-yl)isoquinolin-3-yl)-3-(2-hydroxypropan-2-yl)cyclobutane-1-carboxamide,
(1R,3R)-N-(6-((R)-1-cyanospiro[2.2]pentan-1-yl)isoquinolin-3-yl)-3-(2-hydroxypropan-2-yl)cyclobutane-1-carboxamide,
(1R,3R)-N-(6-((S)-1-cyanospiro[2.2]pentan-1-yl)isoquinolin-3-yl)-3-(2-hydroxypropan-2-yl)cyclobutane-1-carboxamide,
(1S,3S)-N-(6-((R)-1-cyanospiro[2.2]pentan-1-yl)isoquinolin-3-yl)-3-(2-hydroxypropan-2-yl)cyclobutane-1-carboxamide,
(1S,3S)-N-(6-((S)-1-cyanospiro[2.2]pentan-1-yl)isoquinolin-3-yl)-3-(2-hydroxypropan-2-yl)cyclobutane-1-carboxamide,
N-(6-(1-cyanospiro[2.2]pentan-1-yl)isoquinolin-3-yl)-2,2,6,6-tetramethyltetrahydro-2H-pyran-4-carboxamide,
N-(6-((R)-1-cyanospiro[2.2]pentan-1-yl)isoquinolin-3-yl)-2,2,6,6-tetramethyltetrahydro-2H-pyran-4-carboxamide,
N-(6-((S)-1-cyanospiro[2.2]pentan-1-yl)isoquinolin-3-yl)-2,2,6,6-tetramethyltetrahydro-2H-pyran-4-carboxamide,
N-(6-(1-cyanospiro[2.2]pentan-1-yl)isoquinolin-3-yl)-5-isopropoxytetrahydro-2H-pyran-2-carboxamide,
N-(6-((R)-1-cyanospiro[2.2]pentan-1-yl)isoquinolin-3-yl)-5R-isopropoxytetrahydro-2H-pyran-2R-carboxamide,
N-(6-((S)-1-cyanospiro[2.2]pentan-1-yl)isoquinolin-3-yl)-5R-isopropoxytetrahydro-2H-pyran-2R-carboxamide,
N-(6-((R)-1-cyanospiro[2.2]pentan-1-yl)isoquinolin-3-yl)-5S-isopropoxytetrahydro-2H-pyran-2R-carboxamide,
N-(6-((S)-1-cyanospiro[2.2]pentan-1-yl)isoquinolin-3-yl)-5S-isopropoxytetrahydro-2H-pyran-2R-carboxamide,
N-(6-((R)-1-cyanospiro[2.2]pentan-1-yl)isoquinolin-3-yl)-5R-isopropoxytetrahydro-2H-pyran-2S-carboxamide,
N-(6-((S)-1-cyanospiro[2.2]pentan-1-yl)isoquinolin-3-yl)-5R-isopropoxytetrahydro-2H-pyran-2S-carboxamide,
N-(6-((R)-1-cyanospiro[2.2]pentan-1-yl)isoquinolin-3-yl)-5S-isopropoxytetrahydro-2H-pyran-2S-carboxamide,
N-(6-((R)-1-cyanospiro[2.2]pentan-1-yl)isoquinolin-3-yl)-5S-isopropoxytetrahydro-2H-pyran-2S-carboxamide,
(1R,3R,4R)-N-(6-(1-cyanospiro[2.2]pentan-1-yl)isoquinolin-3-yl)-3-fluoro-4-hydroxycyclopentane-1-carboxamide,
(1R,3R,4R)-N-(6-((R)-1-cyanospiro[2.2]pentan-1-yl)isoquinolin-3-yl)-3-fluoro-4-hydroxycyclopentane-1-carboxamide,
(1R,3R,4R)-N-(6-((S)-1-cyanospiro[2.2]pentan-1-yl)isoquinolin-3-yl)-3-fluoro-4-hydroxycyclopentane-1-carboxamide,
N-(6-(1-cyanospiro[2.2]pentan-1-yl)isoquinolin-3-yl)-1-(2,2,2-trifluoroacetyl)piperidine-4-carboxamide,
N-(6-((R)-1-cyanospiro[2.2]pentan-1-yl)isoquinolin-3-yl)-1-(2,2,2-trifluoroacetyl)piperidine-4-carboxamide,
N-(6-((S)-1-cyanospiro[2.2]pentan-1-yl)isoquinolin-3-yl)-1-(2,2,2-trifluoroacetyl)piperidine-4-carboxamide,
(2R)-N-(6-(1-cyanospiro[2.2]pentan-1-yl)isoquinolin-3-yl)-4-methylmorpholine-2-carboxamide,
(2R)-N-(6-((R)-1-cyanospiro[2.2]pentan-1-yl)isoquinolin-3-yl)-4-methylmorpholine-2-carboxamide,
(2R)-N-(6-((S)-1-cyanospiro[2.2]pentan-1-yl)isoquinolin-3-yl)-4-methylmorpholine-2-carboxamide,
N-(6-(1-cyanospiro[2.2]pentan-1-yl)isoquinolin-3-yl)-1-propionylpiperidine-4-carboxamide,
N-(6-((R)-1-cyanospiro[2.2]pentan-1-yl)isoquinolin-3-yl)-1-propionylpiperidine-4-carboxamide,
N-(6-((S)-1-cyanospiro[2.2]pentan-1-yl)isoquinolin-3-yl)-1-propionylpiperidine-4-carboxamide,
4-chloro-N-(6-(1-cyanospiro[2.2]pentan-1-yl)isoquinolin-3-yl)-1a,6b-dihydro-1H-cyclopropa[4,5]furo[3,2-c]pyridine-1-carboxamide,
(1S,1aR,6bS)-4-chloro-N-(6-((R)-1-cyanospiro[2.2]pentan-1-yl)isoquinolin-3-yl)-1a,6b-dihydro-1H-cyclopropa[4,5]furo[3,2-c]pyridine-1-carboxamide,
(1R,1aS,6bR)-4-chloro-N-(6-((R)-1-cyanospiro[2.2]pentan-1-yl)isoquinolin-3-yl)-1a,6b-dihydro-1H-cyclopropa[4,5]furo[3,2-c]pyridine-1-carboxamide,
(1S,1aR,6bS)-4-chloro-N-(6-((S)-1-cyanospiro[2.2]pentan-1-yl)isoquinolin-3-yl)-1a,6b-dihydro-1H-cyclopropa[4,5]furo[3,2-c]pyridine-1-carboxamide,
(1R,1aS,6bR)-4-chloro-N-(6-((S)-1-cyanospiro[2.2]pentan-1-yl)isoquinolin-3-yl)-1a,6b-dihydro-1H-cyclopropa[4,5]furo[3,2-c]pyridine-1-carboxamide,
N-(7-chloro-6-(1-(4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1R,2R,3R)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1R,2S,3R)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1S,2R,3S)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1S,2S,3S)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1R,2R,3R)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1R,2S,3R)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1S,2R,3S)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(1S,2S,3S)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-4-yl)cyclopropane-1-carboxamide,
(R)-N-(7-chloro-6-(1-(oxetan-3-yl)piperidin-4-yl)isoquinolin-3-yl)-4,4,4-trifluoro-3-hydroxybutanamide,
N-(7-chloro-6-(1-(oxetan-3-yl)piperidin-4-yl)isoquinolin-3-yl)-1-methyl-1H-pyrazole-4-carboxamide,
N-(7-chloro-6-(1-(oxetan-3-yl)piperidin-4-yl)isoquinolin-3-yl)-2-methyloxazole-4-carboxamide,
N-(7-chloro-6-(1-(oxetan-3-yl)piperidin-4-yl)isoquinolin-3-yl)oxazole-4-carboxamide,
N-(7-chloro-6-(1-(4-hydroxy-3-methyltetrahydrofuran-3-yl)piperidin-4-yl)isoquinolin-3-yl)-2-(tetrahydro-2H-pyran-4-yl)acetamide,
N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)acetamide,
N-(7-chloro-6-(1-((3S,4S)-4-hydroxy-3-methyltetrahydrofuran-3-yl)piperidin-4-yl)isoquinolin-3-yl)-2-(tetrahydro-2H-pyran-4-yl)acetamide,
N-(7-chloro-6-(1-(4-hydroxy-3-methyltetrahydrofuran-3-yl)piperidin-4-yl)isoquinolin-3-yl)-2-(1,3-dimethyl-1H-pyrazol-4-yl)acetamide,
N-(7-chloro-6-(1-((3R,4R)-4-hydroxy-3-methyltetrahydrofuran-3-yl)piperidin-4-yl)isoquinolin-3-yl)-2-(1,3-dimethyl-1H-pyrazol-4-yl)acetamide,
N-(7-chloro-6-(1-((3S,4S)-4-hydroxy-3-methyltetrahydrofuran-3-yl)piperidin-4-yl)isoquinolin-3-yl)-2-(1,3-dimethyl-1H-pyrazol-4-yl)acetamide,
N-(6-(1-cyanospiro[2.2]pentan-1-yl)isoquinolin-3-yl)-2-(pyridin-3-yl)acetamide,
(R)-N-(6-(1-cyanospiro[2.2]pentan-1-yl)isoquinolin-3-yl)-2-(pyridin-3-yl)acetamide,
(S)-N-(6-(1-cyanospiro[2.2]pentan-1-yl)isoquinolin-3-yl)-2-(pyridin-3-yl)acetamide,
N-(6-(1-cyanospiro[2.2]pentan-1-yl)isoquinolin-3-yl)-2-(pyridin-3-yl)acetamide,
(R)-N-(6-(1-cyanospiro[2.2]pentan-1-yl)isoquinolin-3-yl)-2-(pyridin-3-yl)acetamide,
(S)-N-(6-(1-cyanospiro[2.2]pentan-1-yl)isoquinolin-3-yl)-2-(pyridin-3-yl)acetamide,
N-(6-(1-cyanospiro[2.2]pentan-1-yl)isoquinolin-3-yl)-2-(1-(difluoromethyl)-1H-pyrazol-4-yl)acetamide,
(R)-N-(6-(1-cyanospiro[2.2]pentan-1-yl)isoquinolin-3-yl)-2-(1-(difluoromethyl)-1H-pyrazol-4-yl)acetamide,
(S)N-(6-(1-cyanospiro[2.2]pentan-1-yl)isoquinolin-3-yl)-2-(1-(difluoromethyl)-1H-pyrazol-4-yl)acetamide,
N-(6-(1-cyanospiro[2.2]pentan-1-yl)isoquinolin-3-yl)-2-(4-(2-hydroxypropan-2-yl)phenyl)acetamide,
(R)-N-(6-(1-cyanospiro[2.2]pentan-1-yl)isoquinolin-3-yl)-2-(4-(2-hydroxypropan-2-yl)phenyl)acetamide,
(S)-N-(6-(1-cyanospiro[2.2]pentan-1-yl)isoquinolin-3-yl)-2-(4-(2-hydroxypropan-2-yl)phenyl)acetamide, 2,2,2-trifluoroacetate salt
N-(6-(1-cyanospiro[2.2]pentan-1-yl)isoquinolin-3-yl)-2-(3-(2-hydroxypropan-2-yl)phenyl)acetamide,
(R)-N-(6-(1-cyanospiro[2.2]pentan-1-yl)isoquinolin-3-yl)-2-(3-(2-hydroxypropan-2-yl)phenyl)acetamide,
(S)-N-(6-(1-cyanospiro[2.2]pentan-1-yl)isoquinolin-3-yl)-2-(3-(2-hydroxypropan-2-yl)phenyl)acetamide,
N-(6-(1-cyanospiro[2.2]pentan-1-yl)isoquinolin-3-yl)-2-(4-isopropylmorpholin-2-yl)acetamide,
(R)-N-(6-(1-cyanospiro[2.2]pentan-1-yl)isoquinolin-3-yl)-2-(4-isopropylmorpholin-2-yl)acetamide,
(S)-N-(6-(1-cyanospiro[2.2]pentan-1-yl)isoquinolin-3-yl)-2-(4-isopropylmorpholin-2-yl)acetamide,
N-(6-(1-cyanospiro[2.2]pentan-1-yl)isoquinolin-3-yl)-2-(4-methylmorpholin-2-yl)acetamide,
(R)-N-(6-(1-cyanospiro[2.2]pentan-1-yl)isoquinolin-3-yl)-2-(4-methylmorpholin-2-yl)acetamide,
(S)-N-(6-(1-cyanospiro[2.2]pentan-1-yl)isoquinolin-3-yl)-2-(4-methylmorpholin-2-yl)acetamide,
N-(7-chloro-6-(1-(4-hydroxy-3-methyltetrahydrofuran-3-yl)piperidin-4-yl)isoquinolin-3-yl)-1-cyclopropyl-1H-pyrazole-4-carboxamide,
N-(7-chloro-6-(1-((3R,4R)-4-hydroxy-3-methyltetrahydrofuran-3-yl)piperidin-4-yl)isoquinolin-3-yl)-1-cyclopropyl-1H-pyrazole-4-carboxamide,
N-(7-chloro-6-(1-((3S,4S)-4-hydroxy-3-methyltetrahydrofuran-3-yl)piperidin-4-yl)isoquinolin-3-yl)-1-cyclopropyl-1H-pyrazole-4-carboxamide,
N-(7-chloro-6-(1-(4-hydroxy-3-methyltetrahydrofuran-3-yl)piperidin-4-yl)isoquinolin-3-yl)-1-cyclopropyl-1H-pyrazole-3-carboxamide,
N-(7-chloro-6-(1-((3R,4R)-4-hydroxy-3-methyltetrahydrofuran-3-yl)piperidin-4-yl)isoquinolin-3-yl)-1-cyclopropyl-1H-pyrazole-3-carboxamide,
N-(7-chloro-6-(1-((3S,4S)-4-hydroxy-3-methyltetrahydrofuran-3-yl)piperidin-4-yl)isoquinolin-3-yl)-1-cyclopropyl-1H-pyrazole-3-carboxamide,
5-chloro-N-(7-chloro-6-(1-(4-hydroxy-3-methyltetrahydrofuran-3-yl)piperidin-4-yl)isoquinolin-3-yl)-1-cyclopropyl-1H-pyrazole-4-carboxamide,
5-chloro-N-(7-chloro-6-(1-((3R,4R)-4-hydroxy-3-methyltetrahydrofuran-3-yl)piperidin-4-yl)isoquinolin-3-yl)-1-cyclopropyl-1H-pyrazole-4-carboxamide,
5-chloro-N-(7-chloro-6-(1-((3S,4S)-4-hydroxy-3-methyltetrahydrofuran-3-yl)piperidin-4-yl)isoquinolin-3-yl)-1-cyclopropyl-1H-pyrazole-4-carboxamide,
5-chloro-N-(7-chloro-6-(1-(4-fluoro-3-methyltetrahydrofuran-3-yl)piperidin-4-yl)isoquinolin-3-yl)-1-cyclopropyl-1H-pyrazole-4-carboxamide,
5-chloro-N-(7-chloro-6-(1-((3R,4R)-4-fluoro-3-methyltetrahydrofuran-3-yl)piperidin-4-yl)isoquinolin-3-yl)-1-cyclopropyl-1H-pyrazole-4-carboxamide,
5-chloro-N-(7-chloro-6-(1-((3S,4S)-4-fluoro-3-methyltetrahydrofuran-3-yl)piperidin-4-yl)isoquinolin-3-yl)-1-cyclopropyl-1H-pyrazole-4-carboxamide,
1-(bicyclo[1.1.1]pentan-1-yl)-N-(7-chloro-6-(1-(4-hydroxy-3-methyltetrahydrofuran-3-yl)piperidin-4-yl)isoquinolin-3-yl)-1H-pyrazole-4-carboxamide,
1-(bicyclo[1.1.1]pentan-1-yl)-N-(7-chloro-6-(1-((3R,4R)-4-hydroxy-3-methyltetrahydrofuran-3-yl)piperidin-4-yl)isoquinolin-3-yl)-1H-pyrazole-4-carboxamide,
1-(bicyclo[1.1.1]pentan-1-yl)-N-(7-chloro-6-(1-((3S,4S)-4-hydroxy-3-methyltetrahydrofuran-3-yl)piperidin-4-yl)isoquinolin-3-yl)-1H-pyrazole-4-carboxamide,
1-(bicyclo[1.1.1]pentan-1-yl)-N-(7-chloro-6-(1-(4-fluoro-3-methyltetrahydrofuran-3-yl)piperidin-4-yl)isoquinolin-3-yl)-1H-pyrazole-4-carboxamide,
1-(bicyclo[1.1.1]pentan-1-yl)-N-(7-chloro-6-(1-((3R,4R)-4-fluoro-3-methyltetrahydrofuran-3-yl)piperidin-4-yl)isoquinolin-3-yl)-1H-pyrazole-4-carboxamide,
1-(bicyclo[1.1.1]pentan-1-yl)-N-(7-chloro-6-(1-((3S,4S)-4-fluoro-3-methyltetrahydrofuran-3-yl)piperidin-4-yl)isoquinolin-3-yl)-1H-pyrazole-4-carboxamide,
(S,S)-, and/or (S,R)-, and/or (R,S)-, and/or (R,R)-N-(6-(1-cyanospiro[2.2]pentan-1-yl)isoquinolin-3-yl)spiro[2.2]pentane-1-carboxamide,
N-(7-chloro-6-(1-(oxetan-3-yl)piperidin-4-yl)isoquinolin-3-yl)-3-(difluoromethoxy)cyclobutane-1-carboxamide,
(1R,3R)-N-(7-chloro-6-(1-(oxetan-3-yl)piperidin-4-yl)isoquinolin-3-yl)-3-(difluoromethoxy)cyclobutane-1-carboxamide,
(1S,3S)-N-(7-chloro-6-(1-(oxetan-3-yl)piperidin-4-yl)isoquinolin-3-yl)-3-(difluoromethoxy)cyclobutane-1-carboxamide,
N-(6-(1-cyanospiro[2.2]pentan-1-yl)isoquinolin-3-yl)-3-(difluoromethoxy)cyclobutane-1-carboxamide,
(1R,3R)-N-(6-((R)1-cyanospiro[2.2]pentan-1-yl)isoquinolin-3-yl)-3-(difluoromethoxy)cyclobutane-1-carboxamide,
(1R,3R)-N-(6-((S)1-cyanospiro[2.2]pentan-1-yl)isoquinolin-3-yl)-3-(difluoromethoxy)cyclobutane-1-carboxamide,
(1S,3S)-N-(6-((R)1-cyanospiro[2.2]pentan-1-yl)isoquinolin-3-yl)-3-(difluoromethoxy)cyclobutane-1-carboxamide,
(1S,3S)-N-(6-((S)1-cyanospiro[2.2]pentan-1-yl)isoquinolin-3-yl)-3-(difluoromethoxy)cyclobutane-1-carboxamide,
N-(7-chloro-6-(1-(3-methyloxetan-3-yl)piperidin-4-yl)isoquinolin-3-yl)-2-cyanocyclopropane-1-carboxamide,
(1R,2R)-N-(7-chloro-6-(1-(3-methyloxetan-3-yl)piperidin-4-yl)isoquinolin-3-yl)-2-cyanocyclopropane-1-carboxamide,
(1R,2S)-N-(7-chloro-6-(1-(3-methyloxetan-3-yl)piperidin-4-yl)isoquinolin-3-yl)-2-cyanocyclopropane-1-carboxamide,
(1S,2R)-N-(7-chloro-6-(1-(3-methyloxetan-3-yl)piperidin-4-yl)isoquinolin-3-yl)-2-cyanocyclopropane-1-carboxamide,
(1S,2S)-N-(7-chloro-6-(1-(3-methyloxetan-3-yl)piperidin-4-yl)isoquinolin-3-yl)-2-cyanocyclopropane-1-carboxamide,
N-(7-chloro-6-(1-(4-hydroxy-3-methyltetrahydrofuran-3-yl)piperidin-4-yl)isoquinolin-3-yl)-2-ethoxycyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-ethoxycyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-ethoxycyclopropane-1-carboxamide,
(1R,2S)-N-(7-chloro-6-(1-((3R,4R)-4-hydroxy-3-methyltetrahydrofuran-3-yl)piperidin-4-yl)isoquinolin-3-yl)-2-ethoxycyclopropane-1-carboxamide,
(1R,2S)-N-(7-chloro-6-(1-((3S,4S)-4-hydroxy-3-methyltetrahydrofuran-3-yl)piperidin-4-yl)isoquinolin-3-yl)-2-ethoxycyclopropane-1-carboxamide,
(1S,2R)-N-(7-chloro-6-(1-((3R,4R)-4-hydroxy-3-methyltetrahydrofuran-3-yl)piperidin-4-yl)isoquinolin-3-yl)-2-ethoxycyclopropane-1-carboxamide,
(1S,2R)-N-(7-chloro-6-(1-((3S,4S)-4-hydroxy-3-methyltetrahydrofuran-3-yl)piperidin-4-yl)isoquinolin-3-yl)-2-ethoxycyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-ethoxycyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-ethoxycyclopropane-1-carboxamide,
N-(7-chloro-6-(1-(4-hydroxy-3-methyltetrahydrofuran-3-yl)piperidin-4-yl)isoquinolin-3-yl)-2,2-dimethylcyclopropane-1-carboxamide,
(R)-N-(7-chloro-6-(1-((3R,4R)-4-hydroxy-3-methyltetrahydrofuran-3-yl)piperidin-4-yl)isoquinolin-3-yl)-2,2-dimethylcyclopropane-1-carboxamide,
(R)-N-(7-chloro-6-(1-((3S,4S)-4-hydroxy-3-methyltetrahydrofuran-3-yl)piperidin-4-yl)isoquinolin-3-yl)-2,2-dimethylcyclopropane-1-carboxamide,
(S)-N-(7-chloro-6-(1-((3R,4R)-4-hydroxy-3-methyltetrahydrofuran-3-yl)piperidin-4-yl)isoquinolin-3-yl)-2,2-dimethylcyclopropane-1-carboxamide,
(S)-N-(7-chloro-6-(1-((3S,4S)-4-hydroxy-3-methyltetrahydrofuran-3-yl)piperidin-4-yl)isoquinolin-3-yl)-2,2-dimethylcyclopropane-1-carboxamide,
Trans-N-(7-chloro-6-(1-(4-hydroxy-3-methyltetrahydrofuran-3-yl)piperidin-4-yl)isoquinolin-3-yl)-3-(2-hydroxypropan-2-yl)cyclobutane-1-carboxamide,
(1R,3R)-N-(7-chloro-6-(1-((3R,4R)-4-hydroxy-3-methyltetrahydrofuran-3-yl)piperidin-4-yl)isoquinolin-3-yl)-3-(2-hydroxypropan-2-yl)cyclobutane-1-carboxamide,
(1R,3R)-N-(7-chloro-6-(1-((3S,4S)-4-hydroxy-3-methyltetrahydrofuran-3-yl)piperidin-4-yl)isoquinolin-3-yl)-3-(2-hydroxypropan-2-yl)cyclobutane-1-carboxamide,
(1S,3S)-N-(7-chloro-6-(1-((3R,4R)-4-hydroxy-3-methyltetrahydrofuran-3-yl)piperidin-4-yl)isoquinolin-3-yl)-3-(2-hydroxypropan-2-yl)cyclobutane-1-carboxamide,
(1S,3S)-N-(7-chloro-6-(1-((3S,4S)-4-hydroxy-3-methyltetrahydrofuran-3-yl)piperidin-4-yl)isoquinolin-3-yl)-3-(2-hydroxypropan-2-yl)cyclobutane-1-carboxamide,
N-(7-chloro-6-(1-(4-hydroxy-3-methyltetrahydrofuran-3-yl)piperidin-4-yl)isoquinolin-3-yl)-3-oxabicyclo[3.1.0]hexane-6-carboxamide,
(R)-N-(7-chloro-6-(1-((3R,4R)-4-hydroxy-3-methyltetrahydrofuran-3-yl)piperidin-4-yl)isoquinolin-3-yl)-3-oxabicyclo[3.1.0]hexane-6-carboxamide,
(S)-N-(7-chloro-6-(1-((3R,4R)-4-hydroxy-3-methyltetrahydrofuran-3-yl)piperidin-4-yl)isoquinolin-3-yl)-3-oxabicyclo[3.1.0]hexane-6-carboxamide,
(R)-N-(7-chloro-6-(1-((3S,4S)-4-hydroxy-3-methyltetrahydrofuran-3-yl)piperidin-4-yl)isoquinolin-3-yl)-3-oxabicyclo[3.1.0]hexane-6-carboxamide,
(S)-N-(7-chloro-6-(1-((3S,4S)-4-hydroxy-3-methyltetrahydrofuran-3-yl)piperidin-4-yl)isoquinolin-3-yl)-3-oxabicyclo[3.1.0]hexane-6-carboxamide,
N-(7-chloro-6-(1-(4-hydroxy-3-methyltetrahydrofuran-3-yl)piperidin-4-yl)isoquinolin-3-yl)-6,6-difluorospiro[2.5]octane-1-carboxamide,
(R)-N-(7-chloro-6-(1-((3R,4R)-4-hydroxy-3-methyltetrahydrofuran-3-yl)piperidin-4-yl)isoquinolin-3-yl)-6,6-difluorospiro[2.5]octane-1-carboxamide,
(S)-N-(7-chloro-6-(1-((3R,4R)-4-hydroxy-3-methyltetrahydrofuran-3-yl)piperidin-4-yl)isoquinolin-3-yl)-6,6-difluorospiro[2.5]octane-1-carboxamide,
(R)-N-(7-chloro-6-(1-((3S,4S)-4-hydroxy-3-methyltetrahydrofuran-3-yl)piperidin-4-yl)isoquinolin-3-yl)-6,6-difluorospiro[2.5]octane-1-carboxamide,
(S)-N-(7-chloro-6-(1-((3R,4R)-4-hydroxy-3-methyltetrahydrofuran-3-yl)piperidin-4-yl)isoquinolin-3-yl)-6,6-difluorospiro[2.5]octane-1-carboxamide,
N-(7-chloro-6-(1-(4-hydroxy-3-methyltetrahydrofuran-3-yl)piperidin-4-yl)isoquinolin-3-yl)-2-(2-methoxypropan-2-yl)cyclopropane-1-carboxamide,
1R,2R)-N-(7-chloro-6-(1-((3R,4R)-4-hydroxy-3-methyltetrahydrofuran-3-yl)piperidin-4-yl)isoquinolin-3-yl)-2-(2-methoxypropan-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(2-methoxypropan-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(2-methoxypropan-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(2-methoxypropan-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(2-hydroxypropan-2-yl)cyclopropane-1-carboxamide,
N-(7-chloro-6-(1-(3-methyltetrahydrofuran-3-yl)piperidin-4-yl)isoquinolin-3-yl)-6-oxaspiro[2.5]octane-1-carboxamide,
(R)-N-(7-chloro-6-(1-(3R-methyltetrahydrofuran-3-yl)piperidin-4-yl)isoquinolin-3-yl)-6-oxaspiro[2.5]octane-1-carboxamide,
(R)-N-(7-chloro-6-(1-(3S-methyltetrahydrofuran-3-yl)piperidin-4-yl)isoquinolin-3-yl)-6-oxaspiro[2.5]octane-1-carboxamide,
(S)-N-(7-chloro-6-(1-(3R-methyltetrahydrofuran-3-yl)piperidin-4-yl)isoquinolin-3-yl)-6-oxaspiro[2.5]octane-1-carboxamide,
(S)-N-(7-chloro-6-(1-(3S-methyltetrahydrofuran-3-yl)piperidin-4-yl)isoquinolin-3-yl)-6-oxaspiro[2.5]octane-1-carboxamide,
Rac-N-(7-chloro-6-(1-(3-methyltetrahydrofuran-3-yl)piperidin-4-yl)isoquinolin-3-yl)-2-(2-hydroxypropan-2-yl)cyclopropane-1-carboxamide,
N-(7-chloro-6-(1-(4-hydroxy-3-methyltetrahydrofuran-3-yl)piperidin-4-yl)isoquinolin-3-yl)tetrahydro-2H-pyran-3-carboxamide,
(R)-N-(7-chloro-6-(1-((3R,4R)-4-hydroxy-3-methyltetrahydrofuran-3-yl)piperidin-4-yl)isoquinolin-3-yl)tetrahydro-2H-pyran-3-carboxamide,
(S)-N-(7-chloro-6-(1-((3R,4R)-4-hydroxy-3-methyltetrahydrofuran-3-yl)piperidin-4-yl)isoquinolin-3-yl)tetrahydro-2H-pyran-3-carboxamide,
(R)-N-(7-chloro-6-(1-((3S,4S)-4-hydroxy-3-methyltetrahydrofuran-3-yl)piperidin-4-yl)isoquinolin-3-yl)tetrahydro-2H-pyran-3-carboxamide,
(S)-N-(7-chloro-6-(1-((3S,4S)-4-hydroxy-3-methyltetrahydrofuran-3-yl)piperidin-4-yl)isoquinolin-3-yl)tetrahydro-2H-pyran-3-carboxamide,
(2R,5S)-N-(7-chloro-6-(1-(4-hydroxy-3-methyltetrahydrofuran-3-1)piperidin-4-yl)isoquinolin-3-yl)-5-isopropoxytetrahydro-2H-pyran-2-carboxamide,
(2R,5S)-N-(7-chloro-6-(1-((3R,4R)-4-hydroxy-3-methyltetrahydrofuran-3-1)piperidin-4-yl)isoquinolin-3-yl)-5-isopropoxytetrahydro-2H-pyran-2-carboxamide,
(2R,5S)-N-(7-chloro-6-(1-((3S,4S)-4-hydroxy-3-methyltetrahydrofuran-3-1)piperidin-4-yl)isoquinolin-3-yl)-5-isopropoxytetrahydro-2H-pyran-2-carboxamide,
N-(7-chloro-6-(1-(4-hydroxy-3-methyltetrahydrofuran-3-yl)piperidin-4-yl)isoquinolin-3-yl)tetrahydrofuran-2-carboxamide,
(2R)-N-(7-chloro-6-(1-((3R,4R)-4-hydroxy-3-methyltetrahydrofuran-3-yl)piperidin-4-yl)isoquinolin-3-yl)tetrahydrofuran-2-carboxamide,
(2S)-N-(7-chloro-6-(1-((3R,4R)-4-hydroxy-3-methyltetrahydrofuran-3-yl)piperidin-4-yl)isoquinolin-3-yl)tetrahydrofuran-2-carboxamide,
(2R)-N-(7-chloro-6-(1-((3S,4S)-4-hydroxy-3-methyltetrahydrofuran-3-yl)piperidin-4-yl)isoquinolin-3-yl)tetrahydrofuran-2-carboxamide,
(2S)-N-(7-chloro-6-(1-((3S,4S)-4-hydroxy-3-methyltetrahydrofuran-3-yl)piperidin-4-yl)isoquinolin-3-yl)tetrahydrofuran-2-carboxamide,
N-(7-chloro-6-(1-(4-hydroxy-3-methyltetrahydrofuran-3-yl)piperidin-4-yl)isoquinolin-3-yl)-5,5-dimethyltetrahydrofuran-3-carboxamide,
(R)-N-(7-chloro-6-(1-((3R,4R)-4-hydroxy-3-methyltetrahydrofuran-3-yl)piperidin-4-yl)isoquinolin-3-yl)-5,5-dimethyltetrahydrofuran-3-carboxamide,
(R)-N-(7-chloro-6-(1-((3S,4S)-4-hydroxy-3-methyltetrahydrofuran-3-yl)piperidin-4-yl)isoquinolin-3-yl)-5,5-dimethyltetrahydrofuran-3-carboxamide,
(S)-N-(7-chloro-6-(1-((3R,4R)-4-hydroxy-3-methyltetrahydrofuran-3-yl)piperidin-4-yl)isoquinolin-3-yl)-5,5-dimethyltetrahydrofuran-3-carboxamide,
S)-N-(7-chloro-6-(1-((3S,4S)-4-hydroxy-3-methyltetrahydrofuran-3-yl)piperidin-4-yl)isoquinolin-3-yl)-5,5-dimethyltetrahydrofuran-3-carboxamide,
N-(6-(1-cyanospiro[2.2]pentan-1-yl)isoquinolin-3-yl)-2-(2-hydroxypropan-2-yl)cyclopropane-1-carboxamide,
(1R,2R)-N-(6-((R)-1-cyanospiro[2.2]pentan-1-yl)isoquinolin-3-yl)-2-(2-hydroxypropan-2-yl)cyclopropane-1-carboxamide,
(1R,2R)-N-(6-((S)-1-cyanospiro[2.2]pentan-1-yl)isoquinolin-3-yl)-2-(2-hydroxypropan-2-yl)cyclopropane-1-carboxamide,
(1S,2S)-N-(6-((R)-1-cyanospiro[2.2]pentan-1-yl)isoquinolin-3-yl)-2-(2-hydroxypropan-2-yl)cyclopropane-1-carboxamide,
(1S,2S)-N-(6-((S)-1-cyanospiro[2.2]pentan-1-yl)isoquinolin-3-yl)-2-(2-hydroxypropan-2-yl)cyclopropane-1-carboxamide,
N-(7-chloro-6-(1-(4-hydroxy-3-methyltetrahydrofuran-3-yl)piperidin-4-yl)isoquinolin-3-yl)-2-cyanocyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-cyanocyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-cyanocyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-cyanocyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-cyanocyclopropane-1-carboxamide,
N-(7-chloro-6-(1-(4-hydroxy-3-methyltetrahydrofuran-3-yl)piperidin-4-yl)isoquinolin-3-yl)-2,2-dimethyltetrahydro-2H-pyran-4-carboxamide,
(R)-N-(7-chloro-6-(1-((3R,4R)-4-hydroxy-3-methyltetrahydrofuran-3-yl)piperidin-4-yl)isoquinolin-3-yl)-2,2-dimethyltetrahydro-2H-pyran-4-carboxamide,
(R)-N-(7-chloro-6-(1-((3S,4S)-4-hydroxy-3-methyltetrahydrofuran-3-yl)piperidin-4-yl)isoquinolin-3-yl)-2,2-dimethyltetrahydro-2H-pyran-4-carboxamide,
(S)-N-(7-chloro-6-(1-((3R,4R)-4-hydroxy-3-methyltetrahydrofuran-3-yl)piperidin-4-yl)isoquinolin-3-yl)-2,2-dimethyltetrahydro-2H-pyran-4-carboxamide,
(S)-N-(7-chloro-6-(1-((3S,4S)-4-hydroxy-3-methyltetrahydrofuran-3-yl)piperidin-4-yl)isoquinolin-3-yl)-2,2-dimethyltetrahydro-2H-pyran-4-carboxamide,
N-(7-chloro-6-(1-(4-hydroxy-3-methyltetrahydrofuran-3-yl)piperidin-4-yl)isoquinolin-3-yl)-2-(1-methyl-1H-pyrazol-4-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)cyclopropane-1-carboxamide,
N-(6-(1-cyanospiro[2.2]pentan-1-yl)isoquinolin-3-yl)-2-(pyridin-2-yl)cyclopropane-1-carboxamide,
(1R,2R)-N-(6-((R)-1-cyanospiro[2.2]pentan-1-yl)isoquinolin-3-yl)-2-(pyridin-2-yl)cyclopropane-1-carboxamide,
(1R,2R)-N-(6-((S)-1-cyanospiro[2.2]pentan-1-yl)isoquinolin-3-yl)-2-(pyridin-2-yl)cyclopropane-1-carboxamide,
(1S,2S)-N-(6-((R)-1-cyanospiro[2.2]pentan-1-yl)isoquinolin-3-yl)-2-(pyridin-2-yl)cyclopropane-1-carboxamide,
(1S,2S)-N-(6-((S)-1-cyanospiro[2.2]pentan-1-yl)isoquinolin-3-yl)-2-(pyridin-2-yl)cyclopropane-1-carboxamide,
N-(6-(1-cyanospiro[2.2]pentan-1-yl)isoquinolin-3-yl)-2-(pyridin-4-yl)cyclopropane-1-carboxamide,
(1R,2R)-N-(6-((R)-1-cyanospiro[2.2]pentan-1-yl)isoquinolin-3-yl)-2-(pyridin-4-yl)cyclopropane-1-carboxamide,
(1R,2R)-N-(6-((S)-1-cyanospiro[2.2]pentan-1-yl)isoquinolin-3-yl)-2-(pyridin-4-yl)cyclopropane-1-carboxamide,
(1S,2S)-N-(6-((R)-1-cyanospiro[2.2]pentan-1-yl)isoquinolin-3-yl)-2-(pyridin-4-yl)cyclopropane-1-carboxamide,
(1S,2S)-N-(6-((S)-1-cyanospiro[2.2]pentan-1-yl)isoquinolin-3-yl)-2-(pyridin-4-yl)cyclopropane-1-carboxamide,
N-(6-(1-cyanospiro[2.2]pentan-1-yl)isoquinolin-3-yl)-1-(2,2,2-trifluoroethyl)pyrrolidine-3-carboxamide,
(R)-N-(6-((R)-1-cyanospiro[2.2]pentan-1-yl)isoquinolin-3-yl)-1-(2,2,2-trifluoroethyl)pyrrolidine-3-carboxamide,
(R)-N-(6-((S)-1-cyanospiro[2.2]pentan-1-yl)isoquinolin-3-yl)-1-(2,2,2-trifluoroethyl)pyrrolidine-3-carboxamide,
(S)-N-(6-((R)-1-cyanospiro[2.2]pentan-1-yl)isoquinolin-3-yl)-1-(2,2,2-trifluoroethyl)pyrrolidine-3-carboxamide,
(S)-N-(6-((S)-1-cyanospiro[2.2]pentan-1-yl)isoquinolin-3-yl)-1-(2,2,2-trifluoroethyl)pyrrolidine-3-carboxamide,
N-(6-(1-cyanospiro[2.2]pentan-1-yl)isoquinolin-3-yl)-2-(pyridin-3-yl)cyclopropane-1-carboxamide,
(1R,2R)-N-(6-((R)-1-cyanospiro[2.2]pentan-1-yl)isoquinolin-3-yl)-2-(pyridin-3-yl)cyclopropane-1-carboxamide,
(1R,2R)-N-(6-((S)-1-cyanospiro[2.2]pentan-1-yl)isoquinolin-3-yl)-2-(pyridin-3-yl)cyclopropane-1-carboxamide,
(1S,2S)-N-(6-((R)-1-cyanospiro[2.2]pentan-1-yl)isoquinolin-3-yl)-2-(pyridin-3-yl)cyclopropane-1-carboxamide,
(1S,2S)-N-(6-((S)-1-cyanospiro[2.2]pentan-1-yl)isoquinolin-3-yl)-2-(pyridin-3-yl)cyclopropane-1-carboxamide,
N-(6-(1-cyanospiro[2.2]pentan-1-yl)isoquinolin-3-yl)-2-(1-methyl-5-(trifluoromethyl)-1H-pyrazol-4-yl)cyclopropane-1-carboxamide,
(1R,2R)-N-(6-((R)-1-cyanospiro[2.2]pentan-1-yl)isoquinolin-3-yl)-2-(1-methyl-5-(trifluoromethyl)-1H-pyrazol-4-yl)cyclopropane-1-carboxamide,
(1R,2R)-N-(6-((S)-1-cyanospiro[2.2]pentan-1-yl)isoquinolin-3-yl)-2-(1-methyl-5-(trifluoromethyl)-1H-pyrazol-4-yl)cyclopropane-1-carboxamide,
(1S,2S)-N-(6-((R)-1-cyanospiro[2.2]pentan-1-yl)isoquinolin-3-yl)-2-(1-methyl-5-(trifluoromethyl)-1H-pyrazol-4-yl)cyclopropane-1-carboxamide,
(1S,2S)-N-(6-((S)-1-cyanospiro[2.2]pentan-1-yl)isoquinolin-3-yl)-2-(1-methyl-5-(trifluoromethyl)-1H-pyrazol-4-yl)cyclopropane-1-carboxamide,
N-(6-(1-(4-hydroxy-3-methyltetrahydrofuran-3-yl)piperidin-4-yl)-7-methylisoquinolin-3-yl)-2,2-dimethyltetrahydro-2H-pyran-4-carboxamide,
(R)-N-(6-(1-((3R,4R)-4-hydroxy-3-methyltetrahydrofuran-3-yl)piperidin-4-yl)-7-methylisoquinolin-3-yl)-2,2-dimethyltetrahydro-2H-pyran-4-carboxamide, (R)-N-(6-(1-((3S,4S)-4-hydroxy-3-methyltetrahydrofuran-3-yl)piperidin-4-yl)-7-methylisoquinolin-3-yl)-2,2-dimethyltetrahydro-2H-pyran-4-carboxamide,
(S)-N-(6-(1-((3R,4R)-4-hydroxy-3-methyltetrahydrofuran-3-yl)piperidin-4-yl)-7-methylisoquinolin-3-yl)-2,2-dimethyltetrahydro-2H-pyran-4-carboxamide,
(S)-N-(6-(1-((3S,4S)-4-hydroxy-3-methyltetrahydrofuran-3-yl)piperidin-4-yl)-7-methylisoquinolin-3-yl)-2,2-dimethyltetrahydro-2H-pyran-4-carboxamide,
N-(6-(1-(4-hydroxy-3-methyltetrahydrofuran-3-yl)piperidin-4-yl)-7-methylisoquinolin-3-yl)-2,2-dimethyltetrahydro-2H-pyran-4-carboxamide,
(R)-N-(6-(1-((3R,4R)-4-hydroxy-3-methyltetrahydrofuran-3-yl)piperidin-4-yl)-7-methylisoquinolin-3-yl)-2,2-dimethyltetrahydro-2H-pyran-4-carboxamide,
(R)-N-(6-(1-((3S,4S)-4-hydroxy-3-methyltetrahydrofuran-3-yl)piperidin-4-yl)-7-methylisoquinolin-3-yl)-2,2-dimethyltetrahydro-2H-pyran-4-carboxamide,
(S)-N-(6-(1-((3R,4R)-4-hydroxy-3-methyltetrahydrofuran-3-yl)piperidin-4-yl)-7-methylisoquinolin-3-yl)-2,2-dimethyltetrahydro-2H-pyran-4-carboxamide,
(S)-N-(6-(1-((3S,4S)-4-hydroxy-3-methyltetrahydrofuran-3-yl)piperidin-4-yl)-7-methylisoquinolin-3-yl)-2,2-dimethyltetrahydro-2H-pyran-4-carboxamide,
2-(difluoromethyl)-N-(6-(1-(4-hydroxy-3-methyltetrahydrofuran-3-yl)piperidin-4-yl)-7-methylisoquinolin-3-yl)cyclopropane-1-carboxamide,
(1R,2R)-2-(difluoromethyl)-N-(6-(1-((3R,4R)-4-hydroxy-3-methyltetrahydrofuran-3-yl)piperidin-4-yl)-7-methylisoquinolin-3-yl)cyclopropane-1-carboxamide,
(1R,2R)-2-(difluoromethyl)-N-(6-(1-((3S,4S)-4-hydroxy-3-methyltetrahydrofuran-3-yl)piperidin-4-yl)-7-methylisoquinolin-3-yl)cyclopropane-1-carboxamide,
(1S,2S)-2-(difluoromethyl)-N-(6-(1-((3R,4R)-4-hydroxy-3-methyltetrahydrofuran-3-yl)piperidin-4-yl)-7-methylisoquinolin-3-yl)cyclopropane-1-carboxamide,
(1S,2S)-2-(difluoromethyl)-N-(6-(1-((3S,4S)-4-hydroxy-3-methyltetrahydrofuran-3-yl)piperidin-4-yl)-7-methylisoquinolin-3-yl)cyclopropane-1-carboxamide,
N-(6-(1-cyanospiro[2.2]pentan-1-yl)isoquinolin-3-yl)-2,2-dimethyltetrahydro-2H-pyran-4-carboxamide,
(R)-N-(6-((R)-1-cyanospiro[2.2]pentan-1-yl)isoquinolin-3-yl)-2,2-dimethyltetrahydro-2H-pyran-4-carboxamide,
(R)-N-(6-((S)-1-cyanospiro[2.2]pentan-1-yl)isoquinolin-3-yl)-2,2-dimethyltetrahydro-2H-pyran-4-carboxamide,
(S)-N-(6-((R)-1-cyanospiro[2.2]pentan-1-yl)isoquinolin-3-yl)-2,2-dimethyltetrahydro-2H-pyran-4-carboxamide,
(S)-N-(6-((S)-1-cyanospiro[2.2]pentan-1-yl)isoquinolin-3-yl)-2,2-dimethyltetrahydro-2H-pyran-4-carboxamide,
N-(7-chloro-6-(1-(4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5R)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5S)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5R)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5S)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5R)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5S)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5R)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5S)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
N-(6-(1-cyanospiro[2.2]pentan-1-yl)isoquinolin-3-yl)-5-oxaspiro[2.4]heptane-1-carboxamide,
(1R,2R)-N-(6-((R)-1-cyanospiro[2.2]pentan-1-yl)isoquinolin-3-yl)-5-oxaspiro[2.4]heptane-1-carboxamide,
(1R,2S)-N-(6-((R)-1-cyanospiro[2.2]pentan-1-yl)isoquinolin-3-yl)-5-oxaspiro[2.4]heptane-1-carboxamide,
(1S,2R)-N-(6-((R)-1-cyanospiro[2.2]pentan-1-yl)isoquinolin-3-yl)-5-oxaspiro[2.4]heptane-1-carboxamide,
(1S,2S)-N-(6-((R)-1-cyanospiro[2.2]pentan-1-yl)isoquinolin-3-yl)-5-oxaspiro[2.4]heptane-1-carboxamide,
(1R,2R)-N-(6-((S)-1-cyanospiro[2.2]pentan-1-yl)isoquinolin-3-yl)-5-oxaspiro[2.4]heptane-1-carboxamide,
(1R,2S)-N-(6-((S)-1-cyanospiro[2.2]pentan-1-yl)isoquinolin-3-yl)-5-oxaspiro[2.4]heptane-1-carboxamide,
(1S,2R)-N-(6-((S)-1-cyanospiro[2.2]pentan-1-yl)isoquinolin-3-yl)-5-oxaspiro[2.4]heptane-1-carboxamide,
(1S,2S)-N-(6-((S)-1-cyanospiro[2.2]pentan-1-yl)isoquinolin-3-yl)-5-oxaspiro[2.4]heptane-1-carboxamide,
N-(7-chloro-6-(1-(4-fluoro-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(pyridin-2-yl)cyclopropane-1-carboxamide,
N-(6-(1-cyanospiro[2.2]pentan-1-yl)isoquinolin-3-yl)-2-(1-methyl-1H-pyrazol-3-yl)cyclopropane-1-carboxamide,
(1R,2R)-N-(6-((R)-1-cyanospiro[2.2]pentan-1-yl)isoquinolin-3-yl)-2-(1-methyl-1H-pyrazol-3-yl)cyclopropane-1-carboxamide,
(1R,2R)-N-(6-((S)-1-cyanospiro[2.2]pentan-1-yl)isoquinolin-3-yl)-2-(1-methyl-1H-pyrazol-3-yl)cyclopropane-1-carboxamide,
(1S,2S)-N-(6-((R)-1-cyanospiro[2.2]pentan-1-yl)isoquinolin-3-yl)-2-(1-methyl-1H-pyrazol-3-yl)cyclopropane-1-carboxamide,
(1S,2S)-N-(6-((S)-1-cyanospiro[2.2]pentan-1-yl)isoquinolin-3-yl)-2-(1-methyl-1H-pyrazol-3-yl)cyclopropane-1-carboxamide,
N-(6-(1-cyanospiro[2.2]pentan-1-yl)isoquinolin-3-yl)-2-methyl-3-(1-methyl-1H-pyrazol-4-yl)cyclopropane-1-carboxamide,
(1R,2R,3R)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3 S)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3R)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3S)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3R)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3S)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3R)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3S)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
N-(6-(1-cyanospiro[2.2]pentan-1-yl)isoquinolin-3-yl)-1-(1-methyl-1H-pyrazol-4-yl)pyrrolidine-3-carboxamide,
(R)-N-(6-((R)-1-cyanospiro[2.2]pentan-1-yl)isoquinolin-3-yl)-1-(1-methyl-1H-pyrazol-4-yl)pyrrolidine-3-carboxamide,
(R)-N-(6-((S)-1-cyanospiro[2.2]pentan-1-yl)isoquinolin-3-yl)-1-(1-methyl-1H-pyrazol-4-yl)pyrrolidine-3-carboxamide,
(S)-N-(6-((R)-1-cyanospiro[2.2]pentan-1-yl)isoquinolin-3-yl)-1-(1-methyl-1H-pyrazol-4-yl)pyrrolidine-3-carboxamide,
(S)-N-(6-((S)-1-cyanospiro[2.2]pentan-1-yl)isoquinolin-3-yl)-1-(1-methyl-1H-pyrazol-4-yl)pyrrolidine-3-carboxamide,
2-(5-chloro-1-methyl-1H-pyrazol-4-yl)-N-(6-(1-cyanospiro[2.2]pentan-1-yl)isoquinolin-3-yl)cyclopropane-1-carboxamide,
(1R,2R-(5-chloro-1-methyl-1H-pyrazol-4-yl))-N-(6-((R)-1-cyanospiro[2.2]pentan-1-yl)isoquinolin-3-yl)cyclopropane-1-carboxamide,
(1R,2R-(5-chloro-1-methyl-1H-pyrazol-4-yl))-N-(6-((S)-1-cyanospiro[2.2]pentan-1-yl)isoquinolin-3-yl)cyclopropane-1-carboxamide,
(1S,2S-(5-chloro-1-methyl-1H-pyrazol-4-yl))-N-(6-((R)-1-cyanospiro[2.2]pentan-1-yl)isoquinolin-3-yl)cyclopropane-1-carboxamide,
(1S,2S-(5-chloro-1-methyl-1H-pyrazol-4-yl))-N-(6-((S)-1-cyanospiro[2.2]pentan-1-yl)isoquinolin-3-yl)cyclopropane-1-carboxamide,
N-(7-chloro-6-(1-(4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2R,3R)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2R,3 S)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2S,3R)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2S,3S)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2R,3R)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2R,3 S)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2S,3R)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2S,3S)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
N-(7-chloro-6-(1-(4-fluoro-3-methyltetrahydrofuran-3-yl)piperidin-4-yl)isoquinolin-3-yl)-5-ethoxyspiro[2.3]hexane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-5-ethoxyspiro[2.3]hexane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-5-ethoxyspiro[2.3]hexane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-5-ethoxyspiro[2.3]hexane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-5-ethoxyspiro[2.3]hexane-1-carboxamide,
N-(7-chloro-6-(1-(4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
N-(7-chloro-6-(1-(4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
1R,2R)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
N-(7-chloro-6-(1-(4-hydroxy-3-methyltetrahydrofuran-3-yl)piperidin-4-yl)isoquinolin-3-yl)-2-methyl-2-(pyridin-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-methyl-2-(pyridin-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-methyl-2-(pyridin-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-methyl-2-(pyridin-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-methyl-2-(pyridin-2-yl)cyclopropane-1-carboxamide,
N-(7-chloro-6-(1-(4-fluoro-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(tetrahydro-2H-pyran-4-yl)cyclopropane-1-carboxamide,
N-(7-chloro-6-(1-(4-fluoro-3-methyltetrahydrofuran-3-yl)piperidin-4-yl)isoquinolin-3-yl)-2-cyanocyclobutane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1R,2S)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1S,2R)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1R,2S)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1S,2R)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
N-(7-chloro-6-(1-(4-fluoro-3-methyltetrahydrofuran-3-yl)piperidin-4-yl)isoquinolin-3-yl)spiro[2.2]pentane-1-carboxamide,
(R)-N-(7-chloro-6-(1-((3R,4R)-4-fluoro-3-methyltetrahydrofuran-3-yl)piperidin-4-yl)isoquinolin-3-yl)spiro[2.2]pentane-1-carboxamide,
(R)-N-(7-chloro-6-(1-((3S,4S)-4-fluoro-3-methyltetrahydrofuran-3-yl)piperidin-4-yl)isoquinolin-3-yl)spiro[2.2]pentane-1-carboxamide,
(S)-N-(7-chloro-6-(1-((3R,4R)-4-fluoro-3-methyltetrahydrofuran-3-yl)piperidin-4-yl)isoquinolin-3-yl)spiro[2.2]pentane-1-carboxamide,
(S)-N-(7-chloro-6-(1-((3S,4S)-4-fluoro-3-methyltetrahydrofuran-3-yl)piperidin-4-yl)isoquinolin-3-yl)spiro[2.2]pentane-1-carboxamide,
N-(7-chloro-6-(spiro[2.2]pentan-1-yl)isoquinolin-3-yl)-2-ethyl-3-(1-methyl-1H-pyrazol-4-yl)cyclopropane-1-carboxamide,
(1R,2R,3R)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1R,2R,3R)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1S,2R,3S)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1S,2R,3S)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1R,2S,3R)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1R,2S,3R)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1S,2S,3S)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1S,2S,3S)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
N-(7-chloro-6-(spiro[2.2]pentan-1-yl)isoquinolin-3-yl)-2-(1-methyl-1H-pyrazol-4-yl)cyclopropane-1-carboxamide,
(1R,2R)-N-(7-chloro-6R-(spiro[2.2]pentan-1-yl)isoquinolin-3-yl)-2-(1-methyl-1H-pyrazol-4-yl)cyclopropane-1-carboxamide,
(1R,2R)-N-(7-chloro-6S-(spiro[2.2]pentan-1-yl)isoquinolin-3-yl)-2-(1-methyl-1H-pyrazol-4-yl)cyclopropane-1-carboxamide,
(1S,2S)-N-(7-chloro-6R-(spiro[2.2]pentan-1-yl)isoquinolin-3-yl)-2-(1-methyl-1H-pyrazol-4-yl)cyclopropane-1-carboxamide,
(1S,2S)-N-(7-chloro-6S-(spiro[2.2]pentan-1-yl)isoquinolin-3-yl)-2-(1-methyl-1H-pyrazol-4-yl)cyclopropane-1-carboxamide,
N-(7-chloro-6-(1-(4-hydroxy-3-methyltetrahydrofuran-3-yl)piperidin-4-yl)isoquinolin-3-yl)-2-cyclopropylpropanamide,
(R)-N-(7-chloro-6-(1-((3R,4R)-4-hydroxy-3-methyltetrahydrofuran-3-yl)piperidin-4-yl)isoquinolin-3-yl)-2-cyclopropylpropanamide,
(S)-N-(7-chloro-6-(1-((3R,4R)-4-hydroxy-3-methyltetrahydrofuran-3-yl)piperidin-4-yl)isoquinolin-3-yl)-2-cyclopropylpropanamide,
(R)-N-(7-chloro-6-(1-((3S,4S)-4-hydroxy-3-methyltetrahydrofuran-3-yl)piperidin-4-yl)isoquinolin-3-yl)-2-cyclopropylpropanamide,
(S)-N-(7-chloro-6-(1-((3S,4S)-4-hydroxy-3-methyltetrahydrofuran-3-yl)piperidin-4-yl)isoquinolin-3-yl)-2-cyclopropylpropanamide,
N-(7-chloro-6-(1-(4-hydroxy-3-methyltetrahydrofuran-3-yl)piperidin-4-yl)isoquinolin-3-yl)-2-(tetrahydro-2H-pyran-4-yl)propenamide,
(R)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)propenamide,
(S)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)propenamide,
(R)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-4-yl)propenamide,
(S)-N-(7-chloro-6-(1-((3 S,4S)-4-hydroxy-3-methyltetrahydrofuran-3-yl)piperidin-4-yl)isoquinolin-3-yl)-2-(tetrahydro-2H-pyran-4-yl)propenamide,
N-(7-chloro-6-(1-(4-hydroxy-3-methyltetrahydrofuran-3-yl)piperidin-4-yl)isoquinolin-3-yl)-2-(1-methyl-1H-pyrazol-4-yl)propenamide,
(R)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)propenamide,
(S)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)propenamide,
(R)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)propenamide,
(S)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)propenamide,
N-(6-(1-cyanospiro[2.2]pentan-1-yl)isoquinolin-3-yl)-2-(1-methyl-1H-pyrazol-4-yl)propenamide,
(R)-N-(6-(1R-cyanospiro[2.2]pentan-1-yl)isoquinolin-3-yl)-2-(1-methyl-1H-pyrazol-4-yl)propenamide,
(S)-N-(6-(1R-cyanospiro[2.2]pentan-1-yl)isoquinolin-3-yl)-2-(1-methyl-1H-pyrazol-4-yl)propenamide,
(R)-N-(6-(1S-cyanospiro[2.2]pentan-1-yl)isoquinolin-3-yl)-2-(1-methyl-1H-pyrazol-4-yl)propenamide,
(S)-N-(6-(1S-cyanospiro[2.2]pentan-1-yl)isoquinolin-3-yl)-2-(1-methyl-1H-pyrazol-4-yl)propenamide,
N-(6-(1-cyanospiro[2.2]pentan-1-yl)isoquinolin-3-yl)-2-(l-propionylpiperidin-4-yl)propenamide,
(R)-N-(6-(1R-cyanospiro[2.2]pentan-1-yl)isoquinolin-3-yl)-2-(l-propionylpiperidin-4-yl)propenamide,
(R)-N-(6-(1S-cyanospiro[2.2]pentan-1-yl)isoquinolin-3-yl)-2-(1-propionylpiperidin-4-yl)propenamide,
(S)-N-(6-(1R-cyanospiro[2.2]pentan-1-yl)isoquinolin-3-yl)-2-(1-propionylpiperidin-4-yl)propenamide,
(S)-N-(6-(1S-cyanospiro[2.2]pentan-1-yl)isoquinolin-3-yl)-2-(1-propionylpiperidin-4-yl)propenamide,
N-(6-(1-cyanospiro[2.2]pentan-1-yl)isoquinolin-3-yl)-2,2-dimethyltetrahydro-2H-pyran-4-carboxamide,
(R)-N-(6-((R)-1-cyanospiro[2.2]pentan-1-yl)isoquinolin-3-yl)-2,2-dimethyltetrahydro-2H-pyran-4-carboxamide,
(R)-N-(6-((S)-1-cyanospiro[2.2]pentan-1-yl)isoquinolin-3-yl)-2,2-dimethyltetrahydro-2H-pyran-4-carboxamide,
(S)-N-(6-((R)-1-cyanospiro[2.2]pentan-1-yl)isoquinolin-3-yl)-2,2-dimethyltetrahydro-2H-pyran-4-carboxamide,
(S)-N-(6-((S)-1-cyanospiro[2.2]pentan-1-yl)isoquinolin-3-yl)-2,2-dimethyltetrahydro-2H-pyran-4-carboxamide,
N-(7-chloro-6-(1-(4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5R)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5S)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5R)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5S)-N-(7-chloro-6-(1-((3R,4R)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5R)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2R,5S)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5R)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
(2S,5S)-N-(7-chloro-6-(1-((3S,4S)-4-hydroxy-3-methyltetrahydrofuran-3-yl)piperidin-4-yl)isoquinolin-3-yl)-6-(trifluoromethyl)tetrahydro-2H-pyran-3-carboxamide,
N-(6-(1-cyanospiro[2.2]pentan-1-yl)isoquinolin-3-yl)-5-oxaspiro[2.4]heptane-1-carboxamide,
(1R,2R)-N-(6-((R)-1-cyanospiro[2.2]pentan-1-yl)isoquinolin-3-yl)-5-oxaspiro[2.4]heptane-1-carboxamide,
(1R,2S)-N-(6-((R)-1-cyanospiro[2.2]pentan-1-yl)isoquinolin-3-yl)-5-oxaspiro[2.4]heptane-1-carboxamide,
(1S,2R)-N-(6-((R)-1-cyanospiro[2.2]pentan-1-yl)isoquinolin-3-yl)-5-oxaspiro[2.4]heptane-1-carboxamide,
(1S,2S)-N-(6-((R)-1-cyanospiro[2.2]pentan-1-yl)isoquinolin-3-yl)-5-oxaspiro[2.4]heptane-1-carboxamide,
(1R,2R)-N-(6-((S)-1-cyanospiro[2.2]pentan-1-yl)isoquinolin-3-yl)-5-oxaspiro[2.4]heptane-1-carboxamide,
(1R,2S)-N-(6-((S)-1-cyanospiro[2.2]pentan-1-yl)isoquinolin-3-yl)-5-oxaspiro[2.4]heptane-1-carboxamide,
(1S,2R)-N-(6-((S)-1-cyanospiro[2.2]pentan-1-yl)isoquinolin-3-yl)-5-oxaspiro[2.4]heptane-1-carboxamide,
(1S,2S)-N-(6-((S)-1-cyanospiro[2.2]pentan-1-yl)isoquinolin-3-yl)-5-oxaspiro[2.4]heptane-1-carboxamide,
N-(7-chloro-6-(1-(4-fluoro-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(pyridin-2-yl)cyclopropane-1-carboxamide,
N-(6-(1-cyanospiro[2.2]pentan-1-yl)isoquinolin-3-yl)-2-(1-methyl-1H-pyrazol-3-yl)cyclopropane-1-carboxamide,
(1R,2R)-N-(6-((R)-1-cyanospiro[2.2]pentan-1-yl)isoquinolin-3-yl)-2-(1-methyl-1H-pyrazol-3-yl)cyclopropane-1-carboxamide,
(1R,2R)-N-(6-((S)-1-cyanospiro[2.2]pentan-1-yl)isoquinolin-3-yl)-2-(1-methyl-1H-pyrazol-3-yl)cyclopropane-1-carboxamide,
(1S,2S)-N-(6-((R)-1-cyanospiro[2.2]pentan-1-yl)isoquinolin-3-yl)-2-(1-methyl-1H-pyrazol-3-yl)cyclopropane-1-carboxamide,
(1S,2S)-N-(6-((S)-1-cyanospiro[2.2]pentan-1-yl)isoquinolin-3-yl)-2-(1-methyl-1H-pyrazol-3-yl)cyclopropane-1-carboxamide,
N-(6-(1-cyanospiro[2.2]pentan-1-yl)isoquinolin-3-yl)-2-methyl-3-(1-methyl-1H-pyrazol-4-yl)cyclopropane-1-carboxamide,
(1R,2R,3R)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3 S)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3R)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1R,2R,3 S)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3R)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3S)-N-(6-((R)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3R)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
(1S,2S,3S)-N-(6-((S)-1-cyanospiro[2.2]pentan-1-yl)isoquinolin-3-yl)-2-methyl-3-(1-methyl-1H-pyrazol-4-yl)cyclopropane-1-carboxamide,
N-(6-(1-cyanospiro[2.2]pentan-1-yl)isoquinolin-3-yl)-1-(1-methyl-1H-pyrazol-4-yl)pyrrolidine-3-carboxamide,
(R)-N-(6-((R)-1-cyanospiro[2.2]pentan-1-yl)isoquinolin-3-yl)-1-(1-methyl-1H-pyrazol-4-yl)pyrrolidine-3-carboxamide,
(R)-N-(6-((S)-1-cyanospiro[2.2]pentan-1-yl)isoquinolin-3-yl)-1-(1-methyl-1H-pyrazol-4-yl)pyrrolidine-3-carboxamide,
(S)-N-(6-((R)-1-cyanospiro[2.2]pentan-1-yl)isoquinolin-3-yl)-1-(1-methyl-1H-pyrazol-4-yl)pyrrolidine-3-carboxamide,
(S)-N-(6-((S)-1-cyanospiro[2.2]pentan-1-yl)isoquinolin-3-yl)-1-(1-methyl-1H-pyrazol-4-yl)pyrrolidine-3-carboxamide,
2-(5-chloro-1-methyl-1H-pyrazol-4-yl)-N-(6-(1-cyanospiro[2.2]pentan-1-yl)isoquinolin-3-yl)cyclopropane-1-carboxamide,
(1R,2R-(5-chloro-1-methyl-1H-pyrazol-4-yl))-N-(6-((R)-1-cyanospiro[2.2]pentan-1-yl)isoquinolin-3-yl)cyclopropane-1-carboxamide,
(1R,2R-(5-chloro-1-methyl-1H-pyrazol-4-yl))-N-(6-((S)-1-cyanospiro[2.2]pentan-1-yl)isoquinolin-3-yl)cyclopropane-1-carboxamide,
(1S,2S-(5-chloro-1-methyl-1H-pyrazol-4-yl))-N-(6-((R)-1-cyanospiro[2.2]pentan-1-yl)isoquinolin-3-yl)cyclopropane-1-carboxamide,
(1S,2S-(5-chloro-1-methyl-1H-pyrazol-4-yl))-N-(6-((S)-1-cyanospiro[2.2]pentan-1-yl)isoquinolin-3-yl)cyclopropane-1-carboxamide,
N-(7-chloro-6-(1-(4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2R,3R)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2R,3S)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2S,3R)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2S,3S)-N-(7-chloro-6-(1-((3R,4R)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2R,3R)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2R,3 S)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1R,2S,3R)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
(1S,2S,3S)-N-(7-chloro-6-(1-((3S,4S)-4-fluoro-3-methyltetrahydrofuran-3-yl)piperidin-4-yl)isoquinolin-3-yl)-2-methyl-3-(1-methyl-1H-pyrazol-4-yl)cyclopropane-1-carboxamide,
N-(7-chloro-6-(1-(4-fluoro-3-methyltetrahydrofuran-3-yl)piperidin-4-yl)isoquinolin-3-yl)-5-ethoxyspiro[2.3]hexane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-5-ethoxyspiro[2.3]hexane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-5-ethoxyspiro[2.3]hexane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-5-ethoxyspiro[2.3]hexane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-5-ethoxyspiro[2.3]hexane-1-carboxamide,
N-(7-chloro-6-(1-(4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-3-yl)cyclopropane-1-carboxamide,
N-(7-chloro-6-(1-(4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
1R,2R)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-ethyl-3-(1-methyl-1H-pyrazol-3-yl)cyclopropane-1-carboxamide,
N-(7-chloro-6-(1-(4-hydroxy-3-methyltetrahydrofuran-3-yl)piperidin-4-yl)isoquinolin-3-yl)-2-methyl-2-(pyridin-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-methyl-2-(pyridin-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-methyl-2-(pyridin-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-methyl-2-(pyridin-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-methyl-2-(pyridin-2-yl)cyclopropane-1-carboxamide,
N-(7-chloro-6-(1-(4-fluoro-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(tetrahydro-2H-pyran-4-yl)cyclopropane-1-carboxamide,
N-(7-chloro-6-(1-(4-fluoro-3-methyltetrahydrofuran-3-yl)piperidin-4-yl)isoquinolin-3-yl)-2-cyanocyclobutane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1R,2S)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1S,2R)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-cyanocyclobutane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1R,2S)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1S,2R)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-cyanocyclobutane-1-carboxamide,
N-(7-chloro-6-(1-(4-fluoro-3-methyltetrahydrofuran-3-yl)piperidin-4-yl)isoquinolin-3-yl)spiro[2.2]pentane-1-carboxamide,
(R)-N-(7-chloro-6-(1-((3R,4R)-4-fluoro-3-methyltetrahydrofuran-3-yl)piperidin-4-yl)isoquinolin-3-yl)spiro[2.2]pentane-1-carboxamide,
(R)-N-(7-chloro-6-(1-((3S,4S)-4-fluoro-3-methyltetrahydrofuran-3-yl)piperidin-4-yl)isoquinolin-3-yl)spiro[2.2]pentane-1-carboxamide,
(S)-N-(7-chloro-6-(1-((3R,4R)-4-fluoro-3-methyltetrahydrofuran-3-yl)piperidin-4-yl)isoquinolin-3-yl)spiro[2.2]pentane-1-carboxamide,
(S)-N-(7-chloro-6-(1-((3S,4S)-4-fluoro-3-methyltetrahydrofuran-3-yl)piperidin-4-yl)isoquinolin-3-yl)spiro[2.2]pentane-1-carboxamide,
N-(7-chloro-6-(spiro[2.2]pentan-1-yl)isoquinolin-3-yl)-2-ethyl-3-(1-methyl-1H-pyrazol-4-yl)cyclopropane-1-carboxamide,
(1R,2R,3R)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1R,2R,3R)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1S,2R,3 S)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1S,2R,3 S)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1R,2S,3R)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1R,2S,3R)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
(1S,2S,3S)-N-(7-chloro-6R-(spiro[2.2]pentan-1-yl)isoquinolin-3-yl)-2-ethyl-3-(1-methyl-1H-pyrazol-4-yl)cyclopropane-1-carboxamide,
(1S,2S,3S)-N-(7-chloro-6S-(spiro[2.2]pentan-1-yl)isoquinolin-3-yl)-2-ethyl-3-(1-methyl-1H-pyrazol-4-yl)cyclopropane-1-carboxamide,
N-(7-chloro-6-(spiro[2.2]pentan-1-yl)isoquinolin-3-yl)-2-(1-methyl-1H-pyrazol-4-yl)cyclopropane-1-carboxamide,
(1R,2R)-N-(7-chloro-6R-(spiro[2.2]pentan-1-yl)isoquinolin-3-yl)-2-(1-methyl-1H-pyrazol-4-yl)cyclopropane-1-carboxamide,
(1R,2R)-N-(7-chloro-6S-(spiro[2.2]pentan-1-yl)isoquinolin-3-yl)-2-(1-methyl-1H-pyrazol-4-yl)cyclopropane-1-carboxamide,
(1S,2S)-N-(7-chloro-6R-(spiro[2.2]pentan-1-yl)isoquinolin-3-yl)-2-(1-methyl-1H-pyrazol-4-yl)cyclopropane-1-carboxamide,
(1S,2S)-N-(7-chloro-6S-(spiro[2.2]pentan-1-yl)isoquinolin-3-yl)-2-(1-methyl-1H-pyrazol-4-yl)cyclopropane-1-carboxamide,
N-(7-chloro-6-(1-(4-hydroxy-3-methyltetrahydrofuran-3-yl)piperidin-4-yl)isoquinolin-3-yl)-2-cyclopropylpropanamide,
(R)-N-(7-chloro-6-(1-((3R,4R)-4-hydroxy-3-methyltetrahydrofuran-3-yl)piperidin-4-yl)isoquinolin-3-yl)-2-cyclopropylpropanamide,
(S)-N-(7-chloro-6-(1-((3R,4R)-4-hydroxy-3-methyltetrahydrofuran-3-yl)piperidin-4-yl)isoquinolin-3-yl)-2-cyclopropylpropanamide,
(R)-N-(7-chloro-6-(1-((3S,4S)-4-hydroxy-3-methyltetrahydrofuran-3-yl)piperidin-4-yl)isoquinolin-3-yl)-2-cyclopropylpropanamide,
(S)-N-(7-chloro-6-(1-((3S,4S)-4-hydroxy-3-methyltetrahydrofuran-3-yl)piperidin-4-yl)isoquinolin-3-yl)-2-cyclopropylpropanamide,
N-(7-chloro-6-(1-(4-hydroxy-3-methyltetrahydrofuran-3-yl)piperidin-4-yl)isoquinolin-3-yl)-2-(tetrahydro-2H-pyran-4-yl)propenamide,
(R)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)propenamide,
(S)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)propenamide,
(R)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-4-yl)propenamide,
(S)-N-(7-chloro-6-(1-((3 S,4S)-4-hydroxy-3-methyltetrahydrofuran-3-yl)piperidin-4-yl)isoquinolin-3-yl)-2-(tetrahydro-2H-pyran-4-yl)propenamide,
N-(7-chloro-6-(1-(4-hydroxy-3-methyltetrahydrofuran-3-yl)piperidin-4-yl)isoquinolin-3-yl)-2-(1-methyl-1H-pyrazol-4-yl)propenamide,
(R)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)propenamide,
(S)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-4-yl)propenamide,
(R)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)propenamide,
(S)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-4-yl)propenamide,
N-(6-(1-cyanospiro[2.2]pentan-1-yl)isoquinolin-3-yl)-2-(1-methyl-1H-pyrazol-4-yl)propenamide,
(R)-N-(6-(1R-cyanospiro[2.2]pentan-1-yl)isoquinolin-3-yl)-2-(1-methyl-1H-pyrazol-4-yl)propenamide,
(S)-N-(6-(1R-cyanospiro[2.2]pentan-1-yl)isoquinolin-3-yl)-2-(1-methyl-1H-pyrazol-4-yl)propenamide,
(R)-N-(6-(1S-cyanospiro[2.2]pentan-1-yl)isoquinolin-3-yl)-2-(1-methyl-1H-pyrazol-4-yl)propenamide,
(S)-N-(6-(1S-cyanospiro[2.2]pentan-1-yl)isoquinolin-3-yl)-2-(1-methyl-1H-pyrazol-4-yl)propenamide,
N-(6-(1-cyanospiro[2.2]pentan-1-yl)isoquinolin-3-yl)-2-(1-propionylpiperidin-4-yl)propenamide,
(R)-N-(6-(1R-cyanospiro[2.2]pentan-1-yl)isoquinolin-3-yl)-2-(1-propionylpiperidin-4-yl)propenamide,
(R)-N-(6-(1S-cyanospiro[2.2]pentan-1-yl)isoquinolin-3-yl)-2-(1-propionylpiperidin-4-yl)propenamide,
(S)-N-(6-(1R-cyanospiro[2.2]pentan-1-yl)isoquinolin-3-yl)-2-(1-propionylpiperidin-4-yl)propenamide,
(S)-N-(6-(1S-cyanospiro[2.2]pentan-1-yl)isoquinolin-3-yl)-2-(1-propionylpiperidin-4-yl)propenamide,
N-(7-chloro-6-(1-(4-hydroxy-3-methyltetrahydrofuran-3-yl)piperidin-4-yl)isoquinolin-3-yl)-2-(difluoromethyl)cyclopropane-1-carboxamide,
(2R)-N-(7-chloro-6-(1-((3R,4R)-4-hydroxy-3-methyltetrahydrofuran-3-yl)piperidin-4-yl)isoquinolin-3-yl)tetrahydrofuran-2-carboxamide,
(2R)-N-(7-chloro-6-(1-((3S,4S)-4-hydroxy-3-methyltetrahydrofuran-3-yl)piperidin-4-yl)isoquinolin-3-yl)tetrahydrofuran-2-carboxamide,
1-((1H-imidazol-1-yl)methyl)-N-(7-chloro-6-(1-(4-hydroxy-3-methyltetrahydrofuran-3-yl)piperidin-4-yl)isoquinolin-3-yl)cyclopropane-1-carboxamide,
1-((1H-imidazol-1-yl)methyl)-N-(7-chloro-6-(1-((3R,4R)-4-hydroxy-3-methyltetrahydrofuran-3-yl)piperidin-4-yl)isoquinolin-3-yl)cyclopropane-1-carboxamide,
1-((1H-imidazol-1-yl)methyl)-N-(7-chloro-6-(1-((3S,4S)-4-hydroxy-3-methyltetrahydrofuran-3-yl)piperidin-4-yl)isoquinolin-3-yl)cyclopropane-1-carboxamide,
N-(7-chloro-6-(1-(4-hydroxy-3-methyltetrahydrofuran-3-yl)piperidin-4-yl)isoquinolin-3-yl)-2-fluorocyclopropane-1-carboxamide,
(1R,2S)-N-(7-chloro-6-(1-((3R,4R)-4-hydroxy-3-methyltetrahydrofuran-3-yl)piperidin-4-yl)isoquinolin-3-yl)-2-fluorocyclopropane-1-carboxamide,
(1R,2S)-N-(7-chloro-6-(1-((3S,4S)-4-hydroxy-3-methyltetrahydrofuran-3-yl)piperidin-4-yl)isoquinolin-3-yl)-2-fluorocyclopropane-1-carboxamide,
(1S,2R)-N-(7-chloro-6-(1-((3R,4R)-4-hydroxy-3-methyltetrahydrofuran-3-yl)piperidin-4-yl)isoquinolin-3-yl)-2-fluorocyclopropane-1-carboxamide,
(1S,2R)-N-(7-chloro-6-(1-((3S,4S)-4-hydroxy-3-methyltetrahydrofuran-3-yl)piperidin-4-yl)isoquinolin-3-yl)-2-fluorocyclopropane-1-carboxamide,
N-(7-chloro-6-(1-(4-hydroxytetrahydrofuran-3-yl)piperidin-4-yl)isoquinolin-3-yl)-1-methyl-1H-pyrazole-4-carboxamide,
N-(7-chloro-6-(1-((3R,4R)-4-hydroxytetrahydrofuran-3-yl)piperidin-4-yl)isoquinolin-3-yl)-1-methyl-1H-pyrazole-4-carboxamide,
N-(7-chloro-6-(1-((3S,4S)-4-hydroxytetrahydrofuran-3-yl)piperidin-4-yl)isoquinolin-3-yl)-1-methyl-1H-pyrazole-4-carboxamide,
N-(7-chloro-6-(1-(4-hydroxy-3-methyltetrahydrofuran-3-yl)piperidin-4-yl)isoquinolin-3-yl)-2-(pyridin-2-yl)acetamide,
N-(7-chloro-6-(1-((3R,4R)-4-hydroxy-3-methyltetrahydrofuran-3-yl)piperidin-4-yl)isoquinolin-3-yl)-2-(pyridin-2-yl)acetamide,
N-(7-chloro-6-(1-((3S,4S)-4-hydroxy-3-methyltetrahydrofuran-3-yl)piperidin-4-yl)isoquinolin-3-yl)-2-(pyridin-2-yl)acetamide,
N-(7-chloro-6-(1-(4-hydroxy-3-methyltetrahydrofuran-3-yl)piperidin-4-yl)isoquinolin-3-yl)-2-(pyridin-3-yl)acetamide,
N-(7-chloro-6-(1-((3R,4R)-4-hydroxy-3-methyltetrahydrofuran-3-yl)piperidin-4-yl)isoquinolin-3-yl)-2-(pyridin-3-yl)acetamide,
N-(7-chloro-6-(1-((3S,4S)-4-hydroxy-3-methyltetrahydrofuran-3-yl)piperidin-4-yl)isoquinolin-3-yl)-2-(pyridin-3-yl)acetamide,
N-(7-chloro-6-(1-(4-hydroxy-3-methyltetrahydrofuran-3-yl)piperidin-4-yl)isoquinolin-3-yl)-2-(pyridin-4-yl)acetamide,
N-(7-chloro-6-(1-((3R,4R)-4-hydroxy-3-methyltetrahydrofuran-3-yl)piperidin-4-yl)isoquinolin-3-yl)-2-(pyridin-4-yl)acetamide,
N-(7-chloro-6-(1-((3S,4S)-4-hydroxy-3-methyltetrahydrofuran-3-yl)piperidin-4-yl)isoquinolin-3-yl)-2-(pyridin-4-yl)acetamide,
2-(4-chloro-1H-pyrazol-1-yl)-N-(7-chloro-6-(1-(4-hydroxy-3-methyltetrahydrofuran-3-yl)piperidin-4-yl)isoquinolin-3-yl)acetamide,
2-(4-chloro-1H-pyrazol-1-yl)-N-(7-chloro-6-(1-((3R,4R)-4-hydroxy-3-methyltetrahydrofuran-3-yl)piperidin-4-yl)isoquinolin-3-yl)acetamide,
2-(4-chloro-1H-pyrazol-1-yl)-N-(7-chloro-6-(1-((3S,4S)-4-hydroxy-3-methyltetrahydrofuran-3-yl)piperidin-4-yl)isoquinolin-3-yl)acetamide,
N-(7-chloro-6-(1-(4-hydroxy-3-methyltetrahydrofuran-3-yl)piperidin-4-yl)isoquinolin-3-yl)-2-(3-(trifluoromethyl)-1H-pyrazol-1-yl)acetamide,
N-(7-chloro-6-(1-((3R,4R)-4-hydroxy-3-methyltetrahydrofuran-3-yl)piperidin-4-yl)isoquinolin-3-yl)-2-(3-(trifluoromethyl)-1H-pyrazol-1-yl)acetamide,
N-(7-chloro-6-(1-((3S,4S)-4-hydroxy-3-methyltetrahydrofuran-3-yl)piperidin-4-yl)isoquinolin-3-yl)-2-(3-(trifluoromethyl)-1H-pyrazol-1-yl)acetamide,
N-(7-chloro-6-(1-(4-hydroxy-3-methyltetrahydrofuran-3-yl)piperidin-4-yl)isoquinolin-3-yl)-2-(1-(difluoromethyl)-1H-pyrazol-4-yl)acetamide,
N-(7-chloro-6-(1-((3R,4R)-4-hydroxy-3-methyltetrahydrofuran-3-yl)piperidin-4-yl)isoquinolin-3-yl)-2-(1-(difluoromethyl)-1H-pyrazol-4-yl)acetamide,
N-(7-chloro-6-(1-((3S,4S)-4-hydroxy-3-methyltetrahydrofuran-3-yl)piperidin-4-yl)isoquinolin-3-yl)-2-(1-(difluoromethyl)-1H-pyrazol-4-yl)acetamide,
2-(3-chloro-1-methyl-1H-pyrazol-4-yl)-N-(7-chloro-6-(1-(4-hydroxy-3-methyltetrahydrofuran-3-yl)piperidin-4-yl)isoquinolin-3-yl)acetamide,
2-(3-chloro-1-methyl-1H-pyrazol-4-yl)-N-(7-chloro-6-(1-((3R,4R)-4-hydroxy-3-methyltetrahydrofuran-3-yl)piperidin-4-yl)isoquinolin-3-yl)acetamide,
2-(3-chloro-1-methyl-1H-pyrazol-4-yl)-N-(7-chloro-6-(1-((3S,4S)-4-hydroxy-3-methyltetrahydrofuran-3-yl)piperidin-4-yl)isoquinolin-3-yl)acetamide,
(R or S)-N-(7-chloro-6-(1-((3R,4R or 3R,4R)-4-hydroxytetrahydrofuran-3-yl)piperidin-4-yl)isoquinolin-3-yl)-6-oxaspiro[2.5]octane-1-carboxamide,
N-(7-chloro-6-(1-(4-hydroxytetrahydrofuran-3-yl)piperidin-4-yl)isoquinolin-3-yl)-6-oxaspiro[2.5]octane-1-carboxamide,
(R)-N-(7-chloro-6-(1-((3R,4R)-4-hydroxytetrahydrofuran-3-yl)piperidin-4-yl)isoquinolin-3-yl)-6-oxaspiro[2.5]octane-1-carboxamide,
(R)-N-(7-chloro-6-(1-((3S,4S)-4-hydroxytetrahydrofuran-3-yl)piperidin-4-yl)isoquinolin-3-yl)-6-oxaspiro[2.5]octane-1-carboxamide,
(S)-N-(7-chloro-6-(1-((3R,4R)-4-hydroxytetrahydrofuran-3-yl)piperidin-4-yl)isoquinolin-3-yl)-6-oxaspiro[2.5]octane-1-carboxamide,
(S)-N-(7-chloro-6-(1-((3S,4S)-4-hydroxytetrahydrofuran-3-yl)piperidin-4-yl)isoquinolin-3-yl)-6-oxaspiro[2.5]octane-1-carboxamide,
N-(7-chloro-6-(1-(4-hydroxy-3-methyltetrahydrofuran-3-yl)piperidin-4-yl)isoquinolin-3-yl)spiro[2.2]pentane-1-carboxamide,
(R)-N-(7-chloro-6-(1-((3R,4R)-4-hydroxy-3-methyltetrahydrofuran-3-yl)piperidin-4-yl)isoquinolin-3-yl)spiro[2.2]pentane-1-carboxamide,
(R)-N-(7-chloro-6-(1-((3S,4S)-4-hydroxy-3-methyltetrahydrofuran-3-yl)piperidin-4-yl)isoquinolin-3-yl)spiro[2.2]pentane-1-carboxamide,
(S)-N-(7-chloro-6-(1-((3R,4R)-4-hydroxy-3-methyltetrahydrofuran-3-yl)piperidin-4-yl)isoquinolin-3-yl)spiro[2.2]pentane-1-carboxamide,
(S)-N-(7-chloro-6-(1-((3S,4S)-4-hydroxy-3-methyltetrahydrofuran-3-yl)piperidin-4-yl)isoquinolin-3-yl)spiro[2.2]pentane-1-carboxamide,
N-(7-chloro-6-(1-(4-hydroxy-3-methyltetrahydrofuran-3-yl)piperidin-4-yl)isoquinolin-3-yl)spiro[2.3]hexane-1-carboxamide,
(R)-N-(7-chloro-6-(1-((3R,4R)-4-hydroxy-3-methyltetrahydrofuran-3-yl)piperidin-4-yl)isoquinolin-3-yl)spiro[2.3]hexane-1-carboxamide,
(R)-N-(7-chloro-6-(1-((3S,4S)-4-hydroxy-3-methyltetrahydrofuran-3-yl)piperidin-4-yl)isoquinolin-3-yl)spiro[2.3]hexane-1-carboxamide,
(S)-N-(7-chloro-6-(1-((3R,4R)-4-hydroxy-3-methyltetrahydrofuran-3-yl)piperidin-4-yl)isoquinolin-3-yl)spiro[2.3]hexane-1-carboxamide,
(S)-N-(7-chloro-6-(1-((3S,4S)-4-hydroxy-3-methyltetrahydrofuran-3-yl)piperidin-4-yl)isoquinolin-3-yl)spiro[2.3]hexane-1-carboxamide,
Trans-N-(7-chloro-6-(1-(4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((4R,3R)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((4R,3R)-4-hydroxy-3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-((4S,3S)-4-hydroxy-3-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-((4S,3S)-4-hydroxy-3-methyltetrahydrofuran-3-yl)piperidin-4-yl)isoquinolin-3-yl)-2-(2-hydroxypropan-2-yl)cyclopropane-1-carboxamide,
N-(7-chloro-6-(1-(4-hydroxy-3-methyltetrahydrofuran-3-yl)piperidin-4-yl)isoquinolin-3-yl)-2-oxabicyclo[4.1.0]heptane-7-carboxamide,
(1S,6S,7R)-N-(7-chloro-6-(1-((3R,4R)-4-hydroxy-3-methyltetrahydrofuran-3-yl)piperidin-4-yl)isoquinolin-3-yl)-2-oxabicyclo[4.1.0]heptane-7-carboxamide,
(1R,6R,7R)-N-(7-chloro-6-(1-((3R,4R)-4-hydroxy-3-methyltetrahydrofuran-3-yl)piperidin-4-yl)isoquinolin-3-yl)-2-oxabicyclo[4.1.0]heptane-7-carboxamide,
(1S,6S,7R)-N-(7-chloro-6-(1-((3S,4S)-4-hydroxy-3-methyltetrahydrofuran-3-yl)piperidin-4-yl)isoquinolin-3-yl)-2-oxabicyclo[4.1.0]heptane-7-carboxamide,
(1R,6R,7R)-N-(7-chloro-6-(1-((3S,4S)-4-hydroxy-3-methyltetrahydrofuran-3-yl)piperidin-4-yl)isoquinolin-3-yl)-2-oxabicyclo[4.1.0]heptane-7-carboxamide,
N-(7-chloro-6-(1-(4-hydroxy-3-methyltetrahydrofuran-3-yl)piperidin-4-yl)isoquinolin-3-yl)-2-(3-methyl-1,2,4-oxadiazol-5-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(3-methyl-1,2,4-oxadiazol-5-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(3-methyl-1,2,4-oxadiazol-5-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(3-methyl-1,2,4-oxadiazol-5-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(3-methyl-1,2,4-oxadiazol-5-yl)cyclopropane-1-carboxamide,
N-(7-chloro-6-(1-(4-hydroxy-3-methyltetrahydrofuran-3-yl)piperidin-4-yl)isoquinolin-3-yl)-5-oxaspiro[3.4]octane-7-carboxamide,
(7S)-N-(7-chloro-6-(1-((3R,4R)-4-hydroxy-3-methyltetrahydrofuran-3-yl)piperidin-4-yl)isoquinolin-3-yl)-5-oxaspiro[3.4]octane-7-carboxamide,
(7S)-N-(7-chloro-6-(1-((3S,4S)-4-hydroxy-3-methyltetrahydrofuran-3-yl)piperidin-4-yl)isoquinolin-3-yl)-5-oxaspiro[3.4]octane-7-carboxamide,
(7R)-N-(7-chloro-6-(1-((3R,4R)-4-hydroxy-3-methyltetrahydrofuran-3-yl)piperidin-4-yl)isoquinolin-3-yl)-5-oxaspiro[3.4]octane-7-carboxamide,
(7R)-N-(7-chloro-6-(1-((3S,4S)-4-hydroxy-3-methyltetrahydrofuran-3-yl)piperidin-4-yl)isoquinolin-3-yl)-5-oxaspiro[3.4]octane-7-carboxamide,
N-(7-chloro-6-(1-(4-hydroxy-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(pyridin-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(pyridin-4-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(pyridin-4-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(pyridin-4-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(pyridin-4-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(pyridin-4-yl)cyclopropane-1-carboxamide,
N-(7-chloro-6-(1-(4-hydroxy-3-methyltetrahydrofuran-3-yl)piperidin-4-yl)isoquinolin-3-yl)-5-oxaspiro[3.5]nonane-7-carboxamide,
(7S)-N-(7-chloro-6-(1-(3R,4R)-4-hydroxy-3-methyltetrahydrofuran-3-yl)piperidin-4-yl)isoquinolin-3-yl)-5-oxaspiro[3.5]nonane-7-carboxamide,
(7R)-N-(7-chloro-6-(1-((3R,4R)-4-hydroxy-3-methyltetrahydrofuran-3-yl)piperidin-4-yl)isoquinolin-3-yl)-5-oxaspiro[3.5]nonane-7-carboxamide,
(7S)-N-(7-chloro-6-(1-(3S,4S)-4-hydroxy-3-methyltetrahydrofuran-3-yl)piperidin-4-yl)isoquinolin-3-yl)-5-oxaspiro[3.5]nonane-7-carboxamide,
(7R)-N-(7-chloro-6-(1-((3S,4S)-4-hydroxy-3-methyltetrahydrofuran-3-yl)piperidin-4-yl)isoquinolin-3-yl)-5-oxaspiro[3.5]nonane-7-carboxamide,
N-(7-chloro-6-(1-(4-hydroxy-3-methyltetrahydrofuran-3-yl)piperidin-4-yl)isoquinolin-3-yl)-6,6-dimethyltetrahydro-2H-pyran-3-carboxamide,
(3S)-N-(7-chloro-6-(1-((3R,4R)-4-hydroxy-3-methyltetrahydrofuran-3-yl)piperidin-4-yl)isoquinolin-3-yl)-6,6-dimethyltetrahydro-2H-pyran-3-carboxamide,
(3R)-N-(7-chloro-6-(1-((3R,4R)-4-hydroxy-3-methyltetrahydrofuran-3-yl)piperidin-4-yl)isoquinolin-3-yl)-6,6-dimethyltetrahydro-2H-pyran-3-carboxamide,
(3S)-N-(7-chloro-6-(1-((3S,4S)-4-hydroxy-3-methyltetrahydrofuran-3-yl)piperidin-4-yl)isoquinolin-3-yl)-6,6-dimethyltetrahydro-2H-pyran-3-carboxamide,
(3R)-N-(7-chloro-6-(1-((3S,4S)-4-hydroxy-3-methyltetrahydrofuran-3-yl)piperidin-4-yl)isoquinolin-3-yl)-6,6-dimethyltetrahydro-2H-pyran-3-carboxamide,
N-(7-chloro-6-(1-(4-hydroxy-3-methyltetrahydrofuran-3-yl)piperidin-4-yl)isoquinolin-3-yl)-2-(dimethylamino)bicyclo[3.1.0]hexane-6-carboxamide,
(1R,5S,6R)-N-(7-chloro-6-(1-((3R,4R)-4-hydroxy-3-methyltetrahydrofuran-3-yl)piperidin-4-yl)isoquinolin-3-yl)-2-(dimethylamino)bicyclo[3.1.0]hexane-6-carboxamide,
(1R,5S,6S)-N-(7-chloro-6-(1-((3R,4R)-4-hydroxy-3-methyltetrahydrofuran-3-yl)piperidin-4-yl)isoquinolin-3-yl)-2-(dimethylamino)bicyclo[3.1.0]hexane-6-carboxamide,
(1S,5R,6R)-N-(7-chloro-6-(1-((3R,4R)-4-hydroxy-3-methyltetrahydrofuran-3-yl)piperidin-4-yl)isoquinolin-3-yl)-2-(dimethylamino)bicyclo[3.1.0]hexane-6-carboxamide,
(1S,5R,6S)-N-(7-chloro-6-(1-((3R,4R)-4-hydroxy-3-methyltetrahydrofuran-3-yl)piperidin-4-yl)isoquinolin-3-yl)-2-(dimethylamino)bicyclo[3.1.0]hexane-6-carboxamide,
(1R,5S,6R)-N-(7-chloro-6-(1-((3S,4S)-4-hydroxy-3-methyltetrahydrofuran-3-yl)piperidin-4-yl)isoquinolin-3-yl)-2-(dimethylamino)bicyclo[3.1.0]hexane-6-carboxamide,
(1R,5S,6S)-N-(7-chloro-6-(1-((3S,4S)-4-hydroxy-3-methyltetrahydrofuran-3-yl)piperidin-4-yl)isoquinolin-3-yl)-2-(dimethylamino)bicyclo[3.1.0]hexane-6-carboxamide,
(1S,5R,6R)-N-(7-chloro-6-(1-((3S,4S)-4-hydroxy-3-methyltetrahydrofuran-3-yl)piperidin-4-yl)isoquinolin-3-yl)-2-(dimethylamino)bicyclo[3.1.0]hexane-6-carboxamide,
(1S,5R,6S)-N-(7-chloro-6-(1-((3S,4S)-4-hydroxy-3-methyltetrahydrofuran-3-yl)piperidin-4-yl)isoquinolin-3-yl)-2-(dimethylamino)bicyclo[3.1.0]hexane-6-carboxamide,
N-(7-chloro-6-(1-(4-hydroxy-3-methyltetrahydrofuran-3-yl)piperidin-4-yl)isoquinolin-3-yl)-5-ethoxyspiro[2.3]hexane-1-carboxamide,
(1R)-N-(7-chloro-6-(1-((3R,4R)-4-hydroxy-3-methyltetrahydrofuran-3-yl)piperidin-4-yl)isoquinolin-3-yl)-5-ethoxyspiro[2.3]hexane-1-carboxamide,
(1R)-N-(7-chloro-6-(1-((3S,4S)-4-hydroxy-3-methyltetrahydrofuran-3-yl)piperidin-4-yl)isoquinolin-3-yl)-5-ethoxyspiro[2.3]hexane-1-carboxamide,
(1S)-N-(7-chloro-6-(1-((3R,4R)-4-hydroxy-3-methyltetrahydrofuran-3-yl)piperidin-4-yl)isoquinolin-3-yl)-5-ethoxyspiro[2.3]hexane-1-carboxamide,
(1S)-N-(7-chloro-6-(1-((3S,4S)-4-hydroxy-3-methyltetrahydrofuran-3-yl)piperidin-4-yl)isoquinolin-3-yl)-5-ethoxyspiro[2.3]hexane-1-carboxamide,
N-(7-chloro-6-(1-(4-hydroxy-3-methyltetrahydrofuran-3-yl)piperidin-4-yl)isoquinolin-3-yl)-3-oxabicyclo[4.1.0]heptane-7-carboxamide,
(1R,6S,7R)-N-(7-chloro-6-(1-((3R,4R)-4-hydroxy-3-methyltetrahydrofuran-3-yl)piperidin-4-yl)isoquinolin-3-yl)-3-oxabicyclo[4.1.0]heptane-7-carboxamide,
(1R,6S,7R)-N-(7-chloro-6-(1-((3S,4S)-4-hydroxy-3-methyltetrahydrofuran-3-yl)piperidin-4-yl)isoquinolin-3-yl)-3-oxabicyclo[4.1.0]heptane-7-carboxamide,
(1R,6S,7S)-N-(7-chloro-6-(1-((3R,4R)-4-hydroxy-3-methyltetrahydrofuran-3-yl)piperidin-4-yl)isoquinolin-3-yl)-3-oxabicyclo[4.1.0]heptane-7-carboxamide,
(1R,6S,7S)-N-(7-chloro-6-(1-((3S,4S)-4-hydroxy-3-methyltetrahydrofuran-3-yl)piperidin-4-yl)isoquinolin-3-yl)-3-oxabicyclo[4.1.0]heptane-7-carboxamide,
(1S,6R,7R)-N-(7-chloro-6-(1-((3R,4R)-4-hydroxy-3-methyltetrahydrofuran-3-yl)piperidin-4-yl)isoquinolin-3-yl)-3-oxabicyclo[4.1.0]heptane-7-carboxamide,
(1S,6R,7R)-N-(7-chloro-6-(1-((3S,4S)-4-hydroxy-3-methyltetrahydrofuran-3-yl)piperidin-4-yl)isoquinolin-3-yl)-3-oxabicyclo[4.1.0]heptane-7-carboxamide,
(1S,6R,7S)-N-(7-chloro-6-(1-((3R,4R)-4-hydroxy-3-methyltetrahydrofuran-3-yl)piperidin-4-yl)isoquinolin-3-yl)-3-oxabicyclo[4.1.0]heptane-7-carboxamide,
(1S,6R,7S)-N-(7-chloro-6-(1-((3S,4S)-4-hydroxy-3-methyltetrahydrofuran-3-yl)piperidin-4-yl)isoquinolin-3-yl)-3-oxabicyclo[4.1.0]heptane-7-carboxamide,
N-(7-chloro-6-(1-(4-hydroxy-3-methyltetrahydrofuran-3-yl)piperidin-4-yl)isoquinolin-3-yl)-2-(pyrimidin-5-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(pyrimidin-5-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(pyrimidin-5-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(pyrimidin-5-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(pyrimidin-5-yl)cyclopropane-1-carboxamide,
N-(7-chloro-6-(1-(4-hydroxy-3-methyltetrahydrofuran-3-yl)piperidin-4-yl)isoquinolin-3-yl)-2-(1-methyl-5-(trifluoromethyl)-1H-pyrazol-4-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(1-methyl-5-(trifluoromethyl)-1H-pyrazol-4-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(1-methyl-5-(trifluoromethyl)-1H-pyrazol-4-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(1-methyl-5-(trifluoromethyl)-1H-pyrazol-4-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(1-methyl-5-(trifluoromethyl)-1H-pyrazol-4-yl)cyclopropane-1-carboxamide,
N-(7-chloro-6-(1-(4-hydroxy-3-methyltetrahydrofuran-3-yl)piperidin-4-yl)isoquinolin-3-yl)-2-(4-fluoropyridin-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(4-fluoropyridin-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(4-fluoropyridin-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(4-fluoropyridin-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(4-fluoropyridin-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(pyridin-3-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(pyridin-3-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(pyridin-3-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(pyridin-3-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(pyridin-3-yl)cyclopropane-1-carboxamide,
N-(7-chloro-6-(1-(4-hydroxy-3-methyltetrahydrofuran-3-yl)piperidin-4-yl)isoquinolin-3-yl)-2-(difluoromethyl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
(1R,2S)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1R,2S)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
(1S,2R)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1S,2R)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
N-(7-chloro-6-(1-(4-hydroxy-3-methyltetrahydrofuran-3-yl)piperidin-4-yl)isoquinolin-3-yl)-2,2-dimethyl-3-(pyridin-2-yl)cyclopropane-1-carboxamide,
(1R,3R)-N-(7-chloro-6-(1-((3R,4R)-4-hydroxy-3-methyltetrahydrofuran-3-yl)piperidin-4-yl)isoquinolin-3-yl)-2,2-dimethyl-3-(pyridin-2-yl)cyclopropane-1-carboxamide,
(1R,3R)-N-(7-chloro-6-(1-((3S,4S)-4-hydroxy-3-methyltetrahydrofuran-3-yl)piperidin-4-yl)isoquinolin-3-yl)-2,2-dimethyl-3-(pyridin-2-yl)cyclopropane-1-carboxamide,
(1S,3S)-N-(7-chloro-6-(1-((3R,4R)-4-hydroxy-3-methyltetrahydrofuran-3-yl)piperidin-4-yl)isoquinolin-3-yl)-2,2-dimethyl-3-(pyridin-2-yl)cyclopropane-1-carboxamide,
(1S,3S)-N-(7-chloro-6-(1-((3S,4S)-4-hydroxy-3-methyltetrahydrofuran-3-yl)piperidin-4-yl)isoquinolin-3-yl)-2,2-dimethyl-3-(pyridin-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(2-fluoropropan-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(2-fluoropropan-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(2-fluoropropan-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(2-fluoropropan-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(2-fluoropropan-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-3-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(1-methyl-1H-pyrazol-3-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(1-methyl-1H-pyrazol-3-yl)cyclopropane-1-carboxamide,
N-(7-chloro-6-(1-(4-hydroxy-3-methyltetrahydrofuran-3-yl)piperidin-4-yl)isoquinolin-3-yl)-2-methyl-3-(pyridin-2-yl)cyclopropane-1-carboxamide,
(1S,2R,3R)-N-(7-chloro-6-(1-((3R,4R)-4-hydroxy-3-methyltetrahydrofuran-3-yl)piperidin-4-yl)isoquinolin-3-yl)-2-methyl-3-(pyridin-2-yl)cyclopropane-1-carboxamide,
(1S,2R,3R)-N-(7-chloro-6-(1-((3S,4S)-4-hydroxy-3-methyltetrahydrofuran-3-yl)piperidin-4-yl)isoquinolin-3-yl)-2-methyl-3-(pyridin-2-yl)cyclopropane-1-carboxamide,
(1S,2S,3R)-N-(7-chloro-6-(1-((3R,4R)-4-hydroxy-3-methyltetrahydrofuran-3-yl)piperidin-4-yl)isoquinolin-3-yl)-2-methyl-3-(pyridin-2-yl)cyclopropane-1-carboxamide,
(1S,2S,3R)-N-(7-chloro-6-(1-((3S,4S)-4-hydroxy-3-methyltetrahydrofuran-3-yl)piperidin-4-yl)isoquinolin-3-yl)-2-methyl-3-(pyridin-2-yl)cyclopropane-1-carboxamide,
(1R,2R,3S)-N-(7-chloro-6-(1-((3R,4R)-4-hydroxy-3-methyltetrahydrofuran-3-yl)piperidin-4-yl)isoquinolin-3-yl)-2-methyl-3-(pyridin-2-yl)cyclopropane-1-carboxamide,
(1R,2R,3S)-N-(7-chloro-6-(1-((3S,4S)-4-hydroxy-3-methyltetrahydrofuran-3-yl)piperidin-4-yl)isoquinolin-3-yl)-2-methyl-3-(pyridin-2-yl)cyclopropane-1-carboxamide,
(1R,2S,3S)-N-(7-chloro-6-(1-((3R,4R)-4-hydroxy-3-methyltetrahydrofuran-3-yl)piperidin-4-yl)isoquinolin-3-yl)-2-methyl-3-(pyridin-2-yl)cyclopropane-1-carboxamide,
(1R,2S,3S)-N-(7-chloro-6-(1-((3S,4S)-4-hydroxy-3-methyltetrahydrofuran-3-yl)piperidin-4-yl)isoquinolin-3-yl)-2-methyl-3-(pyridin-2-yl)cyclopropane-1-carboxamide,
N-(7-chloro-6-(1-(4-hydroxy-3-methyltetrahydrofuran-3-yl)piperidin-4-yl)isoquinolin-3-yl)-2-ethyl-3-(pyridin-2-yl)cyclopropane-1-carboxamide,
(1S,2R,3R)-N-(7-chloro-6-(1-((3R,4R)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1S,2R,3R)-N-(7-chloro-6-(1-((3S,4S)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1S,2S,3R)-N-(7-chloro-6-(1-((3R,4R)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1S,2S,3R)-N-(7-chloro-6-(1-((3S,4S)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1R,2R,3S)-N-(7-chloro-6-(1-((3R,4R)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1R,2R,3S)-N-(7-chloro-6-(1-((3S,4S)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1R,2S,3S)-N-(7-chloro-6-(1-((3R,4R)-4-hydroxy-3-methyltetrahydrofuran-3-yl)piperidin-4-yl)isoquinolin-3-yl)-2-ethyl-3-(pyridin-2-yl)cyclopropane-1-carboxamide,
(1R,2S,3S)-N-(7-chloro-6-(1-((3S,4S)-4-hydroxy-3-methyltetrahydrofuran-3-yl)piperidin-4-yl)isoquinolin-3-yl)-2-ethyl-3-(pyridin-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(5-methylthiophen-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(5-methylthiophen-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(5-methylthiophen-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(5-methylthiophen-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(5-methylthiophen-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(thiophen-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(thiophen-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(thiophen-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(thiophen-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(thiophen-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(1-isobutyl-1H-pyrazol-5-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(1-isobutyl-1H-pyrazol-5-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(1-isobutyl-1H-pyrazol-5-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(1-isobutyl-1H-pyrazol-5-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(1-isobutyl-1H-pyrazol-5-yl)cyclopropane-1-carboxamide,
N-(7-chloro-6-(1-(4-hydroxy-3-methyltetrahydrofuran-3-yl)piperidin-4-yl)isoquinolin-3-yl)-2-(1-isopropyl-1H-pyrazol-5-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(1-isopropyl-1H-pyrazol-5-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(1-isopropyl-1H-pyrazol-5-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(1-isopropyl-1H-pyrazol-5-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(1-isopropyl-1H-pyrazol-5-yl)cyclopropane-1-carboxamide,
N-(7-chloro-6-(1-(4-hydroxy-3-methyltetrahydrofuran-3-yl)piperidin-4-yl)isoquinolin-3-yl)-2-(furan-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(furan-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(furan-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(furan-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(furan-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-4-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-4-yl)cyclopropane-1-carboxamide,
N-(7-chloro-6-(1-(4-hydroxy-3-methyltetrahydrofuran-3-yl)piperidin-4-yl)isoquinolin-3-yl)-2-(tetrahydrofuran-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(tetrahydrofuran-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(tetrahydrofuran-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(tetrahydrofuran-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(tetrahydrofuran-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(tetrahydro-2H-pyran-2-yl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-2-yl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-2-yl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(tetrahydro-2H-pyran-2-yl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(tetrahydro-2H-pyran-2-yl)cyclopropane-1-carboxamide,
N-(7-chloro-6-(1-(4-hydroxy-3-methyltetrahydrofuran-3-yl)piperidin-4-yl)isoquinolin-3-yl)-2-(pyridin-2-yl)cyclobutane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(pyridin-2-yl)cyclobutane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(pyridin-2-yl)cyclobutane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(pyridin-2-yl)cyclobutane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(pyridin-2-yl)cyclobutane-1-carboxamide,
N-(7-chloro-6-(1-(4-hydroxy-3-methyltetrahydrofuran-3-yl)piperidin-4-yl)isoquinolin-3-yl)-3-(pyridin-2-yl)cyclobutane-1-carboxamide,
(1R,3R)-N-(7-chloro-6-(1-((3R,4R)-4-hydroxy-3-methyltetrahydrofuran-3-yl)piperidin-4-yl)isoquinolin-3-yl)-3-(pyridin-2-yl)cyclobutane-1-carboxamide,
(1R,3R)-N-(7-chloro-6-(1-((3S,4S)-4-hydroxy-3-methyltetrahydrofuran-3-yl)piperidin-4-yl)isoquinolin-3-yl)-3-(pyridin-2-yl)cyclobutane-1-carboxamide,
(1S,3S)-N-(7-chloro-6-(1-((3R,4R)-4-hydroxy-3-methyltetrahydrofuran-3-yl)piperidin-4-yl)isoquinolin-3-yl)-3-(pyridin-2-yl)cyclobutane-1-carboxamide,
(1S,3S)-N-(7-chloro-6-(1-((3S,4S)-4-hydroxy-3-methyltetrahydrofuran-3-yl)piperidin-4-yl)isoquinolin-3-yl)-3-(pyridin-2-yl)cyclobutane-1-carboxamide,
(1R,2R or 1S,2S)-N-(7-chloro-6-((cis or trans)-4-(3-fluoroazetidin-1-yl)cyclohexyl)isoquinolin-3-yl)-2-(pyridin-2-yl)cyclopropane-1-carboxamide,
N-(7-chloro-6-(4-(3,3-difluoroazetidin-1-yl)cyclohexyl)isoquinolin-3-yl)-2-(pyridin-2-yl)cyclopropane-1-carboxamide,
(1R,2R)-N-(7-chloro-6-(cis-4-(3,3-difluoroazetidin-1-yl)cyclohexyl)isoquinolin-3-yl)-2-(pyridin-2-yl)cyclopropane-1-carboxamide,
(1R,2R)-N-(7-chloro-6-(trans-4-(3,3-difluoroazetidin-1-yl)cyclohexyl)isoquinolin-3-yl)-2-(pyridin-2-yl)cyclopropane-1-carboxamide,
(1S,2S)-N-(7-chloro-6-(cis-4-(3,3-difluoroazetidin-1-yl)cyclohexyl)isoquinolin-3-yl)-2-(pyridin-2-yl)cyclopropane-1-carboxamide,
(1S,2S)-N-(7-chloro-6-(trans-4-(3,3-difluoroazetidin-1-yl)cyclohexyl)isoquinolin-3-yl)-2-(pyridin-2-yl)cyclopropane-1-carboxamide,
N-(7-chloro-6-(4-(3-fluoroazetidin-1-yl)cyclohexyl)isoquinolin-3-yl)-2-(1-methyl-1H-pyrazol-4-yl)cyclopropane-1-carboxamide,
(1S,2S)-N-(7-chloro-6-(cis-4-(3-fluoroazetidin-1-yl)cyclohexyl)isoquinolin-3-yl)-2-(1-methyl-1H-pyrazol-4-yl)cyclopropane-1-carboxamide,
(1S,2S)-N-(7-chloro-6-(trans-4-(3-fluoroazetidin-1-yl)cyclohexyl)isoquinolin-3-yl)-2-(1-methyl-1H-pyrazol-4-yl)cyclopropane-1-carboxamide,
(1R,2R)-N-(7-chloro-6-(cis-4-(3-fluoroazetidin-1-yl)cyclohexyl)isoquinolin-3-yl)-2-(1-methyl-1H-pyrazol-4-yl)cyclopropane-1-carboxamide,
(1R,2R)-N-(7-chloro-6-(trans-4-(3-fluoroazetidin-1-yl)cyclohexyl)isoquinolin-3-yl)-2-(1-methyl-1H-pyrazol-4-yl)cyclopropane-1-carboxamide,
N-(7-chloro-6-(4-(3-fluoropyrrolidin-1-yl)cyclohexyl)isoquinolin-3-yl)-2,2-dimethyltetrahydro-2H-pyran-4-carboxamide,
(4R)-N-(7-chloro-6-(cis-4-((R)-3-fluoropyrrolidin-1-yl)cyclohexyl)isoquinolin-3-yl)-2,2-dimethyltetrahydro-2H-pyran-4-carboxamide,
(4S)-N-(7-chloro-6-(cis-4-((R)-3-fluoropyrrolidin-1-yl)cyclohexyl)isoquinolin-3-yl)-2,2-dimethyltetrahydro-2H-pyran-4-carboxamide,
(4R)-N-(7-chloro-6-(trans-4-((R)-3-fluoropyrrolidin-1-yl)cyclohexyl)isoquinolin-3-yl)-2,2-dimethyltetrahydro-2H-pyran-4-carboxamide,
(4S)-N-(7-chloro-6-(trans-4-((R)-3-fluoropyrrolidin-1-yl)cyclohexyl)isoquinolin-3-yl)-2,2-dimethyltetrahydro-2H-pyran-4-carboxamide,
(4R)-N-(7-chloro-6-(cis-4-((S)-3-fluoropyrrolidin-1-yl)cyclohexyl)isoquinolin-3-yl)-2,2-dimethyltetrahydro-2H-pyran-4-carboxamide,
(4S)-N-(7-chloro-6-(cis-4-((S)-3-fluoropyrrolidin-1-yl)cyclohexyl)isoquinolin-3-yl)-2,2-dimethyltetrahydro-2H-pyran-4-carboxamide,
(4R)-N-(7-chloro-6-(trans-4-((S)-3-fluoropyrrolidin-1-yl)cyclohexyl)isoquinolin-3-yl)-2,2-dimethyltetrahydro-2H-pyran-4-carboxamide,
(4S)-N-(7-chloro-6-(trans-4-((S)-3-fluoropyrrolidin-1-yl)cyclohexyl)isoquinolin-3-yl)-2,2-dimethyltetrahydro-2H-pyran-4-carboxamide,
N-(7-chloro-6-(4-(3-fluoropyrrolidin-1-yl)cyclohexyl)isoquinolin-3-yl)-6,6-dimethyltetrahydro-2H-pyran-3-carboxamide,
(5R)-N-(7-chloro-6-(cis-4-((R)-3-fluoropyrrolidin-1-yl)cyclohexyl)isoquinolin-3-yl)-6,6-dimethyltetrahydro-2H-pyran-3-carboxamide,
(5S)-N-(7-chloro-6-(cis-4-((R)-3-fluoropyrrolidin-1-yl)cyclohexyl)isoquinolin-3-yl)-6,6-dimethyltetrahydro-2H-pyran-3-carboxamide,
(5R)-N-(7-chloro-6-(trans-4-((R)-3-fluoropyrrolidin-1-yl)cyclohexyl)isoquinolin-3-yl)-6,6-dimethyltetrahydro-2H-pyran-3-carboxamide,
(5S)-N-(7-chloro-6-(trans-4-((R)-3-fluoropyrrolidin-1-yl)cyclohexyl)isoquinolin-3-yl)-6,6-dimethyltetrahydro-2H-pyran-3-carboxamide,
(5R)-N-(7-chloro-6-(cis-4-((S)-3-fluoropyrrolidin-1-yl)cyclohexyl)isoquinolin-3-yl)-6,6-dimethyltetrahydro-2H-pyran-3-carboxamide,
(5S)-N-(7-chloro-6-(cis-4-((S)-3-fluoropyrrolidin-1-yl)cyclohexyl)isoquinolin-3-yl)-6,6-dimethyltetrahydro-2H-pyran-3-carboxamide,
(5R)-N-(7-chloro-6-(trans-4-((S)-3-fluoropyrrolidin-1-yl)cyclohexyl)isoquinolin-3-yl)-6,6-dimethyltetrahydro-2H-pyran-3-carboxamide,
(5S)-N-(7-chloro-6-(trans-4-((S)-3-fluoropyrrolidin-1-yl)cyclohexyl)isoquinolin-3-yl)-6,6-dimethyltetrahydro-2H-pyran-3-carboxamide,
N-(7-chloro-6-(3-(3-fluoroazetidin-1-yl)cyclobutyl)isoquinolin-3-yl)-2-(1-methyl-1H-pyrazol-4-yl)cyclopropane-1-carboxamide,
(1R,2R)-N-(7-chloro-6-(cis-3-(3-fluoroazetidin-1-yl)cyclobutyl)isoquinolin-3-yl)-2-(1-methyl-1H-pyrazol-4-yl)cyclopropane-1-carboxamide,
(1R,2R)-N-(7-chloro-6-(trans-3-(3-fluoroazetidin-1-yl)cyclobutyl)isoquinolin-3-yl)-2-(1-methyl-1H-pyrazol-4-yl)cyclopropane-1-carboxamide,
(1S,2S)-N-(7-chloro-6-(cis-3-(3-fluoroazetidin-1-yl)cyclobutyl)isoquinolin-3-yl)-2-(1-methyl-1H-pyrazol-4-yl)cyclopropane-1-carboxamide,
(1S,2S)-N-(7-chloro-6-(trans-3-(3-fluoroazetidin-1-yl)cyclobutyl)isoquinolin-3-yl)-2-(1-methyl-1H-pyrazol-4-yl)cyclopropane-1-carboxamide,
(R)-N-(7-chloro-6-(1-((3R,4R)-4-fluoro-3-methyltetrahydrofuran-3-yl) piperidin-4-yl) isoquinolin-3-yl)-5-oxaspiro [2.4] heptane-1-carboxamide,
(S)-N-(7-chloro-6-(1-((3R,4R)-4-fluoro-3-methyltetrahydrofuran-3-yl) piperidin-4-yl) isoquinolin-3-yl)-5-oxaspiro [2.4] heptane-1-carboxamide,
(R)-N-(7-chloro-6-(1-((3S,4S)-4-fluoro-3-methyltetrahydrofuran-3-yl) piperidin-4-yl) isoquinolin-3-yl)-5-oxaspiro [2.4] heptane-1-carboxamide (S)-N-(7-chloro-6-(1-((3S,4S)-4-fluoro-3-methyltetrahydrofuran-3-yl) piperidin-4-yl) isoquinolin-3-yl)-5-oxaspiro [2.4] heptane-1-carboxamide,
N-(7-chloro-6-(1-(3,4-dimethyltetrahydrofuran-3-yl)piperidin-4-yl)isoquinolin-3-yl)-6-oxaspiro[2.5]octane-1-carboxamide,
(1S)-N-(7-chloro-6-(1-(3R,4R-dimethyltetrahydrofuran-3-yl)piperidin-4-yl)isoquinolin-3-yl)-6-oxaspiro[2.5]octane-1-carboxamide,
(1R)-N-(7-chloro-6-(1-(3R,4R-dimethyltetrahydrofuran-3-yl)piperidin-4-yl)isoquinolin-3-yl)-6-oxaspiro[2.5]octane-1-carboxamide,
(1S)-N-(7-chloro-6-(1-(3S,4S-dimethyltetrahydrofuran-3-yl)piperidin-4-yl)isoquinolin-3-yl)-6-oxaspiro[2.5]octane-1-carboxamide,
(1R)-N-(7-chloro-6-(1-(3S,4S-dimethyltetrahydrofuran-3-yl)piperidin-4-yl)isoquinolin-3-yl)-6-oxaspiro[2.5]octane-1-carboxamide,
N-(7-chloro-6-(1-(3-methyltetrahydrofuran-3-yl)piperidin-4-yl)isoquinolin-3-yl)-3-oxabicyclo[3.1.0]hexane-6-carboxamide,
(1R)-N-(7-chloro-6-(1-(3R-methyltetrahydrofuran-3-yl)piperidin-4-yl)isoquinolin-3-yl)-3-oxabicyclo[3.1.0]hexane-6-carboxamide,
(1R)-N-(7-chloro-6-(1-(3S-methyltetrahydrofuran-3-yl)piperidin-4-yl)isoquinolin-3-yl)-3-oxabicyclo[3.1.0]hexane-6-carboxamide,
(1S)-N-(7-chloro-6-(1-(3R-methyltetrahydrofuran-3-yl)piperidin-4-yl)isoquinolin-3-yl)-3-oxabicyclo[3.1.0]hexane-6-carboxamide,
(1S)-N-(7-chloro-6-(1-(3S-methyltetrahydrofuran-3-yl)piperidin-4-yl)isoquinolin-3-yl)-3-oxabicyclo[3.1.0]hexane-6-carboxamide,
N-(7-chloro-6-(1-(3-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-(3R-methyltetrahydrofuran-3-yl)piperidin-4-yl)isoquinolin-3-yl)-2-(2-hydroxypropan-2-yl)cyclopropane-1-carboxamide,
(1R,2R)-N-(7-chloro-6-(1-(3S-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-(3R-methyltetrahydrofuran-3-yl)piperidin-4-yl)isoquinolin-3-yl)-2-(2-hydroxypropan-2-yl)cyclopropane-1-carboxamide,
(1S,2S)-N-(7-chloro-6-(1-(3S-methyltetrahydrofuran-3-yl)piperidin-4-yl)isoquinolin-3-yl)-2-(2-hydroxypropan-2-yl)cyclopropane-1-carboxamide,
N-(7-chloro-6-(1-(5-cyclopropyl-3-methyltetrahydrofuran-3-yl)piperidin-4-yl)isoquinolin-3-yl)-6-oxaspiro[2.5]octane-1-carboxamide,
(1R)-N-(7-chloro-6-(1-(5R-cyclopropyl-3R-methyltetrahydrofuran-3-yl)piperidin-4-yl)isoquinolin-3-yl)-6-oxaspiro[2.5]octane-1-carboxamide,
(1R)-N-(7-chloro-6-(1-(5R-cyclopropyl-3S-methyltetrahydrofuran-3-yl)piperidin-4-yl)isoquinolin-3-yl)-6-oxaspiro[2.5]octane-1-carboxamide,
(1R)-N-(7-chloro-6-(1-(5S-cyclopropyl-3R-methyltetrahydrofuran-3-yl)piperidin-4-yl)isoquinolin-3-yl)-6-oxaspiro[2.5]octane-1-carboxamide,
(1R)-N-(7-chloro-6-(1-(5S-cyclopropyl-3S-methyltetrahydrofuran-3-yl)piperidin-4-yl)isoquinolin-3-yl)-6-oxaspiro[2.5]octane-1-carboxamide,
(1S)-N-(7-chloro-6-(1-(5R-cyclopropyl-3R-methyltetrahydrofuran-3-yl)piperidin-4-yl)isoquinolin-3-yl)-6-oxaspiro[2.5]octane-1-carboxamide,
(1S)-N-(7-chloro-6-(1-(5R-cyclopropyl-3S-methyltetrahydrofuran-3-yl)piperidin-4-yl)isoquinolin-3-yl)-6-oxaspiro[2.5]octane-1-carboxamide,
(1S)-N-(7-chloro-6-(1-(5S-cyclopropyl-3R-methyltetrahydrofuran-3-yl)piperidin-4-yl)isoquinolin-3-yl)-6-oxaspiro[2.5]octane-1-carboxamide,
(1S)-N-(7-chloro-6-(1-(5S-cyclopropyl-3S-methyltetrahydrofuran-3-yl)piperidin-4-yl)isoquinolin-3-yl)-6-oxaspiro[2.5]octane-1-carboxamide,
N-(7-chloro-6-(1-(3,4,4-trimethyltetrahydrofuran-3-yl)piperidin-4-yl)isoquinolin-3-yl)-6-oxaspiro[2.5]octane-1-carboxamide,
(1R)-N-(7-chloro-6-(1-((S)-3,4,4-trimethyltetrahydrofuran-3-yl)piperidin-4-yl)isoquinolin-3-yl)-6-oxaspiro[2.5]octane-1-carboxamide,
(1R)-N-(7-chloro-6-(1-((R)-3,4,4-trimethyltetrahydrofuran-3-yl)piperidin-4-yl)isoquinolin-3-yl)-6-oxaspiro[2.5]octane-1-carboxamide,
1S)-N-(7-chloro-6-(1-((S)-3,4,4-trimethyltetrahydrofuran-3-yl)piperidin-4-yl)isoquinolin-3-yl)-6-oxaspiro[2.5]octane-1-carboxamide,
(1S)-N-(7-chloro-6-(1-((R)-3,4,4-trimethyltetrahydrofuran-3-yl)piperidin-4-yl)isoquinolin-3-yl)-6-oxaspiro[2.5]octane-1-carboxamide,
N-(7-chloro-6-(1-(1,2-dimethylcyclopentyl)piperidin-4-yl)isoquinolin-3-yl)-6-oxaspiro[2.5]octane-1-carboxamide,
(1R)-N-(7-chloro-6-(1-((R)-1,2-dimethylcyclopentyl)piperidin-4-yl)isoquinolin-3-yl)-6-oxaspiro[2.5]octane-1-carboxamide,
(1R)-N-(7-chloro-6-(1-((S)-1,2-dimethylcyclopentyl)piperidin-4-yl)isoquinolin-3-yl)-6-oxaspiro[2.5]octane-1-carboxamide,
(1S)-N-(7-chloro-6-(1-((R)-1,2-dimethylcyclopentyl)piperidin-4-yl)isoquinolin-3-yl)-6-oxaspiro[2.5]octane-1-carboxamide,
(1S)-N-(7-chloro-6-(1-((S)-1,2-dimethylcyclopentyl)piperidin-4-yl)isoquinolin-3-yl)-6-oxaspiro[2.5]octane-1-carboxamide,
N-(7-chloro-6-(1-(4-methoxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3R,4R)-4-methoxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3S,4S)-4-methoxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3R,4R)-4-methoxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3S,4S)-4-methoxy-3-methyltetrahydrofuran-3-yl)piperidin-4-yl)isoquinolin-3-yl)-6-oxaspiro[2.5]octane-1-carboxamide,
N-(7-chloro-6-(1-(4-fluoro-3-methyltetrahydrofuran-3-yl)piperidin-4-yl)isoquinolin-3-yl)-6-oxaspiro[2.5]octane-1-carboxamide,
(1R)-N-(7-chloro-6-(1-((3R,4R)-4-fluoro-3-methyltetrahydrofuran-3-yl)piperidin-4-yl)isoquinolin-3-yl)-6-oxaspiro[2.5]octane-1-carboxamide,
(1R)-N-(7-chloro-6-(1-((3S,4S)-4-fluoro-3-methyltetrahydrofuran-3-yl)piperidin-4-yl)isoquinolin-3-yl)-6-oxaspiro[2.5]octane-1-carboxamide,
(1S)-N-(7-chloro-6-(1-((3R,4R)-4-fluoro-3-methyltetrahydrofuran-3-yl)piperidin-4-yl)isoquinolin-3-yl)-6-oxaspiro[2.5]octane-1-carboxamide,
(1S)-N-(7-chloro-6-(1-((3S,4S)-4-fluoro-3-methyltetrahydrofuran-3-yl)piperidin-4-yl)isoquinolin-3-yl)-6-oxaspiro[2.5]octane-1-carboxamide,
N-(7-chloro-6-(1-(4-fluoro-3-methyltetrahydrofuran-3-yl)piperidin-4-yl)isoquinolin-3-yl)-2-(2-hydroxypropan-2-yl)cyclopropane-1-carboxamide,
(1R)-N-(7-chloro-6-(1-((3R,4R)-4-fluoro-3-methyltetrahydrofuran-3-yl)piperidin-4-yl)isoquinolin-3-yl)-2-(2-hydroxypropan-2-yl)cyclopropane-1-carboxamide,
(1R)-N-(7-chloro-6-(1-((3S,4S)-4-fluoro-3-methyltetrahydrofuran-3-yl)piperidin-4-yl)isoquinolin-3-yl)-2-(2-hydroxypropan-2-yl)cyclopropane-1-carboxamide,
(1S)-N-(7-chloro-6-(1-((3R,4R)-4-fluoro-3-methyltetrahydrofuran-3-yl)piperidin-4-yl)isoquinolin-3-yl)-2-(2-hydroxypropan-2-yl)cyclopropane-1-carboxamide,
(1S)-N-(7-chloro-6-(1-((3S,4S)-4-fluoro-3-methyltetrahydrofuran-3-yl)piperidin-4-yl)isoquinolin-3-yl)-2-(2-hydroxypropan-2-yl)cyclopropane-1-carboxamide,
N-(7-chloro-6-(1-(4-fluoro-3-methyltetrahydrofuran-3-yl)piperidin-4-yl)isoquinolin-3-yl)-2-(1-methyl-1H-pyrazol-4-yl)cyclopropane-1-carboxamide,
(1R,2R)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-4-yl)cyclopropane-1-carboxamide,
(1R,2R)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-4-yl)cyclopropane-1-carboxamide,
(1S,2S)-N-(7-chloro-6-(1-((3R,4R)-4-fluoro-3-methyltetrahydrofuran-3-yl)piperidin-4-yl)isoquinolin-3-yl)-2-(1-methyl-1H-pyrazol-4-yl)cyclopropane-1-carboxamide,
(1S,2S)-N-(7-chloro-6-(1-((3S,4S)-4-fluoro-3-methyltetrahydrofuran-3-yl)piperidin-4-yl)isoquinolin-3-yl)-2-(1-methyl-1H-pyrazol-4-yl)cyclopropane-1-carboxamide,
N-(7-chloro-6-(1-(3-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-(3R-methyltetrahydrofuran-3-yl)piperidin-4-yl)isoquinolin-3-yl)-2-(pyridin-2-yl)cyclopropane-1-carboxamide,
(1R,2R)-N-(7-chloro-6-(1-(3S-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-(3R-methyltetrahydrofuran-3-yl)piperidin-4-yl)isoquinolin-3-yl)-2-(pyridin-2-yl)cyclopropane-1-carboxamide,
(1S,2S)-N-(7-chloro-6-(1-(3S-methyltetrahydrofuran-3-yl)piperidin-4-yl)isoquinolin-3-yl)-2-(pyridin-2-yl)cyclopropane-1-carboxamidboxamide,
N-(7-chloro-6-(1-(4-fluoro-3-methyltetrahydrofuran-3-yl)piperidin-4-yl)isoquinolin-3-yl)-2,2-dimethyltetrahydro-2H-pyran-4-carboxamide,
(4R)-N-(7-chloro-6-(1-((3R,4R)-4-fluoro-3-methyltetrahydrofuran-3-yl)piperidin-4-yl)isoquinolin-3-yl)-2,2-dimethyltetrahydro-2H-pyran-4-carboxamide,
(4R)-N-(7-chloro-6-(1-((3S,4S)-4-fluoro-3-methyltetrahydrofuran-3-yl)piperidin-4-yl)isoquinolin-3-yl)-2,2-dimethyltetrahydro-2H-pyran-4-carboxamide,
(4S)-N-(7-chloro-6-(1-((3R,4R)-4-fluoro-3-methyltetrahydrofuran-3-yl)piperidin-4-yl)isoquinolin-3-yl)-2,2-dimethyltetrahydro-2H-pyran-4-carboxamide,
(4S)-N-(7-chloro-6-(1-((3S,4S)-4-fluoro-3-methyltetrahydrofuran-3-yl)piperidin-4-yl)isoquinolin-3-yl)-2,2-dimethyltetrahydro-2H-pyran-4-carboxamide,
N-(7-chloro-6-(1-(4-fluoro-3-methyltetrahydrofuran-3-yl)piperidin-4-yl)isoquinolin-3-yl)-3-oxabicyclo[4.1.0]heptane-7-carboxamide,
(1R,6R)-N-(7-chloro-6-(1-((3R,4R)-4-fluoro-3-methyltetrahydrofuran-3-yl)piperidin-4-yl)isoquinolin-3-yl)-3-oxabicyclo[4.1.0]heptane-7-carboxamide,
(1R,6R)-N-(7-chloro-6-(1-((3S,4S)-4-fluoro-3-methyltetrahydrofuran-3-yl)piperidin-4-yl)isoquinolin-3-yl)-3-oxabicyclo[4.1.0]heptane-7-carboxamide,
(1S,6S)-N-(7-chloro-6-(1-((3R,4R)-4-fluoro-3-methyltetrahydrofuran-3-yl)piperidin-4-yl)isoquinolin-3-yl)-3-oxabicyclo[4.1.0]heptane-7-carboxamide,
(1S,6S)-N-(7-chloro-6-(1-((3S,4S)-4-fluoro-3-methyltetrahydrofuran-3-yl)piperidin-4-yl)isoquinolin-3-yl)-3-oxabicyclo[4.1.0]heptane-7-carboxamide,
N-(7-chloro-6-(1-(3-methyltetrahydrofuran-3-yl)piperidin-4-yl)isoquinolin-3-yl)-2,2-dimethyltetrahydro-2H-pyran-4-carboxamide,
(4R)-N-(7-chloro-6-(1-(3R-methyltetrahydrofuran-3-yl)piperidin-4-yl)isoquinolin-3-yl)-2,2-dimethyltetrahydro-2H-pyran-4-carboxamide,
(4R)-N-(7-chloro-6-(1-(3S-methyltetrahydrofuran-3-yl)piperidin-4-yl)isoquinolin-3-yl)-2,2-dimethyltetrahydro-2H-pyran-4-carboxamide,
(4S)-N-(7-chloro-6-(1-(3R-methyltetrahydrofuran-3-yl)piperidin-4-yl)isoquinolin-3-yl)-2,2-dimethyltetrahydro-2H-pyran-4-carboxamide,
(4S)-N-(7-chloro-6-(1-(3S-methyltetrahydrofuran-3-yl)piperidin-4-yl)isoquinolin-3-yl)-2,2-dimethyltetrahydro-2H-pyran-4-carboxamide,
N-(7-chloro-6-(1-(3-methyltetrahydrofuran-3-yl)piperidin-4-yl)isoquinolin-3-yl)-3-oxabicyclo[4.1.0]heptane-7-carboxamide,
(1R,6R)-N-(7-chloro-6-(1-(3R-methyltetrahydrofuran-3-yl)piperidin-4-yl)isoquinolin-3-yl)-3-oxabicyclo[4.1.0]heptane-7-carboxamide,
(1R,6R)-N-(7-chloro-6-(1-(3S-methyltetrahydrofuran-3-yl)piperidin-4-yl)isoquinolin-3-yl)-3-oxabicyclo[4.1.0]heptane-7-carboxamide,
(1S,6S)-N-(7-chloro-6-(1-(3R-methyltetrahydrofuran-3-yl)piperidin-4-yl)isoquinolin-3-yl)-3-oxabicyclo[4.1.0]heptane-7-carboxamide,
(1S,6S)-N-(7-chloro-6-(1-(3S-methyltetrahydrofuran-3-yl)piperidin-4-yl)isoquinolin-3-yl)-3-oxabicyclo[4.1.0]heptane-7-carboxamide,
N-(7-chloro-6-(1-(4-methoxytetrahydrofuran-3-yl)piperidin-4-yl)isoquinolin-3-yl)-6-oxaspiro[2.5]octane-1-carboxamide,
(R)-N-(7-chloro-6-(1-((3R,4R)-4-methoxytetrahydrofuran-3-yl)piperidin-4-yl)isoquinolin-3-yl)-6-oxaspiro[2.5]octane-1-carboxamide,
(S)-N-(7-chloro-6-(1-((3R,4R)-4-methoxytetrahydrofuran-3-yl)piperidin-4-yl)isoquinolin-3-yl)-6-oxaspiro[2.5]octane-1-carboxamide,
(R)-N-(7-chloro-6-(1-((3S,4S)-4-methoxytetrahydrofuran-3-yl)piperidin-4-yl)isoquinolin-3-yl)-6-oxaspiro[2.5]octane-1-carboxamide,
(S)-N-(7-chloro-6-(1-((3S,4S)-4-methoxytetrahydrofuran-3-yl)piperidin-4-yl)isoquinolin-3-yl)-6-oxaspiro[2.5]octane-1-carboxamide,
N-(7-chloro-6-(1-(4-fluoro-3-methyltetrahydrofuran-3-yl)piperidin-4-yl)isoquinolin-3-yl)-2-methyl-2-(1-methyl-1H-pyrazol-4-yl)propenamide,
N-(7-chloro-6-(1-((3R,4R)-4-fluoro-3-methyltetrahydrofuran-3-yl)piperidin-4-yl)isoquinolin-3-yl)-2-methyl-2-(1-methyl-1H-pyrazol-4-yl)propenamide,
N-(7-chloro-6-(1-((3S,4S)-4-fluoro-3-methyltetrahydrofuran-3-yl)piperidin-4-yl)isoquinolin-3-yl)-2-methyl-2-(1-methyl-1H-pyrazol-4-yl)propenamide,
N-(7-chloro-6-(1-(oxetan-3-yl)piperidin-4-yl)isoquinolin-3-yl)acetamide,
N-(7-chloro-6-(1-(3-methyloxetan-3-yl)piperidin-4-yl)isoquinolin-3-yl)cyclopropanecarboxamide,
N-(7-chloro-6-(1-(oxetan-3-yl)piperidin-4-yl)isoquinolin-3-yl)-1-methylcyclopropane-1-carboxamide,
N-(7-chloro-6-(1-(oxetan-3-yl)piperidin-4-yl)isoquinolin-3-yl)cyclobutanecarboxamide,
N-(7-chloro-6-(1-(oxetan-3-yl)piperidin-4-yl)isoquinolin-3-yl)cyclopentanecarboxamide,
N-(7-chloro-6-(1-(oxetan-3-yl)piperidin-4-yl)isoquinolin-3-yl)cyclopropanecarboxamide,
N-(7-chloro-6-(1-(oxetan-3-yl)piperidin-4-yl)isoquinolin-3-yl)acetamide,
N-(7-chloro-6-(1-(3-methyloxetan-3-yl)piperidin-4-yl)isoquinolin-3-yl)bicyclo[1.1.1]pentane-1-carboxamide,
N-(7-chloro-6-(1-(oxetan-3-yl)piperidin-4-yl)isoquinolin-3-yl)-1-fluorocyclopropane-1-carboxamide,
Rac-N-(7-chloro-6-(1-(oxetan-3-yl)piperidin-4-yl)isoquinolin-3-yl)-3-methoxycyclobutane-1-carboxamide,
N-(7-chloro-6-(tetrahydro-2H-pyran-4-yl)isoquinolin-3-yl)-6-oxaspiro[2.5]octane-1-carboxamide,
N-(7-chloro-6-(1-(4-hydroxytetrahydrofuran-3-yl)piperidin-4-yl)isoquinolin-3-yl)-5-oxaspiro[2.4]heptane-1-carboxamide,
N-(7-chloro-6-(1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
N-(7-fluoro-6-(1-(4-hydroxy-3-methyltetrahydrofuran-3-yl)piperidin-4-yl)isoquinolin-3-yl)-6-oxaspiro[2.5]octane-1-carboxamide,
(1R)-N-(7-fluoro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1R)-N-(7-fluoro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
(1S)-N-(7-fluoro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1S)-N-(7-fluoro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
N-(7-fluoro-6-(1-(4-hydroxy-3-methyltetrahydrofuran-3-yl)piperidin-4-yl)isoquinolin-3-yl)-2-(pyridin-2-yl)cyclopropane-1-carboxamide,
(1R,2R)-N-(7-fluoro-6-(1-((3R,4R)-4-hydroxy-3-methyltetrahydrofuran-3-yl)piperidin-4-yl)isoquinolin-3-yl)-2-(pyridin-2-yl)cyclopropane-1-carboxamide,
(1R,2R)-N-(7-fluoro-6-(1-((3S,4S)-4-hydroxy-3-methyltetrahydrofuran-3-yl)piperidin-4-yl)isoquinolin-3-yl)-2-(pyridin-2-yl)cyclopropane-1-carboxamide,
(1S,2S)-N-(7-fluoro-6-(1-((3R,4R)-4-hydroxy-3-methyltetrahydrofuran-3-yl)piperidin-4-yl)isoquinolin-3-yl)-2-(pyridin-2-yl)cyclopropane-1-carboxamide,
(1S,2S)-N-(7-fluoro-6-(1-((3S,4S)-4-hydroxy-3-methyltetrahydrofuran-3-yl)piperidin-4-yl)isoquinolin-3-yl)-2-(pyridin-2-yl)cyclopropane-1-carboxamide,
N-(7-chloro-6-(1-(4-hydroxytetrahydrofuran-3-yl)piperidin-4-yl)isoquinolin-3-yl)-5-oxaspiro[2.4]heptane-1-carboxamide,
(1R,2R)-N-(7-chloro-6-(1-((3R,4R)-4-hydroxytetrahydrofuran-3-yl)piperidin-4-yl)isoquinolin-3-yl)-5-oxaspiro[2.4]heptane-1-carboxamide,
(1R,2R)-N-(7-chloro-6-(1-((3 S,4S)-4-hydroxytetrahydrofuran-3-yl)piperidin-4-yl)isoquinolin-3-yl)-5-oxaspiro[2.4]heptane-1-carboxamide,
(1R,2S)-N-(7-chloro-6-(1-((3R,4R)-4-hydroxytetrahydrofuran-3-yl)piperidin-4-yl)isoquinolin-3-yl)-5-oxaspiro[2.4]heptane-1-carboxamide,
(1R,2S)-N-(7-chloro-6-(1-((3 S,4S)-4-hydroxytetrahydrofuran-3-yl)piperidin-4-yl)isoquinolin-3-yl)-5-oxaspiro[2.4]heptane-1-carboxamide,
(1S,2R)-N-(7-chloro-6-(1-((3R,4R)-4-hydroxytetrahydrofuran-3-yl)piperidin-4-yl)isoquinolin-3-yl)-5-oxaspiro[2.4]heptane-1-carboxamide,
(1S,2R)-N-(7-chloro-6-(1-((3 S,4S)-4-hydroxytetrahydrofuran-3-yl)piperidin-4-yl)isoquinolin-3-yl)-5-oxaspiro[2.4]heptane-1-carboxamide,
(1S,2S)-N-(7-chloro-6-(1-((3R,4R)-4-hydroxytetrahydrofuran-3-yl)piperidin-4-yl)isoquinolin-3-yl)-5-oxaspiro[2.4]heptane-1-carboxamide,
(1S,2S)-N-(7-chloro-6-(1-((3S,4S)-4-hydroxytetrahydrofuran-3-yl)piperidin-4-yl)isoquinolin-3-yl)-5-oxaspiro[2.4]heptane-1-carboxamide,
(S)-N-(7-chloro-6-(1-(oxetan-3-yl)piperidin-4-yl)isoquinolin-3-yl)spiro[2.2]pentane-1-carboxamide and (R)-N-(7-chloro-6-(1-(oxetan-3-yl)piperidin-4-yl)isoquinolin-3-yl)spiro[2.2]pentane-1-carboxamide,
N-(7-chloro-6-(1-(oxetan-3-yl)piperidin-4-yl)isoquinolin-3-yl)-2-methylcyclopropane-1-carboxamide,
(1R,2R)-N-(7-chloro-6-(1-(oxetan-3-yl)piperidin-4-yl)isoquinolin-3-yl)-2-methylcyclopropane-1-carboxamide,
(1R,2S)-N-(7-chloro-6-(1-(oxetan-3-yl)piperidin-4-yl)isoquinolin-3-yl)-2-methylcyclopropane-1-carboxamide,
(1S,2R)-N-(7-chloro-6-(1-(oxetan-3-yl)piperidin-4-yl)isoquinolin-3-yl)-2-methylcyclopropane-1-carboxamide,
(1S,2S)-N-(7-chloro-6-(1-(oxetan-3-yl)piperidin-4-yl)isoquinolin-3-yl)-2-methylcyclopropane-1-carboxamide,
N-(7-chloro-6-(1-(oxetan-3-yl)piperidin-4-yl)isoquinolin-3-yl)-6-oxaspiro[2.5]octane-1-carboxamide,
(1R)-N-(7-chloro-6-(1-(oxetan-3-yl)piperidin-4-yl)isoquinolin-3-yl)-6-oxaspiro[2.5]octane-1-carboxamide,
(1S)-N-(7-chloro-6-(1-(oxetan-3-yl)piperidin-4-yl)isoquinolin-3-yl)-6-oxaspiro[2.5]octane-1-carboxamide,
N-(7-chloro-6-(1-(2-methyltetrahydrofuran-3-yl)piperidin-4-yl)isoquinolin-3-yl)cyclopropanecarboxamide,
N-(7-chloro-6-(1-((2R,3R)-2-methyltetrahydrofuran-3-yl)piperidin-4-yl)isoquinolin-3-yl)cyclopropanecarboxamide,
N-(7-chloro-6-(1-((2R,3 S)-2-methyltetrahydrofuran-3-yl)piperidin-4-yl)isoquinolin-3-yl)cyclopropanecarboxamide,
N-(7-chloro-6-(1-((2S,3R)-2-methyltetrahydrofuran-3-yl)piperidin-4-yl)isoquinolin-3-yl)cyclopropanecarboxamide,
N-(7-chloro-6-(1-((2S,3S)-2-methyltetrahydrofuran-3-yl)piperidin-4-yl)isoquinolin-3-yl)cyclopropanecarboxamide,
N-(7-chloro-6-(1-methoxypropan-2-yl)isoquinolin-3-yl)-6-oxaspiro[2.5]octane-1-carboxamide,
(1R)-N-(7-chloro-6-((R)-1-methoxypropan-2-yl)isoquinolin-3-yl)-6-oxaspiro[2.5]octane-1-carboxamide,
(1R)-N-(7-chloro-6-((S)-1-methoxypropan-2-yl)isoquinolin-3-yl)-6-oxaspiro[2.5]octane-1-carboxamide,
(1S)-N-(7-chloro-6-((R)-1-methoxypropan-2-yl)isoquinolin-3-yl)-6-oxaspiro[2.5]octane-1-carboxamide,
1S)-N-(7-chloro-6-((S)-1-methoxypropan-2-yl)isoquinolin-3-yl)-6-oxaspiro[2.5]octane-1-carboxamide,
N-(7-chloro-6-(1-methoxypropan-2-yl)isoquinolin-3-yl)-2-ethyl-3-(1-methyl-1H-pyrazol-4-yl)cyclopropane-1-carboxamide,
(1R,2R)-N-(7-chloro-6-((R)-1-methoxypropan-2-yl)isoquinolin-3-yl)-2-ethyl-3-(1-methyl-1H-pyrazol-4-yl)cyclopropane-1-carboxamide,
(1R,2R)-N-(7-chloro-6-((S)-1-methoxypropan-2-yl)isoquinolin-3-yl)-2-ethyl-3-(1-methyl-1H-pyrazol-4-yl)cyclopropane-1-carboxamide,
(1S,2S)-N-(7-chloro-6-((R)-1-methoxypropan-2-yl)isoquinolin-3-yl)-2-ethyl-3-(1-methyl-1H-pyrazol-4-yl)cyclopropane-1-carboxamide,
(1S,2S)-N-(7-chloro-6-((S)-1-methoxypropan-2-yl)isoquinolin-3-yl)-2-ethyl-3-(1-methyl-1H-pyrazol-4-yl)cyclopropane-1-carboxamide,
N-(7-chloro-6-(1-cyanopropan-2-yl)isoquinolin-3-yl)-2-ethyl-3-(1-methyl-1H-pyrazol-4-yl)cyclopropane-1-carboxamide,
(1R,2R,3R)-N-(7-chloro-6-((R)-1-cyanopropan-2-yl)isoquinolin-3-yl)-2-ethyl-3-(1-methyl-1H-pyrazol-4-yl)cyclopropane-1-carboxamide,
(1R,2R,3R)-N-(7-chloro-6-((S)-1-cyanopropan-2-yl)isoquinolin-3-yl)-2-ethyl-3-(1-methyl-1H-pyrazol-4-yl)cyclopropane-1-carboxamide,
(1R,2S,3R)-N-(7-chloro-6-((R)-1-cyanopropan-2-yl)isoquinolin-3-yl)-2-ethyl-3-(1-methyl-1H-pyrazol-4-yl)cyclopropane-1-carboxamide,
(1R,2S,3R)-N-(7-chloro-6-((S)-1-cyanopropan-2-yl)isoquinolin-3-yl)-2-ethyl-3-(1-methyl-1H-pyrazol-4-yl)cyclopropane-1-carboxamide,
(1S,2R,3 S)-N-(7-chloro-6-((R)-1-cyanopropan-2-yl)isoquinolin-3-yl)-2-ethyl-3-(1-methyl-1H-pyrazol-4-yl)cyclopropane-1-carboxamide,
(1S,2R,3 S)-N-(7-chloro-6-((S)-1-cyanopropan-2-yl)isoquinolin-3-yl)-2-ethyl-3-(1-methyl-1H-pyrazol-4-yl)cyclopropane-1-carboxamide,
(1S,2S,3S)-N-(7-chloro-6-((R)-1-cyanopropan-2-yl)isoquinolin-3-yl)-2-ethyl-3-(1-methyl-1H-pyrazol-4-yl)cyclopropane-1-carboxamide,
(1S,2S,3S)-N-(7-chloro-6-((S)-1-cyanopropan-2-yl)isoquinolin-3-yl)-2-ethyl-3-(1-methyl-1H-pyrazol-4-yl)cyclopropane-1-carboxamide,
N-(7-chloro-6-(3-cyanocyclobutyl)isoquinolin-3-yl)-2-ethyl-3-(1-methyl-1H-pyrazol-4-yl)cyclopropane-1-carboxamide,
(1R,2R,3R)-N-(7-chloro-6-(cis-3-cyanocyclobutyl)isoquinolin-3-yl)-2-ethyl-3-(1-methyl-1H-pyrazol-4-yl)cyclopropane-1-carboxamide,
(1R,2R,3R)-N-(7-chloro-6-(trans-3-cyanocyclobutyl)isoquinolin-3-yl)-2-ethyl-3-(1-methyl-1H-pyrazol-4-yl)cyclopropane-1-carboxamide,
(1R,2S,3R)-N-(7-chloro-6-(cis-3-cyanocyclobutyl)isoquinolin-3-yl)-2-ethyl-3-(1-methyl-1H-pyrazol-4-yl)cyclopropane-1-carboxamide,
(1R,2S,3R)-N-(7-chloro-6-(trans-3-cyanocyclobutyl)isoquinolin-3-yl)-2-ethyl-3-(1-methyl-1H-pyrazol-4-yl)cyclopropane-1-carboxamide,
(1S,2R,3 S)-N-(7-chloro-6-(cis-3-cyanocyclobutyl)isoquinolin-3-yl)-2-ethyl-3-(1-methyl-1H-pyrazol-4-yl)cyclopropane-1-carboxamide,
(1S,2R,3 S)-N-(7-chloro-6-(trans-3-cyanocyclobutyl)isoquinolin-3-yl)-2-ethyl-3-(1-methyl-1H-pyrazol-4-yl)cyclopropane-1-carboxamide,
(1S,2S,3S)-N-(7-chloro-6-(cis-3-cyanocyclobutyl)isoquinolin-3-yl)-2-ethyl-3-(1-methyl-1H-pyrazol-4-yl)cyclopropane-1-carboxamide,
(1S,2S,3S)-N-(7-chloro-6-(trans-3-cyanocyclobutyl)isoquinolin-3-yl)-2-ethyl-3-(1-methyl-1H-pyrazol-4-yl)cyclopropane-1-carboxamide,
N-(7-chloro-6-(1-methoxypropan-2-yl)isoquinolin-3-yl)-2-(pyridin-2-yl)cyclopropane-1-carboxamide,
(1R,2R)-N-(7-chloro-6-((R)-1-methoxypropan-2-yl)isoquinolin-3-yl)-2-(pyridin-2-yl)cyclopropane-1-carboxamide,
(1R,2R)-N-(7-chloro-6-((S)-1-methoxypropan-2-yl)isoquinolin-3-yl)-2-(pyridin-2-yl)cyclopropane-1-carboxamide,
(1S,2S)-N-(7-chloro-6-((R)-1-methoxypropan-2-yl)isoquinolin-3-yl)-2-(pyridin-2-yl)cyclopropane-1-carboxamide,
(1S,2S)-N-(7-chloro-6-((S)-1-methoxypropan-2-yl)isoquinolin-3-yl)-2-(pyridin-2-yl)cyclopropane-1-carboxamide,
N-(7-chloro-6-(1-cyanopropan-2-yl)isoquinolin-3-yl)-2-(pyridin-2-yl)cyclopropane-1-carboxamide,
(1R,2R)-N-(7-chloro-6-((R)-1-cyanopropan-2-yl)isoquinolin-3-yl)-2-(pyridin-2-yl)cyclopropane-1-carboxamide,
(1R,2R)-N-(7-chloro-6-((S)-1-cyanopropan-2-yl)isoquinolin-3-yl)-2-(pyridin-2-yl)cyclopropane-1-carboxamide,
(1S,2S)-N-(7-chloro-6-((R)-1-cyanopropan-2-yl)isoquinolin-3-yl)-2-(pyridin-2-yl)cyclopropane-1-carboxamide,
(1S,2S)-N-(7-chloro-6-((S)-1-cyanopropan-2-yl)isoquinolin-3-yl)-2-(pyridin-2-yl)cyclopropane-1-carboxamide,
N-(6-(3-oxabicyclo[4.1.0]heptan-7-yl)-7-chloroisoquinolin-3-yl)-6-oxaspiro[2.5]octane-1-carboxamide,
(R)-(1R,6R)-N-(6-(3-oxabicyclo[4.1.0]heptan-7-yl)-7-chloroisoquinolin-3-yl)-6-oxaspiro[2.5]octane-1-carboxamide,
(S)-(1R,6R)-N-(6-(3-oxabicyclo[4.1.0]heptan-7-yl)-7-chloroisoquinolin-3-yl)-6-oxaspiro[2.5]octane-1-carboxamide,
(R)-(1S,6S)-N-(6-(3-oxabicyclo[4.1.0]heptan-7-yl)-7-chloroisoquinolin-3-yl)-6-oxaspiro[2.5]octane-1-carboxamide,
(S)-(1S,6S)-N-(6-(3-oxabicyclo[4.1.0]heptan-7-yl)-7-chloroisoquinolin-3-yl)-6-oxaspiro[2.5]octane-1-carboxamide,
1,4-trans, (3R,4R)- or 1,4-trans, (3S,4S)- or 1,4-cis, (3R,4R)- or 1,4-cis, (3S,4S)-N-(7-chloro-6-(1-(4-hydroxy-3-methyltetrahydrofuran-3-yl)piperidin-4-yl)isoquinolin-3-yl)-4-methoxycyclohexane-1-carboxamide,
N-(7-chloro-6-(1-((3R,4R or 3S,4S)-4-hydroxytetrahydrofuran-3-yl)piperidin-4-yl)isoquinolin-3-yl)-5-oxaspiro[2.4]heptane-1-carboxamide,
N-(7-chloro-6-(1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3R,4R)-4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-(1-((3S,4S)-4-hydroxy-3-methyltetrahydrofuran-3-yl)piperidin-4-yl)isoquinolin-3-yl)-6-oxaspiro[2.5]octane-1-carboxamide,
N-(7-chloro-6-(1-(4-hydroxy-3-methyltetrahydrofuran-3-yl)piperidin-4-yl)isoquinolin-3-yl)-2-methyl-6-oxaspiro[2.5]octane-1-carboxamide,
1R,2R,3R)-N-(7-chloro-6-(1-((3R,4R)-4-hydroxy-3-methyltetrahydrofuran-3-yl)piperidin-4-yl)isoquinolin-3-yl)-2-methyl-6-oxaspiro[2.5]octane-1-carboxamide,
(1R,2R,3R)-N-(7-chloro-6-(1-((3S,4S)-4-hydroxy-3-methyltetrahydrofuran-3-yl)piperidin-4-yl)isoquinolin-3-yl)-2-methyl-6-oxaspiro[2.5]octane-1-carboxamide,
(1R,2S,3R)-N-(7-chloro-6-(1-((3R,4R)-4-hydroxy-3-methyltetrahydrofuran-3-yl)piperidin-4-yl)isoquinolin-3-yl)-2-methyl-6-oxaspiro[2.5]octane-1-carboxamide,
(1R,2S,3R)-N-(7-chloro-6-(1-((3S,4S)-4-hydroxy-3-methyltetrahydrofuran-3-yl)piperidin-4-yl)isoquinolin-3-yl)-2-methyl-6-oxaspiro[2.5]octane-1-carboxamide,
(1S,2R,3 S)-N-(7-chloro-6-(1-((3R,4R)-4-hydroxy-3-methyltetrahydrofuran-3-yl)piperidin-4-yl)isoquinolin-3-yl)-2-methyl-6-oxaspiro[2.5]octane-1-carboxamide,
(1S,2R,3 S)-N-(7-chloro-6-(1-((3S,4S)-4-hydroxy-3-methyltetrahydrofuran-3-yl)piperidin-4-yl)isoquinolin-3-yl)-2-methyl-6-oxaspiro[2.5]octane-1-carboxamide,
(1S,2S,3S)-N-(7-chloro-6-(1-((3R,4R)-4-hydroxy-3-methyltetrahydrofuran-3-yl)piperidin-4-yl)isoquinolin-3-yl)-2-methyl-6-oxaspiro[2.5]octane-1-carboxamide,
(1S,2S,3S)-N-(7-chloro-6-(1-((3S,4S)-4-hydroxy-3-methyltetrahydrofuran-3-yl)piperidin-4-yl)isoquinolin-3-yl)-2-methyl-6-oxaspiro[2.5]octane-1-carboxamide,
N-(6-(1-(4-hydroxy-3-methyltetrahydrofuran-3-yl)piperidin-4-yl)-7-methylisoquinolin-3-yl)-2-(pyridin-2-yl)cyclopropane-1-carboxamide,
(1R,2R)-N-(6-(1-((3R,4R)-4-hydroxy-3-methyltetrahydrofuran-3-yl)piperidin-4-yl)-7-methylisoquinolin-3-yl)-2-(pyridin-2-yl)cyclopropane-1-carboxamide,
(1R,2R)-N-(6-(1-((3S,4S)-4-hydroxy-3-methyltetrahydrofuran-3-yl)piperidin-4-yl)-7-methylisoquinolin-3-yl)-2-(pyridin-2-yl)cyclopropane-1-carboxamide,
(1S,2S)-N-(6-(1-((3R,4R)-4-hydroxy-3-methyltetrahydrofuran-3-yl)piperidin-4-yl)-7-methylisoquinolin-3-yl)-2-(pyridin-2-yl)cyclopropane-1-carboxamide,
(1S,2S)-N-(6-(1-((3S,4S)-4-hydroxy-3-methyltetrahydrofuran-3-yl)piperidin-4-yl)-7-methylisoquinolin-3-yl)-2-(pyridin-2-yl)cyclopropane-1-carboxamide,
N-(6-(1-(4-hydroxy-3-methyltetrahydrofuran-3-yl)piperidin-4-yl)-7-methylisoquinolin-3-yl)spiro[2.2]pentane-1-carboxamide,
(1R)-N-(6-(1-((3R,4R)-4-hydroxy-3-methyltetrahydrofuran-3-yl)piperidin-4-yl)-7-methylisoquinolin-3-yl)spiro[2.2]pentane-1-carboxamide,
(1R)-N-(6-(1-((3S,4S)-4-hydroxy-3-methyltetrahydrofuran-3-yl)piperidin-4-yl)-7-methylisoquinolin-3-yl)spiro[2.2]pentane-1-carboxamide,
(1S)-N-(6-(1-((3R,4R)-4-hydroxy-3-methyltetrahydrofuran-3-yl)piperidin-4-yl)-7-methylisoquinolin-3-yl)spiro[2.2]pentane-1-carboxamide,
(1S)-N-(6-(1-((3S,4S)-4-hydroxy-3-methyltetrahydrofuran-3-yl)piperidin-4-yl)-7-methylisoquinolin-3-yl)spiro[2.2]pentane-1-carboxamide,
N-(7-chloro-6-(1-(4-hydroxy-3-methyltetrahydrofuran-3-yl)piperidin-4-yl)isoquinolin-3-yl)-2-(difluoromethyl)cyclopropane-1-carboxamide,
(1R,2R)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1R,2R)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
(1S,2S)-N-(7-chloro-6-(1-((3R,4R)-4-hydroxy-3-methyltetrahydrofuran-3-yl)piperidin-4-yl)isoquinolin-3-yl)-2-(difluoromethyl)cyclopropane-1-carboxamide,
(1S,2S)-N-(7-chloro-6-(1-((3S,4S)-4-hydroxy-3-methyltetrahydrofuran-3-yl)piperidin-4-yl)isoquinolin-3-yl)-2-(difluoromethyl)cyclopropane-1-carboxamide,
(1R,2R,3R) or (1R,2S,3R) or (1S,2R,3S) or (1S,2S,3S)-N-(7-chloro-6-((cis or trans)-4-(3-fluoro-3-methylazetidin-1-yl)cyclohexyl)isoquinolin-3-yl)-2-ethyl-3-(1-methyl-1H-pyrazol-4-yl)cyclopropane-1-carboxamide,
N-(7-chloro-6-(4-(3-methoxyazetidin-1-yl)cyclohexyl)isoquinolin-3-yl)-6-oxaspiro[2.5]octane-1-carboxamide,
(1R)-N-(7-chloro-6-(cis-4-(3-methoxyazetidin-1-yl)cyclohexyl)isoquinolin-3-yl)-6-oxaspiro[2.5]octane-1-carboxamide,
(1R)-N-(7-chloro-6-(trans-4-(3-methoxyazetidin-1-yl)cyclohexyl)isoquinolin-3-yl)-6-oxaspiro[2.5]octane-1-carboxamide,
(1S)-N-(7-chloro-6-(cis-4-(3-methoxyazetidin-1-yl)cyclohexyl)isoquinolin-3-yl)-6-oxaspiro[2.5]octane-1-carboxamide,
(1S)-N-(7-chloro-6-(trans-4-(3-methoxyazetidin-1-yl)cyclohexyl)isoquinolin-3-yl)-6-oxaspiro[2.5]octane-1-carboxamide,
N-(7-chloro-6-(4-(3-fluoroazetidin-1-yl)cyclohexyl)isoquinolin-3-yl)-6-oxaspiro[2.5]octane-1-carboxamide,
(1R)-N-(7-chloro-6-(cis-4-(3-fluoroazetidin-1-yl)cyclohexyl)isoquinolin-3-yl)-6-oxaspiro[2.5]octane-1-carboxamide,
(1R)-N-(7-chloro-6-(trans-4-(3-fluoroazetidin-1-yl)cyclohexyl)isoquinolin-3-yl)-6-oxaspiro[2.5]octane-1-carboxamide,
(1S)-N-(7-chloro-6-(cis-4-(3-fluoroazetidin-1-yl)cyclohexyl)isoquinolin-3-yl)-6-oxaspiro[2.5]octane-1-carboxamide,
(1S)-N-(7-chloro-6-(trans-4-(3-fluoroazetidin-1-yl)cyclohexyl)isoquinolin-3-yl)-6-oxaspiro[2.5]octane-1-carboxamide,
N-(7-chloro-6-(4-(3-fluoroazetidin-1-yl)cyclohexyl)isoquinolin-3-yl)-2-ethyl-3-(1-methyl-1H-pyrazol-4-yl)cyclopropane-1-carboxamide,
(1R,2R,3R)-N-(7-chloro-6-(cis-4-(3-fluoroazetidin-1-yl)cyclohexyl)isoquinolin-3-yl)-2-ethyl-3-(1-methyl-1H-pyrazol-4-yl)cyclopropane-1-carboxamide,
(1R,2R,3R)-N-(7-chloro-6-(trans-4-(3-fluoroazetidin-1-yl)cyclohexyl)isoquinolin-3-yl)-2-ethyl-3-(1-methyl-1H-pyrazol-4-yl)cyclopropane-1-carboxamide,
(1R,2S,3R)-N-(7-chloro-6-(cis-4-(3-fluoroazetidin-1-yl)cyclohexyl)isoquinolin-3-yl)-2-ethyl-3-(1-methyl-1H-pyrazol-4-yl)cyclopropane-1-carboxamide,
(1R,2S,3R)-N-(7-chloro-6-(trans-4-(3-fluoroazetidin-1-yl)cyclohexyl)isoquinolin-3-yl)-2-ethyl-3-(1-methyl-1H-pyrazol-4-yl)cyclopropane-1-carboxamide,
(1S,2R,3 S)-N-(7-chloro-6-(cis-4-(3-fluoroazetidin-1-yl)cyclohexyl)isoquinolin-3-yl)-2-ethyl-3-(1-methyl-1H-pyrazol-4-yl)cyclopropane-1-carboxamide,
(1S,2R,3 S)-N-(7-chloro-6-(trans-4-(3-fluoroazetidin-1-yl)cyclohexyl)isoquinolin-3-yl)-2-ethyl-3-(1-methyl-1H-pyrazol-4-yl)cyclopropane-1-carboxamide,
(1S,2S,3S)-N-(7-chloro-6-(cis-4-(3-fluoroazetidin-1-yl)cyclohexyl)isoquinolin-3-yl)-2-ethyl-3-(1-methyl-1H-pyrazol-4-yl)cyclopropane-1-carboxamide,
(1S,2S,3S)-N-(7-chloro-6-(trans-4-(3-fluoroazetidin-1-yl)cyclohexyl)isoquinolin-3-yl)-2-ethyl-3-(1-methyl-1H-pyrazol-4-yl)cyclopropane-1-carboxamide,
N-(7-chloro-6-(1-((3R,4R or 3S,4S)-3-fluoro-4-methyltetrahydro-2H-pyran-4-yl)piperidin-4-yl)isoquinolin-3-yl)-6-oxaspiro[2.5]octane-1-carboxamide,
N-(7-chloro-6-(1-(4-hydroxytetrahydrofuran-3-yl)piperidin-4-yl)isoquinolin-3-yl)cyclopropanecarboxamide,
N-(7-chloro-6-(1-((3R,4R)-4-hydroxytetrahydrofuran-3-yl)piperidin-4-yl)isoquinolin-3-yl)cyclopropanecarboxamide,
N-(7-chloro-6-(1-((3S,4S)-4-hydroxytetrahydrofuran-3-yl)piperidin-4-yl)isoquinolin-3-yl)cyclopropanecarboxamide,
N-(7-chloro-6-(1-(4-hydroxy-3-methyltetrahydrofuran-3-yl)piperidin-4-yl)isoquinolin-3-yl)-1-methyl-1H-pyrazole-4-carboxamide,
N-(7-chloro-6-(1-((3R,4R)-4-hydroxy-3-methyltetrahydrofuran-3-yl)piperidin-4-yl)isoquinolin-3-yl)-1-methyl-1H-pyrazole-4-carboxamide,
N-(7-chloro-6-(1-((3S,4S)-4-hydroxy-3-methyltetrahydrofuran-3-yl)piperidin-4-yl)isoquinolin-3-yl)-1-methyl-1H-pyrazole-4-carboxamide,
(1R,2R or 1S,2S)-N-(6-(1-((3R,4R or 3S,4S)-4-fluoro-3-methyltetrahydrofuran-3-yl)piperidin-4-yl)-7-methylisoquinolin-3-yl)-2-(pyridin-2-yl)cyclopropane-1-carboxamide,
N-(7-chloro-6-(1-(1,1-dioxidothietan-3-yl)piperidin-4-yl)isoquinolin-3-yl)cyclopropanecarboxamide,
N-(7-chloro-6-((3R,4R,3S,4R,3R,4S or 3S,4S)-3-fluoro-1-(oxetan-3-yl)piperidin-4-yl)isoquinolin-3-yl)cyclopropanecarboxamide,
N-(7-chloro-6-(3-fluoro-1-(3-methyloxetan-3-yl)piperidin-4-yl)isoquinolin-3-yl)cyclopropanecarboxamide,
N-(7-chloro-6-((3R,4R)-3-fluoro-1-(3-methyloxetan-3-yl)piperidin-4-yl)isoquinolin-3-yl)cyclopropanecarboxamide,
N-(7-chloro-6-((3R,4S)-3-fluoro-1-(3-methyloxetan-3-yl)piperidin-4-yl)isoquinolin-3-yl)cyclopropanecarboxamide,
N-(7-chloro-6-((3S,4R)-3-fluoro-1-(3-methyloxetan-3-yl)piperidin-4-yl)isoquinolin-3-yl)cyclopropanecarboxamide,
N-(7-chloro-6-((3S,4S)-3-fluoro-1-(3-methyloxetan-3-yl)piperidin-4-yl)isoquinolin-3-yl)cyclopropanecarboxamide,
N-(7-chloro-6-(3-fluoro-1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-((3R,4R)-3-fluoro-1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-((3R,4S)-3-fluoro-1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-((3S,4R)-3-fluoro-1-(4-hydroxy-3-methyltetrahydrofuran-3-yl)piperidin-4-yl)isoquinolin-3-yl)-6-oxaspiro[2.5]octane-1-carboxamide,
(1R)-N-(7-chloro-6-((3S,4S)-3-fluoro-1-(4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-((3R,4R)-3-fluoro-1-(4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-((3R,4S)-3-fluoro-1-(4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-((3S,4R)-3-fluoro-1-(4-hydroxy-3-methyltetrahydrofuran-3-yl)piperidin-4-yl)isoquinolin-3-yl)-6-oxaspiro[2.5]octane-1-carboxamide,
(1S)-N-(7-chloro-6-((3S,4S)-3-fluoro-1-(4-hydroxy-3-methyltetrahydrofuran-3-yl)piperidin-4-yl)isoquinolin-3-yl)-6-oxaspiro[2.5]octane-1-carboxamide,
N-(7-methyl-6-(1-(3-methyloxetan-3-yl)piperidin-4-yl)isoquinolin-3-yl)cyclopropanecarboxamide,
N-(6-(1-(4-hydroxy-3-methyltetrahydrofuran-3-yl)piperidin-4-yl)-7-methylisoquinolin-3-yl)spiro[2.3]hexane-5-carboxamide,
N-(6-(1-((3R,4R)-4-hydroxy-3-methyltetrahydrofuran-3-yl)piperidin-4-yl)-7-methylisoquinolin-3-yl)spiro[2.3]hexane-5-carboxamide,
N-(6-(1-((3S,4S)-4-hydroxy-3-methyltetrahydrofuran-3-yl)piperidin-4-yl)-7-methylisoquinolin-3-yl)spiro[2.3]hexane-5-carboxamide,
N-(6-(1-(3,4-dimethyltetrahydrofuran-3-yl)piperidin-4-yl)-7-methylisoquinolin-3-yl)-6-oxaspiro[2.5]octane-1-carboxamide,
(S)-N-(6-(1-((3S,4R)-3,4-dimethyltetrahydrofuran-3-yl)piperidin-4-yl)-7-methylisoquinolin-3-yl)-6-oxaspiro[2.5]octane-1-carboxamide,
(R)-N-(6-(1-((3S,4R)-3,4-dimethyltetrahydrofuran-3-yl)piperidin-4-yl)-7-methylisoquinolin-3-yl)-6-oxaspiro[2.5]octane-1-carboxamide,
(S)-N-(6-(1-((3R,4S)-3,4-dimethyltetrahydrofuran-3-yl)piperidin-4-yl)-7-methylisoquinolin-3-yl)-6-oxaspiro[2.5]octane-1-carboxamide,
(R)-N-(6-(1-((3R,4S)-3,4-dimethyltetrahydrofuran-3-yl)piperidin-4-yl)-7-methylisoquinolin-3-yl)-6-oxaspiro[2.5]octane-1-carboxamide,
2-(2-fluoropropan-2-yl)-N-(6-(1-(4-hydroxy-3-methyltetrahydrofuran-3-yl)piperidin-4-yl)-7-methylisoquinolin-3-yl)cyclopropane-1-carboxamide,
(1R,2R)-2-(2-fluoropropan-2-yl)-N-(6-(1-((3R,4R)-4-hydroxy-3-methyltetrahydrofuran-3-yl)piperidin-4-yl)-7-methylisoquinolin-3-yl)cyclopropane-1-carboxamide,
(1R,2R)-2-(2-fluoropropan-2-yl)-N-(6-(1-((3S,4S)-4-hydroxy-3-methyltetrahydrofuran-3-yl)piperidin-4-yl)-7-methylisoquinolin-3-yl)cyclopropane-1-carboxamide,
(1S,2S)-2-(2-fluoropropan-2-yl)-N-(6-(1-((3R,4R)-4-hydroxy-3-methyltetrahydrofuran-3-yl)piperidin-4-yl)-7-methylisoquinolin-3-yl)cyclopropane-1-carboxamide,
(1S,2S)-2-(2-fluoropropan-2-yl)-N-(6-(1-((3S,4S)-4-hydroxy-3-methyltetrahydrofuran-3-yl)piperidin-4-yl)-7-methylisoquinolin-3-yl)cyclopropane-1-carboxamide,
N-(6-(1-(4-fluoro-3-methyltetrahydrofuran-3-yl)piperidin-4-yl)-7-methylisoquinolin-3-yl)-5-oxaspiro[2.4]heptane-1-carboxamide,
(1R,2R)-N-(6-(1-((3R,4R)-4-fluoro-3-methyltetrahydrofuran-3-yl)piperidin-4-yl)-7-methylisoquinolin-3-yl)-5-oxaspiro[2.4]heptane-1-carboxamide,
(1R,2R)-N-(6-(1-((3S,4S)-4-fluoro-3-methyltetrahydrofuran-3-yl)piperidin-4-yl)-7-methylisoquinolin-3-yl)-5-oxaspiro[2.4]heptane-1-carboxamide,
(1R,2S)-N-(6-(1-((3R,4R)-4-fluoro-3-methyltetrahydrofuran-3-yl)piperidin-4-yl)-7-methylisoquinolin-3-yl)-5-oxaspiro[2.4]heptane-1-carboxamide,
(1R,2S)-N-(6-(1-((3 S,4S)-4-fluoro-3-methyltetrahydrofuran-3-yl)piperidin-4-yl)-7-methylisoquinolin-3-yl)-5-oxaspiro[2.4]heptane-1-carboxamide,
(1S,2R)-N-(6-(1-((3R,4R)-4-fluoro-3-methyltetrahydrofuran-3-yl)piperidin-4-yl)-7-methylisoquinolin-3-yl)-5-oxaspiro[2.4]heptane-1-carboxamide,
(1S,2R)-N-(6-(1-((3 S,4S)-4-fluoro-3-methyltetrahydrofuran-3-yl)piperidin-4-yl)-7-methylisoquinolin-3-yl)-5-oxaspiro[2.4]heptane-1-carboxamide,
(1S,2S)-N-(6-(1-((3R,4R)-4-fluoro-3-methyltetrahydrofuran-3-yl)piperidin-4-yl)-7-methylisoquinolin-3-yl)-5-oxaspiro[2.4]heptane-1-carboxamide,
(1S,2S)-N-(6-(1-((3S,4S)-4-fluoro-3-methyltetrahydrofuran-3-yl)piperidin-4-yl)-7-methylisoquinolin-3-yl)-5-oxaspiro[2.4]heptane-1-carboxamide,
2-ethyl-N-(6-(1-(4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1R,2R,3R)-2-ethyl-N-(6-(1-((3R,4R)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1R,2R,3R)-2-ethyl-N-(6-(1-((3S,4S)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1R,2S,3R)-2-ethyl-N-(6-(1-((3R,4R)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1R,2S,3R)-2-ethyl-N-(6-(1-((3S,4S)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1S,2R,3 S)-2-ethyl-N-(6-(1-((3R,4R)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1S,2R,3 S)-2-ethyl-N-(6-(1-((3S,4S)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1S,2S,3S)-2-ethyl-N-(6-(1-((3R,4R)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
(1S,2S,3S)-2-ethyl-N-(6-(1-((3S,4S)-4-fluoro-3-methyltetrahydrofuran-3-yl)piperidin-4-yl)-7-methylisoquinolin-3-yl)-3-(1-methyl-1H-pyrazol-4-yl)cyclopropane-1-carboxamide,
N-(6-(1-(4-hydroxy-3-methyltetrahydrofuran-3-yl)piperidin-4-yl)-7-methylisoquinolin-3-yl)-6-oxaspiro[2.5]octane-1-carboxamide,
(R)-N-(6-(1-((3R,4R)-4-hydroxy-3-methyltetrahydrofuran-3-yl)piperidin-4-yl)-7-methylisoquinolin-3-yl)-6-oxaspiro[2.5]octane-1-carboxamide,
(R)-N-(6-(1-((3S,4S)-4-hydroxy-3-methyltetrahydrofuran-3-yl)piperidin-4-yl)-7-methylisoquinolin-3-yl)-6-oxaspiro[2.5]octane-1-carboxamide,
(S)-N-(6-(1-((3R,4R)-4-hydroxy-3-methyltetrahydrofuran-3-yl)piperidin-4-yl)-7-methylisoquinolin-3-yl)-6-oxaspiro[2.5]octane-1-carboxamide,
(S)-N-(6-(1-((3S,4S)-4-hydroxy-3-methyltetrahydrofuran-3-yl)piperidin-4-yl)-7-methylisoquinolin-3-yl)-6-oxaspiro[2.5]octane-1-carboxamide,
(1R,2R or 1S,2S)-N-(6-(1-((3R,4R or 3S,4S)-4-fluoro-3-methyltetrahydrofuran-3-yl)piperidin-4-yl)-7-methylisoquinolin-3-yl)-2-(1-methyl-1H-pyrazol-3-yl)cyclopropane-1-carboxamide,
2-(1-methyl-1H-pyrazol-4-yl)-N-(7-methyl-6-(spiro[2.2]pentan-1-yl)isoquinolin-3-yl)cyclopropane-1-carboxamide,
1R,2R)-2-(1-methyl-1H-pyrazol-4-yl)-N-(7-methyl-6-((R)-spiro[2.2]pentan-1-yl)isoquinolin-3-yl)cyclopropane-1-carboxamide,
(1R,2R)-2-(1-methyl-1H-pyrazol-4-yl)-N-(7-methyl-6-((S)-spiro[2.2]pentan-1-yl)isoquinolin-3-yl)cyclopropane-1-carboxamide,
(1S,2S)-2-(1-methyl-1H-pyrazol-4-yl)-N-(7-methyl-6-((R)-spiro[2.2]pentan-1-yl)isoquinolin-3-yl)cyclopropane-1-carboxamide,
1S,2S)-2-(1-methyl-1H-pyrazol-4-yl)-N-(7-methyl-6-((S)-spiro[2.2]pentan-1-yl)isoquinolin-3-yl)cyclopropane-1-carboxamide,
5-ethoxy-N-(6-(1-(4-fluoro-3-methyltetrahydrofuran-3-yl)piperidin-4-yl)-7-methylisoquinolin-3-yl)spiro[2.3]hexane-1-carboxamide,
(1R)-5-ethoxy-N-(6-(1-((3R,4R)-4-fluoro-3-methyltetrahydrofuran-3-yl)piperidin-4-yl)-7-methylisoquinolin-3-yl)spiro[2.3]hexane-1-carboxamide,
(1R)-5-ethoxy-N-(6-(1-((3S,4S)-4-fluoro-3-methyltetrahydrofuran-3-yl)piperidin-4-yl)-7-methylisoquinolin-3-yl)spiro[2.3]hexane-1-carboxamide,
(1S)-5-ethoxy-N-(6-(1-((3R,4R)-4-fluoro-3-methyltetrahydrofuran-3-yl)piperidin-4-yl)-7-methylisoquinolin-3-yl)spiro[2.3]hexane-1-carboxamide,
(1S)-5-ethoxy-N-(6-(1-((3S,4S)-4-fluoro-3-methyltetrahydrofuran-3-yl)piperidin-4-yl)-7-methylisoquinolin-3-yl)spiro[2.3]hexane-1-carboxamide,
N-(6-(1-(4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1R,2R,3R)-N-(6-(1-((3R,4R)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1R,2R,3R)-N-(6-(1-((3S,4S)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1R,2S,3R)-N-(6-(1-((3R,4R)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1R,2S,3R)-N-(6-(1-((3S,4S)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1S,2R,3 S)-N-(6-(1-((3R,4R)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1S,2R,3 S)-N-(6-(1-((3S,4S)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1S,2S,3S)-N-(6-(1-((3R,4R)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
(1S,2S,3S)-N-(6-(1-((3S,4S)-4-fluoro-3-methyltetrahydrofuran-3-yl)piperidin-4-yl)-7-methylisoquinolin-3-yl)-2-methyl-3-(1-methyl-1H-pyrazol-4-yl)cyclopropane-1-carboxamide,
N-(7-methyl-6-(1-(3-methyltetrahydrofuran-3-yl)piperidin-4-yl)isoquinolin-3-yl)-2-(pyridin-2-yl)cyclopropane-1-carboxamide,
(1R-2R)-N-(7-methyl-6-(1-((R)-3-methyltetrahydrofuran-3-yl)piperidin-4-yl)isoquinolin-3-yl)-2-(pyridin-2-yl)cyclopropane-1-carboxamide,
(1R-2R)-N-(7-methyl-6-(1-((S)-3-methyltetrahydrofuran-3-yl)piperidin-4-yl)isoquinolin-3-yl)-2-(pyridin-2-yl)cyclopropane-1-carboxamide,
(1S-2S)-N-(7-methyl-6-(1-((R)-3-methyltetrahydrofuran-3-yl)piperidin-4-yl)isoquinolin-3-yl)-2-(pyridin-2-yl)cyclopropane-1-carboxamide,
(1S-2S)-N-(7-methyl-6-(1-((S)-3-methyltetrahydrofuran-3-yl)piperidin-4-yl)isoquinolin-3-yl)-2-(pyridin-2-yl)cyclopropane-1-carboxamide N-(6-(1-cyanocyclopropyl)isoquinolin-3-yl)cyclopropanecarboxamide,
N-(6-(1-cyanocyclobutyl)isoquinolin-3-yl)cyclopropanecarboxamide,
(R or S)-N-(6-(1-cyanospiro[2.2]pentan-1-yl)isoquinolin-3-yl)cyclopropanecarboxamide,
N-(6-(1-cyano-2,2-dimethylcyclopropyl)isoquinolin-3-yl)cyclopropanecarboxamide,
(R)-N-(6-(1-cyano-2,2-dimethylcyclopropyl)isoquinolin-3-yl)cyclopropanecarboxamide,
(S)-N-(6-(1-cyano-2,2-dimethylcyclopropyl)isoquinolin-3-yl)cyclopropanecarboxamide,
N-(6-(1-cyano-2-methylcyclopropyl)isoquinolin-3-yl)cyclopropanecarboxamide,
(R)-N-(6-(1-cyano-2-methylcyclopropyl)isoquinolin-3-yl)cyclopropanecarboxamide,
(S)-N-(6-(1-cyano-2-methylcyclopropyl)isoquinolin-3-yl)cyclopropanecarboxamide,
N-(6-(1-cyanospiro[2.2]pentan-1-yl)-7-fluoroisoquinolin-3-yl)cyclopropanecarboxamide,
(R)-N-(6-(1-cyanospiro[2.2]pentan-1-yl)-7-fluoroisoquinolin-3-yl)cyclopropanecarboxamide,
(S)-N-(6-(1-cyanospiro[2.2]pentan-1-yl)-7-fluoroisoquinolin-3-yl)cyclopropanecarboxamide,
N-(6-(1-cyanospiro[2.2]pentan-1-yl)isoquinolin-3-yl)-6-oxaspiro[2.5]octane-1-carboxamide,
(R)-N-(6-((R)-1-cyanospiro[2.2]pentan-1-yl)isoquinolin-3-yl)-6-oxaspiro[2.5]octane-1-carboxamide,
(R)-N-(6-((S)-1-cyanospiro[2.2]pentan-1-yl)isoquinolin-3-yl)-6-oxaspiro[2.5]octane-1-carboxamide,
(S)-N-(6-((R)-1-cyanospiro[2.2]pentan-1-yl)isoquinolin-3-yl)-6-oxaspiro[2.5]octane-1-carboxamide,
(S)-N-(6-((S)-1-cyanospiro[2.2]pentan-1-yl)isoquinolin-3-yl)-6-oxaspiro[2.5]octane-1-carboxamide,
N-(6-(1-cyanospiro[2.2]pentan-1-yl)-7-fluoroisoquinolin-3-yl)-2-(pyridin-2-yl)cyclopropane-1-carboxamide,
(1R,2R)-N-(6-((R)-1-cyanospiro[2.2]pentan-1-yl)-7-fluoroisoquinolin-3-yl)-2-(pyridin-2-yl)cyclopropane-1-carboxamide,
(1R,2R)-N-(6-((S)-1-cyanospiro[2.2]pentan-1-yl)-7-fluoroisoquinolin-3-yl)-2-(pyridin-2-yl)cyclopropane-1-carboxamide,
1S,2S)-N-(6-((R)-1-cyanospiro[2.2]pentan-1-yl)-7-fluoroisoquinolin-3-yl)-2-(pyridin-2-yl)cyclopropane-1-carboxamide,
(1S,2S)-N-(6-((S)-1-cyanospiro[2.2]pentan-1-yl)-7-fluoroisoquinolin-3-yl)-2-(pyridin-2-yl)cyclopropane-1-carboxamide,
(R or S)-N-(6-(1-cyanospiro[2.2]pentan-1-yl)isoquinolin-3-yl)-1-methyl-1H-pyrazole-4-carboxamide,
(R)-N-(6-(1-cyanospiro[2.2]pentan-1-yl)isoquinolin-3-yl)-1-methyl-1H-pyrazole-4-carboxamide,
(S)-N-(6-(1-cyanospiro[2.2]pentan-1-yl)isoquinolin-3-yl)-1-methyl-1H-pyrazole-4-carboxamide,
(R or S)-N-(6-(1-cyanospiro[2.2]pentan-1-yl)isoquinolin-3-yl)-1-propionylazetidine-3-carboxamide,
(1S,2R)-N-(7-chloro-6-(1-(4-hydroxy-3-methyltetrahydrofuran-3-yl)piperidin-4-yl)isoquinolin-3-yl)-2-(2-methyl-2H-1,2,3-triazol-4-yl)cyclopropane-1-carboxamide,
(1S,2S)-N-(7-chloro-6-(1-(4-hydroxy-3-methyltetrahydrofuran-3-yl)piperidin-4-yl)isoquinolin-3-yl)-2-(2-methyl-2H-1,2,3-triazol-4-yl)cyclopropane-1-carboxamide,
(1R,2R or 1S,2S)-N-(7-chloro-6-isopropylisoquinolin-3-yl)-2-(1-methyl-1H-pyrazol-4-yl)cyclopropane-1-carboxamide, and
(1R,2R or 1S,2S)-N-(7-chloro-6-(1-(3-methyloxetan-3-yl)piperidin-4-yl)isoquinolin-3-yl)-2-(2-hydroxypropan-2-yl)cyclopropanecarboxamide.

20. A pharmaceutical composition comprising a compound of claim 1, and a pharmaceutically acceptable carrier.

21. Use of a compound of claim 1 or a pharmaceutically acceptable salt thereof, for the manufacture of a medicament for the treatment of Parkinson's Disease.

22. A compound according to claim 1 for use in therapy.

23. A method of treating Parkinson's Disease comprising administering an effective amount of a compound of claim 1, or a pharmaceutically acceptable salt thereof, to a person in need thereof.

24. A method for the treatment or prophylaxis of an indication in which LRRK2 kinase is involved comprising administering to a subject in need thereof an effective amount of a compound according to claim 1, or a pharmaceutically acceptable salt thereof, said indication selected from:
abnormal motor symptoms associated with Parkinson's disease, non-motor symptoms associated with Parkinson's disease, Lewy body dementia, L-Dopa induced dyskinesias,
Alzheimer's disease, mild cognitive impairment, the transition from mild cognitive impairment to Alzheimer's disease, tauopathy disorders characterized by hyperphosphorylation of tau such as argyrophilic grain disease, Picks disease, corticobasal degeneration, progressive supranuclear palsy, inherited frontotemporal dementia, and Parkinson's disease linked to chromosome 17, neuroinflammation associated with of microglial inflammatory responses associated with multiple sclerosis, HIV-induced dementia, ALS, ischemic stroke, traumatic brain injury and spinal cord injury, lymphomas, leukemias, multiple sclerosis, rheumatoid arthritis, systemic lupus erythematosus, autoimmune hemolytic anemia, pure red cell aplasia, idiopathic thrombocytopenic pupura (ITP), Evans Syndrome, vasculitis, bullous skin disorder, type I diabetes mellitus, Sjorgen's syndrome, Delvic's disease, inflammatory myopathies, and ankylosing spondylitis, renal cancer, breast cancer, lung cancer, prostate cancer, and acute myelogenous leukemia (AML) in subjects expressing the LRRK2 G2019S mutation, papillary renal and thyroid carcinomas in a subject in whom LRRK2 is amplified or overexpressed, Crohn's disease and leprosy.
